(19) United States
(12) Patent Application Publication
(10) Pub. No.: US 2021/0230178 A1
(43) Pub. Date: Jul. 29, 2021

(54) TRIOXACARCIN ANALOGS AND DIMERS AS POTENT ANTICANCER AGENTS

(71) Applicant: William Marsh Rice University, Houston, TX (US)

(72) Inventors: Kyriacos C. NICOLAOU, Houston, TX (US); Pengxi CHEN, Houston, TX (US); Shugao ZHU, Salt Lake City, UT (US); Quan CAI, Houston, TX (US)

(73) Assignee: William Marsh Rice University, Houston, TX (US)

(21) Appl. No.: 16/639,629

(22) PCT Filed: Aug. 16, 2018
(86) PCT No.: PCT/US2018/046804

§ 371 Date: Feb. 17, 2020

Related U.S. Application Data
(60) Provisional application No. 62/546,723, filed on Aug. 17, 2017.

(51) Int. Cl. C07D 493/22; A61K 45/06

(52) U.S. Cl. CPC C07D 493/22; A61K 45/06

(57) ABSTRACT

In one aspect, the present disclosure provides trioxacarcin analogs of the formula: wherein the variables are as defined herein. In another aspect, the present disclosure also provides methods of preparing the compounds disclosed herein as well as dimers of the compounds described herein. In another aspect, the present disclosure also provides pharmaceutical compositions and methods of use of the compounds disclosed herein. Additionally, drug conjugates with cell targeting moieties of the compounds are also provided.

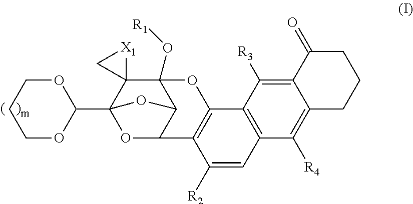

This application claims the benefit of priority to U.S. Provisional Application No. 62/546,723 filed on Aug. 17, 2017, the entire contents of which are hereby incorporated by reference.

The development of this disclosure was funded in part by the Cancer Prevention and Research Institute of Texas (CPRIT) under Grant No. R1226 and the Welch Foundation under Grant No. C-1819.

### BACKGROUND

### 1. Field

This disclosure relates to the fields of medicine, pharmacology, chemistry, and oncology. In particular, new compounds, compositions, methods of treatment, and methods of synthesis relating to analogs and dimers of trioxacarcins are disclosed.

### 2. Related Art

Antibody-drug conjugates (ADCs) constitute a powerful new paradigm for targeted chemotherapy (e.g. Kadcyla® and Adcetris®) (Chari et al., 2014; Dosio et al., 2014, Gerber et al., 2013, Sapra & Shor, 2013, Sievers & Senter, 2013 and Nicolaou, 2014). These new targeted anticancer drugs are molecular constructs containing a specific antibody targeting a particular type of cancer cells and a potent cytotoxic agent (the payload) joined together by a chemical linker which is able to undergo decomposition in vivo. Numerous cancer targeting antibodies have been developed for different cancer types, but available payloads are significantly limited. Generally, these payloads are potent cytotoxic compounds, often with IC₅₀ values in the low picomolar range that can affect cell death once the compound is released from the antibody-drug conjugate within the targeted cell. Trioxacarcin is one possible compound which may be used as the cytotoxic payload in antibody-drug conjugates. Therefore, there remains a need to develop new trioxacarcin analogs with improved properties and dimers thereof.

### SUMMARY

In some aspects, the present disclosure provides trioxacarcin analogs and dimers which may be used as payloads in an antibody-drug conjugate. In some embodiments, the compounds are further defined by the formula:

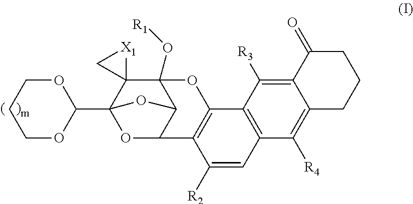

wherein:
m is 0 or 1;
R₁ is alkyl_{(C≤8)}, alkenyl_{(C≤8)}, acyl_{(C≤8)}, or a substituted version of the last three groups; a group of the formula —(CH₂)ₙ_{₁}O(CH₂)ₙ²R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

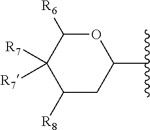

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
X₁ is —O— or —NRₐ—, wherein Rₐ is hydrogen, alkyl_{(C≤8)}, or substituted alkyl_{(C≤8)};
provided that when R₂ is methyl, R₃ is hydroxy, R₄ is methoxy, and X₁ is O, then R₁ is not acetyl, alkyl_{(≤12)}, or substituted alkyl_{(C≤12)};
   a compound of the formula:

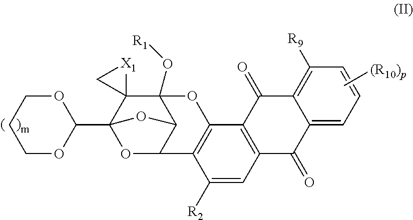

wherein:
m is 0 or 1;
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

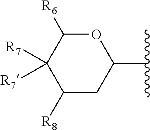

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₉ is hydroxy, alkoxy_{(C≤8)}, substituted alkoxy_{(C≤8)}, acyloxy_{(C≤8)}, or substituted acyloxy_{(C≤8)};
R₁₀ is hydrogen, halo, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)};
p is 1, 2, 3, or 4; and
X₁ is —O— or —NRₐ—, wherein Rₐ is hydrogen, alkyl_{(C≤8)}, or substituted alkyl_{(C≤8)}; or
   a compound of the formula:

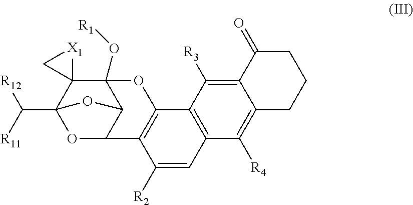

wherein:
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

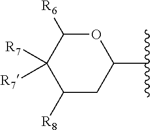

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
R₁₁ is hydrogen, alkyl_{(C≤8)}, substituted alkyl_{(C≤8)}, alkoxy_{(C≤8)}, or substituted alkoxy_{(C≤8)};
R₁₂ is —O(CH₂)_{q}R₁₃, wherein:
   q is 1, 2, 3, 4, or 5;
   R₁₃ is amino, alkylamino_{(C≤8)}, substituted alkylamino_{(C≤8)}, dialkylamino_{(C≤8)}, or substituted dialkylamino_{(C≤8)}; or
a group of the formula:

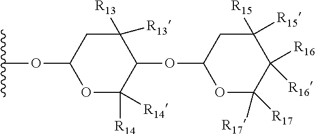

wherein:
   R₁₃, R₁₃′, R₁₄, R₁₄′, R₁₅, R₁₅′, R₁₆, R₁₆′, R₁₇, and R₁₇′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)}; and
a group of the formula:

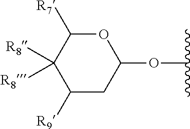

wherein:
   R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₈″ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered hetero-cycloalkanediyl_{(C≤12)} or substituted heterocycloalkanediyl_{(C≤12)} which is optionally substituted with 1, 2, or 3 oxo groups; R₁ and R₂ are taken together and are alkanediyl_{(C≤8)} or substituted alkanediyl_{(C≤8)};
X₁ is —O— or —NRₐ—, wherein Rₐ is hydrogen, alkyl_{(C≤8)}, or substituted alkyl_{(C≤8)};
   a compound of the formula:

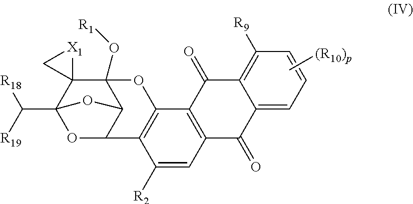

wherein:
R₁ is hydrogen, alkyl_{(C≤8)}, alkenyl_{(C≤8)}, acyl_{(C≤8)}, or a substituted version of the last three groups; or a group of the formula:

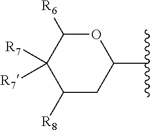

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₉ is hydroxy, alkoxy_{(C≤8)}, substituted alkoxy_{(C≤8)}, acyloxy_{(C≤8)}, or substituted acyloxy_{(C≤8)};
R₁₀ is hydrogen, halo, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)};
p is 1, 2, 3, or 4;
R₁₈ is hydrogen, alkyl_{(C≤8)}, substituted alkyl_{(C≤8)}, alkoxy_{(C≤8)}, or substituted alkoxy_{(C≤8)};
R₁₉ is —O(CH₂)_{q}R₁₃, wherein:
   q is 1, 2, 3, 4, or 5;
   R₁₃ is amino, alkylamino_{(C≤8)}, substituted alkylamino_{(C≤8)}, dialkylamino_{(C≤8)}, or substituted dialkylamino_{(C≤8)}; or
a group of the formula:

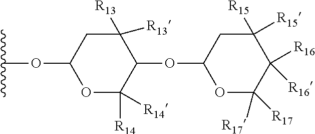

wherein:
   R₁₃, R₁₃′, R₁₄, R₁₄′, R₁₅, R₁₅′, R₁₆, R₁₆′, R₁₇, and R₁₇′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)}; and
a group of the formula:

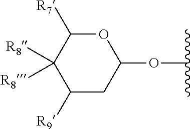

wherein:
   R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₈″ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocyclo-alkanediyl_{(C≤12)} which is optionally substituted with 1, 2, or 3 oxo groups; R₁ and R₂ are taken together and are alkanediyl_{(C≤8)} or substituted alkanediyl_{(C≤8)};
X₁ is —O— or —NRₐ—, wherein Rₐ is hydrogen, alkyl_{(C≤8)}, or substituted alkyl_{(C≤8)}; or a compound of the formula:

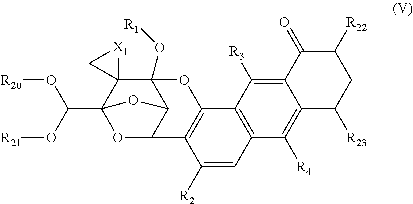

wherein:
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

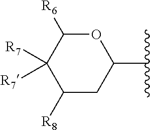

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
R₂₀ is alkyl_{(C≤8)}, substituted alkyl_{(C≤8)}, acyl_{(C≤8)}, or substituted acyl_{(C≤8)};
R₂₁ is alkyl_{(C≤8)} or substituted alkyl_{(C≤8)};
R₂₂ is hydrogen, hydroxy, alkoxy_{(C≤8)}, substituted alkoxy_{(C≤8)}, acyloxy_{(C≤8)}, or substituted acyloxy_{(C≤8)};
R₂₃ is a group of the formula:

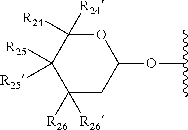

wherein:
   R₂₄, R₂₄′, R₂₆, and R₂₆′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₂₅ and R₂₅′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups; and
X₁ is —O— or —NRₐ—, wherein Rₐ is hydrogen, alkyl_{(C≤8)}, or substituted alkyl_{(C≤8)};
   or a pharmaceutically acceptable salt thereof. In some embodiments, the compounds are further defined as:

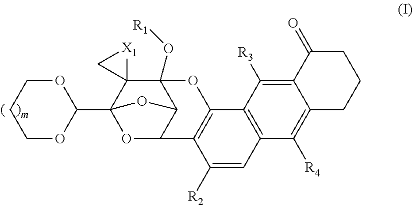

wherein:
m is 0 or 1;
R₁ is alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

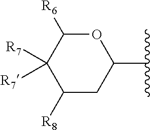

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
X₁ is —O— or —NRₐ—, wherein Rₐ is hydrogen, alkyl_{(C≤8)}, or substituted alkyl_{(C≤8)};
provided that when R₂ is methyl, R₃ is hydroxy, R₄ is methoxy, and X₁ is O, then R₁ is not acetyl, alkyl_{(≥12)}, or substituted alkyl_{(C≤12)};
   or a pharmaceutically acceptable salt thereof. In some embodiments, the compounds are further defined as:

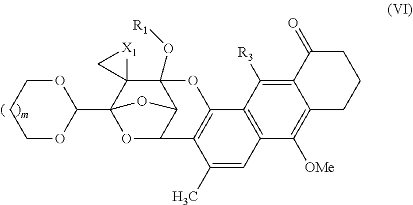

wherein:
m is 0 or 1;
R₁ is alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

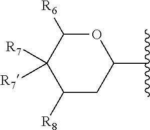

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₃ is hydrogen, hydroxy, alkoxy_{(C≤8)}, substituted alkoxy_{(C≤8)}, acyloxy_{(C≤8)}, or substituted acyloxy_{(C≤8)};
X₁ is —O— or —NRₐ—, wherein Rₐ is hydrogen, alkyl_{(C≤8)}, or substituted alkyl_{(C≤8)};
provided that when R₃ is hydroxy and X₁ is O, then R₁ is not acetyl, alkyl_{(≤12)}, or substituted alkyl_{(C≤12)};
   or a pharmaceutically acceptable salt thereof. In some embodiments, the compound are further defined as:

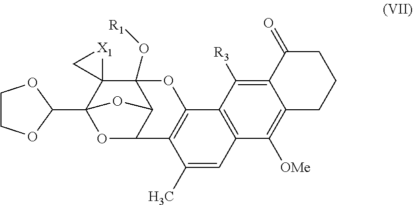

wherein:
R₁ is alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

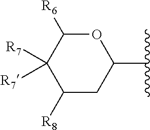

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₃ is hydrogen, hydroxy, alkoxy_{(C≤8)}, substituted alkoxy_{(C≤8)}, acyloxy_{(C≤8)}, or substituted acyloxy_{(C≤8)};
X₁ is —O— or —NRₐ—, wherein Rₐ is hydrogen, alkyl_{(C≤8)}, or substituted alkyl_{(C≤8)};
   or a pharmaceutically acceptable salt thereof. In some embodiments, the compounds are further defined as:

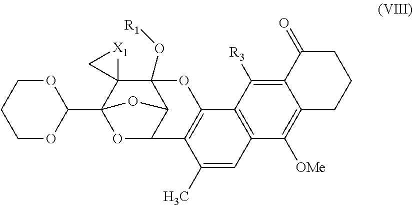

wherein:
R₁ is alkenyl_{(C≤8)} or substituted alkenyl_{(C≤8)}; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

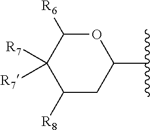

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₃ is hydrogen, hydroxy, alkoxy_{(C≤8)}, substituted alkoxy_{(C≤8)}, acyloxy_{(C≤8)}, or substituted acyloxy_{(C≤8)};
X₁ is —O— or —NRₐ—, wherein Rₐ is hydrogen, alkyl_{(C≤8)}, or substituted alkyl_{(C≤8)};
provided that when R₃ is hydroxy and X₁ is O, then R is not acetyl, alkyl_{(≤12)}, or substituted alkyl_{(C≤12)};
   or a pharmaceutically acceptable salt thereof. In some embodiments, the compounds are further defined as:

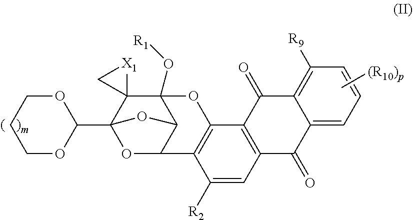

wherein:
m is 0 or 1;
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

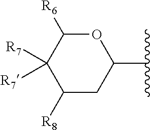

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₉ is hydroxy, alkoxy_{(C≤8)}, substituted alkoxy_{(C≤8)}, acyloxy_{(C≤8)}, or substituted acyloxy_{(C≤8)};
R₁₀ is hydrogen, halo, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)};
p is 1, 2, 3, or 4; and
X₁ is —O— or —NRₐ—, wherein Rₐ is hydrogen, alkyl_{(C≤8)}, or substituted alkyl_{(C≤8)};
   or a pharmaceutically acceptable salt thereof. In some embodiments, the compounds are further defined as:

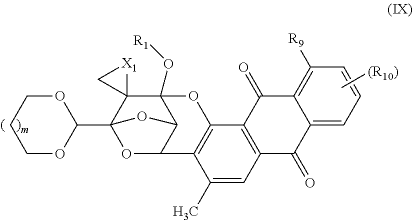

wherein:
m is 0 or 1;
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

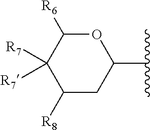

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₉ is hydroxy, alkoxy_{(C≤8)}, substituted alkoxy_{(C≤8)}, acyloxy_{(C≤8)}, or substituted acyloxy_{(C≤8)};
R₁₀ is hydrogen, halo, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)};
p is 1, 2, 3, or 4; and
   or a pharmaceutically acceptable salt thereof. In some embodiments, the compounds are further defined as:

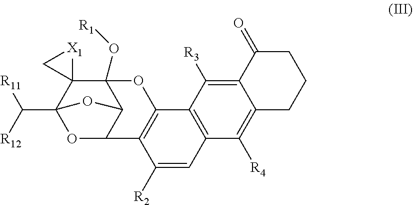

wherein:
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

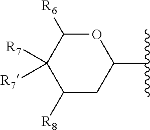

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
R₁₁ is hydrogen, alkyl_{(C≤8)}, substituted alkyl_{(C≤8)}, alkoxy_{(C≤8)}, or substituted alkoxy_{(C≤8)};
R₁₂ is —O(CH₂)_{q}R₁₃, wherein:
   q is 1, 2, 3, 4, or 5;
   R₁₃ is amino, alkylamino_{(C≤8)}, substituted alkylamino_{(C≤8)}, dialkylamino_{(C≤8)}, or substituted dialkylamino_{(C≤8)}; or
a group of the formula:

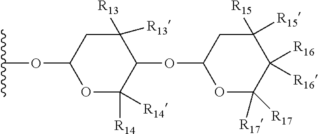

wherein:
   R₁₃, R₁₃′, R₁₄, R₁₄′, R₁₅, R₁₅′, R₁₆, R₁₆′, R₁₇, and R₁₇′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)};
a group of the formula:

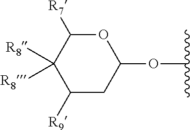

wherein:
   R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₈″ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocyclo-alkanediyl_{(C≤12)} which is optionally substituted with 1, 2, or 3 oxo groups; R₁ and R₂ are taken together and are alkanediyl_{(C≤8)} or substituted alkanediyl_{(C≤8)}; and
X₁ is —O— or —NRₐ—, wherein Rₐ is hydrogen, alkyl_{(C≤8)}, or substituted alkyl_{(C≤8)};
   or a pharmaceutically acceptable salt thereof. In some embodiments, the compounds are further defined as:

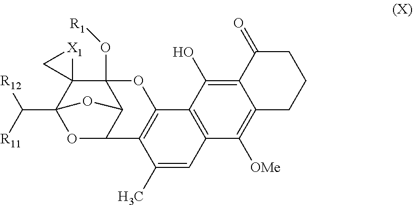

wherein:
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

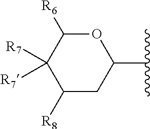

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₁₁ is hydrogen, alkyl_{(C≤8)}, substituted alkyl_{(C≤8)}, alkoxy_{(C≤8)}, or substituted alkoxy_{(C≤8)};
R₁₂ is —O(CH₂)_{q}R₁₃, wherein:
   q is 1, 2, 3, 4, or 5;
   R₁₃ is amino, alkylamino_{(C≤8)}, substituted alkylamino_{(C≤8)}, dialkylamino_{(C≤8)}, or substituted dialkylamino_{(C≤8)}; or
a group of the formula:

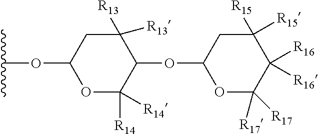

wherein:
   R₁₃, R₁₃′, R₁₄, R₁₄′, R₁₅, R₁₅′, R₁₆, R₁₆′, R₁₇, and R₁₇′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)};
a group of the formula:

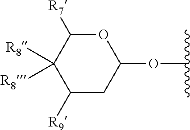

wherein:
   R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₈″ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocyclo-alkanediyl_{(C≤12)} which is optionally substituted with 1, 2, or 3 oxo groups; R₁ and R₂ are taken together and are alkanediyl_{(C≤8)} or substituted alkanediyl_{(C≤8)}; and
X₁ is —O— or —NRₐ—, wherein Rₐ is hydrogen, alkyl_{(C≤8)}, or substituted alkyl_{(C≤8)};
   or a pharmaceutically acceptable salt thereof. In some embodiments, the compounds are further defined as:

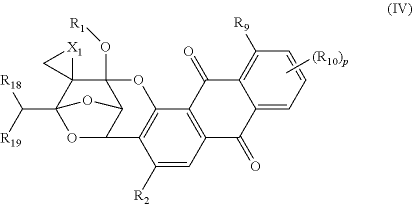

wherein:
R₁ is hydrogen, alkyl_{(C≤8)}, alkenyl_{(C≤8)}, acyl_{(C≤8)}, or a substituted version of the last three groups; or
a group of the formula:

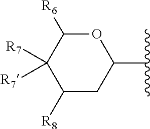

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₉ is hydroxy, alkoxy_{(C≤8)}, substituted alkoxy_{(C≤8)}, acyloxy_{(C≤8)}, or substituted acyloxy_{(C≤8)};
R₁₀ is hydrogen, halo, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)};
p is 1, 2, 3, or 4;
R₁₈ is hydrogen, alkyl_{(C≤8)}, substituted alkyl_{(C≤8)}, alkoxy_{(C≤8)}, or substituted alkoxy_{(C≤8)};
R₁₉ is —O(CH₂)_{q}R₁₃, wherein:
   q is 1, 2, 3, 4, or 5;
   R₁₃ is amino, alkylamino_{(C≤8)}, substituted alkylamino_{(C≤8)}, dialkylamino_{(C≤8)}, or substituted dialkylamino_{(C≤8)}; or
a group of the formula:

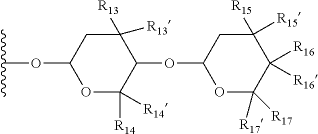

wherein:
   R₁₃, R₁₃′, R₁₄, R₁₄′, R₁₅, R₁₅′, R₁₆, R₁₆′, R₁₇, and R₁₇′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)};
a group of the formula:

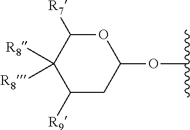

wherein:
   R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₈″ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocyclo-alkanediyl_{(C≤12)} which is optionally substituted with 1, 2, or 3 oxo groups; R₁ and R₂ are taken together and are alkanediyl_{(C≤8)} or substituted alkanediyl_{(C≤8)}; and
X₁ is —O— or —NRₐ—, wherein Rₐ is hydrogen, alkyl_{(C≤8)}, or substituted alkyl_{(C≤8)};
   or a pharmaceutically acceptable salt thereof. In some embodiments, the compounds are further defined as:

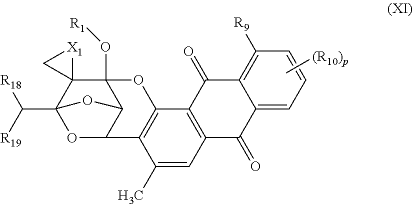

wherein:
R₁ is hydrogen, alkyl_{(C≤8)}, alkenyl_{(C≤8)}, acyl_{(C≤8)}, or a substituted version of the last three groups; or
a group of the formula:

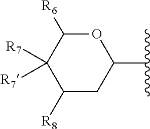

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₉ is hydroxy, alkoxy_{(C≤8)}, substituted alkoxy_{(C≤8)}, acyloxy_{(C≤8)}, or substituted acyloxy_{(C≤8)};
R₁₀ is hydrogen, halo, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)};
p is 1, 2, 3, or 4;
R₁₈ is hydrogen, alkyl_{(C≤8)}, substituted alkyl_{(C≤8)}, alkoxy_{(C≤8)}, or substituted alkoxy_{(C≤8)};
R₁₉ is —O(CH₂)_{q}R₁₃, wherein:
   q is 1, 2, 3, 4, or 5;
   R₁₃ is amino, alkylamino_{(C≤8)}, substituted alkylamino_{(C≤8)}, dialkylamino_{(C≤8)}, or substituted dialkylamino_{(C≤8)}; or
a group of the formula:

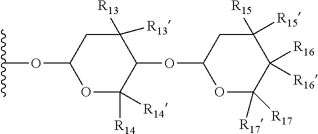

wherein:
   R₁₃, R₁₃′, R₁₄, R₁₄′, R₁₅, R₁₅′, R₁₆, R₁₆′, R₁₇, and R₁₇′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)};
a group of the formula:

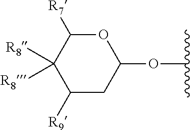

wherein:
   R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₈″ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocyclo-alkanediyl_{(C≤12)} which is optionally substituted with 1, 2, or 3 oxo groups; R₁ and R₂ are taken together and are alkanediyl_{(C≤8)} or substituted alkanediyl_{(C≤8)}; and
      or a pharmaceutically acceptable salt thereof. In some embodiments, the compounds are further defined as:

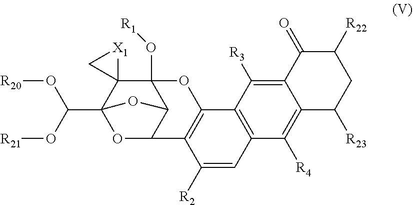

wherein:
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

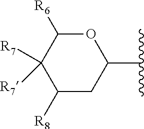

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
R₂₀ is alkyl_{(C≤8)}, substituted alkyl_{(C≤8)}, acyl_{(C≤8)}, or substituted acyl_{(C≤8)};
R₂₁ is alkyl_{(C≤8)} or substituted alkyl_{(C≤8)};
   R₂₂ is hydrogen, hydroxy, alkoxy_{(C≤8)}, substituted alkoxy_{(C≤8)}, acyloxy_{(C≤8)}, or substituted acyloxy_{(C≤8)};
R₂₃ is a group of the formula:

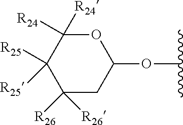

wherein:
   R₂₄, R₂₄′, R₂₆, and R₂₆′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₂₅ and R₂₅′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups; and
X₁ is —O— or —NRₐ—, wherein Rₐ is hydrogen, alkyl_{(C≤8)}, or substituted alkyl_{(C≤8)};
   or a pharmaceutically acceptable salt thereof. In some embodiments, the compounds are further defined as:

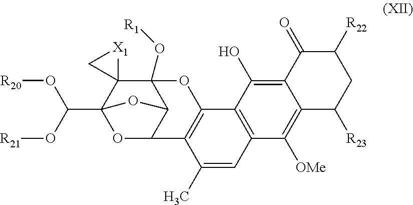

wherein:
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
   n₁ and n₂ are each independently 1, 2, 3, 4, or 5; and
   R₅ are amino, alkylamino_{(C≤8)}, substituted alkylamino_{(C≤8)}, dialkylamino_{(C≤12)}, or substituted dialkylamino_{(C≤12)}; or
a group of the formula:

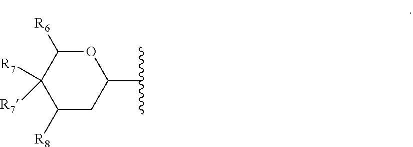

wherein:
   R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
R₂₀ is alkyl_{(C≤8)}, substituted alkyl_{(C≤8)}, acyl_{(C≤8)}, or substituted acyl_{(C≤8)};
R₂₁ is alkyl_{(C≤8)} or substituted alkyl_{(C≤8)};
R₂₂ is hydrogen, hydroxy, alkoxy_{(C≤8)}, substituted alkoxy_{(C≤8)}, acyloxy_{(C≤8)}, or substituted acyloxy_{(C≤8)};
R₂₃ is a group of the formula:

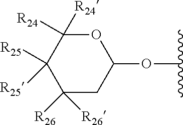

wherein:
   R₂₄, R₂₄′, R₂₆, and R₂₆′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₂₅ and R₂₅′ are hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; and
X₁ is —O— or —NRₐ—, wherein Rₐ is hydrogen, alkyl_{(C≤8)}, or substituted alkyl_{(C≤8)};
   or a pharmaceutically acceptable salt thereof.

In some embodiments, R₂ is alkyl_{(C≤8)} such as methyl. In some embodiments, R₃ is hydrogen. In other embodiments, R₃ is alkoxy_{(C≤8)} such as methoxy. In yet other embodiments, R₃ is acyloxy_{(C≤8)} such as acetoxy. In some embodiments, R₄ is alkoxy_{(C≤8)} such as methoxy. In some embodiments, X₁ is —O—. In other embodiments, X₁ is —NH—. In some embodiments, m is 0. In other embodiments, m is 1. In some embodiments, R₉ is hydroxy. In other embodiments, R₉ is acyloxy_{(C≤8)} such as acetoxy. In some embodiments, R₁₀ is hydrogen. In other embodiments, R₁₀ is halo such as chloro. In some embodiments, p is 1 or 2. In further embodiments, p is 1.

In some embodiments, R₁ is hydrogen. In other embodiments, R₁ is alkyl_{(C≤8)} such as methyl. In other embodiments, R₁ is acyl_{(C≤8)} such as acetoxy. In yet other embodiments, R₁ is alkenyl_{(C≤8)}. In further embodiments, R₁ is 2-propenyl. In other embodiments, R₁ is a group of the formula —(CH₂)ₙ_{₁}O(CH₂)ₙ_{₂}R₅, wherein: n₁ and n₂ are each independently 1, 2, 3, 4, or 5; and R₅ are amino, alkylamino_{(C≤8)}, substituted alkylamino_{(C≤8)}, dialkylamino_{(C≤12)}, or substituted dialkylamino_{(C≤12)}. In further embodiments, n₁ is 1, 2, or 3. In yet further embodiments, n₁ is 2. In other embodiments, n₂ is 1, 2, or 3. In further embodiments, n₂ is 2. In some embodiments, R₅ is amino. In other embodiments, R₅ is alkylamino_{(C≤8)}.

In some embodiments, R₁ is a group of the formula:

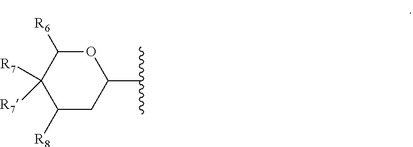

wherein:
R₆ and R₈ are each independently hydrogen, hydroxy, alkyls), substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocyclo-alkanediyl_{(C≤12)} or substituted heterocycloalkanediyl_{(C≤12)} which is optionally substituted with 1, 2, or 3 oxo groups.

In some embodiments, R₆ is hydrogen. In other embodiments, R₆ is alkyl_{(C≤8)} such as methyl. In some embodiments, R₈ is hydrogen. In other embodiments, R₈ is hydroxy. In still other embodiments, R₈ is alkoxy_{(C≤8)} such as methoxy. In yet other embodiments, R₈ is acyloxy_{(C≤8)} such as acetoxy. In some embodiments, R₇ is hydrogen. In other embodiments, R₇ is hydroxy. In some embodiments, R₇′ is hydrogen. In other embodiments, R₇′ is hydroxy. In still other embodiments, R₇′ is substituted alkyl_{(C≤8)} such as hydroxyethyl. In yet other embodiments, R₇′ is acyl_{(C≤8)} such as acetyl. In other embodiments, R₇′ is amino. In still other embodiments, R₇′ is alkylamino_{(C≤8)} such as isopropylamino. In other embodiments, R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups. In further embodiments, R₇ and R₇′ are taken together and are a 5 to 7 membered heterocycloalkanediyl_{(C≤12)} and is substituted with 1 or 2 oxo group. In some embodiments, R₁₁ or R₁₈ are hydrogen. In other embodiments, R₁₁ or R₁₈ are alkyl_{(C≤8)}. In other embodiments, R₁₁ or R₁₈ are alkoxy_{(C≤8)}.

In some embodiments, R₁₂ or Rig are —O(CH₂)_{q}R₁₃, wherein: q is 1, 2, 3, 4, or 5; R₁₃ is amino, alkylamino_{(C≤8)}, substituted alkylamino_{(C≤8)}, dialkylamino_{(C≤8)}, or substituted dialkylamino_{(C≤8)}. In further embodiments, q is 1, 2, or 3. In yet further embodiments, q is 2. In some embodiments, R₁₃ is amino. In some embodiments, R₁₂ or R₁₉ are a group of the formula:

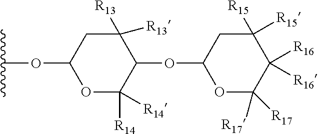

wherein:
R₁₃, R₁₃′, R₁₄, R₁₄′, R₁₅, R₁₅′, R₁₆, R₁₆′, R₁₇, and R₁₇′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)}.

In some embodiments, R₁₃ is hydrogen. In other embodiments, R₁₃ is hydroxy. In other embodiments, R₁₃ is alkyl_{(C≤8)} such as methyl. In still other embodiments, R₁₃ is acyloxy_{(C≤8)} such as acetoxy. In some embodiments, R₁₃′ is hydrogen. In other embodiments, R₁₃′ is hydroxy. In still other embodiments, R₁₃′ is alkyl_{(C≤8)} such as methyl. In yet other embodiments, R₁₃′ is acyloxy_{(C≤8)} such as acetoxy. In some embodiments, R₁₄ is hydrogen. In other embodiments, R₁₄ is hydroxy. In still other embodiments, R₁₄ is alkyl_{(C≤8)} such as methyl. In yet other embodiments, R₁₄ is acyloxy_{(C≤8)} such as acetoxy. In some embodiments, R₁₄′ is hydrogen. In other embodiments, R₁₄′ is hydroxy. In still other embodiments, R₁₄′ is alkyl_{(C≤8)} such as methyl. In yet other embodiments, R₁₄′ is acyloxy_{(C≤8)} such as acetoxy. In some embodiments, R₁₅ is hydrogen. In other embodiments, R₁₅ is hydroxy. In still other embodiments, R₁₅ is alkyl_{(C≤8)} such as methyl. In still other embodiments, R₁₅ is acyloxy_{(C≤8)} such as acetoxy. In some embodiments, R₁₅′ is hydrogen. In other embodiments, R₁₅′ is hydroxy. In still other embodiments, R₁₅′ is alkyl_{(C≤8)} such as methyl. In still other embodiments, R₁₅′ is acyloxy_{(C≤8)} such as acetoxy. In some embodiments, R₁₆ is hydrogen. In other embodiments, R₁₆ is hydroxy. In still other embodiments, R₁₆ is alkyl_{(C≤8)} such as methyl. In yet other embodiments, R₁₆ is acyloxy_{(C≤8)} such as acetoxy. In some embodiments, R₁₆′ is hydrogen. In other embodiments, R₁₆′ is hydroxy. In still other embodiments, R₁₆′ is alkyl_{(C≤8)} such as methyl. In yet other embodiments, R₁₆′ is acyloxy_{(C≤8)} such as acetoxy. In some embodiments, R₁₇ is hydrogen. In other embodiments, R₁₇ is hydroxy. In still other embodiments, R₁₇ is alkyl_{(C≤8)} such as methyl. In yet other embodiments, R₁₇ is acyloxy_{(C≤8)} such as acetoxy. In some embodiments, R₁₇′ is hydrogen. In other embodiments, R₁₇′ is hydroxy. In still other embodiments, R₁₇′ is alkyl_{(C≤8)} such as methyl. In yet other embodiments, R₁₇′ is acyloxy_{(C≤8)} such as acetoxy.

In some embodiments, R₁₂ or R₁₉ are a group of the formula:

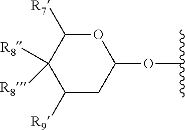

wherein:
R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₈″ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups; R₁ and R₂ are taken together and are alkanediyl_{(C≤8)} or substituted alkanediyl_{(C≤8)}.

In some embodiments, R₇′ is hydrogen. In some embodiments, R₉′ is alkoxy_{(C≤8)} such as methoxy. In some embodiments, R₈″ is hydrogen. In some embodiments, R₈′″ is amino. In some embodiments, R₂₀ is alkyl_{(C≤8)}. In other embodiments, R₂₀ is acyl_{(C≤8)}. In some embodiments, R₂₁ is alkyl_{(C≤8)}. In some embodiments, R₂₂ is hydroxy.

In some embodiments, R₂₃ is a group of the formula:

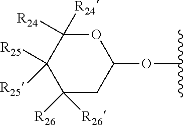

wherein:
R₂₄, R₂₄′, R₂₆, and R₂₆′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₂₅ and R₂₅′ are hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}.

In some embodiments, R₂₄ is hydrogen. In some embodiments, R₂₄′ is alkyl_{(C≤8)} such as methyl. In some embodiments, R₂₅ is hydrogen. In some embodiments, R₂₅′ is acyloxy_{(C≤8)} such as acetoxy. In some embodiments, R₂₆ is alkyl_{(C≤8)} such as methyl. In some embodiments, R₂₆′ is hydroxy.

In some embodiments, the compound is further defined as:

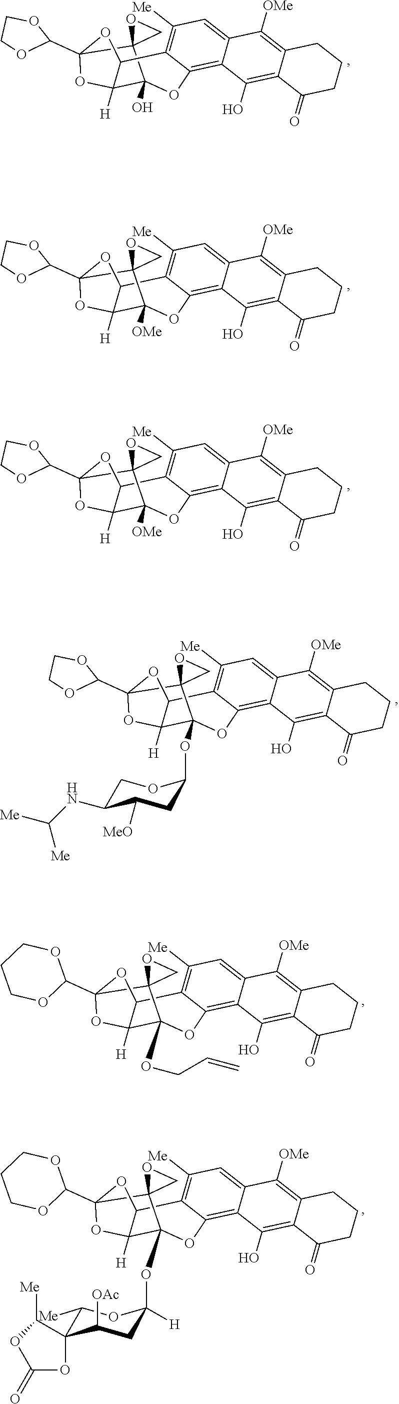

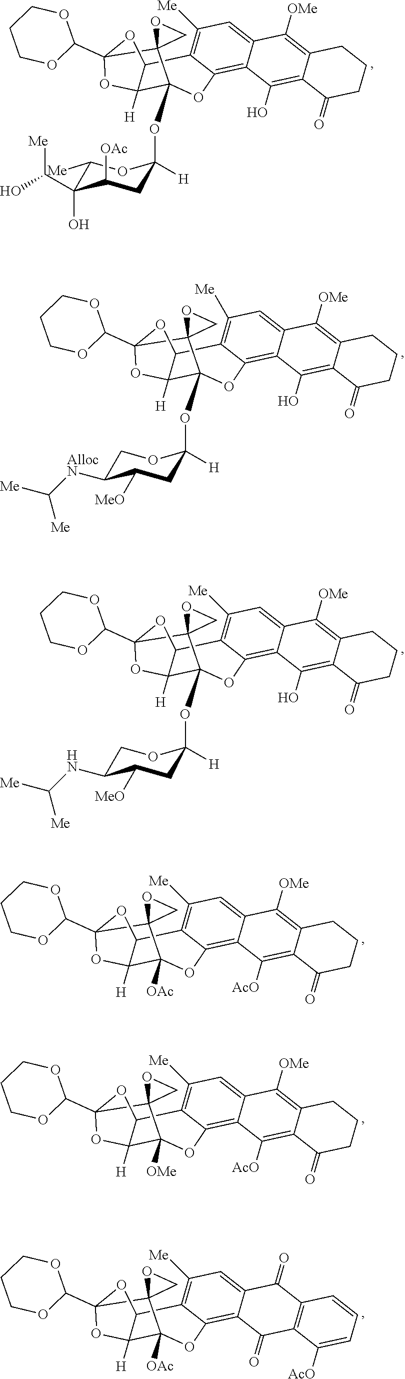

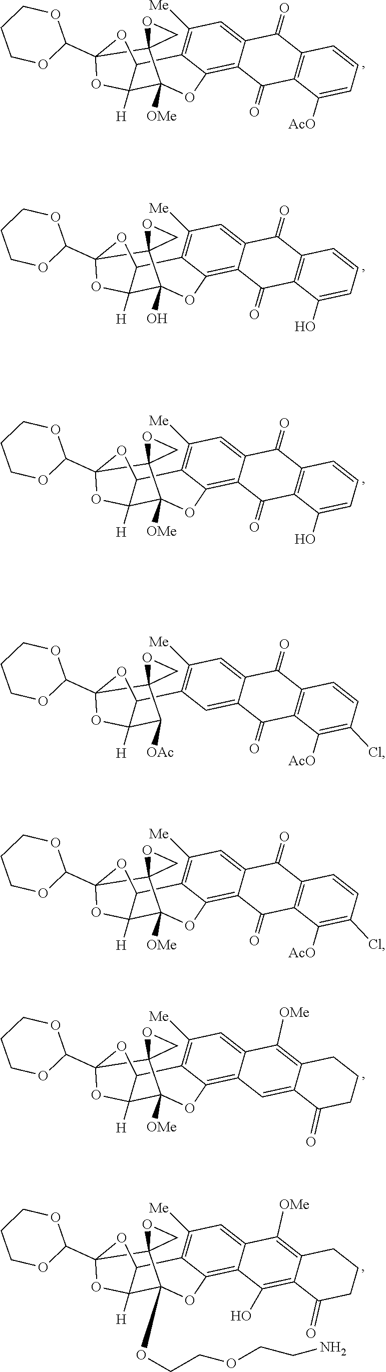

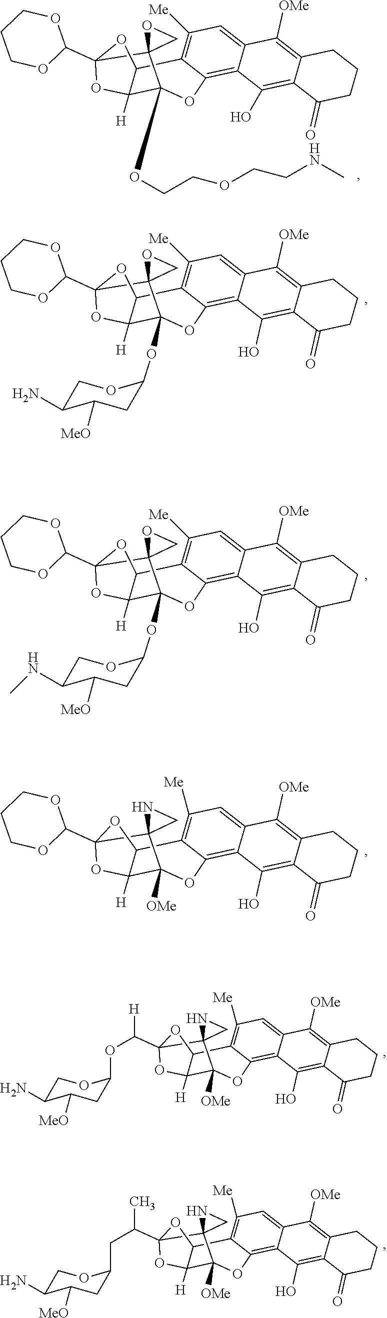

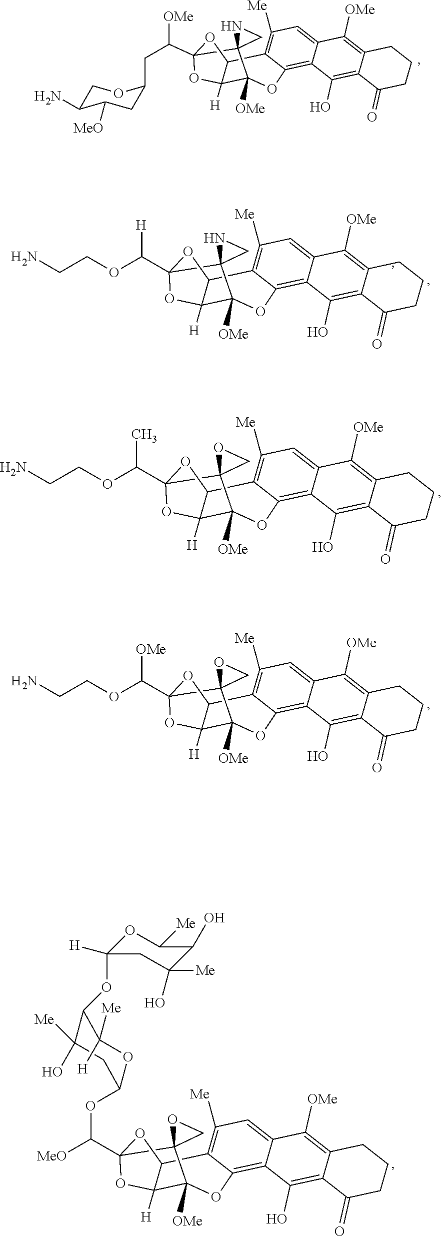

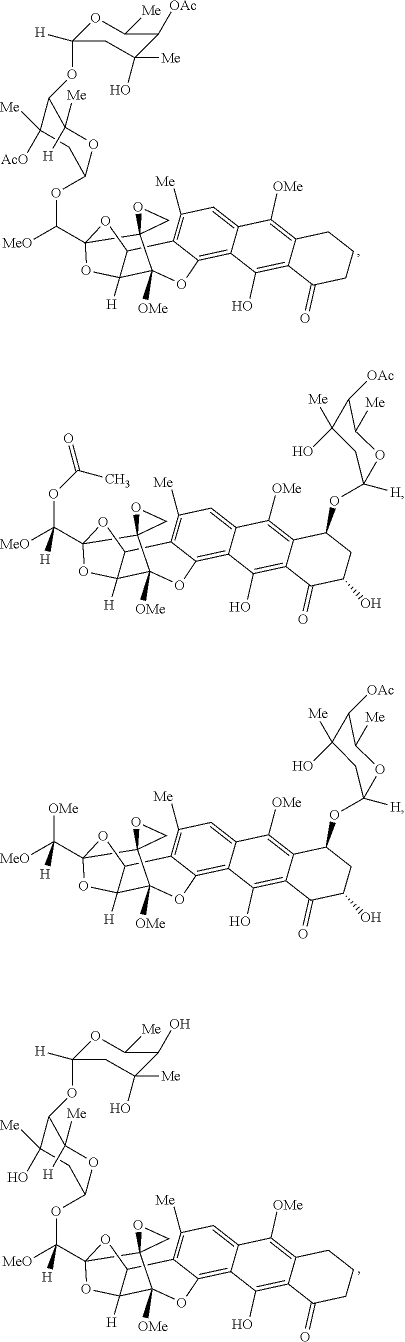

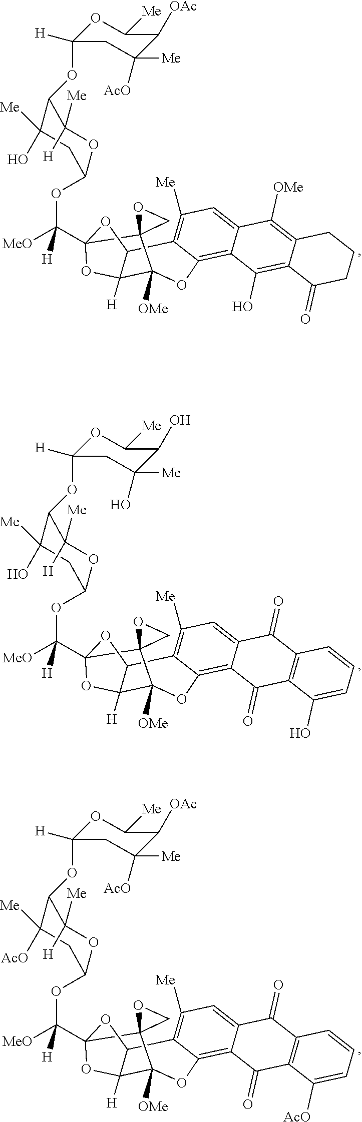

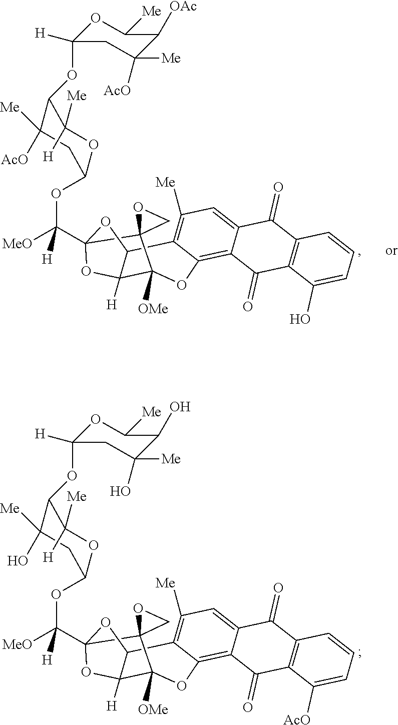

or a pharmaceutically acceptable salt thereof.

In another aspect, the present disclosure provides compounds further defined as:

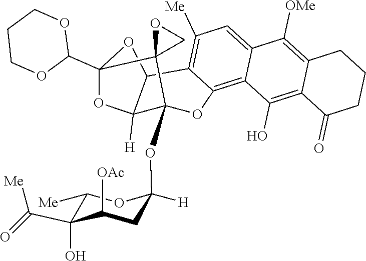

or a pharmaceutically acceptable salt thereof.

In another aspect, the present disclosure provides dimers further defined as:

Y₁-L-Y₁′ (XIII)

wherein:

Y₁ and Y₁′ are each independently a compound of the formula:

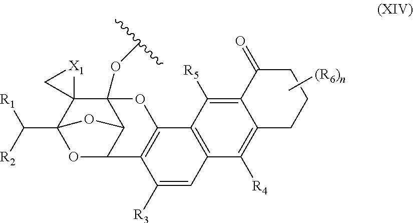

wherein:
   R₁ and R₂ are each independently hydrogen, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; a group of the formula: —O(CH₂)_{q}R₁₃, wherein:
   q is 1, 2, 3, 4, or 5;
   R₁₃ is amino, alkylamino_{(C≤8)}, substituted alkylamino_{(C≤8)}, dialkylamino_{(C≤8)}, or substituted dialkylamino_{(C≤8)}; or
a group of the formula:

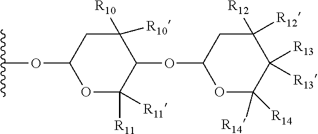

wherein:
   R₁₀, R₁₀′, R₁₁, R₁₁′, R₁₂, R₁₂′, R₁₃, R₁₃′, R₁₄, and R₁₄′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)}; or
a group of the formula:

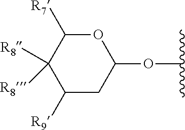

wherein:
   R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₈″ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocyclo-alkanediyl_{(C≤12)} or substituted heterocycloalkanediyl_{(C≤12)} which is optionally substituted with 1, 2, or 3 oxo groups; R₁ and R₂ are taken together and are alkanediyl_{(C≤8)} or substituted alkane-diyl_{(C≤8)};
R₃ is alkyl_{(C≤8)} or substituted alkyl_{(C≤8)};
R₄ is hydrogen, hydroxy, alkoxy_{(C≤8)}, substituted alkoxy_{(C≤8)}, acyloxy_{(C≤8)}, or substituted acyloxy_{(C≤8)};
R₅ is hydroxy, alkoxy_{(C≤8)}, substituted alkoxy_{(C≤8)}, acyloxy_{(C≤8)}, or substituted acyloxy_{(C≤8)};
R₆ is hydrogen, hydroxy, alkoxy_{(C≤8)}, substituted alkoxy_{(C≤8)}, acyloxy_{(C≤8)}, or substituted acyloxy_{(C≤8)}, or a group of the formula:

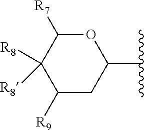

wherein:
   R₇ and R₉ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₈ and R₈′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkane-diyl_{(C≤12)} or substituted heterocycloalkanediyl_{(C≤12)} which is optionally substituted with 1, 2, or 3 oxo groups;
n is 1, 2, 3, 4, 5 or 6; and
X₁ is —O— or —NRₐ—, wherein Rₐ is hydrogen, alkyl_{(C≤8)}, or substituted alkyl_{(C≤8)}; or
   a compound of the formula:

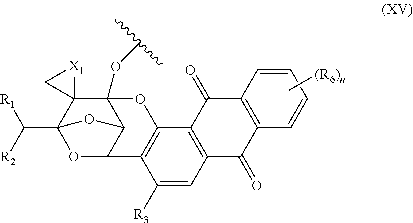

wherein:
R₁ and R₂ are each independently hydrogen, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; a group of the formula: —O(CH₂)_{q}R₁₃, wherein:
   q is 1, 2, 3, 4, or 5;
   R₁₃ is amino, alkylamino_{(C≤8)}, substituted alkylamino_{(C≤8)}, dialkylamino_{(C≤8)}, or substituted dialkylamino_{(C≤8)}; or
a group of the formula:

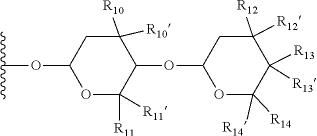

wherein:
   R₁₀, R₁₀′, R₁₁, R₁₁′, R₁₂, R₁₂′, R₁₃, R₁₃′, R₁₄, and R₁₄′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)};
a group of the formula:

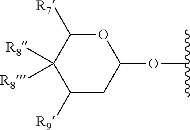

wherein:
   R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₈″ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocyclo-alkanediyl_{(C≤12)} or substituted heterocycloalkanediyl_{(C≤12)} which is optionally substituted with 1, 2, or 3 oxo groups; R₁ and R₂ are taken together and are alkanediyl_{(C≤8)} or substituted alkanediyl_{(C≤8)};
R₁ and R₂ are taken together and are alkoxydiyl_{(C≤8)} or substituted alkoxydiyl_{(C≤8)};
R₃ is alkyl_{(C≤8)} or substituted alkyl_{(C≤8)};
R₄ is hydrogen, hydroxy, alkoxy_{(C≤8)}, substituted alkoxy_{(C≤8)}, acyloxy_{(C≤8)}, or substituted acyloxy_{(C≤8)};
R₅ is hydroxy, alkoxy_{(C≤8)}, substituted alkoxy_{(C≤8)}, acyloxy_{(C≤8)}, or substituted acyloxy_{(C≤8)};
R₆ is hydrogen, hydroxy, alkoxy_{(C≤8)}, substituted alkoxy_{(C≤8)}, acyloxy_{(C≤8)}, or substituted acyloxy_{(C≤8)}, or a group of the formula:

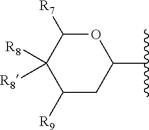

wherein:
   R₇ and R₉ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
   R₈ and R₈′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocyclo-alkanediyl_{(C≤12)} or substituted heterocycloalkanediyl_{(C≤12)} which is optionally substituted with 1, 2, or 3 oxo groups; and
n is 1, 2, 3, or 4; and
X₁ is —O— or —NRₐ—, wherein Rₐ is hydrogen, alkyl_{(C≤8)}, or substituted alkyl_{(C≤8)}; and
L is alkanediyl_{(C≤12)}, alkenediyl_{(C≤12)}, alkynediyl_{(C≤12)}, arenediyl_{(C≤12)}, heteroarenediyl_{(C≤12)}, heterocycloalkanediyl_{(C≤12)}, alkoxydiyl_{(C≤12)}, alkylaminodiyl_{(C≤12)}, or a substituted version of any of these groups; or a linker comprising an amino acid chain containing from 1 to 20 amino acids;
   or a pharmaceutically acceptable salt thereof.

In some embodiments, L is alkenediyl_{(C≤12)} or substituted alkenediyl_{(C≤12)}. In further embodiments, L is 2-butene, either (Z)-2-butene or (E)-2-butene. In other embodiments, L is alkylaminodiyl_{(C≤12)} such as —CH₂CH₂NHCH₂CH₂—. In some embodiments, R₁ and R₂ are taken together and are alkoxydiyl_{(C≤8)} such as —OCH₂CH₂CH₂— or —OCH₂CH₂—. In some embodiments, R₃ is alkyl_{(C≤8)} such as methyl. In some embodiments, R₄ is alkoxy_{(C≤8)} such as methoxy. In some embodiments, R₅ is hydroxy. In some embodiments, R₆ is hydrogen. In some embodiments, n is 0 or 1. In further embodiments, n is 0. In some embodiments, X₁ is —O—.

In some embodiments, the compound is further defined as:

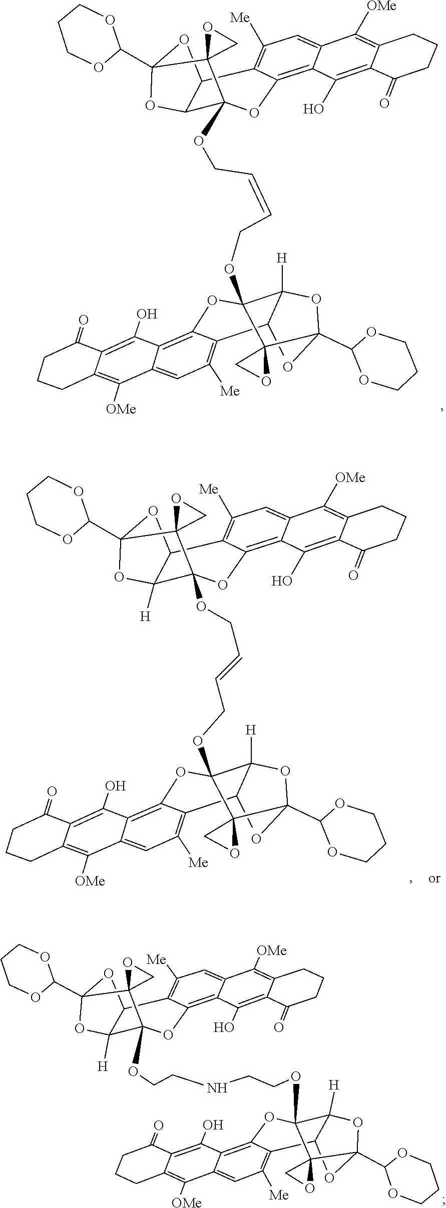

or a pharmaceutically acceptable salt thereof.

In another aspect, the present disclosure provides pharmaceutical compositions comprising a compound described herein and a pharmaceutically acceptable carrier. In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In some embodiments, the pharmaceutical composition is formulated as a unit dose.

In another aspect, the present disclosure provides methods of treating a disease or disorder in a patient in need thereof comprising administering to the patient a pharmaceutically effective amount of a compound or composition described herein. In some embodiments, the disease or disorder is cancer. In further embodiments, the cancer is a carcinoma, sarcoma, lymphoma, leukemia, melanoma, mesothelioma, multiple myeloma, or seminoma. In still further embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

In some embodiments, the method comprises a second therapeutic agent or modality. In further embodiments, the second therapeutic agent or modality is an immunotherapy, surgery, another chemotherapeutic compound, or radiation therapy. In some embodiments, the compound is administered once. In other embodiments, the compound is administered two or more times.

In still another aspect, the present disclosure provides conjugates of the formula:

(A-L)-X (XVI)

wherein:

A is a compound or dimer according described herein;

L is a covalent bond or a linker;

n is 1, 2, 3, 4, 5, 6, 7, 8, 9, 10, 11, or 12; and

X is a cell targeting moiety.

In yet another aspect, the present disclosure provides methods of preparing compounds of the formula:

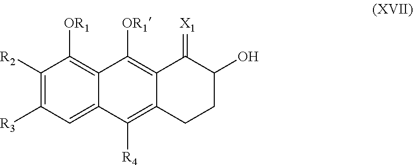

wherein:
R₁ and R₁′ are each independently hydroxy protecting groups or are taken together and are —SiR_{b}R_{c}—, wherein R_{b} and R_{c} are each independently alkyl_{(C≤8)} or substituted alkyl_{(C≤8)};
R₂ is hydrogen or halo;
R₃ is alkyl_{(C≤8)} or substituted alkyl_{(C≤8)};
R₄ is hydroxy, alkoxy_{(C≤8)}, or substituted alkoxy_{(C≤8)}; and
X₁ is O, S, or NR_{c}, wherein R_{c} is hydrogen, alkyl_{(C≤8)}, or substituted alkyl_{(C≤8)};
   comprising reacting an oxaziridine reagent and a compound of the formula:

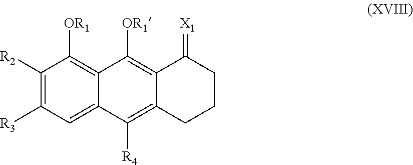

wherein:
R₁ and R₁′ are each independently hydroxy protecting groups or are taken together and are —SiR_{b}R_{c}—, wherein R_{b} and R_{c} are each independently alkyl_{(C≤8)} or substituted alkyl_{(C≤8)};
R₂ is hydrogen or halo;
R₃ is alkyl_{(C≤8)} or substituted alkyl_{(C≤8)};
R₄ is hydroxy, alkoxy_{(C≤8)}, or substituted alkoxy_{(C≤8)}; and
X₁ is O, S, or NR_{c}, wherein R_{c} is hydrogen, alkyl_{(C≤8)}, or substituted alkyl_{(C≤8)};
in the presence of a base under conditions sufficient to achieve a reaction.

In some embodiments, the oxaziridine reagent is further defined by the formula:

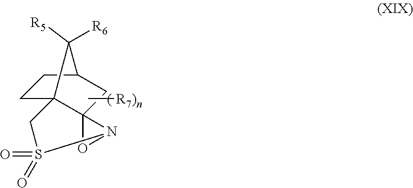

wherein:
R₅ and R₆ are each independently alkyl_{(C≤8)} or substituted alkyl_{(C≤8)};
R₇ is hydrogen, halo, alkyl_{(C≤8)}, or substituted alkyl_{(C≤8)}; and
n is 1, 2, 3, 4, or 5.
   In further embodiments, the oxaziridine reagent is defined as:

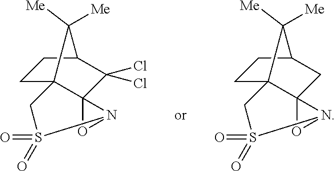 [SMILES: CC1(C)C2CCC13CS(=O)(=O)N1OC13C2(Cl)Cl ; CC1(C)C2CCC13CS(=O)(=O)N1OC13C2]

In some embodiments, the base is a strong base. In further embodiments, the base is an amide base such as a lithium amide base. In some embodiments, the conditions comprise a solvent. In further embodiments, the solvent is an ether_{(C1-8)} such as tetrahydrofuran. In some embodiments, the conditions comprise a temperature from about −100° C. to about −20° C. In further embodiments, the temperature is about −78° C. In some embodiments, the conditions comprise reacting the oxaziridine reagent, the compound of formula XVIII, and the base for a time period from about 10 minutes to about 8 hours. In further embodiments, the time period is about 30 minutes to about 2 hours.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description.

FIG. 1A-C show the 72 hour killing assay for Triox 8 and known antibody-drug conjugate payloads MMAE and DC-45-A2 for MES SA (FIG. 1A), MES SA DX (FIG. 1B), and 293T (FIG. 1C).

FIG. 2A-C show the 72 hour killing assay for Trioxacarcin A, Trioxacacrin D, Trx1, Trx2, and known antibody-drug conjugate payloads MMAE and DC-45-A2 for MES SA (FIG. 2A), MES SA DX (FIG. 2B), and 293T (FIG. 2C).

FIG. 3A-C show the 72 hour killing assay for Trioxacarcin C, Trx3, Trx4, Trx5, and known antibody-drug conjugate payload MMAE for MES SA (FIG. 3A), MES SA DX (FIG. 3B), and 293T (FIG. 3C).

FIG. 4A-C show the 72 hour killing assay for Trx7, Trx6, and known antibody-drug conjugate payload MMAE for MES SA (FIG. 4A), MES SA DX (FIG. 4B), and 293T (FIG. 4C).

FIG. 5A-C show the 72 hour killing assay for Trx11, Trx8, Trx9, Trx10, and known antibody-drug conjugate payload MMAE for MES SA (FIG. 5A), MES SA DX (FIG. 5B), and 293T (FIG. 5C).

FIG. 6A-C show the 72 hour killing assay for Trx12, Trx13, and Trx14 for MES SA (FIG. 6A), MES SA DX (FIG. 6B), and 293T (FIG. 6C).

FIG. 7A-C show the 72 hour killing assay for Trx16, Trx15, and known antibody-drug conjugate payload MMAE for MES SA (FIG. 7A), MES SA DX (FIG. 7B), and 293T (FIG. 7C).

FIG. 8A-C show the 72 hour killing assay for Trx19, Trx18, Trx23, Trx22, Trx20, Trx21, and known antibody-drug conjugate payload MMAE for MES SA (FIG. 8A), MES SA DX (FIG. 8B), and 293T (FIG. 8C).

FIG. 9A-C show the 72 hour killing assay for Trx26, Trx28, Trx30, and known antibody-drug conjugate payload MMAE for MES SA (FIG. 9A), MES SA DX (FIG. 9B), and 293T (FIG. 9C).

FIG. 10A-C show the 72 hour killing assay for Trx29, Trx31, and known antibody-drug conjugate payload MMAE for MES SA (FIG. 10A), MES SA DX (FIG. 10B), and 293T (FIG. 10C).

FIG. 11A-C show the 72 hour killing assay for Trx27, Trx17, Trx24, Trx25, and known antibody-drug conjugate payload MMAE for MES SA (FIG. 11A), MES SA DX (FIG. 11B), and 293T (FIG. 11C).

FIG. 12A-C show the 72 hour killing assay for Trx32, Trx33, Trx13, and known antibody-drug conjugate payload MMAE for MES SA (FIG. 12A), MES SA DX (FIG. 12B), and 293T (FIG. 12C).

FIG. 13A-C show the 72 hour killing assay for Trx34 (nabla) and known antibody-drug conjugate payload MMAE (star) for MES SA (FIG. 13A), MES SA DX (FIG. 13B), and 293T (FIG. 13C).

FIG. 14A-C show the 72 hour killing assay for Trx35 (triangle) for MES SA (FIG. 14A), MES SA DX (FIG. 14B), and 293T (FIG. 14C).

### DESCRIPTION OF ILLUSTRATIVE EMBODIMENTS

The present disclosure provides derivatives of trioxacarcin and dimers thereof which may be used in antibody-drug conjugates. In some aspects, these compounds may contain modifications which increase the activity, chemical stability, or both. Also, provided herein are methods of using these compounds, antibody-drug conjugates thereof, and compositions thereof.

### I. COMPOUNDS AND FORMULATIONS THEREOF

A. Compounds

The compounds provided by the present disclosure are shown, for example, above in the summary section and in the examples and claims below. They may be made using the methods outlined in the Examples section. The trioxacarcin analogs and dimers described herein can be synthesized according to the methods described, for example, in the Examples section below. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

The trioxacarcin analogs and dimers described herein may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated.

Compounds may occur as racemates and racemic mixtures, single enantiomers, diastereomeric mixtures and individual diastereomers. In some embodiments, a single diastereomer is obtained. The chiral centers of the compounds of the present disclosure can have the (S) or the (R) configuration.

Chemical formulas used to represent the trioxacarcin analogs and dimers described herein will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

The trioxacarcin analogs and dimers described herein may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the trioxacarcin analogs and dimers described herein are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The trioxacarcin analogs and dimers described herein may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the trioxacarcin analogs and dimers described herein may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

Those skilled in the art of organic chemistry will appreciate that many organic compounds can form complexes with solvents in which they are reacted or from which they are precipitated or crystallized. These complexes are known as “solvates.” For example, a complex with water is known as a “hydrate.” Solvates of the trioxacarcin analogs and dimers described herein are within the scope of the disclosure. It will also be appreciated by those skilled in organic chemistry that many organic compounds can exist in more than one crystalline form. For example, crystalline form may vary from solvate to solvate. Thus, all crystalline forms of the trioxacarcin analogs and dimers described herein are within the scope of the present disclosure.

B. Formulations

In some embodiments of the present disclosure, the trioxacarcin analogs and dimers described herein are included a pharmaceutical formulation. Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., the trioxacarcin analogs and dimers described herein) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated, or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

### II. CANCER AND OTHER HYPERPROLIFERATIVE DISEASES

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In this disclosure, the trioxacarcin analogs and dimers described herein may be used to lead to decreased cell counts and as such can potentially be used to treat a variety of types of cancer lines. In some aspects, it is anticipated that the trioxacarcin analogs and dimers described herein may be used to treat virtually any malignancy.

Cancer cells that may be treated with the compounds of the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### III. CELL TARGETING MOIETIES

In some aspects, the present disclosure provides compounds conjugated directly or through linkers to a cell targeting moiety. In some embodiments, the conjugation of the compound to a cell targeting moiety increases the efficacy of the compound in treating a disease or disorder. Cell targeting moieties according to the embodiments may be, for example, an antibody, a growth factor, a hormone, a peptide, an aptamer, a small molecule such as a hormone, an imaging agent, or cofactor, or a cytokine. For instance, a cell targeting moiety according the embodiments may bind to a liver cancer cell such as a Hep3B cell. It has been demonstrated that the gp240 antigen is expressed in a variety of melanomas but not in normal tissues. Thus, in some embodiments, the compounds of the present disclosure may be used in conjugates with an antibody for a specific antigen that is expressed by a cancer cell but not in normal tissues.

In certain additional embodiments, it is envisioned that cancer cell targeting moieties bind to multiple types of cancer cells. For example, the 8H9 monoclonal antibody and the single chain antibodies derived therefrom bind to a glycoprotein that is expressed on breast cancers, sarcomas and neuroblastomas (Onda, et al., 2004). Another example is the cell targeting agents described in U.S. Patent Publication No. 2004/005647 and in Winthrop, et al. (2003) that bind to MUC-1, an antigen that is expressed on a variety cancer types. Thus, it will be understood that in certain embodiments, cell targeting constructs according the embodiments may be targeted against a plurality of cancer or tumor types.

Additionally, certain cell surface molecules are highly expressed in tumor cells, including hormone receptors such as human chorionic gonadotropin receptor and gonadotropin releasing hormone receptor (Nechushtan et al., 1997). Therefore, the corresponding hormones may be used as the cell-specific targeting moieties in cancer therapy. Additionally, the cell targeting moiety that may be used include a cofactor, a sugar, a drug molecule, an imaging agent, or a fluorescent dye. Many cancerous cells are known to over express folate receptors and thus folic acid or other folate derivatives may be used as conjugates to trigger cell-specific interaction between the conjugates of the present disclosure and a cell (Campbell, et al., 1991; Weitman, et al., 1992).

Since a large number of cell surface receptors have been identified in hematopoietic cells of various lineages, ligands or antibodies specific for these receptors may be used as cell-specific targeting moieties. IL-2 may also be used as a cell-specific targeting moiety in a chimeric protein to target IL-2R+ cells. Alternatively, other molecules such as B7-1, B7-2 and CD40 may be used to specifically target activated T cells (The Leucocyte Antigen Facts Book, 1993, Barclay, et al. (eds.), Academic Press). Furthermore, B cells express CD19, CD40 and IL-4 receptor and may be targeted by moieties that bind these receptors, such as CD40 ligand, IL-4, IL-5, IL-6 and CD28. The elimination of immune cells such as T cells and B cells is particularly useful in the treatment of lymphoid tumors.

Other cytokines that may be used to target specific cell subsets include the interleukins (IL-1 through IL-15), granulocyte-colony stimulating factor, macrophage-colony stimulating factor, granulocyte-macrophage colony stimulating factor, leukemia inhibitory factor, tumor necrosis factor, transforming growth factor, epidermal growth factor, insulin-like growth factors, and/or fibroblast growth factor (Thompson (ed.), 1994, The Cytokine Handbook, Academic Press, San Diego). In some aspects, the targeting polypeptide is a cytokine that binds to the Fn14 receptor, such as TWEAK (see, e.g., Winkles, 2008; Zhou, et al., 2011 and Burkly, et al., 2007, incorporated herein by reference).

A skilled artisan recognizes that there are a variety of known cytokines, including hematopoietins (four-helix bundles) (such as EPO (erythropoietin), IL-2 (T-cell growth factor), IL-3 (multicolony CSF), IL-4 (BCGF-1, BSF-1), IL-5 (BCGF-2), IL-6 IL-4 (IFN-β2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)); interferons (such as IFN-γ, IFN-α, and IFN-β); immunoglobin superfamily (such as B7.1 (CD80), and B7.2 (B70, CD86)); TNF family (such as TNF-α (cachectin), TNF-β (lymphotoxin, LT, LT-α), LT-β, CD40 ligand (CD40L), Fas ligand (FasL), CD27 ligand (CD27L), CD30 ligand (CD30L), and 4-1BBL)); and those unassigned to a particular family (such as TGF-β, IL 1α, IL-1β, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)).

Furthermore, the Fc portion of the heavy chain of an antibody may be used to target Fc receptor-expressing cells such as the use of the Fc portion of an IgE antibody to target mast cells and basophils.

Furthermore, in some aspects, the cell-targeting moiety may be a peptide sequence or a cyclic peptide. Examples, cell- and tissue-targeting peptides that may be used according to the embodiments are provided, for instance, in U.S. Pat. Nos. 6,232,287; 6,528,481; 7,452,964; 7,671,010; 7,781,565; 8,507,445; and 8,450,278, each of which is incorporated herein by reference.

Thus, in some embodiments, cell targeting moieties are antibodies or avimers. Antibodies and avimers can be generated against virtually any cell surface marker thus, providing a method for targeted to delivery of GrB to virtually any cell population of interest. Methods for generating antibodies that may be used as cell targeting moieties are detailed below. Methods for generating avimers that bind to a given cell surface marker are detailed in U.S. Patent Publications Nos. 2006/0234299 and 2006/0223114, each incorporated herein by reference.

Additionally, it is contemplated that the compounds described herein may be conjugated to a nanoparticle or other nanomaterial. Some non-limiting examples of nanoparticles include metal nanoparticles such as gold or silver nanoparticles or polymeric nanoparticles such as poly-L-lactic acid or poly(ethylene) glycol polymers. Nanoparticles and nanomaterials which may be conjugated to the instant compounds include those described in U.S. Patent Publications Nos. 2006/0034925, 2006/0115537, 2007/0148095, 2012/0141550, 2013/0138032, and 2014/0024610 and PCT Publication No. 2008/121949, 2011/053435, and 2014/087413, each incorporated herein by reference.

### IV. THERAPIES

A. Pharmaceutical Formulations and Routes of Administration

Where clinical applications are contemplated, it will be necessary to prepare pharmaceutical compositions in a form appropriate for the intended application. In some embodiments, such formulation with the trioxacarcin analogs and dimers described herein is contemplated. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like.

The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the trioxacarcin analogs and dimers described herein may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 mL of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the appropriate regulatory agencies for the safety of pharmaceutical agents.

B. Methods of Treatment

In particular, the compositions that may be used in treating cancer in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that induces death of cancerous cells (e.g., induces apoptosis of a cancer cell), as assayed by identifying a reduction in hematological parameters (complete blood count—CBC), or cancer cell growth or proliferation. In some embodiments, amounts of the trioxacarcin analogs and dimers used to induce apoptosis of the cancer cells is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the trioxacarcin analogs and dimers may be more efficacious and thus a smaller dose is required to achieve a similar effect. Such a dose is typically administered once a day for a few weeks or until sufficient reducing in cancer cells has been achieved.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In one embodiment, the disclosure provides a method of monitoring treatment progress. The method includes the step of determining a level of changes in hematological parameters and/or cancer stem cell (CSC) analysis with cell surface proteins as diagnostic markers (which can include, for example, but are not limited to CD34, CD38, CD90, and CD117) or diagnostic measurement (e.g., screen, assay) in a subject suffering from or susceptible to a disorder or symptoms thereof associated with cancer in which the subject has been administered a therapeutic amount of a composition as described herein. The level of marker determined in the method can be compared to known levels of marker in either healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

C. Combination Therapies

It is envisioned that the trioxacarcin analogs and dimers described herein may be used in combination therapies with one or more cancer therapies or a compound which mitigates one or more of the side effects experienced by the patient. It is common in the field of cancer therapy to combine therapeutic modalities. The following is a general discussion of therapies that may be used in conjunction with the therapies of the present disclosure.

To treat cancers using the methods and compositions of the present disclosure, one would generally contact a tumor cell or subject with a compound and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the trioxacarcin analogs and dimers described herein may precede or follow the other treatment by intervals ranging from minutes to weeks. One would generally ensure that a significant period of time did not expire between the times of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. The following is a general discussion of cancer therapies that may be used combination with the compounds of the present disclosure.

1. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ₁ and calicheamicin ω₁; dynemicin, including dynemicin A; uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichloro-triethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ; IL-1, GM-CSF and TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believed to be particularly efficacious in reducing the reoccurance of the tumor. Additionally, the compounds of the present disclosure can also be used in a neoadjuvant setting.

5. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents may be used in combination with the present disclosure to improve the anti-hyerproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 41.1° C.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the appropriate pharmaceutical agent regulatory agencies.

It also should be pointed out that any of the foregoing therapies may prove useful by themselves in treating cancer.

### V. SYNTHETIC METHODS

In some aspects, the trioxacarcin analogs and dimers described herein can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

A. Process Scale-Up

The synthetic methods described herein can be further modified and optimized for preparative, pilot- or large-scale production, either batch of continuous, using the principles and techniques of process chemistry as applied by a person skilled in the art. Such principles and techniques are taught, for example, in Practical Process Research & Development (2000), which is incorporated by reference herein. The synthetic method described herein may be used to produce preparative scale amounts of the trioxacarcin analogs and dimers described herein.

B. Chemical Definitions

When used in the context of a chemical group: “hydrogen” means —H; “hydroxy” means —OH; “oxo” means=O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means=NH; “cyano” means —CN; “isocyanate” means —N═C═O; “azido” means —N₃; “hydrazine” means —NHNH₂; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means=S; “hydroxysulfonyl” means —SO₃H, “sulfonyl” means —S(O)₂—; and “sulfinyl” means —S(O)—.

In the context of chemical formulas, the symbol “-” means a single bond, “ [symbol] ” means a double bond, and “ [symbol] ” means triple bond. The symbol “ [symbol] ” represents an optional bond, which if present is either single or double. The symbol “ [symbol] ” represents a single bond or a double bond. Thus, the formula

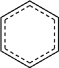

covers, for example,

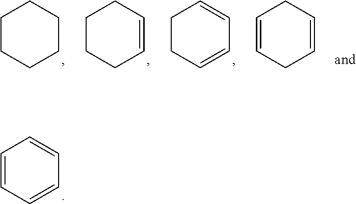

And it is understood that no one such ring atom forms part of more than one double bond. Furthermore, it is noted that the covalent bond symbol “-”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

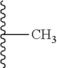

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond [e.g., either (E) or (Z)] is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

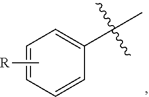

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

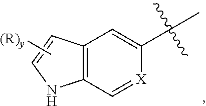

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn−n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. When any of the chemical groups or compound classes defined herein is modified by the term “substituted”, any carbon atom(s) in the moiety replacing a hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group refers to a planar unsaturated ring of atoms with 4n+2 electrons in a fully conjugated cyclic 7c system.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂CH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e., —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

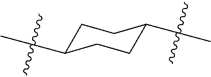 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. The term “alkynediyl” when used without the “substituted” modifier refers to a divalent unsaturated aliphatic group, with two carbon atoms as points of attachment, a linear or branched, a linear or branched acyclic structure, either no or one or more nonaromatic carbon-carbon double bond, at least one carbon-carbon triple bonds, and no atoms other than carbon and hydrogen. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more aromatic ring structure, wherein the ring atoms are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

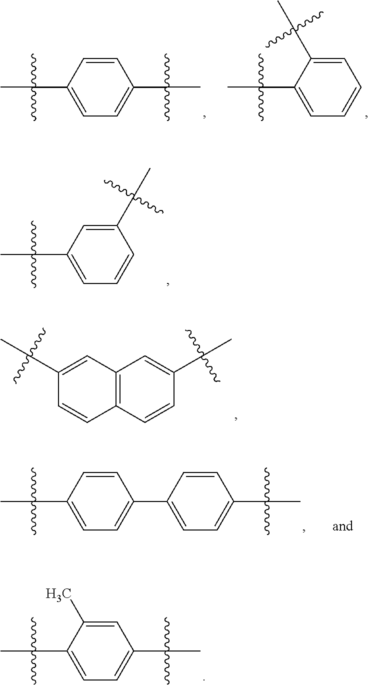

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, the aromatic ring structures being one, two, three, or four ring structures each containing from three to nine ring atoms, and wherein the heteroaryl group consists of no atoms other than carbon, hydrogen, aromatic nitrogen, aromatic oxygen and aromatic sulfur. If more than one ring is present, the rings may be fused or unfused. Unfused rings are connected with a covalent bond. As used herein, the term heteroaryl does not preclude the presence of one or more alkyl or aryl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings are connected with a covalent bond. As used herein, the term heteroarenediyl does not preclude the presence of one or more alkyl or aryl groups (carbon number limitation permitting) attached to the aromatic ring or aromatic ring system. Non-limiting examples of heteroarenediyl groups include:

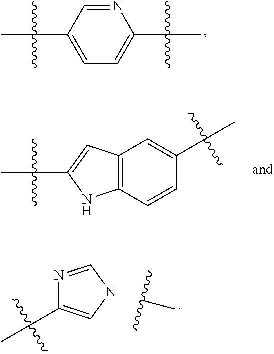

The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, the non-aromatic ring structures being one, two, three, or four ring structures each containing from three to nine ring atoms, and wherein the heterocycloalkyl group consists of no atoms other than carbon, hydrogen, nitrogen, oxygen and sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “heterocycloalkanediyl” when used without the “substituted” modifier refers to an divalent cyclic group, with two carbon atoms, two nitrogen atoms, or one carbon atom and one nitrogen atom as the two points of attachment, said atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings are connected with a covalent bond. As used herein, the term heterocycloalkanediyl does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

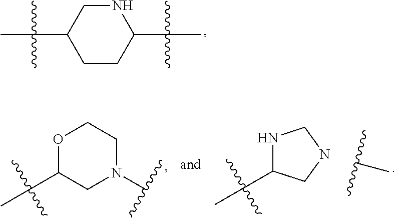

The term “N-heterocycloalkyl” refers to a heterocycloalkyl group with a nitrogen atom as the point of attachment. N-pyrrolidinyl is an example of such a group. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H4CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃(tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group or a heterocycloalkane group wherein at least one of the heteroatoms is an oxygen atom. The term “alkoxydiyl” when used without the “substituted” modifier refers to a divalent unsaturated aliphatic group, with zero, one, or two carbon atoms as points of attachment with the remaining points of attachment being oxygen atoms, a linear or branched, a linear or branched acyclic structure containing at least one oxygen atom in the chain, no nonaromatic carbon-carbon double bond, no carbon-carbon triple bonds, and no atoms other than carbon, oxygen and hydrogen. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. The term “alkylaminodiyl” when used without the “substituted” modifier refers to a divalent unsaturated aliphatic group, with zero, one, or two carbon atoms as points of attachment with the remaining points of attachment being nitrogen atoms, a linear or branched, a linear or branched acyclic structure containing at least one nitrogen atom in the chain, no nonaromatic carbon-carbon double bond, no carbon-carbon triple bonds, and no atoms other than carbon, nitrogen and hydrogen. The term alkylaminodiyl does not preclude the attachment of one or more additional alkyl groups on the nitrogen atoms to form tertiary amines carbon limit permitting. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

As indicated above in some aspects the cell-targeting moiety is an antibody. As used herein, the term “antibody” is intended to include immunoglobulins and fragments thereof which are specifically reactive to the designated protein or peptide, or fragments thereof. Suitable antibodies include, but are not limited to, human antibodies, primatized antibodies, de-immunized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, antibody-like molecules (e.g., anticalins), and antibody fragments. As used herein, the term “antibodies” also includes intact monoclonal antibodies, polyclonal antibodies, single domain antibodies (e.g., shark single domain antibodies (e.g., IgNAR or fragments thereof), multispecific antibodies (e.g., bi-specific antibodies) formed from at least two intact antibodies, and antibody fragments so long as they exhibit the desired biological activity. Antibody polypeptides for use herein may be of any type (e.g., IgG, IgM, IgA, IgD and IgE). Generally, IgG and/or IgM are preferred because they are the most common antibodies in the physiological situation and because they are most easily made in a laboratory setting. As used herein the term antibody also encompasses an antibody fragment such as a portion of an intact antibody, such as, for example, the antigen-binding or variable region of an antibody. Examples of antibody fragments include Fab, Fab′, F(ab′)₂, Fc and Fv fragments; triabodies; tetrabodies; linear antibodies; single-chain antibody molecules; and multi specific antibodies formed from antibody fragments. The term “antibody fragment” also includes any synthetic or genetically engineered protein that acts like an antibody by binding to a specific antigen to form a complex. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. An oxygen linked antibody is an antibody which has a chemical function group such that the linkage between the antibody and the linker or compound is joined via an oxygen atom. Similarly, a nitrogen linked antibody is an antibody which has a chemical function group such that the linkage between the antibody and the linker or compound is joined via a nitrogen atom.

A “linker” in the context of this application is divalent chemical group which may be used to join one or more molecules to the compound of the instant disclosure. Linkers may also be an amino acid chain wherein the carboxy and amino terminus serve as the points of attachment for the linker. In some embodiments, the linker contains a reactive functional group, such as a carboxyl, an amide, an amine, a hydroxy, a mercapto, an aldehyde, or a ketone on each end that be used to join one or more molecules to the compounds of the instant disclosure. In some non-limiting examples, —CH₂CH₂CH₂CH₂—, —C(O)CH₂CH₂CH₂—, —OCH₂CH₂NH—, —NHCH₂CH₂NH—, and —(OCH₂CH₂)ₙ—, wherein n is between 1-1000, are linkers.

An “amine protecting group” is well understood in the art. An amine protecting group is a group which prevents the reactivity of the amine group during a reaction which modifies some other portion of the molecule and can be easily removed to generate the desired amine. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth). When used herein, a “protected amino group”, is a group of the formula PG_{MA}NH— or PG_{DA}N— wherein PGM is a monovalent amine protecting group, which may also be described as a “monvalently protected amino group” and PG_{DA} is a divalent amine protecting group as described above, which may also be described as a “divalently protected amino group”.

A “hydroxyl protecting group” is well understood in the art. A hydroxyl protecting group is a group which prevents the reactivity of the hydroxyl group during a reaction which modifies some other portion of the molecule and can be easily removed to generate the desired hydroxyl. Hydroxyl protecting groups can be found at least in Greene and Wuts, 1999, which is incorporated herein by reference. Some non-limiting examples of hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. When used herein, a protected hydroxy group is a group of the formula PG_{H}O— wherein PG_{H} is a hydroxyl protecting group as described above.

A “thiol protecting group” is well understood in the art. A thiol protecting group is a group which prevents the reactivity of the mercapto group during a reaction which modifies some other portion of the molecule and can be easily removed to generate the desired mercapto group. Thiol protecting groups can be found at least in Greene and Wuts, 1999, which is incorporated herein by reference. Some non-limiting examples of thiol protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. When used herein, a protected thiol group is a group of the formula PG_{T}S— wherein PG₁ is a thiol protecting group as described above.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedrally substituted carbon centers), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its (R) form, (S) form, or as a mixture of the (R) and (S) forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Improvements in the Original Synthetic Strategy and Streamlined Total Synthesis of Trioxacarcins

In light of certain deficiencies in the original route toward the trioxacarcins (Nicolaou et al., 2015; Nicolaou et al., 2016a), a number of improvements were desired in order to render it more practical as a means to construct designed analogues for biological evaluation. Scheme 3A summarizes the original route toward the tricyclic core 8 (6+7→8), a key building block in the first total synthesis (Nicolaou et al., 2015; Nicolaou et al., 2016a), while Scheme 3B depicts an improved synthesis of this intermediate as will be described below. The first synthesis of tricyclic ketone 8 involved a Hauser-Kraus union of iodocyanophthalide 6 (obtained in seven steps in 24% overall yield from 4-methylsalicyclic acid) with enone 73 (Nicolaou et al., 2015; de Sousa et al., 2002) (obtained in seven steps and 24% overall yield from 1,4-cyclohexadiene), followed by a four-step sequence of further elaboration. Besides its length and overall yield, this sequence suffered from random iodination of the aromatic precursor to iodophthalide 6. In the new strategy (Scheme 3B), the des-iodophthalide precursor 9 (Nicolaou et al., 2009) (obtained in four steps from 4-methylsalicylic acid in 69% overall yield) was used in the Hauser-Kraus fusion (t-BuOLi) with enone 7 to produce, after selective methylation, tricyclic system 10 (Me₂SO₄, 72% overall yield), with the remaining OH group being internally protected by H-bonding with the neighboring carbonyl group. The latter was subjected to MOM cleavage (MgBr₂.Et₂O, 79% yield) to afford phenol 11, whose exposure to NBS furnished selectively the desired bromide 12 in 82% yield. The bis-naphthol moiety within bromide 12 was then protected with t-Bu₂Si(OTf)₂ in the presence of Et₃N, furnishing substrate 13 (71% yield), whose Pd(PPh₃)₄-catalyzed coupling with stannane 14 (Nicolaou et al., 2016b) yielded the desired allylic alcohol 8 in 68% yield. Consisting of nine steps and proceeding in 16% overall yield, from 4-methyl salicylic acid and cyclohexenone 7, this sequence represents a significant improvement over the original synthesis of this advanced intermediate (18 total number of steps from 4-methylsalicylic acid and 1,4-cyclohexadiene, 10% overall yield) (Nicolaou et al., 2015; Nicolaou et al., 2016a).

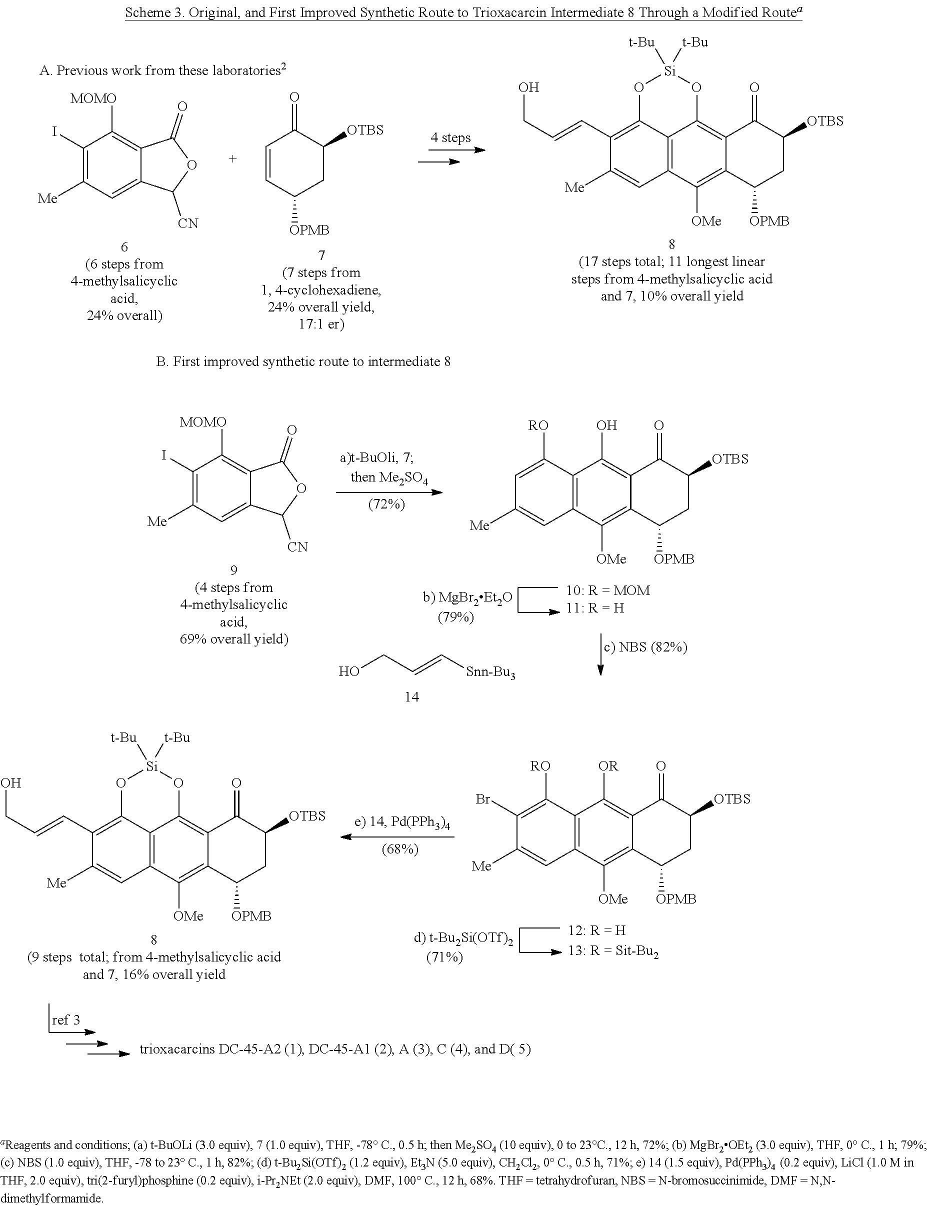

A further advance in improving the synthesis of advanced intermediate 8 was made by replacing building block 7 with cyclohexenone and postponing the introduction of the two required stereocenters (hydroxyl residues) until later in the sequence as shown in Scheme 4. Thus, reaction of cyanophthalide 9 with t-BuOLi at −78° C., followed first by addition of cyclohexenone and then Me₂SO₄ led to tricyclic ketone 15 (72% yield). Removal of the MOM group (MgBr₂.Et₂O, 90% yield) from 15 led to intermediate 16, whose treatment with NBS furnished bromide 17 (85% yield). The latter was exposed to t-Bu₂Si(OTf)₂ and Et₃N, affording substrate 18, whose asymmetric α-hydroxylation was achieved with chiral oxaziridine (−)-19 (Davis and Haque, 1986; Davis et al., 1990) in the presence of LTMP at −78° C. (77% yield and 27:1 er). These reagents and optimized conditions were identified after a systematic investigation of a number of oxaziridine hydroxylating agents [i.e., (−)-19a, (+)-19, and (−)-19] and bases as shown in Table 1. Protection of the so-obtained hydroxy compound (10S)-20 (Scheme 4) as a TBS ether (TBSOTf, 2,6-lutidine, 97% yield) followed by DDQ-induced benzylic oxidation in the presence of chloroacetic acid (as opposed to acetic acid) (It should be noted that usage of acetic acid as solvent leads to prolonged reaction times and the formation of side products (e.g. the corresponding ketone due to overoxidation) resulting in significantly lower yield.) in CH₂Cl₂ furnished stereoselectively, chloroacetate 22 in 87% yield. Exchange of the chloroacetyl group within the latter with PMB (LiOH in MeOH; then PMBTCA) furnished the desired product 13 in 64% overall yield for the last two steps.

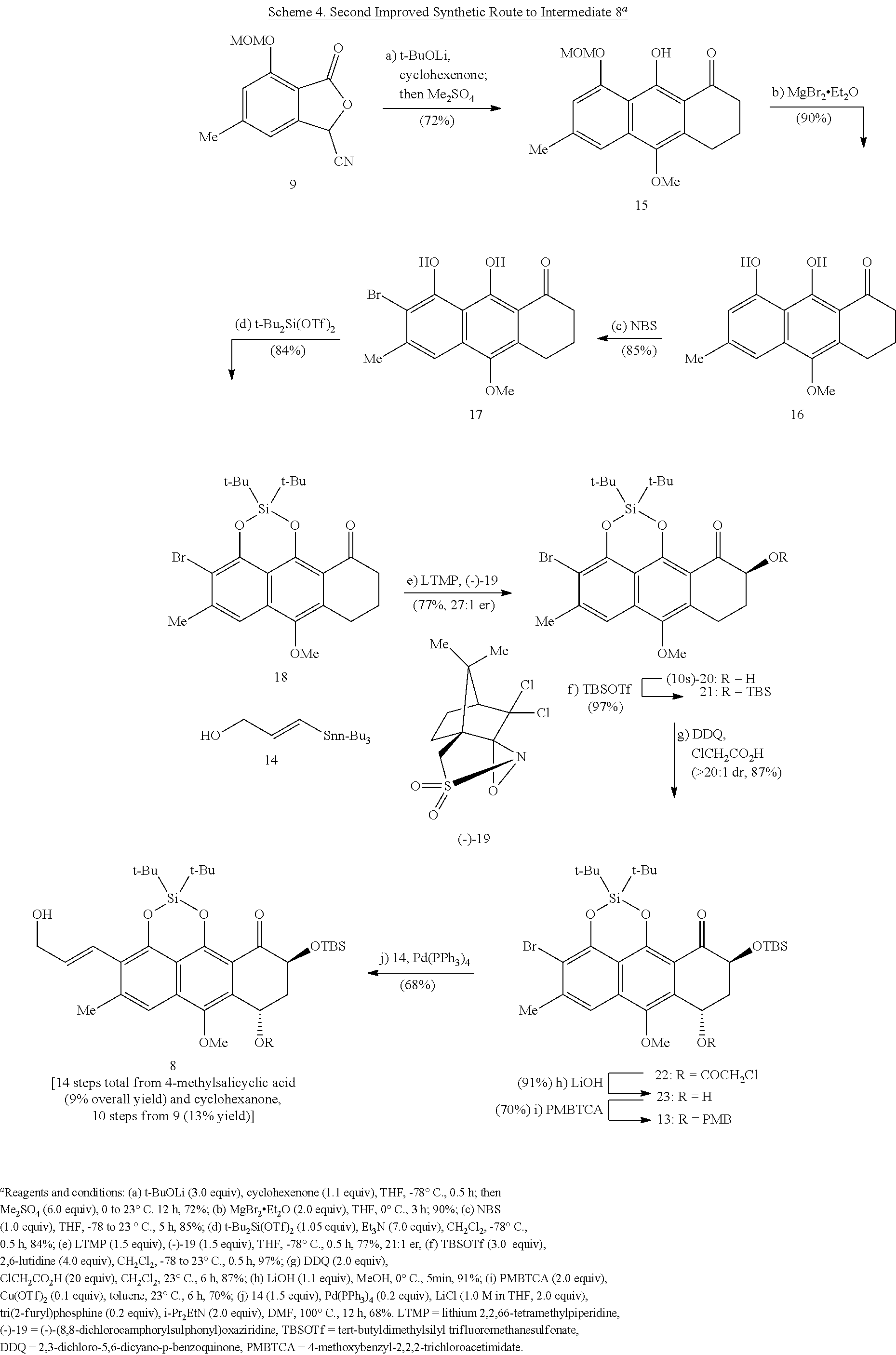

TABLE 1
Optimization of α-Hydroxylation of Bromide 18^{a}

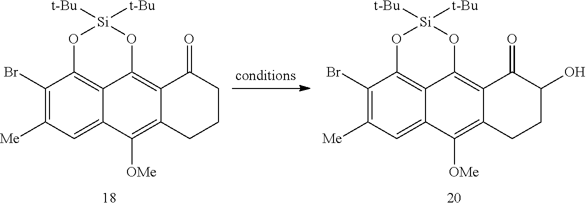 [SMILES: COc1c2c(c3c4c(c(Br)c(C)cc14)O[Si](C(C)(C)C)(C(C)(C)C)O3)C(=O)CCC2 ; COc1c2c(c3c4c(c(Br)c(C)cc14)O[Si](C(C)(C)C)(C(C)(C)C)O3)C(=O)C(O)CC2]

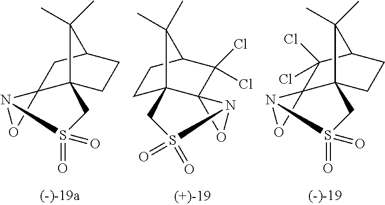

| | | | | | rsm | yield | er |
|---|---|---|---|---|---|---|---|
| entry | oxaziridine | base | ϑ (° C.) | t (h) | (%)^{b} | (%)^{c} | (R):(S)^{d} |
| 1 | (−)-19a | NHMDS | −78→0 | 2 | 41 | 33 | 1.9:1 |
| 2 | (+)-19 | NHMDS | −78 | 1 | 30 | 54 | 21:1 |
| 3 | (+)-19 | LHMDS | −78 | 1 | 48 | 55 | 21:1 |
| 4 | (+)-19 | LDA | −78 | 0.5 | 9 | 66 | 14:1 |
| 5^{e} | (+)-19 | LDA | −78 | 0.5 | 30 | 37 | 2:1 |
| 6 | (+)-19 | LTMP | −78 | 0.5 | 5 | 75 | 17:1 |
| 7 | (−)-19 | LTMP | −78 | 0.5 | 7 | 77 | 1:27 |

^{a}Reactions were carried out on 0.10 mmol scale, with 1.5 equiv base and 1.5 equiv oxaziridine in THF;
^{b}recovered starting material;
^{c}isolated yield;
^{d}absolute configuration of 20 was determined by Mosher ester analysis;
^{e}HMPA as additive.

### 1. Example 2—Design and Synthesis of Trioxacarcin Analogues Trx1-Trx25

Despite their common core scaffold, the naturally occurring trioxacarcins (e.g. 2-5, Scheme 1) are reported to exhibit broadly varying degrees of potencies against different cancer cell lines (Fujimoto and Morimoto, 1983). Reasoning that even small variations of functional groups on the central trioxacarcin core may lead to interesting analogues, synthesis of a number of compounds with relatively small structural changes from the parent compounds was desired (i.e., 2-5, Scheme 1). To this end, advanced intermediates 24-26, 30 and 33 (Schemes 5 and 6, respectively) encountered in our previously accomplished syntheses of trioxacarcins 1-5 (Nicolaou et al., 2016a) were employed. Thus, and as shown in Scheme 5A, mono-glycosylated TBS-derivative 24 (Nicolaou et al., 2016a) was desilylated (Et₃N.3HF, 83% yield) to afford trioxacarcin analogue Trx1. Similarly, TBS-derivative 25 (Nicolaou et al., 2016a) was desilylated to give trioxacarcin analogue Trx2 (Et₃N.3HF, 71% yield) as shown in Scheme 5B.

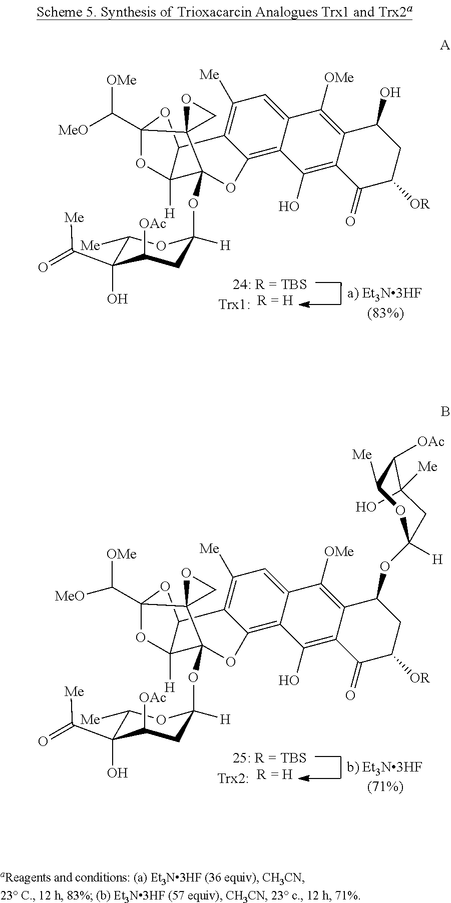

Scheme 6 summarizes the syntheses of trioxacarcin analogues Trx3, Trx4 (panel A), Trx5 (panel B) and Trx6 (panel C). Thus, removal of the PMB protecting group from 26 (DDQ, 83% yield) gave intermediate 27, whose glycosylation with acetylenic glycosyl donor 28 (Nicolaou et al., 2016a) proceeded smoothly under the influence of Ph₃PAuNTf₂ as promoter to afford 29 in 88% yield (Li et al., 2008; Yang et al., 2009; Li et al., 2010; Zhang et al., 2011; Tang et al., 2013; Nie et al., 2014). Upon treatment with Et₃N.3HF in MeCN, TBS ether 29 was converted to Trx3 (85% yield). Exposure of the latter to K₂CO₃ in MeOH then furnished trioxacarcin analogue Trx4 (59% yield). The C7″-epimeric substrate 30 was similarly processed (Scheme 6B) to generate first glycosyl acceptor 31 (DDQ, 75% yield) and then compound 32 (glycosyl donor 28, Ph₃PAuNTf₂, 62% yield) with exclusive α-glycoside bond formation. Finally, removal of the TBS group from 32 (Et₃N.3HF) furnished analogue Trx5 in 89% yield as shown in Scheme 6B. Scheme 6C summarizes the preparation of analogue Trx6 (Et₃N.3HF, 89%) from its previously synthesized precursor 33 (Nicolaou et al., 2016a).

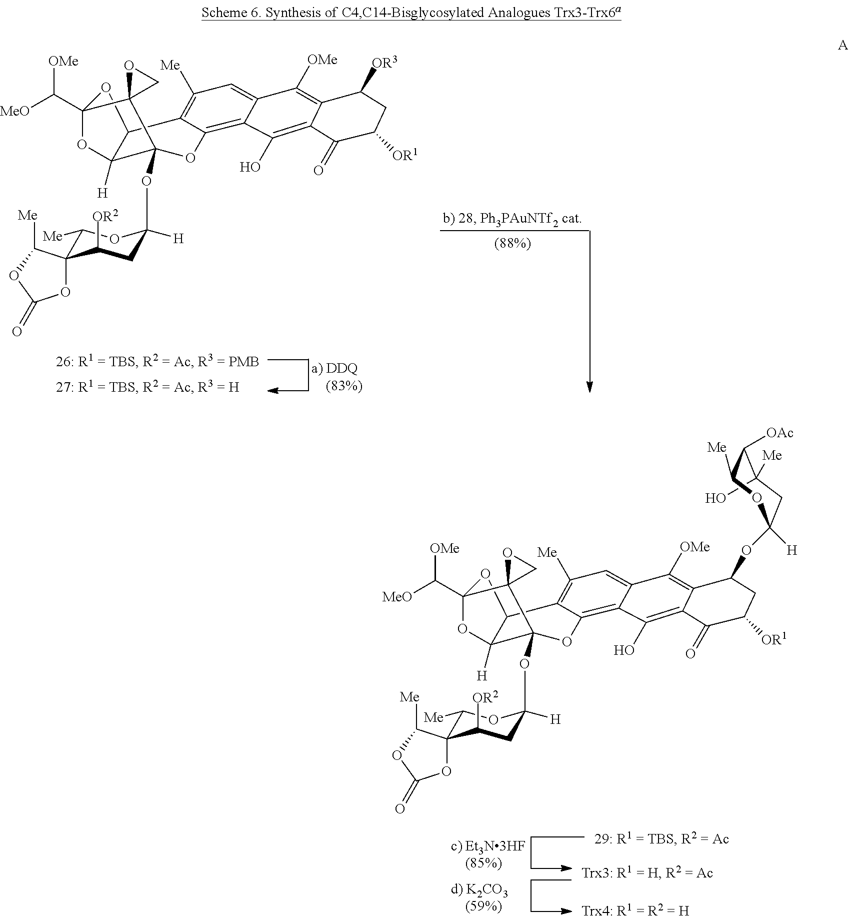

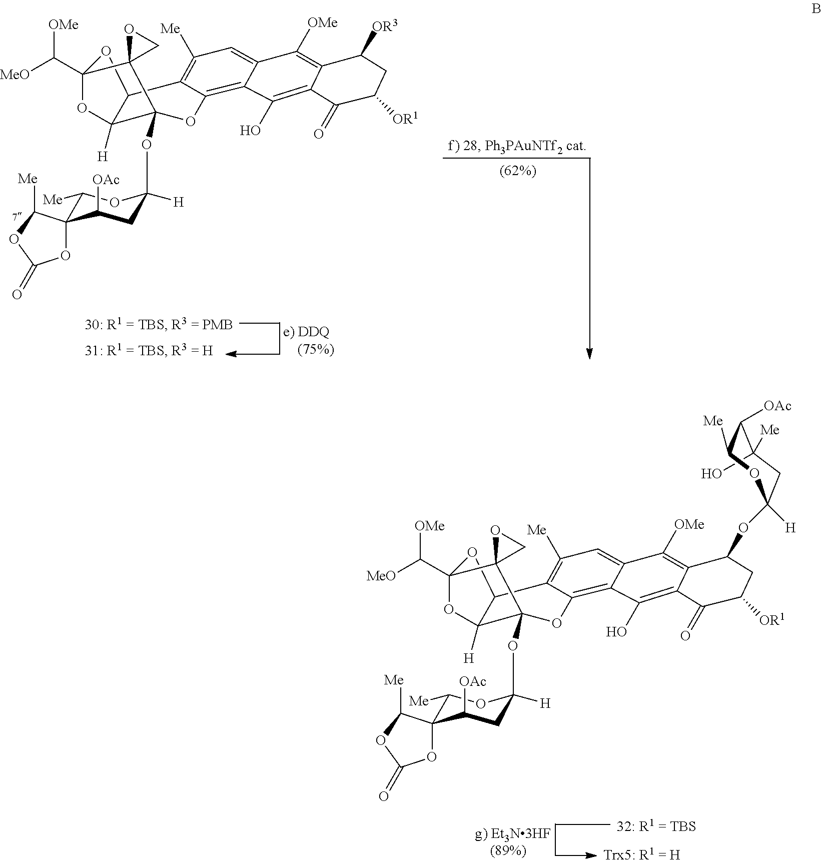

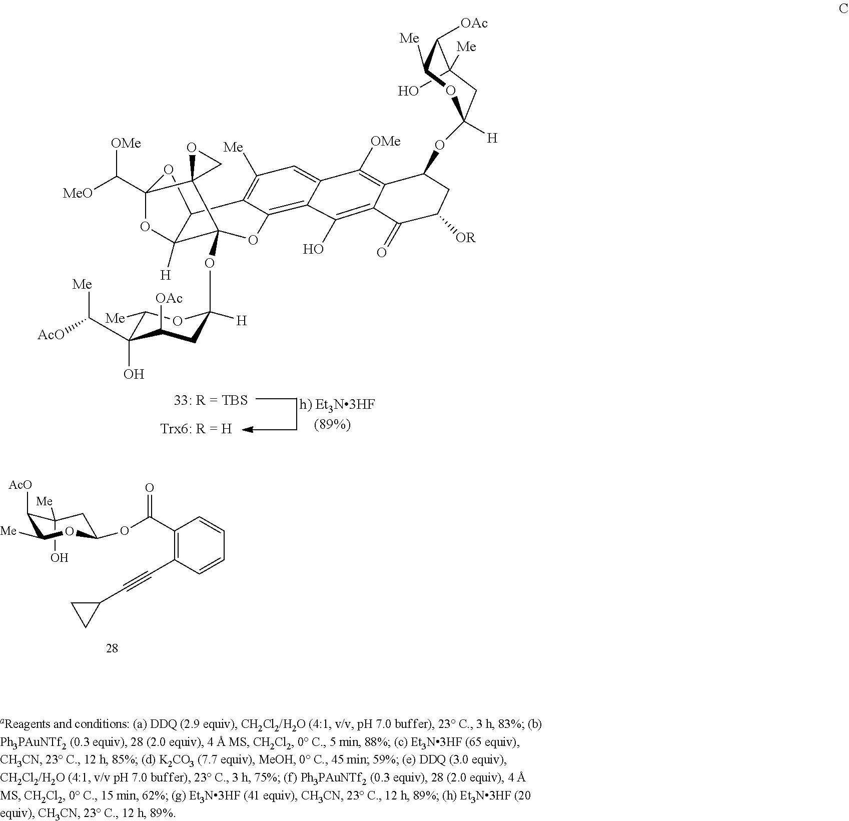

Having prepared these glycosylated and closely related analogues of the naturally occurring trioxacarcins, it was next decided to apply significantly more extensive simplification to the trioxacarcin structure in order to test potency retainment, or even enhancement under such structural modifications. To this end, readily available tricyclic bromoketone 18 (prepared as described above, see Scheme 4) was utilized as the starting point of divergence into a variety of simpler trioxacarcin analogues. Thus, and as shown in Scheme 7 and Table 2, it was found necessary to first optimize the conditions for the coupling of bromide substrate 18 with stannane 14. As summarized in Table 2, a number of palladium catalysts, ligands and bases were tested, leading to the identification of Pd(PPh₃)₄ catalyst, P(2-furyl)₃ ligand, and i-Pr₂EtN/LiCl base combination as the optimal conditions for this reaction (Table 2, entry 8 and Scheme 7, 18→34, 74% yield). Parenthetically, it should be noted these optimized conditions, ultimately, were also employed for the coupling of substrate 8, corresponding to the naturally occurring trioxacarcins, with stannane 14 as described above (see Schemes 3 and 4). With allylic alcohol 34 now available (see Scheme 7), the next step was its oxidation to the corresponding cinnamaldehyde derivative 35, a task achieved through the use of NMO and TPAP catalyst (82% yield). The latter was then subjected to asymmetric epoxidation with Jsrgensen catalyst 36 (Marigo et al., 2005) to afford epoxy aldehyde 37. Baylis-Hillman reaction of crude, and rather labile, aldehyde 37 with enone 38a (DABCO, p-nitrophenol) resulted in the formation of the corresponding alcohol (39a, mixture of diastereoisomers, ca. 5:1 dr), which was protected as a TMS ether (40a, TMSCl, imidazole, −78° C., 35% overall yield for the three steps from α,β-unsaturated aldehyde 35). On exposure to BF₃.Et₂O (CH₂C₂, −30→0° C.), epoxy enone 40a led to polycyclic system 41a in 48% yield (Scheme 7).

TABLE 2
Optimization of Stille Coupling of Bromide 18 with Stannane 14^{a}

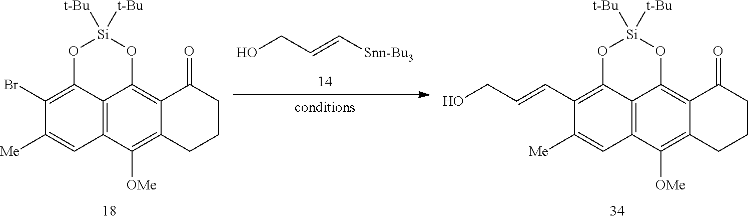

| entry | catalyst | ligand | base | solvent | ϑ (° C.) | yield^{b} (%) |
|---|---|---|---|---|---|---|
| 1 | Pd(PPh₃)₂Cl₂ | — | CsF₂ | DMF | 100 | N.R. |
| 2 | Pd₂(dba)₃ | (o-MePh)₃P | i-Pr₂EtN | DMF | 100 | 15 |
| 3 | Pd(PPh₃)₄ | (o-MePh)₃ | i-Pr₂EtN | DMF | 100 | 22 |
| 4 | Pd(PPh₃)₄ | AsPh₃ | i-Pr₂EtN | DMF | 100 | 31 |
| 5 | Pd(PPh₃)₄ | P(2-furyl)₃ | i-Pr₂EtN | DMF | 100 | 51 |
| 6 | Pd(PPh₃)₄ | P(2-furyl)₃ | i-Pr₂EtN | DMF | 100 | trace |
| 7 | Pd(PPh₃)₄ | P(2-furyl)₃ | i-Pr₂EtN | dioxane | 80 | 12 |
| 8 | Pd(PPh₃)₄ | P(2-furyl)₃ | i-Pr₂EtN, LiCl | DMF | 100 | 74 |

^{a}Reactions were carried out on 0.5-2.0 mmol scale, with 10 mmol % catalyst for 12 h;
^{b}isolated yield.

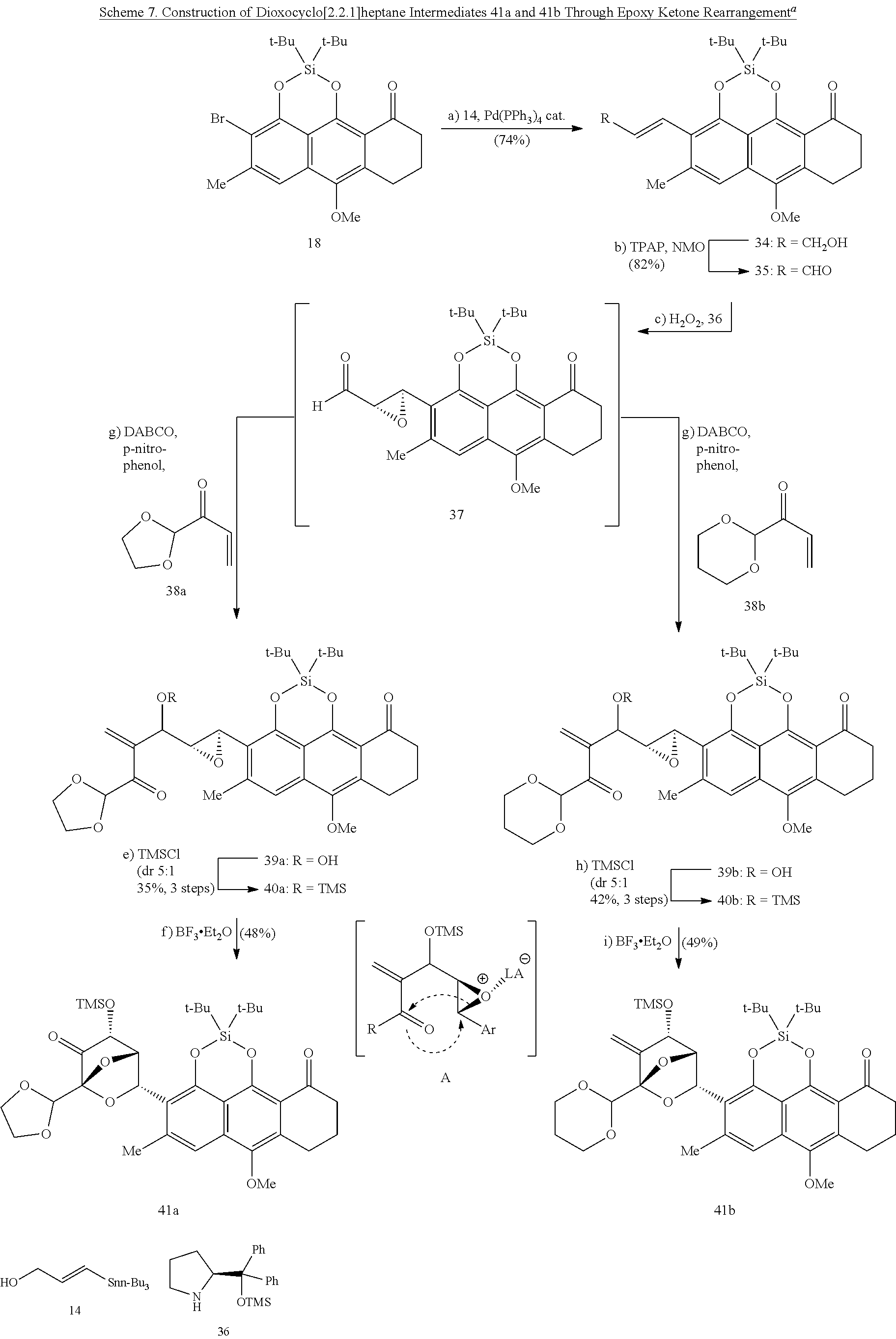

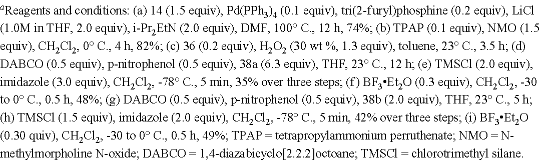

The newly formed 2,7-dioxobicyclo[2.2.1]heptane structural motif within the latter product was apparently formed through a rearrangement involving activation of the epoxide moiety, followed by attack from the carbonyl oxygen, epoxide rapture, and attack of the so-generated alkoxide onto the incipient oxonium species A (in brackets, Scheme 7), as previously described (Nicolaou et al., 2015; Nicolaou et al., 2016a; Gaoni et al., 1968; Wasserman et al., 1986; Naruse et al., 1988; Evans et al., 1991) A similar sequence from epoxy aldehyde 37 and 38b involving Baylis-Hillman reaction/TMS protection (40b via 39b, mixture of diastereoisomers, ca. 5:1 dr, 42% overall yield from 35 for the three steps) and epoxy ketone rearrangement (BF₃.Et₂O) furnished advanced intermediate 41b (6-membered ring ketal) in comparable (49% yield), if not higher, overall yield from 35 than the corresponding sequence involving enone 38a (5-membered ring ketal, see Scheme 7).

Scheme 8 summarizes the conversion of advanced intermediate 41a to trioxacarcin analogue Trx8. Thus, treatment of 41a with TFA led to allylic alcohol 42a through selective desilylation (72% yield). The latter was reacted with NMO/OsO₄ cat. to afford the expected triol 43a, from which the primary tosylate 44a was generated (TsCl, Et₃N, DMAP cat.). Exposure of this dihydroxy tosylate to K₂CO₃ in MeOH furnished epoxy alcohol 45a in 78% overall yield for the three steps from 42a. Oxidation of the latter compound with NMO/TPAP cat. gave, in 94% yield, keto epoxide 46a, whose desilylation (Et₃N.3HF) led to the coveted trioxacarcin analogue Trx8 in 89% yield. It should be noted that attempts to obtain hydroxy epoxide 45a directly from 42a did not prove fruitful, and thus the longer sequence (i.e., 42a→43a→44a→45a) shown in Scheme 8.

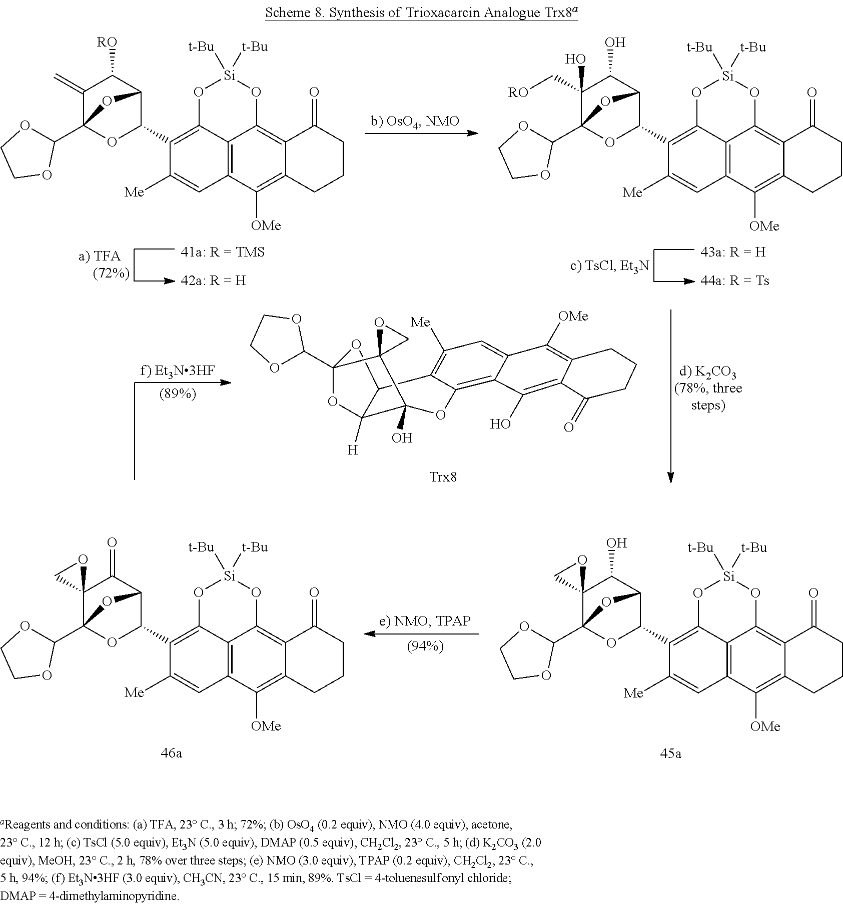

Analogue Trx9 was synthesized from Trx8 through reaction with Mel in the presence of Ag₂O and CaSO₄ (63% yield) as shown in Scheme 9, while Trx10 carrying a methoxymethyl (MOM) group at the anomeric center was prepared from the same starting material (i.e., Trx8) by reacting with MOMCl in the presence of i-Pr₂EtN (90% yield, Scheme 9). Continuing the theme of the cyclic ketal as a replacement of the dimethoxy ketal on the “left side” of the trioxacarcin molecule, and with the intention of testing the effect on potency of a basic nitrogen, we undertook the synthesis of aminosugar containing analogue Trx11 as shown in Scheme 9. Thus, analogue Trx8 was glycosylated with Alloc-protected amino carbohydrate donor 49 (prepared in 83% yield from carboxylic acid 48 and Alloc-protected amino sugar 47 (Nicolaou et al., 2011; Nicolaou et al., 2015) as summarized in Scheme 10) through the action of Ph₃PAuOTf as promoter to afford glycoside 50 (68% yield, α-glycoside bond), whose exposure to Pd(PPh₃)₂Cl₂ cat., n-Bu₃SnH and AcOH led to the desired analogue Trx11 in 69% yield (Scheme 9).

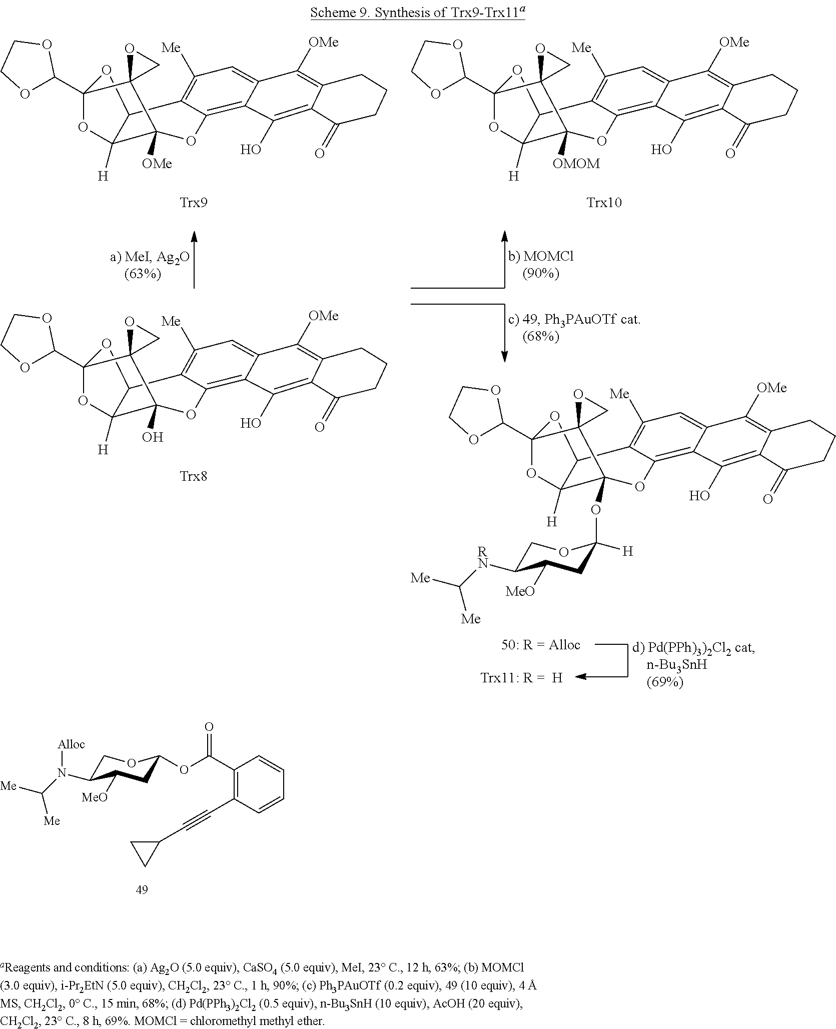

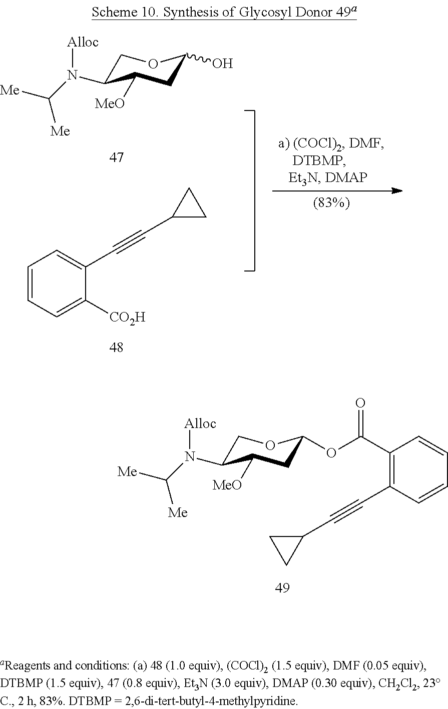

The next series of analogues (Trx12-Trx23) included the 6-membered ring ketal on the “left side” of the molecule. These analogues were synthesized from advanced intermediate 41b whose preparation was discussed above (see Scheme 7). The slight advantage of the route leading to this advanced intermediate, as compared to that leading to its 5-membered ring counterpart (i.e., 41a, Scheme 7), made these analogues more attractive than their 5-membered ring ketal counterparts.

Scheme 11 summarizes the synthesis of trioxacarcin analogue Trx12, which followed the same sequence of reactions as that employed to synthesize its 5-membered ring relative (Trx8, Scheme 8) and proceeded in similar yields.

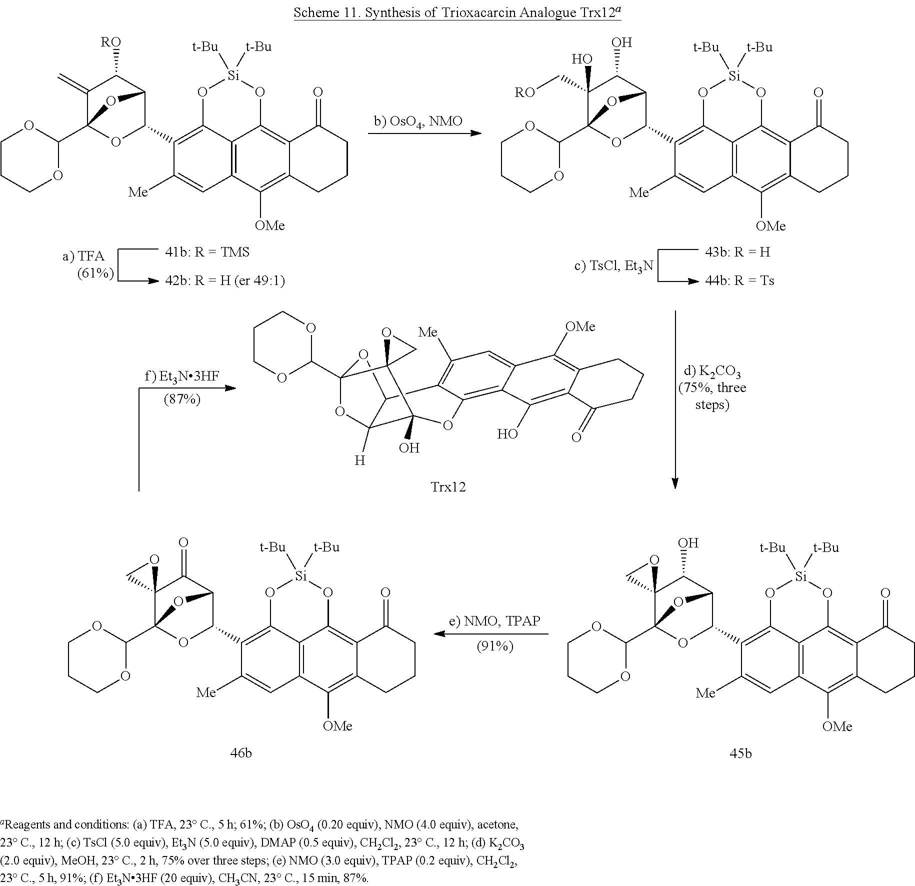

Scheme 12 depicts the synthesis of trioxacarcin analogues Trx13-Trx17 in which the anomeric hydroxyl group was capped with a variety of groups, namely methyl (Trx13), acetate (Trx14), methoxymethyl (MOM, Trx15), 2-methoxyethoxymethyl (MEM, Trx16), and allyl (Trx17) (for reagents, conditions, and yields, see Scheme 12).

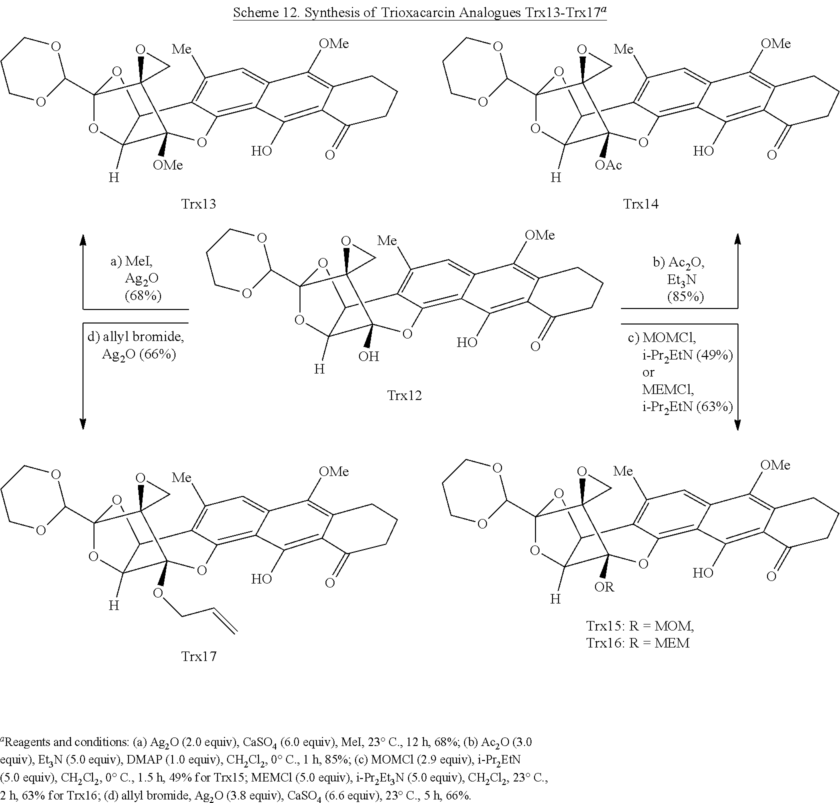

Scheme 13 summarizes the synthesis of monoglycosylated trioxacarcin analogues Trx18-Trx23 from analogue Trx12 and glycosyl donors 49, 51, and 52, (Nicolaou et al., 2016a) respectively. Thus, reaction of Trx12 with glycosyl donor 49 in the presence of Ph₃PAuOTf as promoter furnished Alloc-protected α-glycoside Trx18 (90% yield), from which the desired analogue Trx19 was generated upon treatment with Pd(PPh₃)₂Cl₂ cat., n-Bu₃SnH and AcOH (87% yield). Glycosylation of Trx12 with glycosyl donor 51 under the same conditions produced analogue Trx20 (88% yield), from which Trx21 was generated through acetate cleavage as induced by K₂CO₃ in MeOH (91% yield). Analogue Trx22 was selectively synthesized from Trx12 through coupling with glycosyl donor 52 under the same gold-promoted conditions (Ph₃PAuOTf, 88% yield). Analogue Trx23 was finally prepared from Trx22 by exposure to NaH in ethylene glycol (acetate and carbonate cleavage, 88% yield) as shown in Scheme 13.

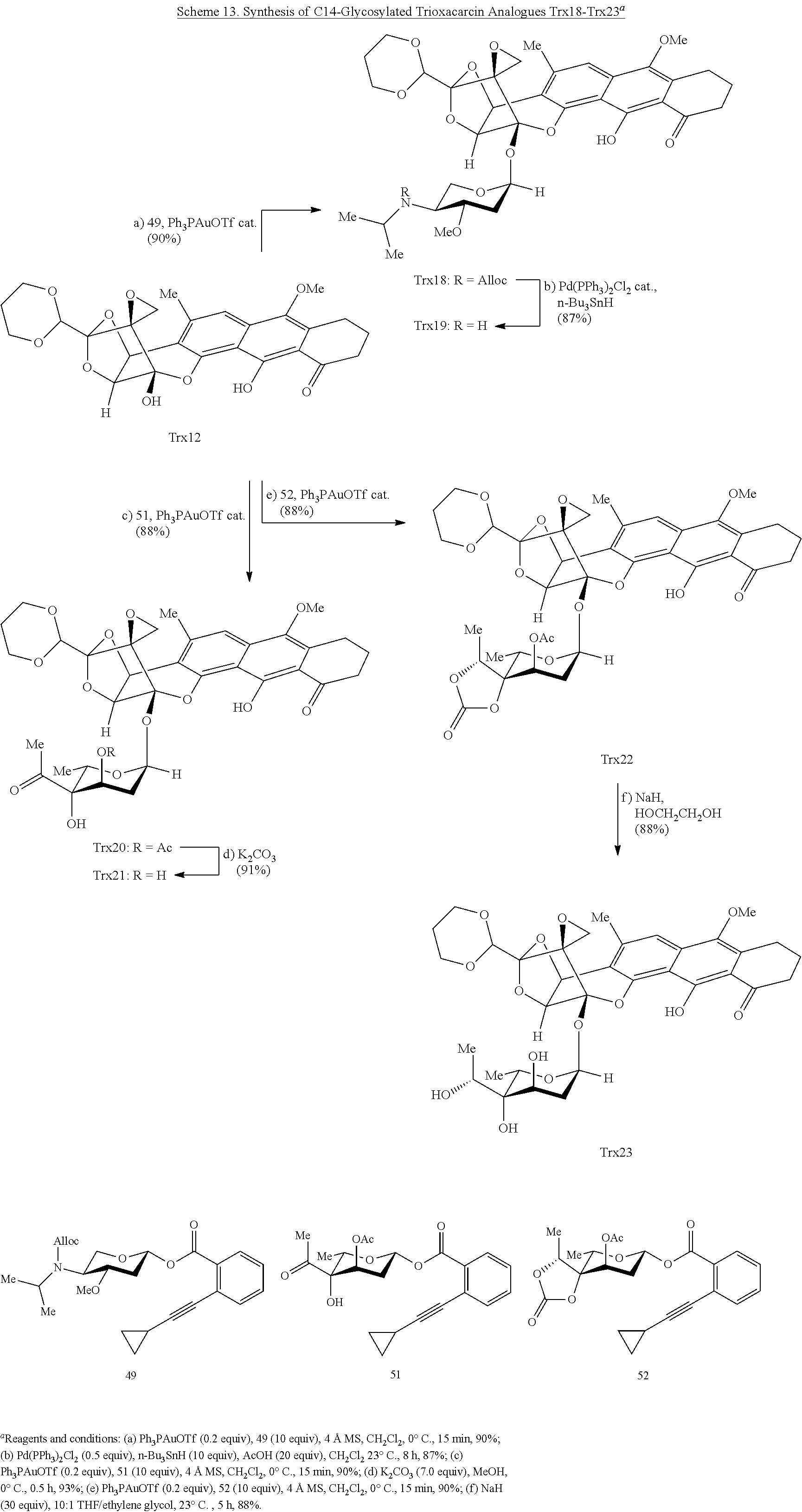

In order to test the biological activity of dimeric trioxacarcins, dimerization of allyl analogue Trx17 was attempted through olefin metathesis as shown in Scheme 12. Thus, exposure of Trx17 to Grubbs I cat. (Grellepois et al., 2005) led to a mixture of (Z) and (E) analogues Trx24 and Trx25 [(Z):(E) ca. 2:1, 47% combined yield, plus 20% recovered starting material], which were chromatographically separated. The two geometrical isomers were distinguished by ¹³C NMR spectroscopic analysis which revealed their identity through their γ-effect (Kleinpeter et al., 2004; Kleinpeter et al., 2005) on their respective ¹³C chemical shifts [major product (Z): δ_{C (allylic)}=61.0 ppm; minor product (E): δ_{C (allylic)}=66.0 ppm]. This technique proved to be preferable since ¹H NMR spectroscopic analysis (i.e. coupling constants of olefinic protons) was not applicable in this case due to the symmetrical nature of these molecules.

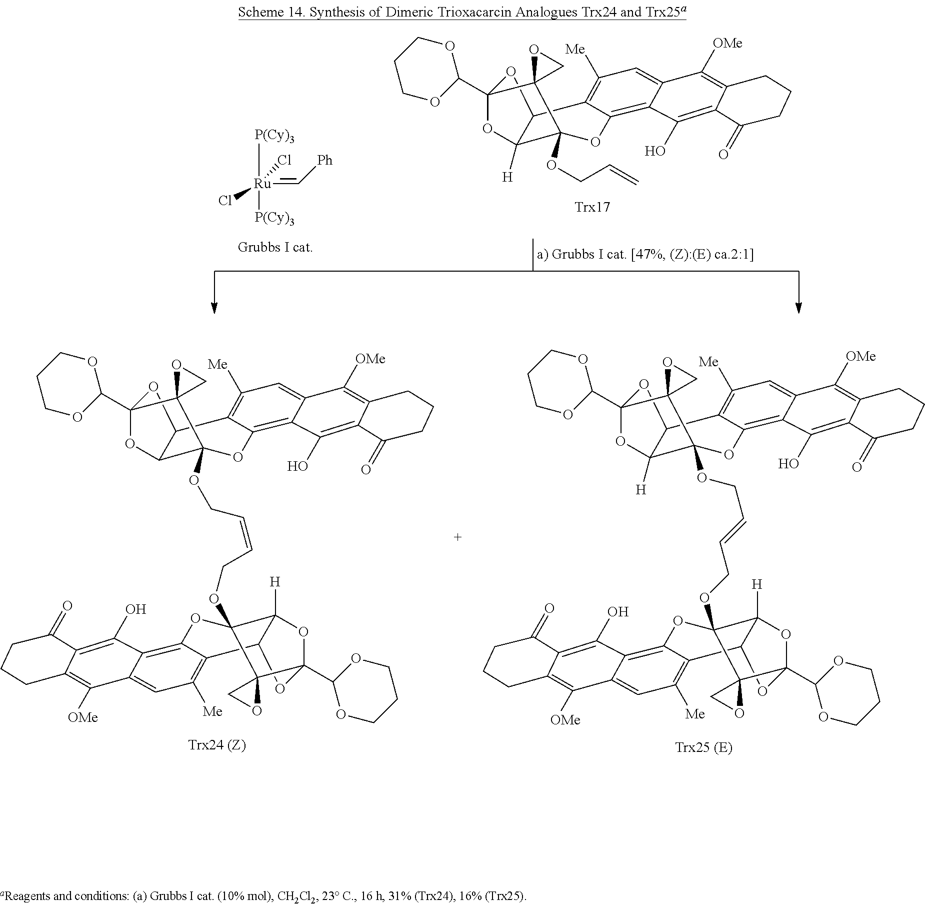

in ethylene glycol (acetate and carbonate cleavage, 88% yield) as shown in Scheme 13.

### 2. Example 3—Design and Synthesis of Anthraquinone Analogues Trx26-Trx33

The idea of producing anthraquinone type analogues of the trioxacarcins starting from the simple analogues Trx12 and Trx3 through aromatizationoxidation procedures was then considered.

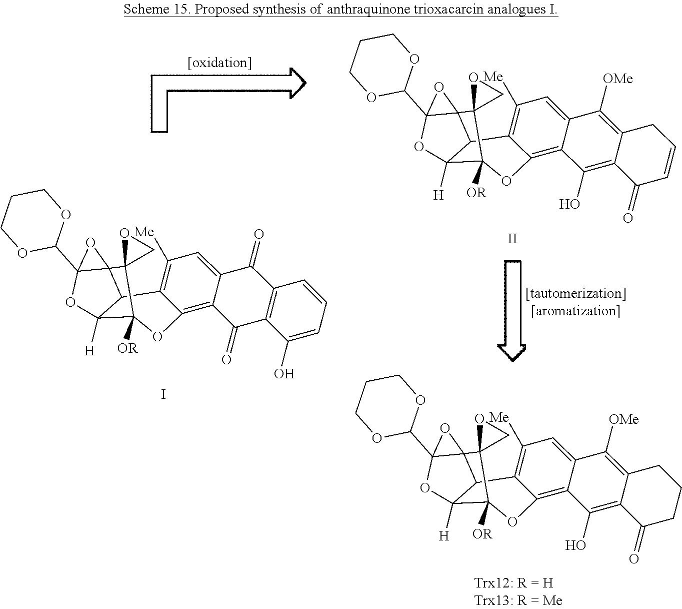

It was reasoned that such compounds may provide a better potential intercalation fit with double stranded DNA, the target of not only the trioxacarcins (Pfoh et al., 2008; Fitzner et al., 2008) but also of other cytotoxic natural products such as doxorubicin (Perez-Arnaiz et al., 2014; Bellamy et al., 1988; Muller et al., 1997) and uncialamycin (Davies et al., 2005). It was projected that these trioxacarcin structures (i.e., I, Scheme 15) may be accessible from Trx12 and/or Trx13 via transient intermediate enone II as shown in retrosynthetic format in Scheme 15. Thus, it was anticipated that oxidative conversion of Trx12/Trx13 to enone II would be followed by aromatization/oxidation (I) to afford the desired anthraquinone system.

Initial attempts to introduce a phenylseleno group adjacent to the carbonyl group failed, presumably due to the deactivating effect on the phenolic OH by the carbonyl moiety exerted through hydrogen bonding. This obstacle was overcome through initial protection of the phenolic group as shown in Scheme 13. Thus, reaction of Trx 12 with Ac₂O, in the presence of Et₃N and catalytic amounts of DMAP, resulted in acetylation of both the phenolic moiety and the tertiary hydroxyl group of the molecule to afford diacetate Trx26 (67% yield). Similar treatment of Trx13 furnished monoacetate Trx27 in 96% yield as shown in Scheme 13. Exposure of Trx26 to 1.2 equiv of PhSeCl followed by treatment of the resulting phenylselenide with H₂O₂(Sharpless et al., 1973) furnished, to our pleasant surprise, directly anthraquinone Trx28 in 48% overall yield. Similar treatment of Trx27 led to anthraquinone Trx29 in 56% overall yield (Scheme 16). Trioxacarcin analogues Trx30 and Trx31 were generated from their acetate precursors Trx28 (78% yield) and Trx29 (83% yield), respectively, through hydrolysis using aq. LiOH as depicted in Scheme 16. Interestingly, when ketone trioxacarcins Trx26 and Trx27 were individually treated with excess PhSeCl, the chloro acetoxy anthraquinone trioxacarcins Trx32 (46% yield) and Trx33 (42% yield) were directly, and respectively, obtained as shown in Scheme 16.

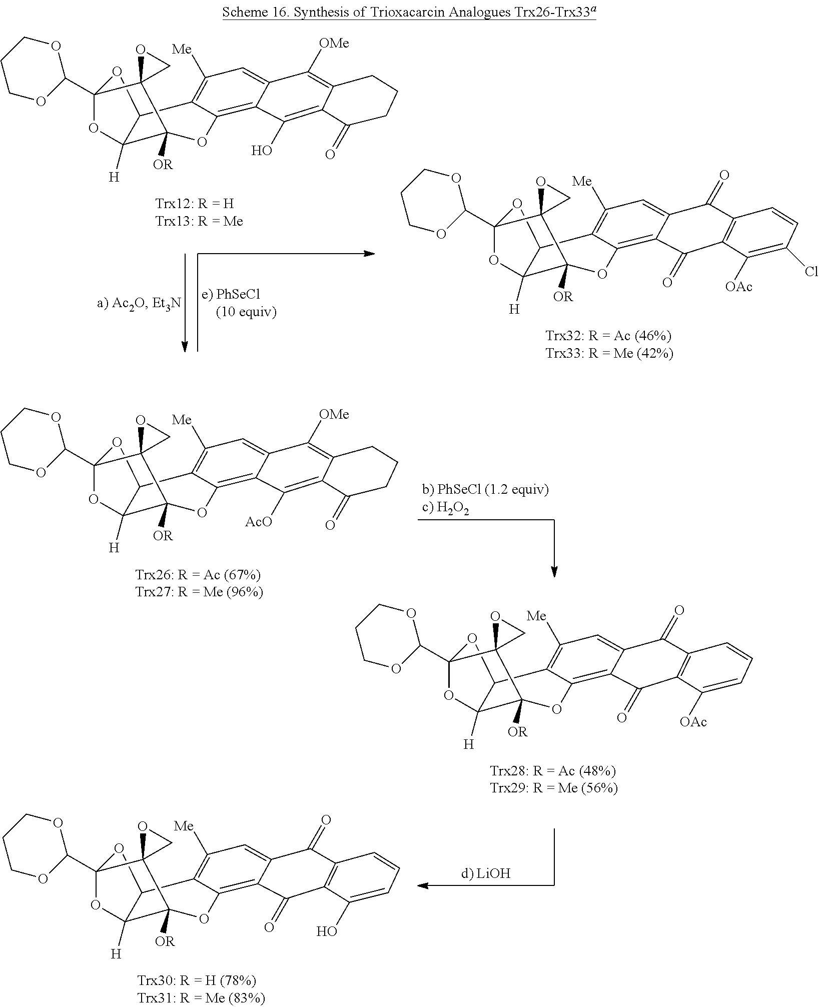

“Reagents and conditions: (a) Ac₂O (58 equiv), Et₃N (320 equiv), DMAP (0.5 equiv), CH₂Cl₂, 23° C., 12 h, 67% for Trx26; 96% for Trx27; (b) PhSeCl (1.2 equiv), EtOAc, 23° C.; (c) H₂O₂ (30 wt % in H₂O, 18 equiv), CDCl₃, air, 0 to 23° C., 2 h, 48% over two steps for Trx28, 56% over two steps for Trx29; (d) LiOH (1 N in H₂O, 56 equiv), 23° C., 1 h, 78% for Trx30, 83% for Trx31; (e) PhSeCl. (10 equiv), EtOAc, 23° C., 3 d, 46% for Trx32, 42% for Trx33.

The formation of anthraquinone Trx28 from keto acetate Trx26 upon sequential treatment with PhSeCl and H₂O₂ is presumed to proceed through the cascade of reactions shown in Scheme 17. Thus, α-phenylselenylation of Trx26, followed by oxidation of the resulting phenyl seleno ketone leads first to phenylselenoxide Trx26a and thence to enone Trx26b through spontaneous syn-elimination (see Scheme 14). Tautomerization/aromatization of the latter intermediate then leads to phenol acetate Trx26c, which is apparently readily transformed to anthraquinone Trx28 via sequential acetate migration and air oxidation. Intermediates Trx26c and Trx26d may exist in equilibrium, which is driven toward Trx26d by the ease of oxidation of the latter. A similar mechanism is assumed for the generation of Trx29 from Trx27 under the same conditions (see Schemes 16 and 17).

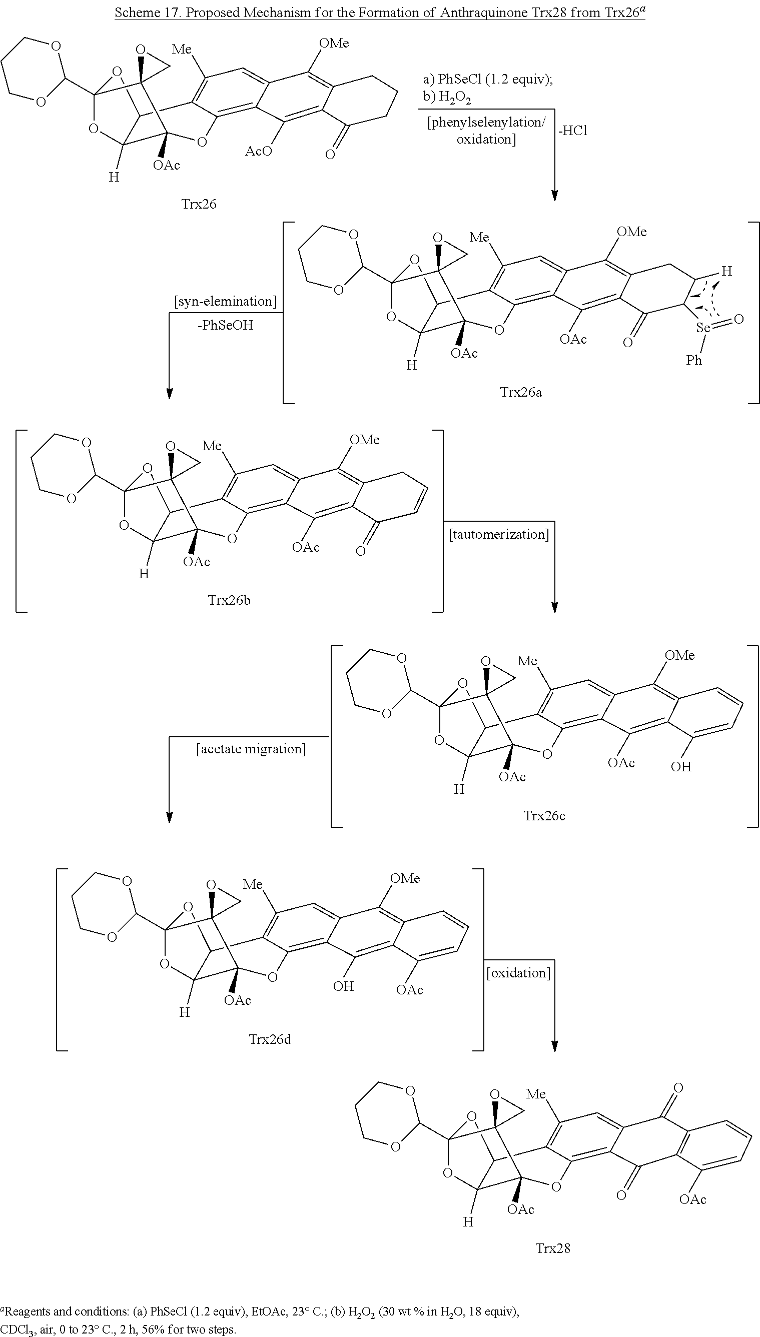

To explain the direct generation of chloro acetoxy quinone Trx33 from keto acetate Trx27 upon exposure to excess PhSeCl (Scheme 16), two conceivable mechanisms were proposed, as shown in Scheme 18 (pathways a and b). It was reasoned that the question as to which of the two possible pathways (a: arrows in red; or b: arrows in green) is operating could be answered through labeling one of the two seleno groups involved in the reaction (Scheme 18) with a methyl group by using tolylselenenyl chloride to initiate the process, and employing phenylselenyl chloride to complete the reaction as demonstrated with model system III shown in Scheme 19. Thus, treatment of the readily accessible tolylseleno ketone III (for preparation, see Supporting Information) with 2.0 equiv of PhSeCl in the presence of CSA (1.0 equiv) led to labile chloro tolylselenide VIIIa (via intermediate IV, ˜25% yield plus 60% recovered starting material) and PhSeSePh (exclusively; no TolSeSePh detected) as expected from mechanism b (path b, green) rather than VIIIb or V and ToSeSePh and/or PhSeSePh as expected had the alternative mechanism (path a, red) been operating (see Scheme 19). Products VIIIa (labile) and PhSeSePh was isolated and characterized by NMR spectroscopic and mass spectrometric analysis. Although similar chlorinations have been reported in the past (Tsuda et al., 1985; Abul-Hajj, 1986; Tsuda et al., 1991; Kende et al., 2002) and tentative mechanisms proposed, the latter lack experimental evidence. The present support for pathway b involving a chloronium species in the chlorination of tolylselenide III (Scheme 19) and phenylselenide Trx27e (Scheme 18) may explain previous observations (Tsuda et al., 1985; Abul-Hajj, 1986; Tsuda et al., 1991; Kende et al., 2002) and inspire new chemistry.

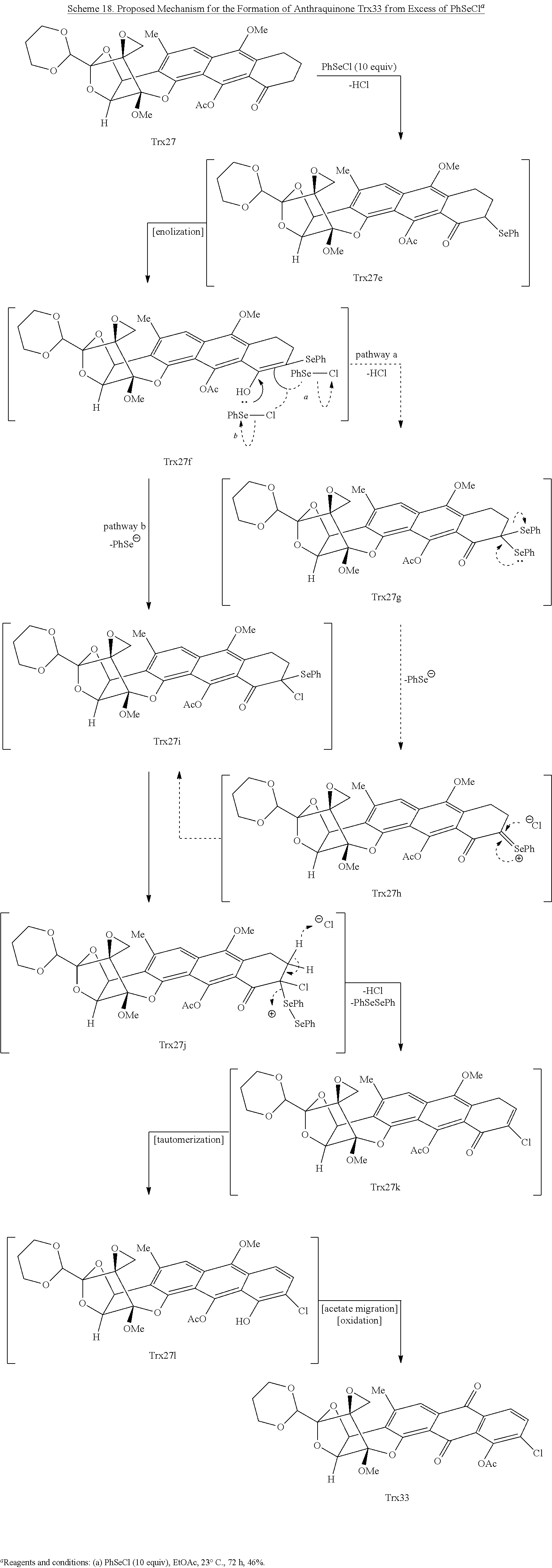

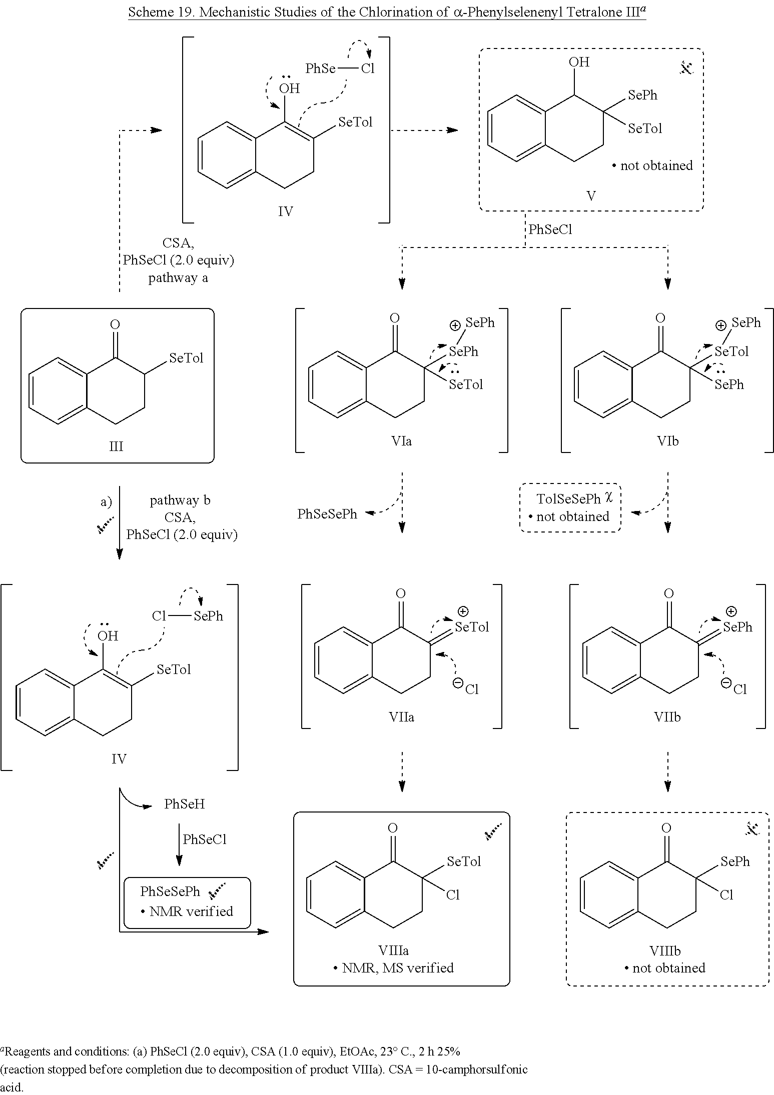

### 3. Example 4—Biological Evaluation of Synthesized Trioxacarcin Analogues

The synthesized trioxacarcin analogues were tested against the cancer cell lines MES SA (human uterine sarcoma), MES SA DX (human uterine sarcoma cell line with marked multidrug resistance) and HEK 293T (human embryonic kidney cancer cell line), alongside MMAE (monomethyl auristatin E) and naturally occurring trioxacarcins DC-45-A1 (2), A (3), D (4) and C (5) as standards for comparison purposes.

As can be seen in Table 3, while monoglycosylated trioxacarcin analogue Trx1 showed only modest activity against the tested cancer cell lines, the bis-glycosylated analogues Trx2-Trx7 demonstrated potent cytotoxic properties [comparable to those of the natural trioxacarcins (2-5)] against the MES SA and HEK 293T cell lines, with Trx2 exhibiting the most impressive potency against MES SA (IC₅₀=2.02 nM) and HEK 293T (IC₅₀=2.82 nM) [but not against the multidrug resistant cell line MES SA DX (IC₅₀>1000 nM)]. More importantly, however, a number of the next series of analogues (Trx8-Trx10), possessing a significantly simpler structure than the natural trioxacarcins and in which the dimethoxy ketal on the “left side” of the molecule had been replaced with a 5-membered ring cyclic ketal, exhibited comparable cytotoxicities to the most potent naturally occurring trioxacarcin tested [i.e., trioxacarcin A (3), Table 3]. Furthermore, an interesting trend within this subgroup of analogues points to the importance of the capping of the tertiary hydroxy group, with Trx9 and Trx10 featuring ether moieties at this position and exhibiting significantly higher potencies as compared to Trx8, their parent compound possessing a free tertiary hydroxy group. Impressively, trioxacarcin analogue Trx11, carrying an amino sugar onto its tertiary hydroxyl group, exhibited even more potent cytotoxic properties (MES SA: IC₅₀=1.07 nM; MES SA DX: IC₅₀=3.03 nM; HEK 293T: IC₅₀=0.92 nM) than its siblings (i.e, Trx9 and Trx10), while showing comparable activity against the multidrug resistant cell line MES SA DX (cf. Trx18 with Trx19, Table 3).

TABLE 3
Cytotoxicity Data Against the Cancer Cell
Lines^{a} MES SA, MES SA DX, and HEK 293T for Trioxacarcin
Analogues Trx1-Trx33 (IC₅₀ Values in nM)^{b}

| | MES | MES | HEK |
|---|---|---|---|
| Compound | SA | SA DX | 293T |
| MMAE^{c} | 0.06 | >70 | 0.08 |
| DC-45-A2 (1) | >2500 | >2500 | >2500 |
| DC-45-A1 (2) [KCN-Triox 12] | 18.08 | >1000 | 14.89 |
| Trioxacarcin A (3) [KCN-Triox 13] | 0.74 | 203.5 | 0.702 |
| Trioxacarcin D (4) [KCN-Triox 14] | 11.06 | >1000 | 8.02 |
| Trioxacarcin C (5) [KCN-Triox 22] | 6.09 | >500 | 5.25 |
| Trx1 [KCN-Triox 15] | 157.4 | >1000 | 95.4 |
| Trx2 [KCN-Triox 16] | 2.02 | >1000 | 2.82 |
| Trx3 [KCN-Triox 17] | 7.22 | >500 | 10.14 |
| Trx4 [KCN-Triox 18] | 8.8 | >500 | 7.92 |
| Trx5 [KCN-Triox 21] | 22.18 | >500 | 24.13 |
| Trx6 [KCN-Triox 19] | 8.8 | >500 | 11.4 |
| Trx7 [KCN-Triox 20] | 10.73 | >500 | 7.72 |
| Trx8 [KCN-Triox 30] | 51.87 | 49.33 | 30.71 |
| Trx9 [KCN-Triox 31] | 4.67 | 2.63 | 2.04 |
| Trx10 [KCN-Triox 32] | 3.88 | 2.18 | 1.51 |
| Trx11 [KCN-Triox 29] | 1.07 | 3.03 | 0.92 |
| Trx12 [KCN-Triox 7] | 3.72 | 5.72 | 2.46 |
| Trx13 [KCN-Triox 8] | 0.56 | 0.42 | 0.40 |
| Trx14 [KCN-Triox 9] | 6.11 | 17.42 | 6.89 |
| Trx15 [KCN-Triox 10] | 1.82 | 2.2 | 1.34 |
| Trx16 [KCN-Triox 11] | 1.56 | 4.1 | 1.09 |
| Trx17 [KCN-Triox 34] | 4.91 | 3.48 | 2.77 |
| Trx18 [KCN-Triox 27] | >1000 | >1000 | 40.24 |
| Trx19 [KCN-Triox 28] | 0.96 | 70.65 | 0.77 |
| Trx20 [KCN-Triox 24] | 5.14 | 52.4 | 4.31 |
| Trx21 [KCN-Triox 23] | 1.47 | 4.67 | 1.26 |
| Trx22 [KCN-Triox 25] | 5.74 | 56.94 | 4.2 |
| Trx23 [KCN-Triox 26] | 5.85 | 33.7 | 2.74 |
| Trx24 [KCN-Triox 35] | >1000 | >1000 | 115.3 |
| Trx25 [KCN-Triox 36] | 235 | 429 | 105 |
| Trx26 [KCN-Triox 39] | 67.44 | 60.29 | 11.62 |
| Trx27 [KCN-Triox 33] | 2.15 | 1.43 | 1.24 |
| Trx28 [KCN-Triox 40] | 11.08 | 11.86 | 11.21 |
| Trx29 [KCN-Triox 37] | 1.44 | 1.05 | 1.87 |
| Trx30 [KCN-Triox 41] | 42.01 | 37.81 | 50.52 |
| Trx31 [KCN-Triox 38] | 1.09 | 0.65 | 1.44 |
| Trx32 [KCN-Triox 42] | 5.96 | 4.41 | 6.73 |
| Trx33 [KCN-Triox 43] | 54.27 | 33.16 | 38.77 |

^{a}MSE SA = uterine sarcoma cell line; MES SA DX = MES SA cell line with marked multidrug resistance; HEK 293T = human embryonic kidney cancer cell line.
^{b}IC₅₀ is the 50% inhibitory concentration of the compound against cell growth;
^{c}MMAE = monomethyl auristatin E. Data obtained at AbbVie Stemcentrx.

The 6-membered ring ketal analogue series Trx13-Trx17 proved even more impressive, not only because of their relative structural simplicity and accessibility but also for leading to the identification of even more potent compounds. Thus, while Trx12 with the free hemiketal moiety and Trx14 carrying an acetate group at this tertiary position proved the least potent of the series, the remaining members of the group showed to be highly potent in all three assays, with Trx13 representing the most potent of all (Trx13: MES SA: IC₅₀=0.53 nM; MES SA DX: IC₅₀=0.38 nM; HEK 293T: IC₅₀=0.50 nM). The cases of Trx18 (MES SA: IC₅₀>1000 nM; MES SA DX: IC₅₀>1000 nM; HEK 293T: IC₅₀=40.24 nM) and Trx19 (MES SA: IC₅₀=0.96 nM; MES SA DX: IC₅₀=70.65 nM; HEK 293T: IC₅₀=0.77 nM) revealed the enhancing role of a basic nitrogen in the molecule and the suppressing effect of its protecting group (Alloc), as evidenced from their distinctively different potencies (see Table 3).

Analogues Trx20-Trx23 also revealed potencies in the range of those exhibited by the natural trioxacarcins 3-5 (except the drug resistant cell line MES SA DX, against which they, interestingly, shared higher potencies than their natural counterparts, see Table 1).

The dimeric analogues Trx24 (Z) and Trx25 (E) were notable in that Trx25 (E) exhibited considerably higher potency (MES SA: IC₅₀=235 nM; MES SA DX: IC₅₀=429 nM; HEK 293T: IC₅₀=105 nM) than Trx24 (Z) (MES SA: IC₅₀>1000 nM; MES SA DX: IC₅₀>1000 nM), except for the HEK 293T cell line, against which it demonstrated comparable potency (IC₅₀=115.3 nM) to that of its (Z)-isomeric sibling (Trx25: IC₅₀=105 nM; Trx24: IC₅₀=115.3 nM). These observations may be attributed to the different orientations of their two domains imposed by the (E)- and (Z)-geometries of their olefinic bonds. Interestingly, acetylation of the phenolic group of the molecule as in analogues Trx26 and Trx27 led to no significant loss of potency against all three cell lines (see Table 2), perhaps suggesting a prodrug behavior of the former (hydrolysis of the acetate).

The final series of trioxacarcin analogues endowed with an anthraquinone moiety in their structures (Trx28-Trx33) revealed a number of highly potent compounds, with Trx31 demonstrating the most impressive cytotoxicities against all cell lines tested (MES SA: IC₅₀=1.09 nM; MES SA DX: IC₅₀=0.65 nM; HEK 293T: IC₅₀=1.44 nM).

### In Vitro Cytotoxicity Assay Description:

Cells were cultured in a T75 flask to ˜50-80% confluency and harvested with trypsin into a single cell suspension. Five hundred (500) cells per well were seeded in tissue culture plates in 50 μL/well culture media and incubated at 37° C. for 18-24 hours. Compounds were diluted as 400× final desired concentrations in DMSO. Serial dilutions in DMSO were then diluted in culture media for a final DMSO concentration of 0.25% and 50 μl/well of the final dilution was added to the cells (Vf=100 W). Upon plating and treatment, cells were returned to the incubator for an additional 72 hours. CellTiter-Glo reagent was prepared per manufacturer's instructions and added at 100 L/well to the cultures. CellTiter-Glo allows for relative enumeration of metabolically active cells by quantifying intracellular ATP concentrations. After 5 minutes of incubation with CellTiter-Glo at ambient room temperature, 125 μl/well of the Cell Titer Glo/cell lysate solution was transferred into black assay plates, which were then read in a luminometer within 30 minutes. Luminescence readings obtained from cultures that did not receive any treatment (cell culture media only) were set as 100% control and all other luminescence values were normalized to these controls (e.g., Normalized RLU, relative luminescence unit).

### Cell Lines:

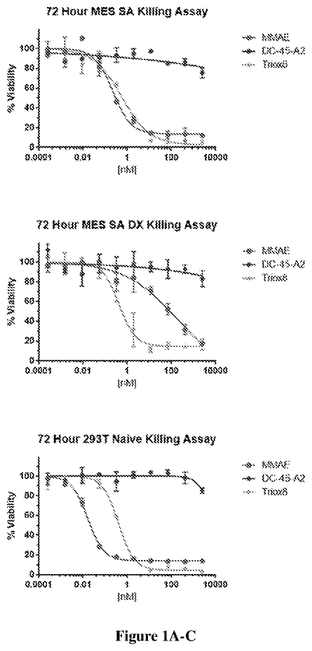

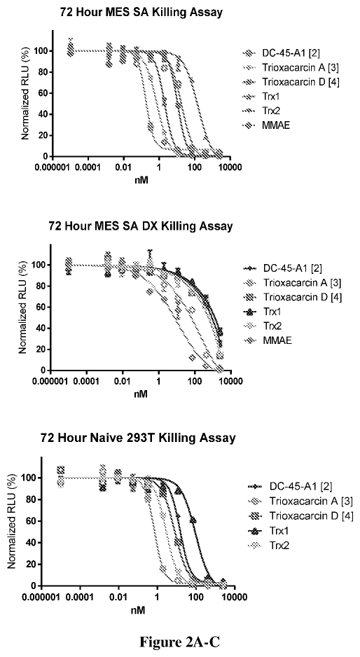

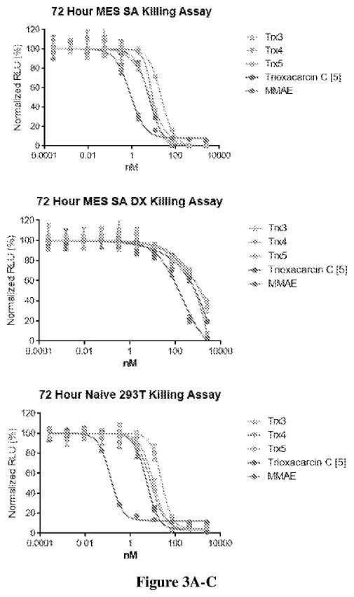

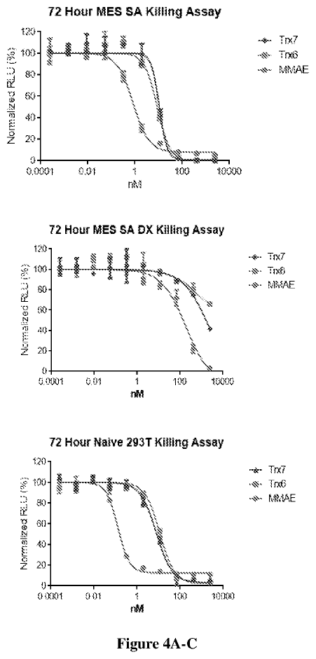

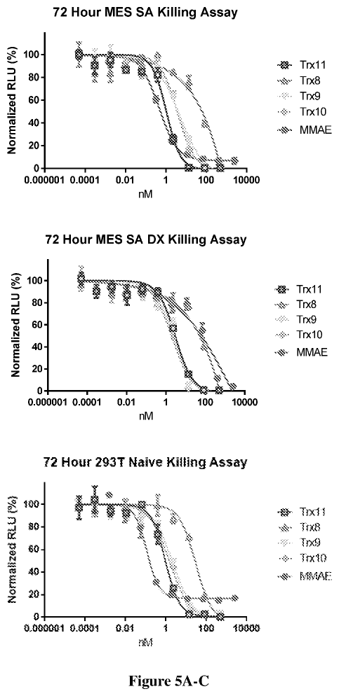

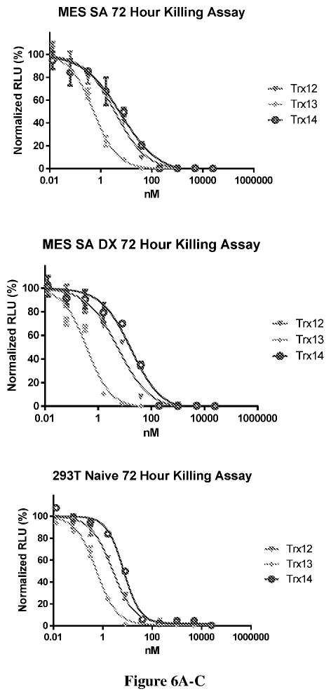

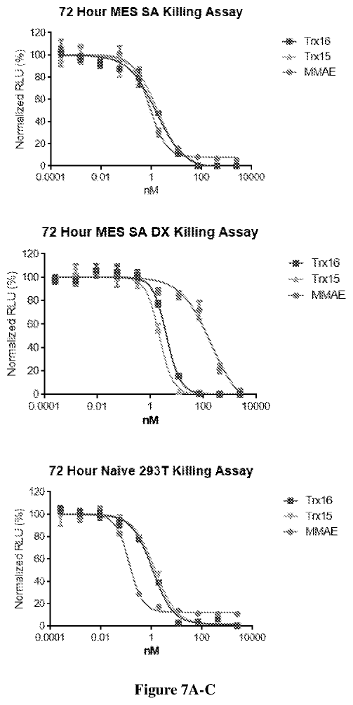

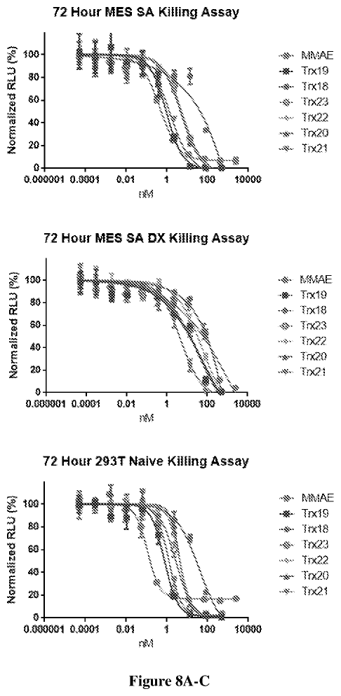

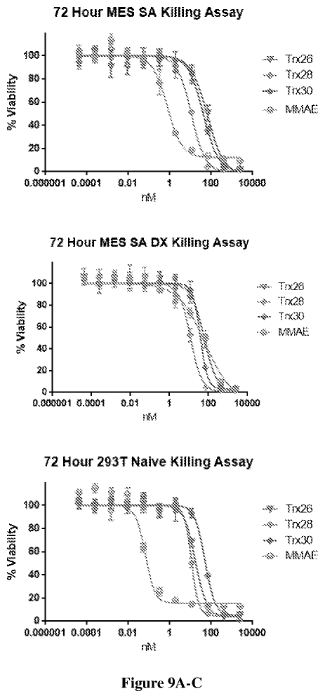

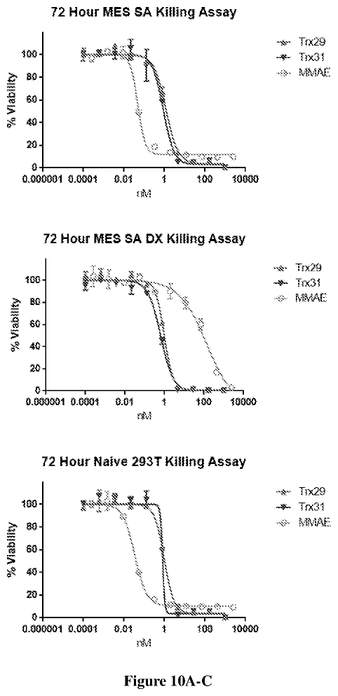

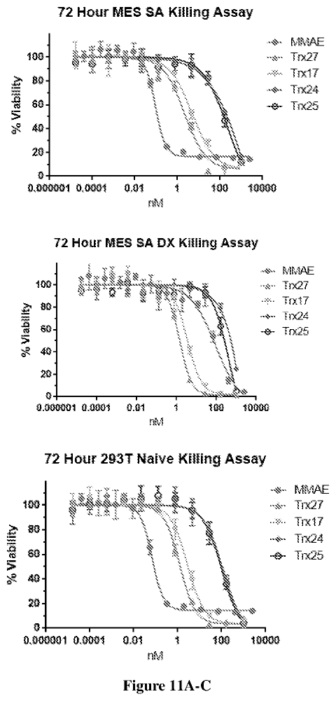

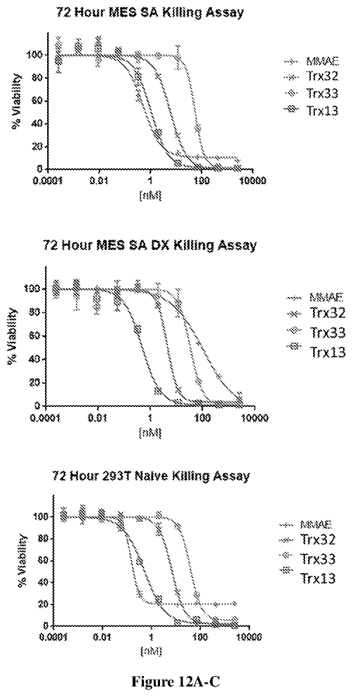

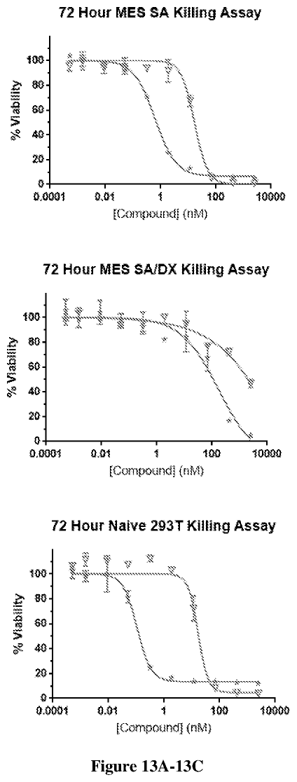

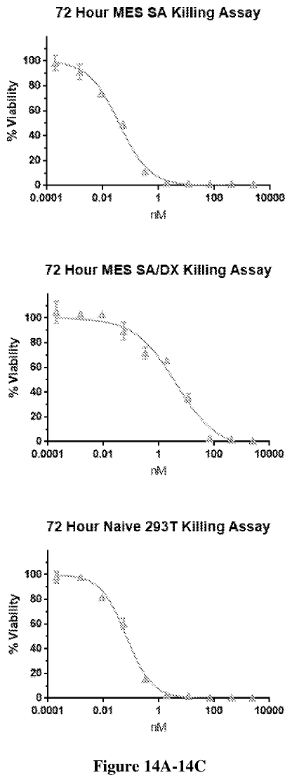

MES SA and MES SA/Dx cells are uterine sarcoma. MES SA Dx cell line was generated from MES SA to achieve upregulation of MDR1. MES-SA/Dx cells exhibit marked cross-resistance to a number of chemotherapeutic agents (including daunorubicin, dactinomycin, vincristine, taxol, colchicine) and moderate cross-resistance to mitomycin C and melphalan. 293T cells are human embryonic kidney cell line. Compounds of the disclosure were tested in 72 hour killing assays, which were performed on MES SA, MES SA DX, and 293T cell lines (FIGS. 1-14).

### 4. Example 5—General Synthetic Methods

Unless otherwise noted, all reactions were performed in flame-dried or oven-dried glassware under nitrogen atmosphere. Non-aqueous reagents were transferred using syringe techniques under nitrogen atmosphere. Tetrahydrofuran (THF), N,N-dimethylformamide (DMF), acetonitrile (MeCN), dichloro-methane (CH₂Cl₂), triethylamine (Et₃N), toluene, and pyridine were obtained anhydrous by degassing with argon and then passing through activated alumina columns to remove water and oxygen. Bulk grade hexanes, pentane, diethyl ether and ethyl acetate for chromatography were used without further treatment. Commercial reagents were obtained at the highest commercially available quality and used without further purification unless otherwise stated. Yields refer to chromatographically and spectroscopically (¹H NMR) homogeneous materials, unless otherwise stated.

Reactions were monitored by standard thin-layer chromatography (TLC) techniques using EMD silica gel 60F₂₅4 pre-coated plates (0.25 mm thickness). Following the run, TLC plates were visualized under UV light and/or by appropriate stains (p-anisaldehyde or cerric ammonium nitrate or potassium permanganate). Flash column chromatography was performed using silica gel (60, particle size 0.035-0.070 mm) obtained from Acros Organics. Preparative thin-layer chromatography (PTLC) separations were carried out using 0.25 or 0.50 mm E. Merck silica gel plates (60F₂₅₄).

Nuclear magnetic resonance (NMR) spectra were recorded on a Bruker Avance III HD 600 MHz instrument equipped with a 5 mm DCH cryoprobe and calibrated using residual undeuterated solvent for ¹H NMR [δ 7.26 (CDCl₃) or 5.32 (CD₂Cl₂) or 7.16 (C₆D₆) ppm] and ¹³C deuterated solvent for ¹³C NMR [δ 77.16 (CDCl₃) or 53.84 (CD₂Cl₂) or 128.06 (C₆D₆) ppm] as internal references at 300 K. The following abbreviations were used to indicate the multiplicities: s, singlet; d, doublet; t, triplet; q, quartet; quint: quintet; m, multiplet; br, broad. NMR coupling constants and signal patterns are reported as J values in Hz and δ values in parts per million (ppm). High resolution mass spectrometric measurements (HRMS) were obtained on Agilent Technologies 6530 Accurate Mass QTof LC/MS (ESI) or Agilent 1200 HPLC-6130 MSD (ESI). Melting points were recorded on a Thomas-Hoover Unimelt capillary melting point apparatus and are uncorrected. IR spectra were recorded on a Perkin-Elmer Spectrum 100 FT-IR spectrometer and are reported in terms of frequency of absorption (cm⁻¹). Optical rotations were recorded on a Schmidt+Haensch POLARTRONIC M100 polarimeter at 589.44 nm using 100 mm cells and the solvent and concentration indicated and are reported in units of 10-1 (deg cm²g⁻¹).

### 5. Example 6—Experimental Procedures and Characterization Data

### (2S,4S)-2-[(tert-Butyldimethylsilyl)oxy]-9-hydroxy-10-methoxy-4-[(4-methoxybenzyl)oxy]-8-(methoxymethoxy)-6-methyl-3,4-dihydroanthracen-1(2H)-one (10)

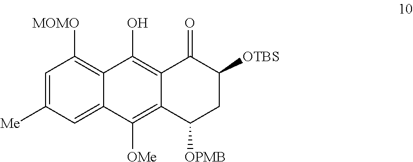

To a stirred solution of cyanophthalide 9 (Nicolaou et al., 2009) (702 mg, 3.01 mmol, 1.0 equiv) in THF (30 ml) at −78° C. was added t-BuOLi (1.0 M in THF, 9.03 ml, 9.03 mmol, 3.0 equiv). After stirring at this temperature for 10 min, a solution of enone 7² (1.09 g, 3.01 mmol, 1.0 equiv) in THF (10 ml) was added dropwise. The resulting reaction mixture was stirred at −78° C. for 0.5 h before Me₂SO₄ (3.6 g, 30.1 mmol, 10 equiv) was added dropwise. The resulting mixture was warmed to −5° C. and stirred at this temperature for 5 h before it was quenched with NH₄Cl (sat. aq., 150 ml). The resulting mixture was extracted with EtOAc (3×40 ml), and the combined organic phases were washed with brine (50 ml), dried over anhydrous Na₂SO₄, filtered and concentrated under reduced pressure. The residue was purified by flash column chromatography (silica gel, EtOAc:hexanes 1:6) to give the title compound (1.26 g, 2.16 mmol, 72%) as a yellow foam. 10: R_{f}=0.58 (silica gel, EtOAc:hexanes 1:4); [α]_{D}²⁵=+30.2 (c=0.2, CH₂Cl₂); FT-IR (film): νₘₐₓ=2952, 1620, 1577, 1514, 1443, 1386, 1250, 1172, 1152, 1046, 870 cm⁻¹; ¹H NMR (600 MHz, CD₂Cl₂) δ=14.79 (s, 1H), 7.52 (s, 1H), 7.27 (d, J=8.6 Hz, 2H), 6.98 (d, J=1.6 Hz, 1H), 6.88-6.65 (m, 2H), 5.30 (s, 2H), 5.18 (t, J=2.9 Hz, 1H), 4.97 (dd, J=12.4, 5.2 Hz, 1H), 4.67 (d, J=11.1 Hz, 1H), 4.55 (d, J=11.0 Hz, 1H), 3.84 (s, 3H), 3.77 (s, 3H), 3.56 (s, 3H), 2.72 (ddd, J=13.4, 5.2, 3.3 Hz, 1H), 2.51 (s, 3H), 2.20-2.12 (m, 1H), 0.97 (s, 9H), 0.23 (s, 3H), 0.17 (s, 3H) ppm; ¹³C NMR (151 MHz, CD₂Cl₂) δ=204.1, 163.0, 159.7, 157.6, 145.0, 142.7, 135.6, 131.0, 129.7, 126.7, 116.7, 116.3, 115.6, 114.1, 108.6, 96.5, 71.1, 69.8, 69.3, 63.0, 56.8, 55.6, 37.1, 26.1, 22.5, 18.8, −4.2, −5.1 ppm; HRMS (ESI-TOF) calcd for C₃₂H₄₂O₈SiNa⁺[M+Na]⁺605.2541, found 605.2529.

### (2S,4S)-2-[(tert-Butyldimethylsilyl)oxy]-8,9-dihydroxy-10-methoxy-4-[(4-methoxybenzyl)oxy]-6-methyl-3,4-dihydroanthracen-1(2H)-one (11)

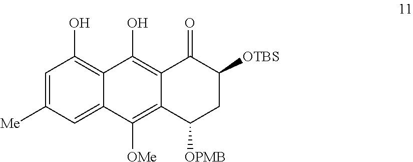

To a stirred solution of 10 (812 mg, 1.39 mmol, 1.0 equiv) in THF (50 ml) at 0° C. was added MgBr₂-Et₂O (1.08 g, 4.17 mmol, 3.0 equiv) in one portion. After stirring at this temperature for 2 h, the reaction was quenched with H₂O (50 ml). The resulting mixture was extracted with EtOAc (3×40 ml), and the combined organic phases were dried over anhydrous Na₂SO₄, filtered and concentrated under reduced pressure. The residue was purified by flash column chromatography (silica gel, EtOAc:hexanes 1:10) to give the title compound (594 mg, 1.08 mmol, 79%) as a yellow foam. 11: R_{f}=0.55 (silica gel, EtOAc:hexanes 1:6); [α]_{D}²⁵=+14.7 (c=1.0, CH₂Cl₂); FT-IR (film): νₘₐₓ=2953, 1634, 1613, 1514, 1443, 1389, 1249, 1155, 1045, 1005, 872 cm⁻¹; ¹H NMR (600 MHz, CDCl₃) δ=15.95 (s, 1H), 9.78 (s, 1H), 7.30 (s, 1H), 7.28 (d, J=8.3 Hz, 2H), 6.87 (d, J=8.2 Hz, 2H), 6.79 (s, 1H), 5.18-5.13 (m, 1H), 5.00 (dd, J=12.3, 5.2 Hz, 1H), 4.67 (d, J=11.1 Hz, 1H), 4.57 (d, J=11.1 Hz, 1H), 3.85 (s, 3H), 3.80 (s, 3H), 2.70 (ddd, J=13.5, 5.2, 3.4 Hz, 1H), 2.49 (s, 3H), 2.17 (td, J=12.8, 12.4, 2.5 Hz, 1H), 0.97 (s, 9H), 0.24 (s, 3H), 0.18 (s, 3H) ppm; ¹³C NMR (151 MHz, CDCl₃) δ=203.9, 162.8, 159.4, 158.6, 145.7, 144.2, 134.6, 130.4, 129.6, 125.3, 114.2, 114.0, 114.0, 113.6, 113.0, 107.1, 70.9, 69.2, 68.8, 62.8, 55.4, 37.1, 26.0, 26.0, 22.6, 18.7, −4.3, −5.1 ppm; HRMS (ESI-TOF) calcd for C₃₀H₃₈O₇SiNa⁺[M+Na]⁺561.2279, found 561.2265.

### (2S,4S)-7-Bromo-2-[(tert-butyldimethylsilyl)oxy]-8,9-dihydroxy-10-methoxy-4-[(4-methoxy-benzyl)oxy]-6-methyl-3,4-dihydroanthracen-1(2H)-one (12)

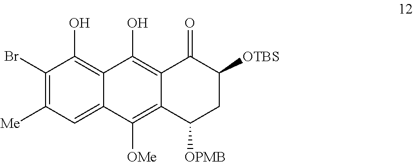

To a stirred solution of phenol 11 (560 mg, 1.04 mmol, 1.0 equiv) in THF (50 ml) at −78° C. was added a solution of NBS (185 mg, 1.04 mmol, 1.0 equiv) in THF (10 ml) dropwise. The resulting reaction mixture was stirred at −78° C. for 0.5 h, then warmed to room temperature and stirred at this temperature for 5 h before it was quenched with NH4Cl (sat. aq., 50 ml). The resulting mixture was extracted with EtOAc (3×50 ml), and the combined organic phases were washed with brine (50 ml), dried over anhydrous Na₂SO₄, filtered and concentrated under reduced pressure. The residue was purified by flash column chromatography (silica gel, EtOAc:hexanes 1:10) to give the title compound (525 mg, 0.85 mmol, 82%) as a yellow oil. 12: R_{f}=0.50 (silica gel, EtOAc:hexanes 1:6); [α]_{D}²⁵=+28.9 (c=0.2, CH₂Cl₂); FT-IR (film): νₘₐₓ=2952, 1622, 1514, 1443, 1316, 1249, 1171, 1055, 1004, 872 cm⁻¹; ¹H NMR (600 MHz, CD₂Cl₂) δ=16.12 (s, 1H), 10.54 (s, 1H), 7.46 (s, 1H), 7.30-7.16 (m, 2H), 6.94-6.73 (m, 2H), 5.18-5.15 (m, 1H), 5.01 (dd, J=12.3, 5.3 Hz, 1H), 4.67 (d, J=11.0 Hz, 1H), 4.55 (d, J=11.0 Hz, 1H), 3.85 (s, 3H), 3.77 (s, 3H), 2.72 (ddd, J=13.6, 5.4, 3.5 Hz, 1H), 2.60 (s, 3H), 2.23-2.12 (m, 1H), 0.97 (s, 9H), 0.23 (s, 3H), 0.18 (s, 3H) ppm; ¹³C NMR (151 MHz, CD₂Cl₂) δ=204.8, 161.8, 159.7, 155.0, 145.9, 143.9, 133.0, 130.7, 129.9, 129.8, 126.4, 114.9, 114.1, 113.6, 110.1, 108.1, 71.3, 71.2, 69.4, 69.1, 69.1, 63.2, 55.6, 37.2, 26.0, 24.7, 18.8, −4.3, −5.1 ppm; HRMS (ESI-TOF) calcd for C₃₀H₃₈BrO₇Si⁺ [M+H]⁺ 617.1565, found 617.1554.

### (8S,10S)-4-Bromo-2,2-di-tert-butyl-10-[(tert-butyldimethylsilyl)oxy]-7-methoxy-8-[(4-methoxybenzyl)oxy]-5-methyl-9,10-dihydroanthra[1,9-de][1,3,2]dioxasilin-11(8H)-one (13)

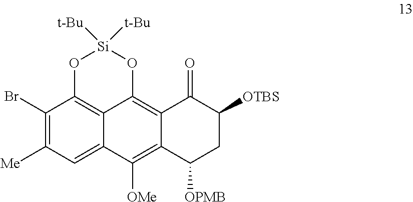

To a stirred solution of 12 (838 mg, 1.36 mmol, 1.0 equiv) in CH₂Cl₂ (30 ml) at 0° C. was added 2,6-lutidine (437 mg, 4.08 mmol, 3.0 equiv), and then t-Bu2Si(OTf)₂ (716 mg, 1.63 mmol, 1.2 equiv) was added dropwise over a period of 10 min. After stirring at this temperature for another 10 min, the reaction was quenched with NH₄Cl (sat. aq., 20 ml) and the resulting mixture was extracted with CH₂Cl₂ (2×50 ml). The combined organic phases were washed with brine (50 ml), dried over anhydrous Na₂SO₄, filtered and concentrated under reduced pressure. The residue was purified by flash column chromatography (silica gel, EtOAc:hexanes 1:6) to give the title compound (730 mg, 0.97 mmol, 71%) as a yellow foam. 13: R_{f}=0.75 (silica gel, EtOAc:hexanes 1:4); [α]_{D}²⁵=+43.0 (c=0.1, CH₂Cl₂); FT-IR (film): νₘₐₓ=2934, 2859, 1698, 1606, 1514, 1472, 1405, 1361, 1250, 1157, 1066, 829 cm⁻¹; ¹H NMR (600 MHz, CD₂Cl₂) δ=7.51 (d, J=1.2 Hz, 1H), 7.37-7.21 (m, 2H), 6.94-6.64 (m, 2H), 5.18 (t, J=2.9 Hz, 1H), 4.83 (dd, J=12.4, 5.2 Hz, 1H), 4.70 (d, J=10.8 Hz, 1H), 4.58 (d, J=10.8 Hz, 1H), 3.88 (s, 3H), 3.77 (s, 3H), 2.73 (ddd, J=13.6, 5.2, 3.1 Hz, 1H), 2.58 (d, J=0.9 Hz, 3H), 2.12 (ddd, J=13.5, 12.3, 2.7 Hz, 1H), 1.14 (s, 9H), 1.11 (s, 9H), 0.95 (s, 9H), 0.21 (s, 3H), 0.14 (s, 3H) ppm; ¹³C NMR (151 MHz, CD₂Cl₂) δ=194.9, 159.7, 150.0, 149.2, 146.8, 140.9, 130.9, 130.7, 130.1, 130.0, 119.4, 116.6, 115.7, 115.2, 114.1, 112.0, 71.6, 71.4, 70.2, 63.1, 55.6, 36.9, 26.2, 26.2, 26.2, 24.6, 21.5, 21.3, 18.9, −4.1, −5.2 ppm; HRMS (ESI-TOF) calcd for C₃₈H₅₃BrO₇Si₂Na⁺ [M+Na]⁺779.2405, found 779.2407.

### (8S,10S)-2,2-Di-tert-butyl-10-[(tert-butyldimethylsilyl)oxy]-4-[(E)-3-hydroxyprop-1-en-1-yl]-7-methoxy-8-[(4-methoxybenzyl)oxy]-5-methyl-9,10-dihydroanthra[1,9-de][1,3,2]dioxasilin-11(8H)-one (8)

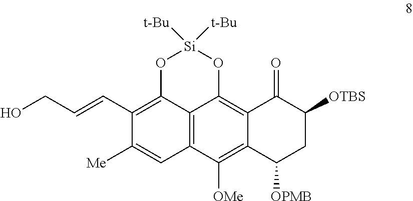

To a stirred mixture of aryl bromide 13 (553 mg, 0.730 mmol, 1.0 equiv), tri(2-furyl)phosphine (33.8 mg, 0.146 mmol, 0.2 equiv) and Pd(PPh₃)₄ (83.8 mg, 0.0725 mmol, 0.1 equiv) in DMF (20 ml) was added stannane 14 (380 mg, 1.10 mmol, 1.5 equiv), After bubbled with argon balloon three times, N,N-diisopropylethylamine (0.253 ml, 1.46 mmol, 2.0 equiv) and LiCl (1 M in THF, 1.46 ml, 1.46 mmol, 2.0 equiv) was added. After stirring at 110° C. for 12 h, the reaction mixture was cooled to 23° C., then was 20 diluted with EtOAc (20 ml) and quenched with water (10 ml). The resulting mixture was washed with brine (3×10 ml), dried over anhydrous Na₂SO₄, filtered and concentrated under reduced pressure. The residue was purified by flash column chromatography (silica gel, EtOAc:hexanes 1:4→1:2) to give the title compound (364 mg, 0.496 mmol, 68%) as a yellow foam. [α]_{D}²⁵=+28.1 (c=0.1, CH₂Cl₂). All spectroscopic data were consistent with those reported in the literature (Nicolaou et al., 2015).

### 9-Hydroxy-10-methoxy-8-(methoxymethoxy)-6-methyl-3,4-dihydroanthracen-1(2H)-one (15)

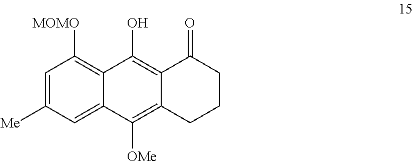

To a stirred solution of cyanophthalide 9 (9.60 g, 41.2 mmol, 1.0 equiv) in THF (100 ml) at −78° C. was added t-BuOLi (1.0 M in THF, 123.6 ml, 123.6 mmol, 3.0 equiv). After stirring at this temperature for 10 min, a solution of cyclohexanone (4.40 ml, 45.3 mmol, 1.1 equiv) in THF (27 ml) was added dropwise. The resulting reaction mixture was stirred at −78° C. for 0.5 h before Me₂SO₄ (23.4 ml, 247 mmol, 6.0 equiv) was added dropwise. Then the resulting mixture was warmed to 23° C. and stirred at this temperature for 5 h before it was quenched with NH4Cl (sat. aq., 150 ml). The resulting mixture was extracted with EtOAc (3×80 ml), and the combined organic phases were washed with brine (150 ml), dried over anhydrous Na₂SO₄, filtered and concentrated under reduced pressure. The residue was purified by flash column chromatography (silica gel, EtOAc:hexanes 1:4) to give the title compound 13 (9.39 g, 29.7 mmol, 72%) as a yellow foam. 15: R_{f}=0.50 (silica gel, EtOAc:hexanes 1:3); FT-IR (film): νₘₐₓ=3375, 2498, 1618, 1577, 1444, 1384, 1330, 1234, 1177, 1039 cm⁻¹; ¹H NMR (600 MHz, CDCl₃) δ=15.02 (s, 1H), 7.46 (d, J=1.6 Hz, 1H), 6.94 (d, J=1.6 Hz, 1H), 5.35 (s, 2H), 3.79 (s, 3H), 3.60 (s, 3H), 3.04 (dd, J=6.9, 5.5 Hz, 2H), 2.73 (dd, J=7.1, 5.8 Hz, 2H), 2.50 (d, J=0.8 Hz, 3H), 2.12-2.05 (m, 2H) ppm; ¹³C NMR (151 MHz, CDCl₃) δ=204.6, 162.8, 157.3, 142.9, 142.1, 135.5, 128.6, 115.3, 114.9, 114.1, 110.7, 96.0, 60.9, 56.6, 39.0, 23.7, 22.6, 22.4 ppm. HRMS (ESI) calcd for C₁₈H₂₀O₅Na⁺ [M+Na]⁺339.1203, found 339.1210.

### 8,9-Dihydroxy-10-methoxy-6-methyl-3,4-dihydroanthracen-1(2H)-one (16)

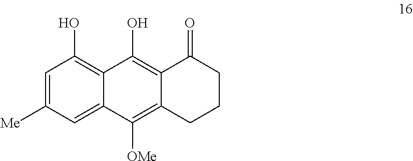 [SMILES: COc1c2c(c(O)c3c(O)cc(C)cc13)C(=O)CCC2]

To a stirred solution of 15 (7.80 g, 24.7 mmol, 1.0 equiv) in THF (100 ml) at 0° C. was added MgBr₂-Et₂O (12.7 g, 49.6 mmol, 2.0 equiv) in one portion. After stirring at this temperature for 3 h, the reaction was quenched with H₂O (50 ml). The resulting mixture was extracted with EtOAc (3×50 ml), and the combined organic phases were dried over anhydrous Na₂SO₄, filtered and concentrated under reduced pressure. The residue was purified by flash column chromatography (silica gel, EtOAc:hexanes 1:4) to give the title compound (6.07 g, 22.3 mmol, 90%) as a yellow foam. 16: R_{f}=0.40 (silica gel, EtOAc:hexanes 1:3); FT-IR (film): νₘₐₓ=3354, 2995, 2959, 2938, 1633, 1616, 1512, 1446, 1395, 1334, 1244, 1170, 1038 cm⁻¹; ¹H NMR (600 MHz, CDCl₃) δ=16.14 (s, 1H), 9.82 (s, 1H), 7.24 (s, 1H), 6.72 (s, 1H), 3.80 (s, 3H), 3.01 (t, J=6.2 Hz, 2H), 2.76-2.64 (m, 2H), 2.47 (s, 3H), 2.09 (quint, J=6.3 Hz, 2H) ppm; ¹³C NMR (151 MHz, CDCl₃) δ=204.6, 163.2, 158.6, 144.2, 143.6, 134.8, 127.5, 113.0, 112.7, 111.5, 109.2, 60.9, 38.0, 23.3, 22.6, 22.4 ppm; HRMS (ESI) calcd for C₁₆H₁₇O₄⁺ [M+H]⁺ 273.1121, found 273.1127.

### 7-Bromo-8,9-dihydroxy-10-methoxy-6-methyl-3,4-dihydroanthracen-1(2H)-one (17)

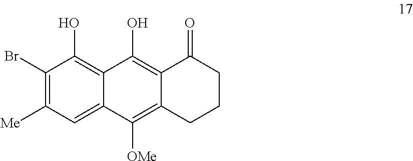 [SMILES: COc1c2c(c(O)c3c(O)c(Br)c(C)cc13)C(=O)CCC2]

To a stirred solution of phenol 16 (5.0 g, 18.4 mmol, 1.0 equiv) in THF (200 ml) at −78° C. was added a solution of NBS (3.27 g, 18.4 mmol, 1.0 equiv) in THF (20 ml) dropwise. The resulting reaction mixture was stirred at −78° C. for 0.5 h, then warmed to room temperature and stirred at this temperature for 5 h before it was quenched with NH₄Cl (sat. aq., 100 ml). The resulting mixture was extracted with EtOAc (3×100 ml), and the combined organic phases were washed with brine (100 ml), dried over anhydrous Na₂SO₄, filtered and concentrated under reduced pressure. The residue was purified by flash column chromatography (silica gel, EtOAc:hexanes 1:10-1:2) to give the title compound 15 (5.47 g, 15.6 mmol, 85%) as a yellow foam. 17: R_{f}=0.16 (silica gel, EtOAc:hexanes 1:3); FT-IR (film): νₘₐₓ=3322, 2948, 1490, 1391, 1256, 1241, 1048 cm⁻¹; ¹H NMR (600 MHz, CDCl₃) δ=16.30 (s, 1H), 10.60 (s, 1H), 7.35 (s, 1H), 3.79 (s, 3H), 3.02-2.99 (m, 2H), 2.74 (t, J=6.4 Hz, 2H), 2.57 (s, 3H), 2.10 (quint, J=6.4 Hz, 2H) ppm; ¹³C NMR (151 MHz, CDCl₃) δ=204.8, 162.2, 154.7, 143.6, 143.5, 132.8, 128.0, 113.8, 113.8, 120.0, 109.8, 108.7, 61.1, 37.9, 24.7, 23.3, 22.2 ppm. HRMS (ESI) calcd for Cl₆H₁₆BrO₄⁺ [M+H]⁺ 351.0226, found 351.0229.

### 4-Bromo-2,2-di-tert-butyl-7-methoxy-5-methyl-9,10-dihydroanthra[1,9-de][1,3,2]dioxasilin-11(8H)-one (18)

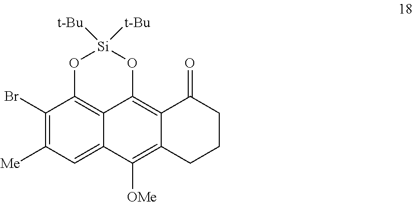 [SMILES: COc1c2c(c3c4c(c(Br)c(C)cc14)O[Si](C(C)(C)C)(C(C)(C)C)O3)C(=O)CCC2]

To a stirred solution of 17 (4.24 g, 12.1 mmol, 1.0 equiv) in CH2Cl2 (150 ml) at −78° C. were added Et₃N (11.8 ml, 84.8 mmol, 7.0 equiv), and then t-Bu₂Si(OTf)₂ (4.8 ml, 14.3 mmol, 1.2 equiv) was added dropwise over a period of 5 min. The reaction mixture was allowed to warm to 0° C. and then quenched with NaHCO₃ (sat. aq., 50 ml) at 0° C. and stirred for 0.5 h at 23° C. The resulting mixture was partitioned, and the aqueous phase was extracted with CH₂Cl₂ (3×100 ml). The combined organic phases were washed with brine (100 ml), dried over anhydrous Na₂SO₄, filtered, and concentrated under reduced pressure. The residue was purified by flash column chromatography (silica gel, EtOAc:hexanes 1:50) to give the title compound (4.96 g, 10.1 mmol, 84%) as a pale yellow powder. 18: R_{f}=0.35 (silica gel, EtOAc:hexanes 1:3); FT-IR (film): νₘₐₓ=2936, 2861, 1681, 1622, 1605, 1561, 1472, 1402, 1361, 1229, 1063 cm⁻¹; ¹H NMR (600 MHz, CDCl₃) δ=7.45 (d, J=1.1 Hz, 1H), 3.83 (s, 3H), 3.08-2.94 (m, 2H), 2.66-2.63 (m, 2H), 2.57 (d, J=1.0 Hz, 3H), 2.08 (quint, J=6.5 Hz, 2H), 1.14 (s, 18H) ppm; ¹³C NMR (151 MHz, CDCl₃) δ=196.6, 149.8, 149.6, 144.5, 140.7, 131.9, 130.5, 116.5, 114.9, 114.7, 114.6, 110.8, 61.2, 61.1, 41.4, 41.3, 41.2, 26.3, 26.3, 26.3, 26.2, 24.6, 24.5, 24.5, 24.4, 24.3, 24.2, 22.5, 21.3, 21.0 ppm; HRMS (ESI) calcd for C₂₄H₃₁BrO₄SiNa⁺ [M+Na]⁺513.1067, found 513.1077.

### General Procedure for α-Hydroxylation of Ketone 18:

To a stirred solution of ketone 18 (0.1 mmol) in THF (1 ml) at −78° C. was added base (0.15 mmol, 1.5 equiv) dropwise. The resulting mixture was stirred at this temperature for 0.5 h and then oxaziridine (0.15 mmol, 1.5 equiv) was added in one portion. The resulting mixture was stirred at −78° C. for 0.5 h before it was quenched with NH₄Cl (sat. aq., 2 ml). The reaction mixture was extracted with EtOAc (3×5 ml), and the combined organic phases were washed with brine (3 ml), dried over anhydrous Na₂SO₄, filtered and concentrated under reduced pressure. The residue was purified by flash column chromatography (silica gel, EtOAc:hexanes 1:10→1:5) to give alcohol (10R)- or (10S)-20. The enantiomeric ratio of 20 was determined by HPLC (Chiralcel OD-H, 25° C., flow rate: 1 mL/min, hexanes/isopropanol: 99.5/0.5, 254 nm): 6.83 min for (10R)-20, 7.41 min for (10S)-20.

### (S)-4-Bromo-2,2-di-tert-butyl-10-hydroxy-7-methoxy-5-methyl-9,10-dihydroanthra[1,9-de][1,3,2]di-oxasilin-11(8H)-one [(10S)-20]

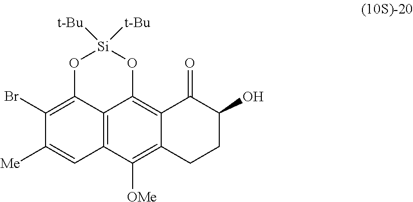 [SMILES: COc1c2c(c3c4c(c(Br)c(C)cc14)O[Si](C(C)(C)C)(C(C)(C)C)O3)C(=O)[C@@H](O)CC2]

(10S)-20 was obtained in 77% yield (38.9 mg, 0.0769 mmol, er 14:1) as a pale yellow foam according to the general procedure with LTMP as base and oxaziridine (−)-19. (10S)-20: R_{f}=0.21 (silica gel, EtOAc:hexanes 1:5); [α]_{D}²⁵=−1.0 (c=0.52, CHCl3); FT-IR (film): νₘₐₓ=3465, 2936, 2862, 1682, 1606, 1562, 1472, 14445, 1404. 1367, 1253 cm; ¹H NMR (600 MHz, CDCl₃) δ=7.45 (s, 1H), 4.32 (ddd, J=13.4, 5.3, 2.5 Hz, 1H), 4.26 (d, J=2.5 Hz, 1H), 3.84 (s, 3H), 3.41 (ddd, J=17.4, 4.6, 2.5 Hz, 1H), 2.93 (ddd, J=17.8, 13.4, 4.9 Hz, 1H), 2.58 (s, 3H), 2.53 (dtd, J=12.6, 5.1, 2.4 Hz, 1H), 1.95 (qd, J=13.2, 4.6 Hz, 1H), 1.17 (s, 9H), 1.12 (s, 9H) ppm; ¹³C NMR (151 MHz, CDCl₃) δ=197.2, 150.3, 149.8, 145.0, 141.4, 130.9, 130.8, 114.8, 114.7, 114.1, 111.1, 74.2, 60.9, 31.0, 26.3, 26.2, 24.6, 22.2, 21.5, 21.1 ppm; HRMS (ESI-TOF) calcd for C₂₄H₃₁BrO₅Si⁺ [M+H]⁺ 507.1197, found 507.1181. (10R)-20 was obtained in 78% yield (39.0 mg, 0.078 mmol, er 14:1) as a pale yellow foam according to the general procedure with LTMP as base and oxaziridine (+)-19. (10R)-20: [α]_{D}²⁵=+1.2 (c=0.25, CHCl₃). Other physical and spectral data are identical with those of (10S)-20.

rac-20 was prepared according to the general procedure with LTMP as base and oxaziridine rac-19 for analysis purposes.

### (10S)-4-Bromo-2,2-di-tert-butyl-10-{[tert-butyl(dimethyl)silyl]oxy}-7-methoxy-5-methyl-9,10-dihydroanthra[1,9-de][1,3,2]dioxasilin-11(8H)-one (21)

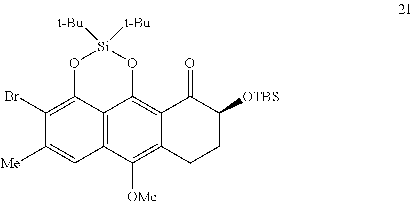 [SMILES: COc1c2c(c3c4c(c(Br)c(C)cc14)O[Si](C(C)(C)C)(C(C)(C)C)O3)C(=O)[C@@H](O[Si](C)(C)C(C)(C)C)CC2]

To a stirred solution of alcohol 20 (39.6 mg, 0.0780 mmol, 1.0 equiv) in CH₂Cl₂ (1 ml) at −78° C. were sequentially added 2,6-lutidine (36 μL, 0.31 mmol, 4.0 equiv) and TBSOTf (54 mg, 0.234 mmol, 3.0 equiv). The resulting mixture was stirred at this temperature for 0.5 h before it was quenched with Na₂CO₃ (sat. aq., 1 ml). The reaction mixture was extracted with CH₂Cl₂ (3 ml), and the combined organic phases were washed with brine (2 ml), dried over anhydrous Na₂SO₄, filtered and concentrated under reduced pressure. The residue was purified by flash column chromatography (silica gel, EtOAc:hexanes 1:10) to give the title compound (47.1 mg, 0.076 mmol, 97%) as a colorless oil. 21: R_{f}=0.50 (silica gel, EtOAc:hexanes 1:5); [α]_{D}²⁵=−1.65 (c=0.48, CHCl₃); FT-IR (film): νₘₐₓ=3464, 2935, 2860, 1700, 1606, 1564, 1472, 1445, 1403, 1361, 1253 cm⁻¹; ¹H NMR (600 MHz, CDCl₃) δ=7.43 (d, J=1.1 Hz, 1H), 4.30 (dd, J=8.7, 3.8 Hz, 1H), 3.83 (s, 3H), 3.28 (ddd, J=17.2, 7.0, 5.2 Hz, 1H), 2.99 (ddd, J=17.3, 7.8, 5.4 Hz, 1H), 2.57 (d, J=1.0 Hz, 3H), 2.28-2.08 (m, 2H), 1.13 (d, J=1.6 Hz, 18H), 0.87 (s, 9H), 0.13 (s, 3H), 0.12 (s, 3H) ppm; ¹³C NMR (151 MHz, CDCl₃) δ=195.3, 149.7, 149.4, 144.6, 140.4, 131.0, 130.3, 116.0, 114.8, 114.6, 110.6, 75.5, 60.8, 31.3, 26.3, 26.2, 26.0, 24.5, 21.3, 21.3, 21.0, 18.6, −4.3, −5.1 ppm; HRMS (ESI-TOF) calcd for C₃₀H₄₆BrO₅Si₂⁺ [M+H]⁺ 621.2062, found 621.2020.

### (8S,10S)-4-Bromo-2,2-di-tert-butyl-10-{[tert-butyl(dimethyl)silyl]oxy}-7-methoxy-5-methyl-11-oxo-8,9,10,11-tetrahydroanthra[1,9-de][1,3,2]dioxasilin-8-yl chloroacetate (22)

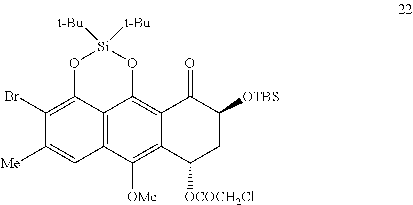

To a stirred mixture of 21 (191 mg, 0.307 mmol, 1.0 equiv) and chloroacetic acid (577 mg, 6.14 mmol, 20.0 equiv) and 4 Å molecule sieves (1.0 g) in CH₂Cl₂ (3 ml) at 0° C. was added DDQ (139 mg, 0.614 mmol, 2.0 equiv). The resulting mixture was stirred at this temperature for 6 h before it was diluted with EtOAc (10 ml) and the resulting mixture was poured into NaHCO₃ (sat. aq., 20 ml). The reaction mixture was filtered through Celite®, then extracted with EtOAc (3×10 ml). The combined organic phases were washed with brine (5 ml), dried over anhydrous Na₂SO₄, filtered and concentrated under reduced pressure. The residue was purified by flash column chromatography (silica gel, EtOAc:hexanes 1:10) to give the title compound (22, 191 mg, 0.268 mmol, 87%) as a colorless oil. 22: R_{f}=0.39 (silica gel, EtOAc:hexanes 1:5); [α]_{D}²⁵=−43.7 (c=0.58, CHCl₃); FT-IR (film): νₘₐₓ=2935, 2897, 2861, 1742, 1705, 1605, 1566 1472, 1407, 1362 cm⁻¹; ¹H NMR (600 MHz, CDCl₃) δ=7.45 (d, J=1.1 Hz, 1H), 6.66 (t, J=3.5 Hz, 1H), 4.67 (dd, J=12.1, 4.7 Hz, 1H), 4.10 (d, J=14.7 Hz, 1H), 4.06 (d, J=14.6 Hz, 1H), 3.86 (s, 3H), 2.58 (d, J=0.9 Hz, 3H), 2.52 (ddd, J=14.3, 4.7, 3.3 Hz, 1H), 2.43 (ddd, J=14.3, 12.1, 3.8 Hz, 1H), 1.15 (s, 9H), 1.13 (s, 9H), 0.92 (s, 9H) ppm; ¹³C NMR (151 MHz, CDCl₃) δ 193.6, 166.7, 149.9, 149.1, 147.2, 141.2, 130.3, 125.8, 116.7, 115.4, 114.6, 112.6, 71.8, 67.8, 62.6, 41.1, 37.4, 26.3, 26.2, 26.0, 24.6, 21.5, 21.1, 18.8, −4.3, −5.3 ppm; HRMS (ESI-TOF) calcd for C32H₄₇BrO₇Si₂⁺ [M+H]⁺ 713.1727, found 713.1729.

### (8S,10S)-4-Bromo-2,2-di-tert-butyl-10-{[tert-butyl(dimethyl)silyl]oxy}-8-hydroxy-7-methoxy-5-methyl-9,10-dihydroanthra[1,9-de][1,3,2]dioxasilin-11(8H)-one (23)

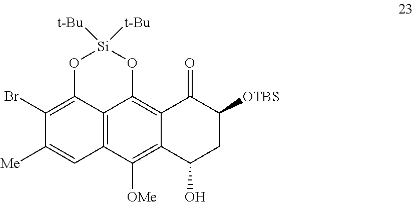 [SMILES: COc1c2c(c3c4c(c(Br)c(C)cc14)O[Si](C(C)(C)C)(C(C)(C)C)O3)C(=O)[C@@H](O[Si](C)(C)C(C)(C)C)C[C@@H]2O]

To a stirred solution of chloroacetate 22 (122 mg, 0.186 mmol, 1.0 equiv) in MeOH (1 ml) at 0° C. was added LiOH (0.45 M in MeOH, 413 μL, 0.186 mmol, 1.2 equiv) dropwise. The resulting mixture was stirred at this temperature for 5 min before it was diluted with EtOAc (2 ml) and quenched with NH₄Cl (sat. aq., 2 ml). The reaction mixture was extracted with EtOAc (3×5 ml), and the combined organic phases were washed with brine (3 ml), dried over anhydrous Na₂SO₄, filtered and concentrated under reduced pressure. The residue was purified by flash column chromatography (silica gel, EtOAc:hexanes 1:10) to give the title compound (109 mg, 0.169 mmol, 91%) as a colorless oil. 23: R_{f}=0.21 (silica gel, EtOAc:hexanes 1:5); [α]_{D}²⁵=−28.9 (c=1.84, CHCl₃); FT-IR (film): νₘₐₓ=3464, 2935, 2860, 1700, 1606, 1564, 1472, 1445, 1403, 1361, 1253 cm⁻¹; ¹H NMR (600 MHz, CDCl₃) δ=7.43 (d, J=1.0 Hz, 1H), 5.44 (td, J=4.8, 2.2 Hz, 1H), 4.68 (dd, J=10.2, 4.0 Hz, 1H), 3.96 (s, 3H), 3.25-3.17 (m, 1H), 2.59 (s, 3H), 2.51-2.43 (m, 1H), 2.38 (dt, J=13.7, 4.4 Hz, 1H), 1.14 (s, 9H), 1.11 (s, 9H), 0.88 (s, 9H), 0.15 (s, 3H), 0.11 (s, 3H) ppm; ¹³C NMR (151 MHz, CDCl₃) δ=194.8, 149.8, 148.9, 146.0, 140.8, 132.0, 130.3, 116.1, 114.8, 114.2, 111.8, 72.7, 63.7, 62.3, 38.9, 26.2, 26.2, 26.0, 24.6, 21.3, 21.3, 18.6, −4.3, −5.2 ppm; HRMS (ESI-TOF) calcd for C₃₀H₄₆BrO₆Si₂⁺ [M+H]⁺ 637.2011, found 637.2009.

### (8S,10S)-4-Bromo-2,2-di-tert-butyl-10-{[tert-butyl(dimethyl)silyl]oxy}-7-methoxy-8-[(4-methoxy-benzyl)oxy]-5-methyl-9,10-dihydroanthra[1,9-de][1,3,2]dioxasilin-11(8H)-one (13)

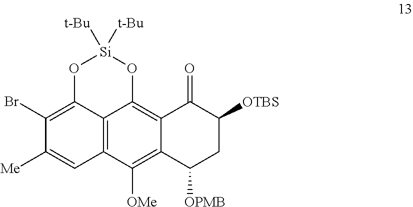

To a stirred solution of 23 (21.4 mg, 0.0335 mmol, 1.0 equiv) in toluene (0.5 ml) at 23° C. were added a solution of freshly prepared 4-methoxybenzyl-2,2,2-trichloroacetimidate (22 mg, 0.077 mmol, 2.3 equiv) in toluene (0.2 ml) and Cu(OTf)₂ (2.0 mg, 0.0055 mmol, 0.2 equiv). The resulting mixture was stirred at this temperature for 6 h before it was directly subject to a flash column chromatography (silica gel, EtOAc:hexanes 1:10) to afford the title compound (13, 17.8 mg, 0.0235 mmol, 70%) as a colorless oil together with the recovered starting material (4.8 mg, 0.0075 mmol, 22%). 13: [α]_{D}²⁵=+37.5 (c=0.6, CH₂Cl₂); Enantiomeric ratio of 13 was determined by HPLC (Chiralcel OD-H, 25° C., flow rate: 1 mL/min, hexanes/isopropanol: 99/1, 254 nm) as 54:1. Other physical and spectral data are identical with those reported in the previous route.

### (4R,5S,6S,8S,10R)-8-{[(1S,8S,10S,13aS)-10-{[tert-Butyl(dimethyl)silyl]oxy}-2-(dimethoxymethyl)-8,12-dihydroxy-7-methoxy-5-methyl-11-oxo-3a,4,8,9,10,11-hexahydro-13aH-spiro[2,4-epoxyfuro[3,2-b]naphtho[2,3-h]chromene-1,2′-oxiran]-13a-yl]oxy}-4,6-dimethyl-2-oxo-1,3,7-trioxaspiro[4.5]dec-10-yl acetate (27)

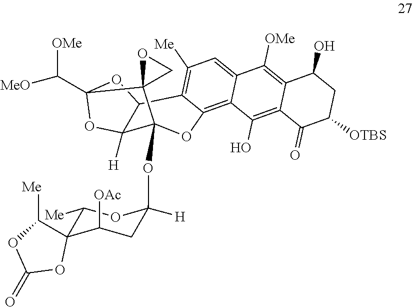

To a stirred solution of mono-glycosylated compound 26 (Nicolaou et al., 2016) (4.1 mg, 4.1 μmol, 1.0 equiv) in CH₂Cl₂ (0.2 ml) and H₂O (0.05 ml, pH 7.0 buffer) at 23° C. in a reaction flask shielded from light using aluminum foil was added DDQ (2.7 mg, 12 μmol, 2.9 equiv). After stirring at this temperature for 3 h, the reaction was quenched with brine (2 ml). The resulting mixture was extracted with CH₂Cl₂ (3×2 ml), dried over anhydrous Na₂SO₄, and concentrated under reduced pressure. The residue was purified by preparative thin layer chromatography (silica gel, hexanes:EtOAc 1:2) to give 27 (3.0 mg, 3.4 μmol, 83%) as an orange foam. 27: R_{f}=0.30 (silica gel, EtOAc:hexanes 1:1); [α]_{D}²⁵=+6.7 (c=0.15, CH₂Cl₂); FT-IR (film): νₘₐₓ=2952, 2856, 1815, 1749, 1621, 1389, 1221, 1082, 1048, 1015, 868 cm⁻¹; ¹H NMR (600 MHz, CDCl₃)=14.43 (s, 1H), 7.44 (d, J=1.1 Hz, 1H), 5.76 (dd, J=4.3, 2.2 Hz, 1H), 5.43 (t, J=3.5 Hz, 1H), 5.22 (d, J=4.1 Hz, 1H), 5.18 (d, J=4.1 Hz, 1H), 5.02 (t, J=3.7 Hz, 1H), 4.90 (dd, J=11.8, 5.0 Hz, 1H), 4.70 (s, 1H), 4.64 (q, J=6.3 Hz, 1H), 4.59 (q, J=6.8 Hz, 1H), 3.91 (s, 3H), 3.61 (s, 3H), 3.46 (s, 3H), 2.82 (d, J=6.0 Hz, 1H), 2.73 (d, J=6.0 Hz, 1H), 2.60 (d, J=1.1 Hz, 3H), 2.50 (ddd, J=13.5, 5.0, 3.6 Hz, 1H), 2.51 (br s, 1H), 2.38-2.29 (m, 2H), 2.28-2.23 (m, 1H), 2.23 (s, 3H), 1.60 (d, J=6.8 Hz, 3H), 1.36 (d, J=6.4 Hz, 3H), 0.96 (s, 9H), 0.23 (s, 3H), 0.16 (s, 3H) ppm; ¹³C NMR (151 MHz, CDCl₃) δ=203.2, 170.1, 162.7, 153.5, 151.8, 143.9, 142.1, 135.3, 129.9, 116.3, 114.9, 114.4, 108.2, 104.7, 101.8, 99.5, 91.4, 81.0, 80.8, 72.2, 70.7, 69.5, 69.2, 68.3, 64.2, 62.8, 62.7, 56.7, 55.8, 47.6, 38.9, 30.4, 26.0, 21.2, 20.5, 18.7, 15.3, 14.9, 2.0, −4.3, −5.2 ppm; HRMS (ESI-TOF) calcd for C₄₂H₅₄O₁₈SiNa⁺ [M+Na]⁺ 897.2972, found 897.2961.

### (4R,5S,6S,8S,10R)-8-{[(1S,8S,10S,13aS)-8-[(4-O-Acetyl-2,6-dideoxy-3-C-methyl-α-L-xylo-hexo-pyranosyl)oxy]-10-{[tert-butyl(dimethyl)silyl]oxy}-2-(dimethoxymethyl)-12-hydroxy-7-methoxy-5-methyl-11-oxo-3a,4,8,9,10,11-hexahydro-13aH-spiro[2,4-epoxyfuro[3,2-b]naphtho[2,3-h]chromene-1,2′-oxiran]-13a-yl]oxy}-4,6-dimethyl-2-oxo-1,3,7-trioxaspiro[4.5]dec-10-yl acetate (29)

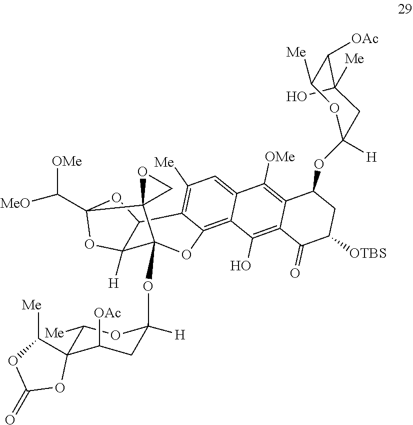

To a stirred mixture of the hydroxy mono-glycosylated product 27 (3.5 mg, 4 μmol, 1.0 equiv), PPh₃AuNTf₂ (0.025 M in CH₂Cl₂, 48 μL, 1.2 μmol, 0.3 equiv) and 4 Å MS (80 mg) in CH₂Cl₂ (0.4 ml) at 0° C. was added a solution of carbohydrate donor 28 (3.0 mg, 8.0 μmol, 2.0 equiv) in CH₂Cl₂ (160 L) dropwise over 0.5 h. The reaction mixture was stirred at this temperature for another 15 min, and then quenched with Et₃N (5 L). The resulting mixture was filtered through Celite® and concentrated under reduced pressure. The residue was purified by preparative thin layer chromatography (silica gel, hexanes:EtOAc 1:2) to give the title compound (3.7 mg, 3.5 μmol, 88%) as an orange foam. 29: R_{f}=0.39 (silica gel, hexanes:EtOAc 2:3); [α]_{D}²⁵=−57.0 (c=0.1, CH₂Cl₂); FT-IR (film): νₘₐₓ=3522, 2933, 2856, 1815, 1748, 1621, 1389, 1223, 1083, 1015, 996, 866 cm⁻¹; NMR (600 MHz, CDCl₃)=δ 7.25-7.22 (m, 1H), 7.20 (d, J=8.6 Hz, 2H), 6.79-6.73 (m, 2H), 5.50 (d, J=1.5 Hz, 1H), 5.33 (d, J=3.8 Hz, 1H), 5.21 (d, J=1.3 Hz, 1H), 5.13 (d, J=3.8 Hz, 1H), 5.09 (t, J=2.9 Hz, 1H), 4.80 (s, 1H), 4.76 (dd, J=12.4, 5.1 Hz, 1H), 4.60 (d, J=10.9 Hz, 1H), 4.50 (d, J=10.9 Hz, 1H), 3.93-3.87 (m, 1H), 3.79 (s, 3H), 3.69 (s, 3H), 3.53 (d, J=4.4 Hz, 6H), 2.62 (ddd, J=13.6, 5.2, 3.1 Hz, 1H), 2.48 (s, 3H), 2.04 (ddd, J=13.6, 12.4, 2.8 Hz, 1H), 1.70 (d, J=9.8 Hz, 1H), 1.05 (s, 9H), 0.98 (s, 9H), 0.85 (s, 9H), 0.13 (s, 3H), 0.04 (s, 3H) ppm; ¹³C NMR (151 MHz, CDCl₃) δ=203.0, 170.5, 170.1, 163.3, 153.4, 151.9, 144.4, 142.4, 135.1, 126.7, 116.8, 115.1, 114.7, 108.4, 104.7, 101.8, 99.5, 98.3, 91.4, 81.0, 80.8, 74.6, 72.2, 70.7, 69.4, 69.2, 68.9, 68.4, 68.3, 64.2, 62.9, 62.8, 56.6, 55.9, 47.6, 38.9, 36.8, 30.4, 25.9, 25.9, 25.9, 21.2, 21.0, 20.5, 18.6, 17.0, 15.3, 14.9, −4.2, −5.3 ppm; HRMS (ESI-TOF) calcd for C₅₁H₆₈O₂₂SiNa⁺ [M+Na]⁺1083.3864, found 1083.3849.

### (4S,5S,6S,8S,10R)-8-{[(1S,8S,10S,13aS)-10-{[tert-Butyl(dimethyl)silyl]oxy}-2-(dimethoxymethyl)-8,12-dihydroxy-7-methoxy-5-methyl-11-oxo-3a,4,8,9,10,11-hexahydro-13aH-spiro[2,4-epoxyfuro[3,2-b]naphtho[2,3-h]chromene-1,2′-oxiran]-13a-yl]oxy}-4,6-dimethyl-2-oxo-1,3,7-trioxaspiro[4.5]dec-10-yl acetate (31)

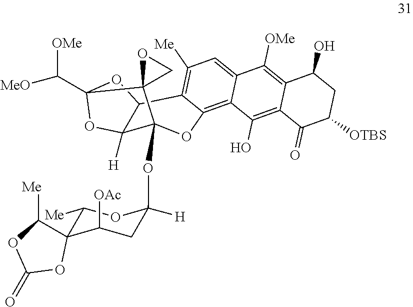

To a stirred solution of glycoside product 30 (Nicolaou et al., 2016) (5.6 mg, 6.73 μmol, 1.0 equiv) in CH₂Cl₂ (0.3 ml) and H₂O (0.075 ml, pH 7.0 buffer) at 23° C. in a reaction flask shielded from light using aluminum foil was added DDQ (4.6 mg, 20 μmol, 3.0 equiv). After stirring at this temperature for 3 h, the reaction was quenched with brine (2 ml). The resulting mixture was extracted with CH₂Cl₂ (3×2 ml), dried over anhydrous Na₂SO₄, and concentrated under reduced pressure. The residue was purified by preparative thin layer chromatography (silica gel, hexanes:EtOAc 1:2) to give 31 (3.7 mg, 4.23 μmol, 75%) as an orange foam. 31: R_{f}=0.29 (silica gel, EtOAc:hexanes 1:1); [α]_{D}²⁵=−6.3 (c=0.16, CH₂Cl₂); FT-IR (film): νₘₐₓ=3505, 2930, 2856, 1815, 1749, 1621, 1389, 1222, 1112, 1080, 1048, 1002, 837 cm⁻¹; ¹H NMR (600 MHz, CDCl₃) δ=14.40 (s, 1H), 7.43 (s, 1H), 5.78 (d, J=4.1 Hz, 1H), 5.43 (t, J=3.5 Hz, 1H), 5.24 (d, J=4.1 Hz, 1H), 5.18 (d, J=4.2 Hz, 1H), 5.10 (s, 1H), 4.90 (dd, J=11.8, 5.0 Hz, 1H), 4.70 (s, 1H), 4.64 (q, J=6.6 Hz, 1H), 4.57 (q, J=6.5 Hz, 1H), 3.90 (s, 3H), 3.61 (s, 3H), 3.45 (s, 3H), 2.80 (d, J=5.9 Hz, 1H), 2.72 (d, J=5.9 Hz, 1H), 2.59 (s, 3H), 2.50 (ddd, J=13.6, 5.0, 3.6 Hz, 1H), 2.47-2.39 (m, 2H), 2.33 (ddd, J=13.5, 11.8, 3.6 Hz, 1H), 2.25 (s, 3H), 2.22-2.15 (m, 1H), 1.39 (d, J=6.5 Hz, 3H), 1.28 (d, J=6.6 Hz, 3H), 0.96 (s, 9H), 0.23 (s, 3H), 0.16 (s, 3H) ppm; ¹³C NMR (151 MHz, CDCl₃) δ=202.9, 170.8, 170.5, 170.2, 163.4, 152.1, 145.0, 143.2, 135.6, 126.6, 116.8, 115.2, 115.0, 107.4, 104.8, 101.8, 99.8, 98.2, 92.3, 74.6, 72.0, 72.0, 71.4, 69.1, 68.9, 68.3, 68.2, 68.1, 67.7, 65.4, 63.1, 62.9, 56.7, 56.5, 47.5, 36.8, 29.9, 25.9, 21.4, 21.2, 21.0, 20.6, 17.0, 15.3, 15.0 ppm; HRMS (ESI-TOF) calcd for C₄₂H₅₄O₁₈SiNa⁺ [M+Na]⁺897.2972, found 897.2961.

### (4S,5S,6S,8S,10R)-8-{[(1S,8S,10S,13aS)-8-[(4-O-Acetyl-2,6-dideoxy-3-C-methyl-α-L-xylo-hexo-pyranosyl)oxy]-10-{[tert-butyl(dimethyl)silyl]oxy}-2-(dimethoxymethyl)-12-hydroxy-7-methoxy-5-methyl-11-oxo-3a,4,8,9,10,11-hexahydro-13aH-spiro[2,4-epoxyfuro[3,2-b]naphtho[2,3-h]chromene-1,2′-oxiran]-13a-yl]oxy}-4,6-dimethyl-2-oxo-1,3,7-trioxaspiro[4.5]dec-10-yl acetate (32)

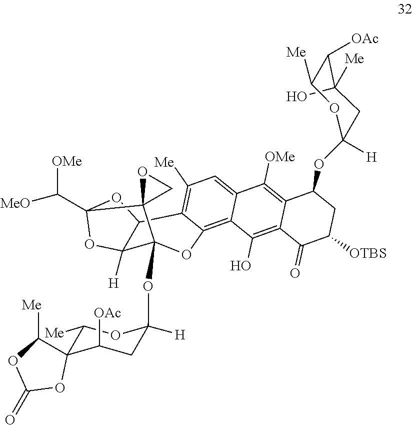

To a stirred solution of hydroxy glycoside product 31 (3.5 mg, 4.0 μmol, 1.0 equiv), were added sequentially PPh₃AuNTf₂ (0.025 M in CH₂Cl₂, 48 μL, 1.2 μmol, 0.3 equiv) and 4 Å MS (80 mg) in CH₂CO₂ (0.4 ml) at 0° C., followed by a solution of carbohydrate donor 28 (3.0 mg, 8.0 μmol, 2.0 equiv) in CH₂CO₂ (160 L) dropwise over 0.5 h. The reaction mixture was stirred at this temperature for another 15 min, and then quenched with Et₃N (5 L). The resulting mixture was filtered through Celite® and concentrated under reduced pressure. The residue was purified by preparative thin layer chromatography (silica gel, hexanes:EtOAc 1:2) to give the title compound (2.6 mg, 2.5 μmol, 62%) as an orange foam. 32: R_{f}=0.18 (silica gel, hexanes:EtOAc 1:1); [α]_{D}²⁵=−82.0 (c=0.1, CH₂CO₂); FT-IR (film): νₘₐₓ=3523, 1816, 1749, 1621, 1388, 1224, 1082, 998 cm⁻¹; ¹H NMR (600 MHz, CDCl₃) δ=14.60 (s, 1H), 7.46 (d, J=1.1 Hz, 1H), 5.77 (d, J=4.2 Hz, 1H), 5.43-5.33 (m, 2H), 5.25 (d, J=4.2 Hz, 1H), 5.18 (d, J=4.2 Hz, 1H), 5.15-5.08 (m, 1H), 4.79 (dd, J=12.6, 5.2 Hz, 1H), 4.75 (s, 1H), 4.70 (s, 1H), 4.64 (q, J=6.5 Hz, 1H), 4.57 (q, J=6.4 Hz, 1H), 4.54-4.49 (m, 1H), 3.82 (s, 3H), 3.80 (s, 1H), 3.61 (s, 3H), 3.45 (s, 3H), 2.82 (d, J=5.9 Hz, 1H), 2.74 (d, J=5.9 Hz, 1H), 2.63-2.53 (m, 4H), 2.46 (dd, J=14.9, 2.8 Hz, 1H), 2.35 (td, J=13.1, 2.8 Hz, 1H), 2.25 (s, 3H), 2.19 (dt, J=15.2, 3.8 Hz, 1H), 2.14 (s, 3H), 1.95 (dd, J=14.6, 4.1 Hz, 1H), 1.63 (d, J=14.6 Hz, 1H), 1.39 (d, J=6.5 Hz, 3H), 1.29 (d, J=6.6 Hz, 3H), 1.23 (d, J=6.6 Hz, 3H), 1.08 (s, 3H), 0.96 (s, 9H), 0.26 (s, 3H), 0.17 (s, 3H) ppm; ¹³C NMR (151 MHz, CDCl₃) δ=202.9, 170.5, 170.0, 163.3, 153.4, 151.9, 144.4, 142.4, 135.1, 126.7, 116.7, 115.2, 114.6, 108.4, 104.7, 101.7, 99.2, 98.2, 91.9, 80.8, 74.6, 72.1, 69.4, 69.2, 68.9, 68.4, 68.3, 66.3, 65.7, 62.9, 62.7, 56.5, 55.8, 47.6, 36.8, 29.8, 25.9, 25.9, 21.3, 21.0, 20.5, 18.6, 17.0, 15.3, 13.6, −4.2, −5.3 ppm; HRMS (ESI-TOF) calcd for C₅₁H₆₈O₂₂SiNa⁺ [M+Na]⁺ 1083.3864, found 1083.3839.

### (E)-2,2-Di-tert-butyl-4-(3-Hydroxyprop-1-en-1-yl)-7-methoxy-5-methyl-9,10-dihydroanthra[1,9-de][1,3,2]dioxasilin-11(8H)-one (34)

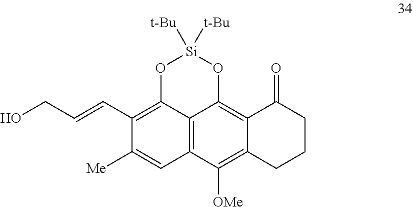 [SMILES: COc1c2c(c3c4c(c(/C=C/CO)c(C)cc14)O[Si](C(C)(C)C)(C(C)(C)C)O3)C(=O)CCC2]

To a stirred mixture of aryl bromide 18 (3.60 g, 7.33 mmol, 1.0 equiv), tri(2-furyl)phosphine (343 mg, 1.48 mmol, 0.2 equiv) and Pd(PPh₃)₄ (844 mg, 0.73 mmol, 0.1 equiv) in DMF (100 ml) was added stannane 14 (3.80 g, 11.0 mmol, 1.5 equiv), After bubbled with argon balloon three times, N,N-diisopropylethylamine (2.55 ml, 14.7 mmol, 2.0 equiv) and LiCl (1 M in THF, 14.7 ml, 14.7 mmol, 2.0 equiv) was added. After stirring at 100° C. for 12 h, the reaction mixture was cooled to 23° C. and then was diluted with EtOAc (200 ml) and quenched with water (100 ml). The resulting mixture was washed with brine (3×50 ml), dried over anhydrous Na₂SO₄, filtered and concentrated under reduced pressure. The residue was purified by flash column chromatography (silica gel, EtOAc:hexanes 1:4→1:2) to give the title compound (2.54 g, 5.42 mmol, 74%) as a yellow foam. 34: R_{f}=0.23 (silica gel, EtOAc:hexanes 1:4); FT-IR (film): νₘₐₓ=3696, 3681, 1678, 1605, 1585, 1401, 1371, 1168, 1058, 1014 cm⁻¹; ¹H NMR (600 MHz, CDCl₃) δ 7.38 (s, 1H), 6.75-6.67 (dt, J=16.1, 1.5 Hz, 1H), 6.58 (dt, J=16.1, 5.8 Hz, 1H), 4.39 (ddd, J=6.0, 5.9, 1.5 Hz, 2H), 3.83 (s, 3H), 3.04 (dd, J=6.9, 5.3 Hz, 2H), 2.64 (dd, J=7.2, 5.8 Hz, 2H), 2.52 (s, 3H), 2.08 (quint, J=6.5 Hz, 2H), 1.40 (t, J=6.0 Hz, 1H), 1.13 (s, 18H) ppm; ¹³C NMR (151 MHz, CDCl₃) δ=196.7, 151.2, 150.7, 144.4, 139.8, 133.9, 131.6, 130.8, 124.8, 120.1, 116.3, 114.8, 114.7, 65.2, 61.1, 41.4, 26.4, 24.3, 22.5, 22.4, 21.3 ppm; HRMS (ESI-TOF) calcd for C₂₇H₃₇O₅Si⁺ [M+H]⁺ 469.2405, found 469.2419.

### (E)-3-(2,2-Di-tert-butyl-7-methoxy-5-methyl-11-oxo-8,9,10,11-tetrahydroanthra[1,9-de][1,3,2]di-oxasilin-4-yl)acrylaldehyde (35)

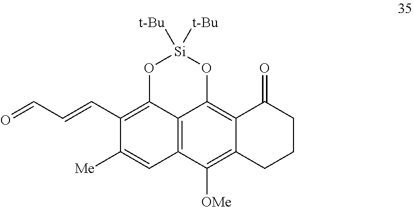 [SMILES: COc1c2c(c3c4c(c(/C=C/C=O)c(C)cc14)O[Si](C(C)(C)C)(C(C)(C)C)O3)C(=O)CCC2]

To a stirred solution of allylic alcohol 34 (2.50 g, 5.34 mmol, 1.0 equiv) in CH₂Cl₂ (50 ml) at 0° C. were added TPAP (187 mg, 0.53 mmol, 0.1 equiv) and NMO (937 mg, 8.01 mmol, 1.5 equiv). After stirring at this temperature for 4 h, the reaction was quenched with NaHCO₃ (20 ml). The resulting mixture was extracted with CH2Cl2 (3×50 ml), and the combined organic phases were dried over anhydrous Na₂SO₄, filtered and concentrated under reduced pressure. The residue was purified by flash column chromatography (silica gel, EtOAc:hexanes 1:6-1:4) to give the title compound (2.04 g, 4.37 mmol, 82%) as a yellow foam. 35: R_{f}=0.55 (silica gel, EtOAc:hexanes 1:4); FT-IR (film): νₘₐₓ=2937, 2862, 1682, 1583, 1472, 1372, 1167, 1130, 1015, 887 cm⁻¹; ¹H NMR (600 MHz, CDCl₃) δ=9.71 (d, J=7.7 Hz, 1H), 7.76 (d, J=16.0 Hz, 1H), 7.42 (d, J=1.2 Hz, 1H), 7.06 (dd, J=16.0, 7.7 Hz, 1H), 3.84 (s, 3H), 3.06 (dd, J=6.9, 5.4 Hz, 2H), 2.65 (dd, J=7.2, 5.8 Hz, 2H), 2.62 (d, J=1.0 Hz, 3H), 2.09 (p, J=6.5 Hz, 2H), 1.14 (s, 18H) ppm; ¹³C NMR (151 MHz, CDCl₃) δ=196.3, 195.5, 154.6, 150.9, 147.0, 144.7, 139.8, 134.4, 132.6, 132.4, 117.5, 117.0, 115.6, 114.5, 61.2, 41.3, 26.3, 26.3, 24.4, 22.4, 22.4, 21.3 ppm; HRMS (ESI-TOF) calcd for C₂₇H₃₅O₅Si⁺ [M+H]⁺ 467.2248, found 467.2259.

### 1-(1,3-Dioxolan-2-yl)prop-2-en-1-one (38a)

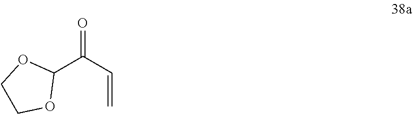

To a stirred solution of 1-(1,3-dioxolan-2-yl)ethan-1-one (4.00 g, 34.5 mmol, 1.0 equiv), paraformaldehyde (3.10 g, 103 mmol, 3.0 equiv) and diisopropylammonium trifluoroacetate (15.5 g, 72.0 mmol, 2.1 equiv) in dry THF (30 ml), trifluoroacetic acid (0.26 ml, 3.40 mmol) was added, and the mixture was heated under reflux for 12 h, the reaction mixture was cooled to 23° C., then was diluted with Et₂O (50 ml) and quenched with NaHCO₃ (sat. aq., 50 ml). The resulting mixture was extracted with Et₂O (3×50 ml). The combined organic phases were dried over anhydrous Na₂SO₄, filtered and concentrated under reduced pressure. The residue was purified by flash column chromatography (silica gel, Et₂O:pentane 1:4→1:2) to give the title compound (2.30 g, 18.0 mmol, 52%) as a colorless oil. R_{f}=0.43 (silica gel, hexanes:EtOAc 3:1); FT-IR (film): νₘₐₓ=2895, 1709, 1697, 1614, 1475, 1406, 1205, 1106, 1058, 1028, 939 cm⁻¹; ¹H NMR (600 MHz, CDCl₃) δ=6.71-6.46 (m, 1H), 6.53-6.31 (m, 1H), 6.04-5.72 (m, 1H), 5.46-5.16 (m, 1H), 4.12-3.74 (m, 4H); ¹³C NMR (151 MHz, CDCl₃) δ=194.0, 131.3, 101.2 (overlap), 65.7 ppm; HRMS (ESI-TOF) calcd for C₆H₉O₃⁺ [M+H]⁺ 129.0552, found 129.0551.

### 1-(1,3-dioxan-2-yl)prop-2-en-1-one (38b)

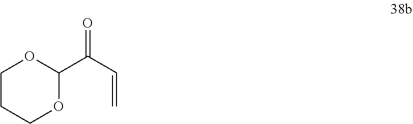

38b was prepared from the corresponding 1-(1,3-dioxan-2-yl)ethan-1-one. R_{f}=0.26 (silica gel, hexanes:EtOAc 3:1); FT-IR (film) νₘₐₓ=2958, 2920, 2851, 1803, 1716, 1615, 1462, 1407, 1378, 1238 cm⁻¹; ¹H NMR (600 MHz, CDCl₃) δ=6.73 (dd, J=17.5, 10.7 Hz, 1H), 6.50 (dd, J=17.6, 1.6 Hz, 1H), 5.88 (dd, J=10.7, 1.6 Hz, 1H), 4.98 (s, 1H), 4.24 (ddd, J=12.2, 4.9, 1.6 Hz, 2H), 3.89 (td, J=12.2, 2.4 Hz, 3H), 2.22-2.14 (m, 1H), 1.45 (dt, J=14.1, 2.3 Hz, 1H) ppm; ¹³C NMR (151 MHz, CDCl₃) δ=191.9, 131.4, 131.0, 100.3, 67.2, 25.8 ppm; HRMS (ESI-TOF) calcd for C₇H₁₁O₃⁺ [M+H]⁺ 143.0703, found 143.0705.

### 2,2-Di-tert-butyl-4-[(3S)-3-{2-(1,3-dioxolan-2-ylcarbonyl)-1-[(trimethylsilyl)oxy]prop-2-en-1-yl}oxiran-2-yl]-7-methoxy-5-methyl-9,10-dihydroanthra[1,9-de][1,3,2]dioxasilin-11(8H)-one (40a)

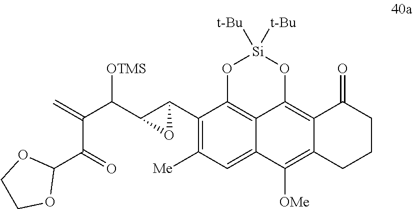

To a stirred solution of aldehyde 35 (400 mg, 0.858 mmol, 1.0 equiv) in toluene (10 ml) at 23° C. were added H₂O₂ (30 wt % in H₂O, 110 μl, 1.08 mmol, 1.3 equiv) and (S)-(−)-α,α-diphenyl-2-pyrrolidine methanol trimethylsilyl ether (0.05 M in toluene, 3.40 ml, 0.171 mmol, 0.2 equiv). After stirring at the same temperature for 4 h, the reaction mixture was diluted with EtOAc (50 ml). The resulting mixture was washed with H₂O (2×10 ml), dried over anhydrous Na₂SO₄, filtered and concentrated under reduced pressure to give the crude epoxide 37, which was taken to the next step without further purification.

To a stirred solution of the so obtained crude epoxide 37 in THF (5 ml) at 23° C. was added DABCO (48.0 mg, 0.429 mmol, 0.5 equiv), 4-nitrophenol (59.6 mg, 0.429 mmol, 0.5 equiv) and enone 38a (690 mg, 5.4 mmol, 6.3 equiv). After stirring at this temperature for 12 h, the reaction mixture was diluted with EtOAc (50 ml). The resulting mixture was washed with brine (2×10 ml) and concentrated under reduced pressure. The residue was purified by flash column chromatography (silica gel, EtOAc:hexanes 1:4→1:2) to give the crude alcohol 39a. To a stirred solution of crude alcohol obtained above in CH₂Cl₂ (10 ml) at −78° C. was added dropwise a solution of imidazole (116 mg, 1.67 mmol, 2.0 equiv) in CH₂Cl₂ (2 ml), followed by TMSCl (108 μl, 0.86 mmol, 1.0 equiv). The resulting reaction mixture was stirred at this temperature for 15 min before it was quenched with NaHCO₃ (sat. aq., 5 ml). The resulting mixture was stirred at 23° C. for 15 min, and then extracted with CH₂Cl₂ (3×5 ml). The combined organic phases were dried over anhydrous Na₂SO₄, filtered and concentrated under reduced pressure. The residue was purified by flash column chromatography (silica gel, EtOAc:hexanes 1:4) to give the title compound (40a, plus C4-epi-40a) (205 mg, 0.30 mmol, ca. 5:1 dr, 35% for the three steps) as a yellow foam. 40a (plus C4-epi-40a): R_{f}=0.20 (silica gel, EtOAc:hexanes 1:4); [α]_{D}²⁵=−66.9 (c=0.2, CH₂Cl₂); FT-IR (film): νₘₐₓ=2935, 2897, 1682, 1614, 1403, 1371, 1252, 1147, 1016, 888 cm⁻¹; H NMR (600 MHz, C₆H₆) δ=7.55 (s, 1H, major), 7.52 (s, 1H, minor), 6.46-6.43 (m, 1H, major), 6.40-6.39 (m, 1H, minor), 6.26 (s, 1H, major), 6.28-6.25 (m, 1H, minor), 5.64 (s, 1H, minor), 5.61 (s, 1H, major), 5.41-5.40 (m, 1H, minor), 5.26 (dd, J=3.8, 1.1 Hz, 1H, major), 4.43 (d, J=2.2 Hz, 1H, major), 4.03 (d, J=2.3 Hz, 1H, minor), 3.80-3.79 (m, 1H, minor), 3.69 (dd, J=3.7, 2.2 Hz, 1H, major), 3.65-3.53 (m, 4H, major+minor), 3.49 (s, 3H, minor), 3.49 (s, 3H, major), 3.41-3.36 (m, 4H, major+minor), 2.78-2.63 (m, 4H, major+minor), 2.55 (s, 3H, major), 2.47 (s, 3H, minor), 2.41-2.31 (m, 4H, major+minor), 1.54 (quint, J=6.4 Hz, 4 H, major+minor), 1.30 (s, 9H, minor), 1.28 (s, 9H, major), 1.27 (s, 9H, minor), 1.26 (s, 9H, major), 0.17 (s, 9H, minor), 0.16 (s, 9H, major) ppm; ¹³C NMR (151 MHz, C₆D₆) δ=194.9 (major+minor), 194.32 (major), 194.24 (minor), 152.8 (minor), 152.3 (major), 150.4 (major+minor), 145.9 (major+minor), 145.18 (minor), 145.11 (major), 141.5 (major), 140.8 (minor), 132.40 (major), 132.35 (minor), 132.0 (major), 131.9 (minor), 129.6 (minor), 129.2 (major), 119.7 (major), 119.6 (minor), 117.08 (major), 117.07 (minor), 115.4 (major+minor), 114.92 (major), 114.91 (minor), 99.9 (major), 99.8 (minor), 69.2 (major), 68.4 (minor), 65.41 (minor), 65.39 (major), 65.33 (minor), 65.31 (major), 62.3 (major), 61.2 (minor), 60.47 (major), 60.45 (minor), 53.1 (major), 51.4 (minor), 41.4 (major+minor), 26.6 (major), 26.5 (major), 24.3 (major+minor), 22.4 (major+minor), 21.7 (minor), 21.6 (major), 21.4 (major), 21.3 (minor), 21.2 (minor), 21.1 (major), 0.2 (minor), 0.1 (major) ppm; HRMS (ESI-TOF) calcd for C₃₆H₅₀O₉Si₂Na⁺ [M+Na]⁺ 705.2886, found 705.2885.

### 2,2-Di-tert-butyl-4-{(1R,3S,4S,5R)-1-(1,3-dioxolan-2-yl)-6-methylene-5-[(trimethylsilyl)oxy]-2,7-dioxabicyclo[2.2.1]hept-3-yl}-7-methoxy-5-methyl-9,10-dihydroanthra[1,9-de][1,3,2]dioxasilin-11(8H)-one (41a)

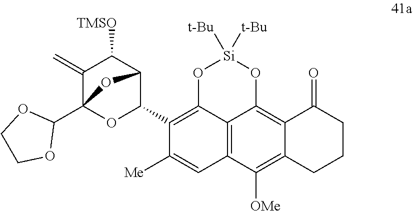

To a flame-dried flask and 4 Å molecule sieves was added the solution of 40a (160 mg, 0.234 mmol, 1.0 equiv) in CH2Cl12 (4 ml). To the stirred mixture at −50° C. was added a solution of BF₃-Et₂O (0.1 M in CH₂Cl₂, 704 μl, 0.0704 mmol, 0.3 equiv). The resulting mixture was allowed to warm to 0° C. and was quenched by NaHCO₃ (sat. aq., 5 ml) and then extracted with EtOAc (3×5 ml). The combined organic phases were dried over anhydrous Na₂SO₄, filtered and concentrated under reduced pressure. The residue was purified by flash column chromatography (silica gel, EtOAc:hexanes 1:4) to afford 41a (76.8 mg, 0.112 mmol, 48%) as a yellow foam. 41a: R_{f}=0.67 (silica gel, EtOAc:hexanes 1:2); [α]_{D}²⁵=+161.0 (c=0.2, CH₂Cl₂); FT-IR (film): νₘₐₓ=2935, 2862, 1681, 1609, 1397, 1368, 1251, 1147, 891, 827 cm⁻¹; ¹H NMR (600 MHz, CDCl₃) δ=7.24 (s, 1H), 5.72 (d, J=3.2 Hz, 1H), 5.52 (s, 1H), 5.40 (d, J=2.5 Hz, 1H), 5.06 (d, J=2.1 Hz, 1H), 5.01 (dd, J=4.9, 3.2 Hz, 1H), 4.71 (dt, J=4.7, 2.3 Hz, 1H), 4.27-4.20 (m, 2H), 4.13-4.01 (m, 2H), 3.79 (s, 3H), 3.07 (ddd, J=16.3, 7.4, 4.8 Hz, 1H), 2.97 (ddd, J=16.3, 7.9, 4.8 Hz, 1H), 2.65-2.60 (m, 5H), 2.14-1.93 (m, 2H), 1.18 (s, 9H), 1.05 (s, 9H), −0.34 (s, 9H) ppm; ¹³C NMR (151 MHz, CDCl₃) δ=196.8, 150.0, 148.9, 147.7, 144.2, 141.8, 130.6, 130.5, 120.0, 116.3, 115.8, 113.7, 108.6, 106.4, 100.3, 80.2, 80.1, 73.5, 66.1, 65.9, 61.0, 41.4, 26.6, 24.4, 24.2, 22.5, 21.7, 20.8, −0.3 ppm; HRMS (ESI-TOF) calcd for C₃₆H₅₁O₉Si₂⁺ [M+H]⁺ 683.3066, found 683.3055.

### 2,2-Di-tert-butyl-4-[(1R,3S,4R,5S)-1-(1,3-dioxolan-2-yl)-5-hydroxy-6-methylene-2,7-dioxabicyclo-[2.2.1]hept-3-yl]-7-methoxy-5-methyl-9,10-dihydroanthra[1,9-de][1,3,2]dioxasilin-11(8H)-one (42a)

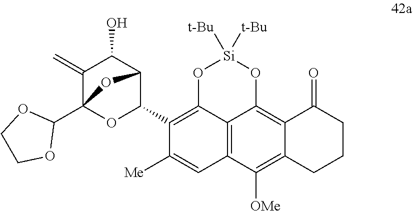 [SMILES: C=C1[C@H](O)[C@H]2O[C@]1(C1OCCO1)O[C@H]2c1c(C)cc2c(OC)c3c(c4c2c1O[Si](C(C)(C)C)(C(C)(C)C)O4)C(=O)CCC3]

41a (50.0 mg, 0.0733 mmol) was dissolved in a solution of TFA (0.1 M in THF:H₂O 5:1, 2.5 ml) at 23° C. After stirring at this temperature for 3 h, the reaction was quenched with NaHCO₃ (sat. aq., 10 ml). The resulting mixture was extracted with CH₂Cl₂(3×5 ml), and the combined organic phases were dried over anhydrous Na₂SO₄, filtered and concentrated under reduced pressure. The residue was purified by flash column chromatography (silica gel, EtOAc:hexanes 1:4→1:2) to give the title compound (32.0 mg, 0.052 mmol, 72%) as a colorless oil. 42a: R_{f}=0.36 (silica gel, EtOAc:hexanes 1:2); [α]_{D}²⁵=+368.6 (c=0.3, CH₂Cl₂); FT-IR (film): νₘₐₓ=3474, 2935, 2862, 1679, 1608, 1556, 1472, 1372, 1066, 887 cm⁻¹; ¹H NMR (600 MHz, CDCl₃) δ=7.35 (s, 1H), 5.69 (d, J=3.3 Hz, 1H), 5.53 (s, 1H), 5.50 (d, J=2.7 Hz, 1H), 5.36-5.31 (m, 1H), 5.22 (d, J=2.3 Hz, 1H), 4.69-4.60 (m, 1H), 4.35-4.17 (m, 2H), 4.14-3.98 (m, 2H), 3.82 (s, 3H), 3.02 (q, J=6.1 Hz, 2H), 2.66 (s, 3H), 2.62 (dd, J=7.1, 5.8 Hz, 2H), 2.06 (p, J=6.3 Hz, 2H), 1.18 (s, 9H), 1.09 (s, 9H), 0.98 (d, J=11.4 Hz, 1H) ppm; ¹³C NMR (151 MHz, CDCl₃) δ=196.6, 150.3, 149.8, 148.1, 144.3, 138.9, 131.8, 131.0, 118.3, 117.8, 116.4, 113.9, 108.5, 107.4, 100.2, 79.8, 79.5, 75.5, 66.2, 66.0, 61.2, 41.3, 26.5, 26.4, 24.9, 24.3, 22.4, 21.4, 21.2 ppm; HRMS (ESI-TOF) calcd for C₃₃H₄₃O₉Si⁺ [M+H]⁺ 611.2671, found 611.2666.

### 2,2-Di-tert-butyl-4-[(1R,2S,3R,4R,5S)-1-(1,3-dioxolan-2-yl)-3-hydroxyspiro[6,7-dioxabicyclo-[2.2.1]heptane-2,2′-oxiran]-5-yl]-7-methoxy-5-methyl-9,10-dihydroanthra[1,9-de][1,3,2]dioxasilin-11(8H)-one (45a)

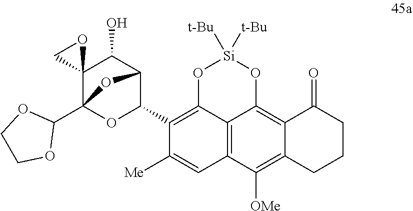 [SMILES: COc1c2c(c3c4c(c([C@@H]5O[C@@]6(C7OCCO7)O[C@@H]5[C@@H](O)[C@@]65CO5)c(C)cc14)O[Si](C(C)(C)C)(C(C)(C)C)O3)C(=O)CCC2]

To a stirred solution of 42a (22.3 mg, 0.037 mmol, 1.0 equiv) in acetone (1.1 ml) at 23° C. were sequentially added OsO₄ (0.08 M aq., 92 μl, 0.0073 mmol, 0.2 equiv) and NMO (0.48 M aq., 305 μl, 0.146 mmol, 4.0 equiv). The mixture was stirred at this temperature for 12 h before it was quenched with Na₂SO₃ (10% aq., 2 ml). The resulting mixture was stirred for another 0.5 h and was extracted with EtOAc (3×5 ml). The combined organic phases were dried over anhydrous Na₂SO₄, filtered and concentrated under reduced pressure. The residue was filtered through a short pad of silica gel to afford triol 43a (20.0 mg, 0.0311 mmol) as a colorless oil, which was used for the nest step without further purification.

To a stirred solution of the so obtained crude triol (20.0 mg, 0.0311 mmol, 1.0 equiv) in CH₂Cl₂ (2 ml) at 23° C. were sequentially added Et₃N (22 μl, 0.155 mmol, 5.0 equiv), 4-dimethylaminopyridine (1.9 mg, 0.016 mmol, 0.5 equiv) and TsCl (29.6 mg, 0.155 mmol, 5.0 equiv). The mixture was stirred at this temperature for 5 h before it was quenched with NH₄Cl (sat. aq., 2 ml). The resulting mixture was extracted with CH₂Cl₂ (3×5 ml), and the combined organic phases were dried over anhydrous Na₂SO₄, filtered and concentrated under reduced pressure. The so obtained residue was filtered through a short pad of silica gel to afford the tosylate (24.8 mg, 31.0 μmol) as a colorless foam, which was used for the nest step without further purification.

To a solution of the so obtained tosylate (24.8 mg, 31.0 μmol, 1.0 equiv) in MeOH (2 ml) at 23° C. was added K₂CO₃ (8.5 mg, 0.062 mmol, 2.0 equiv). The mixture was stirred at this temperature for 2 h and was then directly subjected to flash column chromatography (silica gel, EtOAc:hexanes 1:1) to give epoxyalcohol 45a (18.0 mg, 0.0287 mmol, 78% for the three steps) as a colorless foam. 45a: R_{f}=0.52 (silica gel, hexanes:EtOAc 1:1); [α]_{D}²⁵=+117.3 (c=0.1, CH₂Cl₂); FT-IR (film): νₘₐₓ=3455, 2934, 2861, 1680, 1608, 1557, 1472, 1372, 1172, 1091, 970, 827, 661 cm⁻¹; ¹H NMR (600 MHz, CDCl₃) δ=7.38 (s, 1H), 5.66 (d, J=3.1 Hz, 1H), 5.45 (dd, J=5.1, 3.2 Hz, 1H), 5.43 (s, 1H), 4.35 (dd, J=9.9, 5.0 Hz, 1H), 4.25-4.18 (m, 1H), 4.20-4.13 (m, 1H), 4.06-3.97 (m, 2H), 3.83 (s, 3H), 3.37 (d, J=5.4 Hz, 1H), 3.08 (d, J=5.3 Hz, 1H), 3.03 (q, J=6.3 Hz, 2H), 2.71 (s, 3H), 2.62 (dd, J=7.2, 5.9 Hz, 2H), 2.06 (quint, J=6.4 Hz, 2H), 1.30 (d, J=10.1 Hz, 1H), 1.18 (s, 9H), 1.09 (s, 9H) ppm; ¹³C NMR (151 MHz, CDCl₃) δ=196.6, 150.3, 149.8, 144.4, 139.0, 132.0, 131.0, 117.7, 117.6, 116.5, 113.9, 108.0, 98.9, 79.6, 78.7, 76.5, 68.0, 66.3, 65.8, 61.2, 47.4, 41.3, 26.5, 26.4, 24.3, 24.2, 22.4, 21.4, 21.2 ppm; HRMS (ESI-TOF) calcd for C₃₃H₄₃O₁₀Si⁺ [M+H]⁺ 627.2620, found 627.2644.

### 2,2-Di-tert-butyl-4-[(1R,2S,4S,5S)-1-(1,3-dioxolan-2-yl)-3-oxospiro[6,7-dioxabicyclo[2.2.1]heptane-2,2′-oxiran]-5-yl]-7-methoxy-5-methyl-9,10-dihydroanthra[1,9-de][1,3,2]dioxasilin-11(8H)-one (46a)

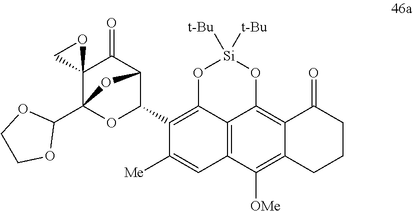 [SMILES: COc1c2c(c3c4c(c([C@@H]5O[C@@]6(C7OCCO7)O[C@@H]5C(=O)[C@@]65CO5)c(C)cc14)O[Si](C(C)(C)C)(C(C)(C)C)O3)C(=O)CCC2]

To a stirred solution of epoxy alcohol 45a (18.0 mg, 0.0287 mmol, 1.0 equiv) in CH₂Cl₂ at 23° C. were sequentially added NMO (10.1 mg, 0.0861 mmol, 3.0 equiv) and TPAP (2.0 mg, 5.7 μmol, 0.2 equiv). The mixture was stirred at this temperature for 1 h and then subjected directly to flash column chromatography (silica gel, EtOAc:hexanes 1:2) to afford 46a (16.8 mg, 0.0269 mmol, 94%) as a yellow foam. 46a: R_{f}=0.7 (silica gel, EtOAc:hexanes 1:2); [α]_{D}²⁵=+284.3 (c=0.21, CH₂Cl₂); FT-IR (film): νₘₐₓ=2934, 2860, 1787, 1682, 1609, 1398, 1372, 1040, 889 cm⁻¹; ¹H NMR (600 MHz, CDCl₃) δ=7.31 (d, J=1.1 Hz, 1H), 5.62 (d, J=3.8 Hz, 1H), 5.55 (s, 1H), 5.50 (d, J=3.8 Hz, 1H), 4.32-4.24 (m, 1H), 4.23-4.17 (m, 1H), 4.11-4.07 (m, 1H), 4.07-4.00 (m, 1H), 3.82 (s, 3H), 3.48 (d, J=6.2 Hz, 1H), 3.12 (d, J=6.2 Hz, 1H), 3.09-2.96 (m, 2H), 2.66-2.61 (m, 2H), 2.57 (s, 3H), 2.07 (quint, J=6.4 Hz, 2H), 1.20 (s, 9H), 1.08 (s, 9H) ppm; ¹³C NMR (151 MHz, CDCl₃) δ=202.3, 196.5, 150.5, 149.8, 144.3, 138.9, 132.2, 131.3, 117.6, 116.5, 116.2, 113.9, 107.5, 98.5, 78.3, 66.5, 65.9, 61.8, 61.2, 49.9, 41.3, 26.6, 26.3, 24.3, 23.9, 22.4, 21.5, 21.2 ppm; HRMS (ESI-TOF) calcd for C₃₃H₄₁O₁₀Si⁺ [M+H]⁺ 625.2464, found 625.2472.

### (1S,2S,3aS,4R,13aR)-2-(1,3-Dioxolan-2-yl)-12,13a-dihydroxy-7-methoxy-5-methyl-3a,4,8,9,10,13a-hexahydro-11H-spiro[2,4-epoxyfuro[3,2-b]naphtho[2,3-h]chromene-1,2′-oxiran]-11-one (Trx8)

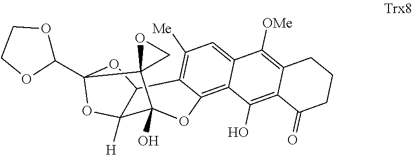

To a solution of epoxide 46a (16.8 mg, 0.0269 mmol, 1.0 equiv) in MeCN (1 ml) at 23° C. was added Et₃N.3HF (11.9 mg, 0.081 mmol, 3.0 equiv). The mixture was stirred at this temperature for 15 min before it was diluted with EtOAc (5 ml) and quenched with NaHCO₃ (sat. aq., 2 ml). The resulting mixture was extracted with EtOAc (3×5 ml), and the combined organic phases were dried over anhydrous Na₂SO₄, filtered and concentrated under reduced pressure. The residue was purified by flash column chromatography (silica gel, EtOAc:hexanes1:1) to afford Trx8 (11.6 mg, 0.0238 mmol, 89%) as a yellow foam. Trx8: R_{f}=0.23 (silica gel, EtOAc:hexanes 1:1); [α]_{D}²⁵=+368.6 (c=0.1, CH₂Cl₂); FT-IR (film): νₘₐₓ=2924, 1621, 1571, 1445, 1388, 1330, 1093, 978 cm⁻¹; ¹H NMR (600 MHz, CDCl₃) δ=14.91 (s, 1H), 7.44 (s, 1H), 5.40 (s, 1H), 5.25 (d, J=4.0 Hz, 1H), 4.85 (d, J=4.0 Hz, 1H), 4.45 (s, 1H), 4.13 (q, J=6.9, 6.4 Hz, 1H), 4.07-4.02 (m, 1H), 4.02-3.98 (m, 1H), 3.92-3.86 (m, 1H), 3.78 (s, 3H), 3.19 (d, J=5.2 Hz, 1H), 3.06 (d, J=5.2 Hz, 1H), 3.05-3.01 (m, 2H), 2.73 (td, J=6.0, 1.8 Hz, 2H), 2.56 (s, 3H), 2.15-2.03 (m, 2H) ppm; ¹³C NMR (151 MHz, CDCl₃) δ=204.6, 163.0, 151.7, 142.6, 141.6, 135.4, 130.5, 116.0, 113.6, 113.4, 111.2, 103.8, 98.7, 98.6, 73.9, 70.1, 69.4, 66.3, 65.9, 61.0, 50.3, 38.9, 23.7, 22.2, 20.4 ppm; HRMS (ESI-TOF) calcd for C₂₅H₂₄O₁₀Na⁺ [M+Na]⁺507.1262, found 507.1260.

### (1S,2S,3aS,4R,13aS)-2-(1,3-Dioxolan-2-yl)-12-hydroxy-7,13a-dimethoxy-5-methyl-3a,4,8,9,10,13a-hexahydro-11H-spiro[2,4-epoxyfuro[3,2-b]naphtho[2,3-h]chromene-1,2′-oxiran]-11-one (Trx9)

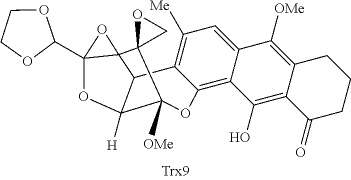 [SMILES: COc1c2c(c(O)c3c4c(c(C)cc13)C1C3OC5(C6OCCO6)OC15[C@]1(CO1)[C@]3(OC)O4)C(=O)CCC2]

To a stirred mixture of Trx8 (2.0 mg, 4.1 μmol, 1.0 equiv) and Me (0.5 ml) were sequentially added Ag₂O (4.8 mg, 21 μmol, 5.0 equiv) and CaSO₄ (2.8 mg, 21 μmol, 5.0 equiv) at 23° C. The mixture was stirred at this temperature for 12 h before it was subjected to a flash column chromatography (silica gel, EtOAc:hexanes 1:1) to afford Trx9 (1.3 mg, 2.61 μmol, 63%) as a yellow foam. Trx9: R_{f}=0.4 (silica gel, EtOAc:hexanes 1:1); [α]_{D}²⁵=+159.7 (c=0.1, CH₂Cl₂); FT-IR (film): νₘₐₓ=2924, 1621, 1571, 1445, 1388, 1235, 1180, 1093, 1073, 1017, 949 cm⁻¹; ¹H NMR (600 MHz, CDCl₃) δ=14.79 (s, 1H), 7.46 (d, J=1.2 Hz, 1H), 5.44 (s, 1H), 5.22 (d, J=4.1 Hz, 1H), 4.84 (d, J=4.1 Hz, 1H), 4.12 (q, J=6.6 Hz, 1H), 4.08-4.01 (m, 1H), 4.03-3.96 (m, 1H), 3.88 (q, J=6.6 Hz, 1H), 3.79 (s, 3H), 3.76 (s, 3H), 3.05 (td, J=5.8, 3.1 Hz, 2H), 2.97 (d, J=5.6 Hz, 1H), 2.88 (d, J=5.6 Hz, 1H), 2.73 (t, J=6.5 Hz, 2H), 2.57 (d, J=1.0 Hz, 3H), 2.10 (quint, J=6.2 Hz, 2H) ppm; ¹³C NMR (151 MHz, CDCl₃) δ=204.6, 163.1, 151.6, 142.6, 141.7, 135.4, 130.5, 115.8, 113.4, 111.2, 104.5, 102.3, 98.7, 72.0, 69.4, 69.1, 66.2, 65.9, 61.1, 53.0, 47.8, 39.0, 23.8, 22.3, 20.4 ppm; HRMS (ESI-TOF) calcd for C₂₆H₂₆O₁₀Na⁺ [M+Na]⁺521.1418, found 521.1416.

### (1S,2S,3aS,4R,13aS)-2-(1,3-Dioxolan-2-yl)-12-hydroxy-7-methoxy-13a-(methoxymethoxy)-5-methyl-3a,4,8,9,10,13a-hexahydro-11H-spiro[2,4-epoxyfuro[3,2-b]naphtho[2,3-h]chromene-1,2′-oxiran]-11-one (Trx10)

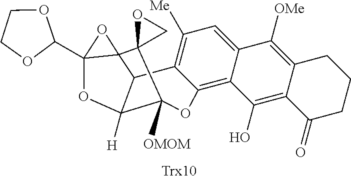

To a stirred solution of Trx8 (3.2 mg, 6.2 μmol, 1.0 equiv) in CH₂Cl₂ (1 ml) at 23° C. were sequentially added N,N-diisopropylethylamine (5.4 μl, 31 μmol, 5.0 equiv) and MOMCl (2.0 μl, 19 μmol, 3.0 equiv). The mixture was stirred at this temperature for 1 h before it was quenched with NaHCO₃ (sat. aq., 2 ml). The resulting mixture was extracted with CH₂Cl₂ (3×5 ml), and the combined organic phases were dried over anhydrous Na₂SO₄, filtered and concentrated under reduced pressure. The residue was purified by flash column chromatography (silica gel, EtOAc:hexanes 1:1) to afford Trx10 (3.0 mg, 5.6 μmol, 90%) as a yellow foam. Trx10: R_{f}=0.33 (silica gel, EtOAc:hexanes 1:1); [α]_{D}²⁵=+142.8 (c=0.1, CH₂Cl₂); FT-IR (film): νₘₐₓ=2923, 1621, 1571, 1388, 1234, 1094, 1004, 984, 927, 913 cm⁻¹; ¹H NMR (600 MHz, CDCl₃) δ=14.75 (s, 1H), 7.45 (d, J=1.2 Hz, 1H), 5.54 (d, J=6.9 Hz, 1H), 5.45 (s, 1H), 5.26-5.08 (m, 2H), 4.93 (d, J=6.9 Hz, 1H), 4.13 (q, J=6.6 Hz, 1H), 4.08-4.03 (m, 1H), 4.03-3.94 (m, 1H), 3.89 (q, J=6.6 Hz, 1H), 3.78 (s, 3H), 3.57 (s, 3H), 3.04 (td, J=5.8, 2.5 Hz, 2H), 2.98 (d, J=5.7 Hz, 1H), 2.86 (d, J=5.7 Hz, 1H), 2.73 (t, J=6.4 Hz, 2H), 2.57 (s, 3H), 2.09 (quint, J=6.4 Hz, 2H) ppm; ¹³C NMR (151 MHz, CDCl₃) δ=204.6, 163.1, 151.6, 142.5, 141.7, 135.4, 130.5, 115.9, 113.6, 113.4, 111.2, 104.3, 101.8, 98.7, 92.8, 73.5, 69.5, 68.7, 66.2, 65.9, 61.1, 56.7, 47.8, 39.0, 23.7, 22.3, 20.4 ppm; HRMS (ESI-TOF) calcd for C₂₇H₂₈O₁₁Na⁺ [M+Na]⁺551.1524, found 551.1526.

### (1S,2S,3aS,4R,13aR)-2-(1,3-Dioxolan-2-yl)-12-hydroxy-7-methoxy-5-methyl-11-oxo-3a,4,8,9,10,11-hexahydro-13aH-spiro[2,4-epoxyfuro[3,2-b]naphtho[2,3-h]chromene-1,2′-oxiran]-13a-yl 2,4-dideoxy-4-(isopropylamino)-3-O-methylpentopyranoside (Trx11)

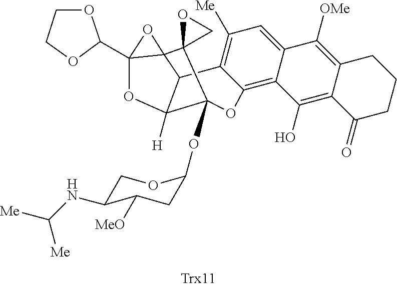 [SMILES: COc1c2c(c(O)c3c4c(c(C)cc13)C1C3OC5(C6OCCO6)OC15[C@]1(CO1)[C@]3(OC1CC(OC)C(NC(C)C)CO1)O4)C(=O)CCC2]

To a stirred solution of Trx8 (6.0 mg, 0.012 mmol, 1.0 equiv), carbohydrate donor 49 (54.6 mg, 0.124 mmol, 10 equiv) and 4 Å MS (250 mg) in CH₂Cl₂ (1.0 ml) at 0° C. was added Ph₃PAuOTf (0.05 M in CH₂Cl₂, 2.48 μmol, 50 μL, 0.2 equiv) dropwise over 5 min. The reaction mixture was stirred at this temperature for 15 min, and then quenched with Et₃N (10 μL). The resulting mixture was filtered through Celite® and concentrated under reduced pressure. The so obtained residue was purified by preparative thin layer chromatography (silica gel, hexanes:EtOAc 1:4) to give the corresponding mono-glycosylated product 50 (6.0 mg) as an orange foam.

To a stirred solution of the above mono-glycosylated product 50 (6.0 mg, 8.3 μmol, 1.0 equiv) in CH₂Cl₂ (0.5 ml) was added Pd(PPh₃)₂Cl₂ (2.9 mg, 4.13 μmol, 0.5 equiv), followed by acetic acid (9.9 mg, 0.17 mmol, 20 equiv) and n-Bu₃SnH (24 mg, 22 μL, 0.086 mmol, 10 equiv) at 23° C. After stirring at this temperature for 8 h, the reaction was quenched with NH₄Cl (sat. aq., 5 ml). The resulting mixture was extracted with CH₂Cl₂ (3×5 ml), the combined organic phases were dried over anhydrous Na₂SO₄ and concentrated under reduced pressure. The residue was purified by preparative thin layer chromatography (silica gel, CH₂Cl₂:MeOH 10:1) to give the title compound (3.8 mg, 5.8 μmol, 47% over 2 steps) as an orange foam. Trx11: R_{f}=0.57 (silica gel, CH₂Cl₂:MeOH 10:1); [α]_{D}²⁵=+256.0 (c=0.1, CH₂Cl₂); FT-IR (film): νₘₐₓ=2958, 1621, 1571, 1445, 1388, 1234, 1108, 1095, 986, 731 cm⁻¹; ¹H NMR (600 MHz, CDCl₃) δ=14.71 (s, 1H), 7.44 (s, 1H), 5.77 (t, J=2.9 Hz, 1H), 5.44 (s, 1H), 5.28 (d, J=4.0 Hz, 1H), 5.16 (d, J=4.2 Hz, 1H), 4.13 (dd, J=12.6, 6.0 Hz, 1H), 4.07-4.02 (m, 1H), 4.02-3.98 (m, 1H), 4.00 (dd, J=12.1, 6.3 Hz, 1H), 3.88 (dd, J=13.1, 6.5 Hz, 2H), 3.83 (br s, 1H), 3.78 (s, 3H), 3.70 (br s, 1H), 3.43 (s, 3H), 3.09-2.99 (m, 2H), 2.95 (br s, 1H), 2.92 (d, J=5.8 Hz, 1H), 2.80 (d, J=5.8 Hz, 1H), 2.80 (br s, 1H), 2.75-2.70 (m, 2H), 2.56 (s, 3H), 2.47-2.39 (m, 1H), 2.13-2.05 (m, 2H), 1.70 (ddd, J=13.2, 10.1, 3.5 Hz, 2H), 1.13 (br s, 6H); ¹³C NMR (151 MHz, CDCl₃) δ=204.4, 163.0, 151.8, 142.4, 141.6, 135.2, 130.3, 115.7, 113.5, 113.3, 111.1, 104.3, 101.9, 98.4, 94.5, 72.6, 69.3, 68.3, 66.0, 65.7, 63.5, 60.9, 56.4, 56.2, 47.3, 47.1, 38.8, 34.0, 24.4, 23.6, 22.7, 22.1, 20.2 ppm; HRMS (ESI-TOF) calcd for C₃₄H₄₁NO₁₂Na⁺ [M+Na]⁺ 678.2521, found 678.2520.

### 4-{[(Allyloxy)carbonyl](isopropyl)amino}-1-O-[2-(cyclopropylethynyl)benzoyl]-2,4-dideoxy-3-O-methyl-β-L-threo-pentopyranose (49)

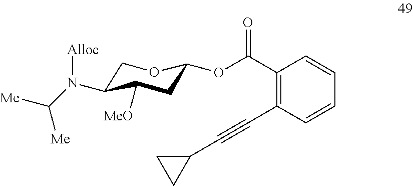

To a stirred solution of o-alkynylbenzoic acid 48 (Ma et al., 2011) (380 mg, 2.04 mmol, 1.0 equiv) and 2,6-di-tert-butyl-4-methylpyridine (629 mg, 3.06 mmol, 1.5 equiv) in CH₂Cl₂ (16 ml) at 0° C. were sequentially added oxalyl chloride (388 mg, 262 μL, 3.06 mmol, 1.5 equiv) and N,N-dimethylformamide (7.5 mg, 7.9 μL, 0.103 mmol, 0.05 equiv). The reaction mixture was allowed to warm to 23° C. and stirred at this temperature for 2 h before all the volatiles were removed under reduced pressure. The o-alkynylbenzoyl chloride so obtained was dissolved in CH₂Cl₂ (15 ml) and to such yellowish solution at 0° C. were sequentially added a solution of Alloc-protected aminosugar 47 (Nicolaou et al., 2011; Nicolaou et al., 2015) (446 mg, 1.63 mmol, 0.8 equiv) in CH₂Cl₂ (1.0 ml), Et₃N (619 mg, 0.853 ml, 6.12 mmol, 3.0 equiv) and 4-dimethylaminopyridine (75 mg, 0.61 mmol, 0.3 equiv). The reaction mixture was stirred at the same temperature for 0.5 h and then 23° C. for 12 h before it was quenched with NaHCO₃ (sat. aq. 15 ml). The resulting mixture was extracted with CH₂Cl₂ (3×20 ml), and the combined organic phases were dried over Na₂SO₄, filtered and concentrated under reduced pressure. The residue was purified by flash column chromatography (silica gel, EtOAc:hexanes 1:7) to give 49 (P-anomeric isomer, J=11.0 Hz, 599 mg, 1.35 mmol, 83%) as a colorless oil. 49: R_{f}=0.48 (silica gel, EtOAc:hexanes 3:7); [α]_{D}²⁵=+27 (c=0.4, CHCl₃); FT-IR (film): 2970, 2934, 2230, 1734, 1689, 1648, 1596, 1566, 1483, 1442, 1367, 1282, 1239 cm⁻¹; ¹H NMR (600 MHz, CDCl₃) δ=7.93 (d, J=7.9 Hz, 1H), 7.48 (d, J=7.6 Hz, 1H), 7.42 (t, J=7.5 Hz, 1H), 7.29 (t, J=7.4 Hz, 1H), 6.03-5.86 (m, 2H), 5.40-5.16 (m, 2H), 4.73-4.52 (m, 2H), 4.38-4.10 (m, 2H), 4.07-3.88 (m, 1H), 3.82 (dd, J=11.5, 5.1 Hz, 1H), 3.35 (s, 3H), 3.32 (br s, 1H), 2.56 (ddd, J=12.4, 4.8, 2.4 Hz, 1H), 1.71 (q, J=11.1 Hz, 1H), 1.53-1.47 (m, 1H), 1.22 (d, J=6.7 Hz, 3H), 1.19 (d, J=6.8 Hz, 3H), 0.93-0.88 (m, 4H). ppm; ¹³C NMR (151 MHz, CDCl₃) δ=164.4, 155.2, 134.4, 133.2, 133.0, 132.1, 130.9, 130.7, 127.1, 125.2, 118.5, 117.3, 99.9, 93.6, 74.6, 73.3, 66.1, 65.8, 65.4, 64.6, 57.2, 56.1, 48.2, 36.2, 21.4, 21.1, 20.5, 9.0, 0.8 ppm (29 signals and broadened peaks were observed because of rotamers around Alloc group); HRMS (ESI-TOF) calcd for C₂₅H₃₁NO₆Na⁺ [M+Na]⁺464.2044, found 464.2032.

### 2,2-Di-tert-butyl-4-[(2R,3S)-3-{2-(1,3-dioxan-2-ylcarbonyl)-1-[(trimethylsilyl)oxy]prop-2-en-1-yl}oxiran-2-yl]-7-methoxy-5-methyl-9,10-dihydroanthra[1,9-de][1,3,2]dioxasilin-11(8H)-one (40b)

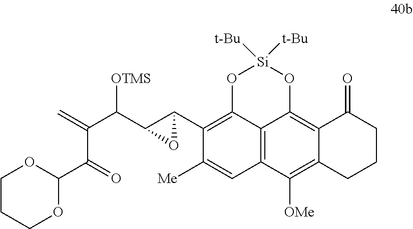

To a stirred solution of aldehyde 35 (224 mg, 0.480 mmol, 1.0 equiv) in toluene (2.88 ml) at 23° C. were added H₂O₂ (30 wt % in H₂O, 65 μl, 0.63 mmol, 1.3 equiv) and (S)-(−)-α,α-diphenyl-2-pyrrolidine methanol trimethylsilyl ether 36 (0.05 M in toluene, 1.92 ml, 0.096 mmol, 0.2 equiv). Four batches were separately processed at the same scale. After stirring at this temperature for 4 h, the four batches of reaction mixture were combined, diluted with EtOAc (10 ml) and washed with H₂O (2×10 ml), dried over anhydrous Na₂SO₄, filtered, and concentrated under reduced pressure to give the crude epoxide, which was used without further purification.

To a solution of the so obtained epoxide 37 in THF (7 ml) at 23° C. was added DABCO (107 mg, 0.96 mmol, 0.5 equiv), 4-nitrophenol (185 mg, 0.96 mmol, 0.5 equiv) and enone 38b (371 mg, 2.88 mmol, 2.0 equiv). After stirring at this temperature for 5 h, the reaction mixture was diluted with EtOAc (30 ml). The resulting mixture was washed with brine (2×10 ml), dried over anhydrous Na₂SO₄, filtered, and concentrated under reduced pressure. The residue was purified by flash column chromatography (silica gel, EtOAc:hexanes 1:4→1:2) to give the alcohol (699 mg, 1.12 mmol) as a pale yellow film.

To a solution of the so obtained alcohol in CH₂Cl₂ (10 ml) at −78° C. were sequentially added a solution of imidazole (152 mg, 2.24 mmol, 2.0 equiv) in CH₂Cl₂ (10 ml) and TMSCl (210 μl, 1.68 mmol, 1.5 equiv). The resulting reaction mixture was stirred at this temperature for 5 min before it was quenched with NaHCO₃ (sat. aq., 5 ml). The resulting mixture was stirred at 23° C. for 15 min and then extracted with CH₂Cl₂ (3×5 ml). The combined organic phases were dried over anhydrous Na₂SO₄, filtered and concentrated under reduced pressure. The residue was purified by flash column chromatography (silica gel, EtOAc:hexanes 1:6) to give the title compound (564 mg, 0.808 mmol, ca. 5:1 dr, 72%) as a pale yellow foam. 40b (plus C4-epi-40b): R_{f}=0.51 (silica gel, hexanes:EtOAc 4:1); [α]_{D}²⁵=−53.5 (c=1.0, CH₂Cl₂); FT-IR (film): νₘₐₓ=2936, 1682, 1559, 1445, 1371, 1236, 1147, 1084, 1060, 1015, 888 cm⁻¹; ¹H NMR (600 MHz, C₆D₆) δ=7.54 (s, 1H, major), 7.51 (s, 1H, minor), 6.95 (d, J=1.3 Hz, 1H, minor), 6.90 (d, J=1.7 Hz, 1 H, major), 6.68 (s, 1H, major), 6.67-6.66 (m, 1H, minor), 5.53 (s, 1H, minor), 5.31 (d, J=3.4 Hz, 1H, major), 5.02 (t, J=1.4, 1 H, major), 5.02 (s, 1H, minor), 4.48 (d, J=2.1 Hz, 1H, major), 4.08 (d, J=2.2 Hz, 1 H, minor), 3.85 (t, J=2.3 Hz, 1H, minor), 3.74 (dd, J=3.5, 2.3 Hz, 1H, major), 3.71-3.62 (m, 4H, major+minor), 3.49 (s, 6H, major+minor), 3.26-3.08 (m, 4H, major+minor), 2.75-2.63 (m, 4H, major+minor), 2.57 (s, 3H, major), 2.51 (s, 3H, minor), 2.42-2.32 (m, 4H, major+minor), 1.72-1.60 (m, 2H, major+minor), 1.54 (p, J=6.3 Hz, 4 H, major+minor), 1.28 (d, J=2.7 Hz, 18 H, major+minor), 1.26 (s, 18H, major+minor), 0.57-0.49 (m, 2H, major+minor), 0.19 (s, 9H, minor), 0.18 (s, 9H, major) ppm; ¹³C NMR (151 MHz, C₆D₆) δ=194.8 (major+minor), 192.1 (minor), 192.0 (major), 152.7 (minor), 152.3 (major), 150.4 (major+minor), 145.1 (major+minor), 144.6 (major), 143.3 (minor), 141.7 (major), 141.1 (minor), 132.3 (major), 132.2 (minor), 132.0 (major), 131.9 (minor), 131.4 (major), 131.1 (minor), 130.2 (minor), 119.83 (major), 119.78 (minor), 117.01 (major), 116.99 (minor), 115.43 (major), 115.41 (minor), 114.92 (major), 114.89 (minor), 102.8 (major+minor), 102.4 (major+minor), 101.5 (major+minor), 69.1 (major), 68.3 (minor), 66.9 (minor), 66.81 (major), 66.77 (minor), 66.7 (major), 66.4 (minor), 62.5 (major), 61.3 (minor), 60.5 (major), 53.1 (major), 51.4 (minor), 41.4 (major+minor), 26.62 (major), 26.59 (minor), 26.57 (major), 25.89 (major), 25.83 (minor), 24.4 (major+minor), 22.5 (major+minor), 21.7 (minor), 21.6 (major), 21.43 (major), 21.36 (minor), 21.24 (minor), 21.19 (major), 0.22 (minor), 0.21 (major) ppm; HRMS (ESI-TOF) calcd for C₃₇H₅₂O₉Si₂Na⁺ [M+Na]⁺719.3042, found 719.4052.

### 2,2-Di-tert-butyl-4-{(1R,3S,4S,5S)-1-(1,3-dioxan-2-yl)-6-methylene-5-[(trimethylsilyl)oxy]-2,7-dioxabicyclo[2.2.1]hept-3-yl}-7-methoxy-5-methyl-9,10-dihydroanthra[1,9-de][1,3,2]dioxasilin-11(8H)-one (41b)

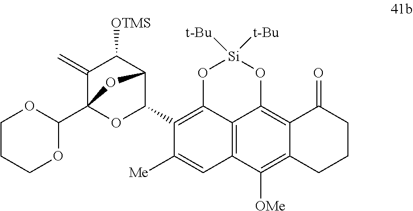

To a stirred solution of epoxy ketone 40b (200 mg, 0.286 mmol, 1.0 equiv) in CH₂Cl₂ (8.6 ml) at −30° C. was added BF₃—OEt₂ (0.1 M in CH₂Cl₂, 860 μL, 0.086 mmol, 0.3 equiv) dropwise. The reaction mixture was allowed to warm to 0° C. over 0.5 h, and was then quenched sequentially with Et₃N (40 μL) and NaHCO₃ (sat. aq., 20 ml). The resulting mixture was extracted with CH₂Cl₂ (3×10 ml), the combined organic phases were dried over anhydrous Na₂SO₄ and concentrated under reduced pressure. The residue was purified by flash column chromatography (silica gel, EtOAc:hexanes 1:7) to give 41b (98 mg, 0.140 mmol, 49%) as a yellow foam. R_{f}=0.62 (silica gel, EtOAc:hexanes 1:2); [α]_{D}²⁵=+119.2 (c=1.0, CH₂Cl₂); FT-IR (film): νₘₐₓ=2935, 2861, 1681, 1609, 1557, 1472, 1366, 1251, 1109, 891, 827 cm⁻¹; ¹H NMR (600 MHz, CDCl₃) δ=7.23 (s, 1H), 5.77 (d, J=3.1 Hz, 1H), 5.44 (d, J=2.5 Hz, 1H), 5.20 (s, 1H), 5.09 (d, J=2.1 Hz, 1H), 5.00 (dd, J=4.9, 3.2 Hz, 1H), 4.73 (dt, 4.8, 2.3, 1H), 4.34 (td, J=11.4, 4.8 Hz, 2H), 4.01-3.91 (m, 2H), 3.79 (s, 3H), 3.08 (ddd, J=16.2, 7.3, 4.5 Hz, 1H), 2.96 (ddd, J=16.2, 8.2, 4.5 Hz, 1H), 2.65 (s, 3H), 2.62 (t, J=6.5 Hz, 2H), 2.37-2.25 (m, 1H), 2.13-1.97 (m, 2H), 1.47 (d, J=13.6 Hz, 1H), 1.17 (s, 9H), 1.02 (s, 9H), −0.33 (s, 9H) ppm; ¹³C NMR (151 MHz, CDCl₃) δ 196.9, 150.0, 148.9, 146.7, 144.2, 142.1, 130.6, 130.5, 119.8, 116.3, 115.8, 113.6, 107.7, 106.9, 98.7, 80.3, 80.1, 73.4, 67.7, 67.6, 61.0, 41.4, 26.6, 26.6, 25.9, 24.3, 24.2, 22.5, 21.7, 20.8, −0.3 ppm; HRMS (ESI-TOF) calcd for C₃₇H₅₂O₉Si₂Na⁺ [M+Na]⁺719.3042, found 719.3037.

### 2,2-Di-tert-butyl-4-[(1R,3S,4R,5S)-1-(1,3-dioxan-2-yl)-5-hydroxy-6-methylene-2,7-dioxabicyclo[2.2.1]hept-3-yl]-7-methoxy-5-methyl-9,10-dihydroanthra[1,9-de][1,3,2]dioxasilin-11(8H)-one (42b)

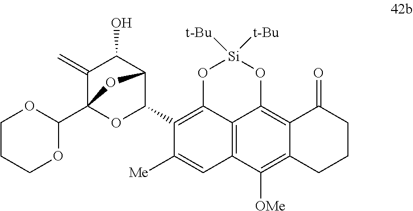 [SMILES: C=C1[C@H](O)[C@H]2O[C@]1(C1OCCCO1)O[C@H]2c1c(C)cc2c(OC)c3c(c4c2c1O[Si](C(C)(C)C)(C(C)(C)C)O4)C(=O)CCC3]

41b (98 mg, 0.140 mmol, 1.0 equiv) was dissolved in a solution of trifluoroacetic acid (0.1 M in THF:H₂O 5:1, 14 ml) at 23° C. After stirring at this temperature for 5 h, the reaction was quenched with NaHCO₃ (sat. aq., 10 ml). The resulting mixture was extracted with CH₂Cl₂ (3×10 ml), the combined organic phases were dried over anhydrous Na₂SO₄ and concentrated under reduced pressure. The residue was purified by flash column chromatography (silica gel, EtOAc:hexanes 1:10-1:4) to give the title compound (42b, 53.3 mg, 0.0854 mmol, 61%) as a yellow foam and recovered starting material 41b (25.5 mg, 0.0364 mmol, 26%). 42b: R_{f}=0.55 (silica gel, EtOAc:hexanes 1:1); [α]_{D}²⁵=+114.2 (c=1.0, CH₂Cl₂); FT-IR (film): νₘₐₓ=3488, 2934, 2860, 1680, 1608, 1555, 1372, 1101, 1017, 827, 661 cm⁻¹; ¹H NMR (600 MHz, CDCl₃) δ=7.35 (s, 1H), 5.74 (d, J=3.3 Hz, 1H), 5.52 (d, J=2.6 Hz, 1H), 5.33-5.28 (m, 1H), 5.25 (d, J=2.2 Hz, 1H), 5.19 (s, 1H), 4.70-4.65 (J=11.3, 4.3 Hz, 1H), 4.34 (dt, J=11.2, 4.8 Hz, 2H), 3.97 (dt, J=11.6, 8.5, 2.5 Hz, 2H), 3.83 (s, 3H), 3.08-2.98 (m, 2H), 2.69 (s, 3H), 2.63 (d, J=6.6 Hz, 2H), 2.37-2.25 (m, 1H), 2.06 (dt, J=12.7, 6.3 Hz, 2H), 1.48 (d, J=13.6 Hz, 1H), 1.18 (s, 9H), 1.07 (s, 9H), 1.01 (d, J=11.3 Hz, 1H) ppm; ¹³C NMR (151 MHz, CDCl₃) δ=196.5, 150.2, 149.7, 147.0, 144.2, 138.8, 131.6, 130.9, 118.0, 117.7, 116.2, 113.8, 107.7, 107.4, 98.3, 79.6, 79.5, 75.2, 67.5, 61.0, 41.2, 26.3, 26.2, 25.7, 24.6, 24.1, 22.3, 21.3, 21.0 ppm; HRMS (ESI-TOF) calcd for C₃₄H₄₄O₉SiNa⁺ [M+Na]⁺647.2647, found 647.2651.

### 2,2-Di-tert-butyl-4-[(1R,2S,3R,4R,5S)-1-(1,3-dioxan-2-yl)-3-hydroxyspiro[6,7-dioxabicyclo[2.2.1]-heptane-2,2′-oxiran]-5-yl]-7-methoxy-5-methyl-9,10-dihydroanthra[1,9-de][1,3,2]dioxasilin-11(8H)-one (45b)

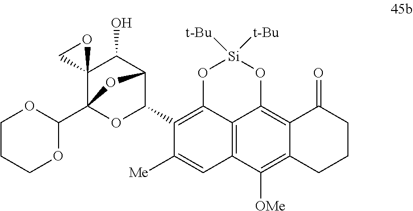 [SMILES: COc1c2c(c3c4c(c([C@@H]5O[C@@]6(C7OCCCO7)O[C@@H]5[C@@H](O)[C@@]65CO5)c(C)cc14)O[Si](C(C)(C)C)(C(C)(C)C)O3)C(=O)CCC2]

To a stirred solution of the 42b (53.3 mg, 0.0864 mmol, 1.0 equiv) in acetone (2.8 ml) at 23° C. was sequentially added OsO₄ (0.08 M aq., 216 μL, 0.0173 mmol, 0.2 equiv) and NMO (0.48 M aq., 720 μL, 0.346 mmol, 4.0 equiv). After stirring at this temperature for 12 h, the reaction was quenched with Na₂SO₃ (10% aq., 10 ml). The resulting mixture was stirred for another 0.5 h, then extracted with CH₂Cl₂ (3×5 ml), and the combined organic phases were dried over anhydrous Na₂SO₄ and concentrated under reduced pressure. The residue was filtered through a short pad of silica gel to afford triol 43b (51.7 mg, 0.0786 mmol) as a colorless oil, which was used for the nest step without further purification.

To a stirred solution of the above triol (51.7 mg, 0.0786 mmol) in CH₂Cl₂ (2.4 ml) at 23° C. were sequentially added Et₃N (39.7 mg, 0.393 mmol, 5.0 equiv), 4-dimethylaminopyridine (4.8 mg, 0.0393 mmol, 0.5 equiv) and TsCl (75.1 mg, 0.393 mmol, 5.0 equiv). After stirring at this temperature for 12 h, the reaction was quenched with NH₄Cl (sat. aq., 5 ml) and the resulting mixture was extracted with CH₂Cl₂ (3×5 ml). The combined organic phases were dried over anhydrous Na₂SO₄ and concentrated under reduced pressure. The residue was filtered through a short pad of silica gel to afford the triol 44b (59.3 mg, 0.0731 mmol) as a colorless oil, which was used for the nest step without further purification.

To a stirred solution of the above tosylate 44b (59.3 mg, 0.0731 mmol) in MeOH (2.0 ml) at 0° C. was added K₂CO₃ (20.2 mg, 0.146 mmol, 2.0 equiv). The resulting reaction mixture was stirred at this temperature for 1 h and was then directly subjected to flash column chromatography (silica gel, EtOAc:hexanes 1:1) to give the title compound (41.5 mg, 0.0648 mmol, 75% for the three steps) as a yellow foam. 45b: R_{f}=0.48 (silica gel, EtOAc:hexanes 1:1); [α]_{D}²⁵=+90.7 (c=1.0, CH₂Cl₂); FT-IR (film): νₘₐₓ=3479, 2935, 2861, 1678, 1608, 1555, 1471, 1371, 1104, 1055, 968, 827, 661 cm⁻¹; ¹H NMR (600 MHz, CDCl₃) δ=7.37 (s, 1H), 5.76 (d, J=3.1 Hz, 1H), 5.40 (dd, J=5.2, 3.1 Hz, 1H), 5.06 (s, 1H), 4.36-4.30 (m, 2H), 4.22 (dd, J=11.6, 5.0 Hz, 1H), 3.95 (dt, J=12.0, 2.5 Hz, 1H), 3.85 (dt, J=12.0, 2.5 Hz, 1H), 3.84 (s, 3H), 3.37 (d, J=5.6 Hz, 1H), 3.09 (d, J=5.6 Hz, 1H), 3.07-2.97 (m, 2H), 2.75 (s, 3H), 2.63 (t, J=6.5 Hz, 2H), 2.37-2.25 (m, 1H), 2.06 (dt, J=13.1, 6.5 Hz, 2H), 1.42 (d, J=13.6 Hz, 1H), 1.24 (d, J=11.8 Hz, 1H), 1.18 (s, 9H), 1.06 (s, 9H) ppm; ¹³C NMR (151 MHz, CDCl₃) δ=196.5, 150.1, 149.6, 144.2, 139.1, 131.8, 130.9, 117.6, 117.2, 116.3, 113.8, 106.8, 96.7, 79.6, 78.2, 76.4, 67.6, 67.4, 67.4, 61.1, 47.7, 41.2, 26.3, 26.3, 25.6, 24.1, 23.9, 22.3, 21.4, 20.9 ppm; HRMS (ESI-TOF) calcd for C₃₄H₄₄O₁₀SiNa⁺ [M+Na]⁺663.2596, found 663.2602.

### 2,2-Di-tert-butyl-4-[(1R,2S,4S,5S)-1-(1,3-dioxan-2-yl)-3-oxospiro[6,7-dioxabicyclo[2.2.1]heptane-2,2′-oxiran]-5-yl]-7-methoxy-5-methyl-9,10-dihydroanthra[1,9-de][1,3,2]dioxasilin-11(8H)-one (46b)

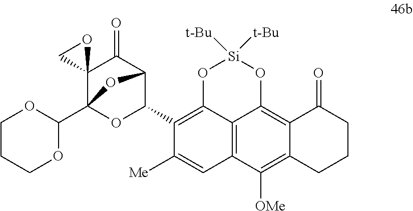 [SMILES: COc1c2c(c3c4c(c([C@@H]5O[C@@]6(C7OCCCO7)O[C@@H]5C(=O)[C@@]65CO5)c(C)cc14)O[Si](C(C)(C)C)(C(C)(C)C)O3)C(=O)CCC2]

To a stirred solution of the epoxy alcohol 45b (20.0 mg, 31.3 μmol, 1.0 equiv) in CH₂Cl₂ at 0° C. were sequentially added NMO (12.7 mg, 93.9 μmol, 3.0 equiv) and TPAP (2.2 mg, 6.3 μmol, 0.2 equiv). The resulting reaction mixture was stirred at this temperature for 1 h, and then directly subjected to flash column chromatography (silica gel, EtOAc:hexanes 1:4) to give the title compound (46b, 18.2 mg, 0.0285 mmol, 91%) as a yellow foam. 46b: R_{f}=0.75 (silica gel, EtOAc:hexanes 1:1); [α]_{D}²⁵=+222.8 (c=1.0, CH₂Cl₂); FT-IR (film): νₘₐₓ=2936, 2861, 1788, 1682, 1609, 1372, 1103, 889 cm; ¹H NMR (600 MHz, CDCl₃) δ=7.30 (s, 1H), 5.72 (d, J=3.8 Hz, 1H), 5.42 (d, J=3.8 Hz, 1H), 5.19 (s, 1H), 4.37 (dd, J=11.3, 4.8 Hz, 1H), 4.25 (dd, J=11.3, 4.9 Hz, 1H), 4.03-3.96 (m, 1H), 3.92-3.86 (m, 1H), 3.82 (s, 3H), 3.48 (d, J=6.5 Hz, 1H), 3.15 (d, J=6.5 Hz, 1H), 3.08-2.96 (m, 2H), 2.66-2.61 (m, 2H), 2.59 (s, 3H), 2.39-2.28 (m, 1H), 2.09-2.03 (m, 2H), 1.47 (d, J=13.8 Hz, 1H), 1.18 (s, 9H), 1.06 (s, 9H) ppm; ¹³C NMR (151 MHz, CDCl₃) δ=202.3, 196.5, 150.4, 149.7, 144.2, 139.0, 132.0, 131.2, 117.4, 116.2, 115.8, 113.7, 106.2, 96.2, 82.3, 78.3, 67.5, 67.5, 61.8, 61.1, 50.3, 41.2, 26.4, 26.2, 25.5, 24.1, 23.5, 22.3, 21.4, 21.0 ppm; HRMS (ESI-TOF) calcd for C₃₄H₄₂O₁₀SiNa⁺ [M+Na]⁺661.2439, found 661.2440.

### (1S,2S,3aS,4R,13aR)-13a-(Allyloxy)-2-(1,3-dioxan-2-yl)-12-hydroxy-7-methoxy-5-methyl-3a,4,8,9,10,13a-hexahydro-11H-spiro[2,4-epoxyfuro[3,2-b]naphtho[2,3-h]chromene-1,2′-oxiran]-11-one (Trx17)

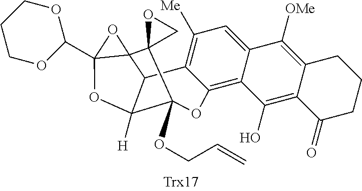 [SMILES: C=CCO[C@@]12Oc3c(c(C)cc4c(OC)c5c(c(O)c34)C(=O)CCC5)C3C1OC1(C4OCCCO4)OC31[C@]21CO1]

To a stirring mixture of Trx12 (2.8 mg, 5.6 μmol) and allyl bromide (0.1 ml) were sequentially added CaSO₄ (5.0 mg, 37 μmol, 6.6 equiv) and Ag₂O (5.0 mg, 22 μmol, 3.8 equiv) at 23° C. The resulting mixture was stirred at this temperature for 5 h before it was subjected to a flash column chromatography (EtOAc:hexanes 1:1) to afford Trx17 (2.0 mg, 3.7 μmol, 66%) as a yellow foam. Trx17: R_{f}=0.34 (silica gel, EtOAc:hexanes 1:2); [α]_{D}²⁵=+79.5 (c=0.258, CHCl₃); FT-IR (film): νₘₐₓ=2957, 2928, 2871, 1728, 1621, 1572, 1462, 1285, 1125 cm⁻¹; ¹H NMR (600 MHz, CDCl₃) δ=14.78 (s, 1H), 7.42 (d, J=0.9 Hz, 1H), 6.09 (ddt, J=17.2, 10.3, 5.7 Hz, 1H), 5.39 (dq, J=17.2, 1.5 Hz, 2H), 5.24 (d, J=4.1 Hz, 1H), 5.21 (dq, J=10.3, 1.4 Hz, 1H), 5.09 (s, 1H), 4.83 (d, J=4.1 Hz, 1H), 4.53 (qdt, J=12.6, 5.6, 1.4 Hz, 2H), 4.29 (ddt, J=11.5, 4.9, 1.6 Hz, 1H), 4.12 (ddt, J=11.6, 5.1, 1.6 Hz, 1H), 3.90 (td, J=12.0, 2.6 Hz, 1H), 3.77 (s, 3H), 3.09-2.97 (m, 2H), 2.95 (d, J=5.8 Hz, 1H), 2.86 (d, J=5.8 Hz, 1H), 2.72 (dd, J=7.4, 5.5 Hz, 2H), 2.60 (d, J=1.0 Hz, 3H), 2.26-2.16 (m, 1H), 2.09 (tdd, J=8.5, 6.9, 4.1 Hz, 2H), 1.37 (d, J=13.6 Hz, 1H) ppm; ¹³C NMR (151 MHz, CDCl₃) δ=204.6, 163.1, 151.5, 142.6, 142.1, 135.4, 134.7, 130.4, 117.4, 115.8, 113.5, 113.4, 111.2, 103.2, 102.1, 96.4, 72.8, 69.3, 67.6, 67.5, 66.8, 61.0, 48.4, 39.0, 25.7, 23.7, 22.3, 20.8 ppm. HRMS (ESI) calcd for C₂₉H₃₀O₁₀Na⁺ [M+Na]⁺561.1731, found 561.1731.

### (1S,13aS)-2-(1,3-Dioxan-2-yl)-12-hydroxy-7-methoxy-5-methyl-11-oxo-3a,4,8,9,10,11-hexahydro-13aH-spiro[2,4-epoxyfuro[3,2-b]naphtho[2,3-h]chromene-1,2′-oxiran]-13a-yl 4-{[(allyloxy)carbonyl](isopropyl)amino}-2,4-dideoxy-3-O-methyl-α-L-threo-pentopyranoside (Trx18)

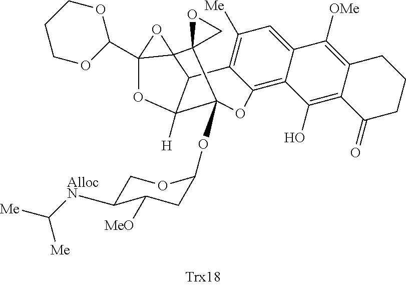

To a stirred solution of Trx12 (5.0 mg, 0.01 mmol, 1.0 equiv), carbohydrate donor 49 (44.1 mg, 0.100 mmol, 10 equiv) and 4 Å MS (250 mg) in CH₂Cl₂ (1.0 ml) at 0° C. was added Ph₃PAuOTf (0.05 M in CH₂Cl₂, 2.0 μmol, 40 μL, 0.2 equiv) dropwise over 5 min. The reaction mixture was stirred at this temperature for 15 min, and then quenched with Et₃N (10 μL). The resulting mixture was filtered through Celite® and concentrated under reduced pressure. The residue was purified by preparative thin layer chromatography (silica gel, hexanes:EtOAc 1:4) to give the title compound (6.1 mg, 8.3 mmol, 83%) as an orange foam. Trx18: R_{f}=0.34 (silica gel, EtOAc:hexanes 2:1); [α]_{D}²⁵=+163.3 (c=0.15, CH₂Cl₂); FT-IR (film): νₘₐₓ=2962, 2930, 1693, 1620, 1444, 1389, 1370, 1094, 987, 925, 734 cm⁻¹; ¹H NMR (600 MHz, CDCl₃) δ=14.73 (s, 1H), 7.42 (s, 1H), 6.22-5.88 (m, 1H), 5.85-5.74 (m, 1H), 5.47-5.18 (m, 4H), 5.19-5.01 (m, 1H), 4.77-4.55 (m, 2H), 4.56-4.37 (m, 2H), 4.31 (dd, J=11.7, 4.8 Hz, 1H), 4.18-4.12 (m, 1H), 3.97-3.83 (m, 1H), 3.84-3.78 (m, 1H), 3.77 (s, 3H), 3.62-3.47 (m, 1H), 3.38 (s, 3H), 3.13-2.99 (m, 2H), 2.93 (d, J=6.0 Hz, 1H), 2.83 (d, J=6.0 Hz, 1H), 2.72 (dd, J=7.3, 5.6 Hz, 2H), 2.60 (s, 3H), 2.58-2.49 (m, 1H), 2.28-2.16 (m, 1H), 2.09 (d, J=6.7 Hz, 2H), 1.39 (d, J=13.6 Hz, 1H), 1.32-1.27 (m, 2H), 1.32-1.12 (m, 6H) ppm; ¹³C NMR (151 MHz, CDCl₃) δ=204.6, 163.2, 152.0, 142.5, 142.2, 135.4, 133.6, 131.6, 130.4, 115.8, 113.4, 113.3, 111.2, 103.2, 101.8, 96.5, 96.4, 94.6, 72.4, 69.6, 68.6, 67.64, 67.56, 65.9, 62.1, 61.0, 47.8, 39.0, 25.7, 23.7, 22.3, 21.2, 20.9, 20.4 ppm; HRMS (ESI-TOF) calcd for C₃₉H₄₇NO₄Na⁺ [M+Na]⁺776.2889, found 776.2885.

### (1S,2R)-2-(1,3-dioxan-2-yl)-12-hydroxy-7-methoxy-5-methyl-11-oxo-3a,4,8,9,10,11-hexahydro-13aH-spiro[2,4-epoxyfuro[3,2-b]naphtho[2,3-h]chromene-1,2′-oxiran]-13a-yl 2,4-dideoxy-4-(iso-propylamino)-3-O-methyl-α-L-threo-pentopyranoside (Trx19)

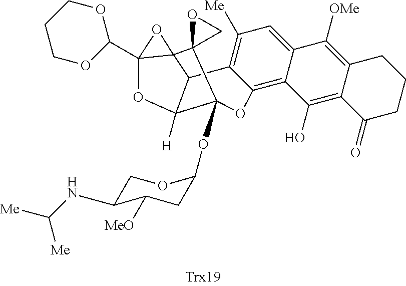 [SMILES: COc1c2c(c(O)c3c4c(c(C)cc13)C1C3OC5(C6OCCCO6)OC15[C@]1(CO1)[C@]3(OC1CC(OC)C(NC(C)C)CO1)O4)C(=O)CCC2]

To a stirred solution of Trx18 (6.1 mg, 8.3 μmol) in CH₂Cl₂ (0.5 ml) was added Pd(PPh₃)₂Cl₂ (2.9 mg, 4.1 μmol, 0.5 equiv), followed by acetic acid (9.9 mg, 165 μmol, 20 equiv) and n-Bu₃SnH (24.0 mg, 82.5 μmol, 10 equiv) at 23° C. After stirring at this temperature for 8 h, the reaction was quenched with NH₄Cl (sat. aq., 5 ml). The resulting mixture was extracted with CH₂Cl₂ (3×5 ml), the combined organic phases were dried over anhydrous Na₂SO₄ and concentrated under reduced pressure. The residue was purified by preparative thin layer chromatography (silica gel, CH₂Cl₂:MeOH 10:1) to give the title compound (4.8 mg, 7.2 μmol, 87%) as an orange foam. Trx19: R_{f}=0.53 (silica gel, CH₂Cl₂:MeOH 10:1); [α]_{D}²⁵=+208.0 (c=0.15, CH₂Cl₂); FT-IR (film): νₘₐₓ=2960, 2855, 1620, 1389, 1108, 1095, 1003, 730 cm⁻¹; ¹H NMR (600 MHz, CDCl₃) δ=14.72 (s, 1H), 7.41 (s, 1H), 5.78 (br s, 1H), 5.28 (br s, 1H), 5.23 (d, J=3.9 Hz, 1H), 5.10 (s, 1H), 4.27 (br s, 1H), 4.11 (dd, J=11.1, 5.5 Hz, 1H), 3.93 (t, J=11.1 Hz, 1H), 3.93 (br s, 1H), 3.82-3.74 (m, 1H), 3.76 (s, 3H), 3.45 (s, 3H), 3.08-2.98 (m, 2H), 2.90 (br s, 1H), 2.90 (d, J=6.0 Hz, 1H), 2.78 (d, J=6.0 Hz, 1H), 2.72 (dd, J=7.4, 5.6 Hz, 2H), 2.60 (s, 3H), 2.48 (br s, 1H), 2.26-2.14 (m, 1H), 2.12-2.05 (m, 2H), 1.71-1.66 (m, 1H), 1.38 (d, J=13.6 Hz, 1H), 1.19 (br s, 6H) ppm; ¹³C NMR (151 MHz, CDCl₃) δ=204.4, 163.0, 142.4, 142.2, 135.2, 130.2, 115.6, 113.4, 113.2, 111.0, 103.0, 101.7, 96.2, 72.3, 69.3, 68.3, 67.5, 67.3, 60.9, 56.3, 56.2, 47.6, 38.8, 25.6, 23.6, 22.1, 20.7 ppm; HRMS (ESI-TOF) calcd for C₃₅H₄₃NO₁₂Na⁺ [M+Na]⁺692.2677, found 692.2677.

### (1S,2R,13aS)-2-(1,3-dioxan-2-yl)-12-hydroxy-7-methoxy-5-methyl-11-oxo-3a,4,8,9,10,11-hexahydro-13aH-spiro[2,4-epoxyfuro[3,2-b]naphtho[2,3-h]chromene-1,2′-oxiran]-13a-yl 4-C-acetyl-3-O-acetyl-2,6-dideoxy-α-L-lyxo-hexopyranoside (Trx20)

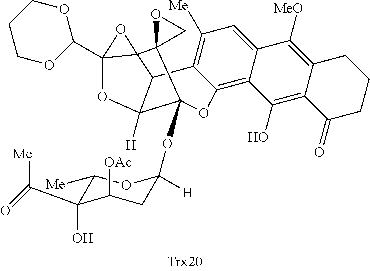 [SMILES: COc1c2c(c(O)c3c4c(c(C)cc13)C1C3OC5(C6OCCCO6)OC15[C@]1(CO1)[C@]3(OC1CC(OC(C)=O)C(O)(C(C)=O)C(C)O1)O4)C(=O)CCC2]

To a stirred solution of Trx12 (5.0 mg, 10 μmol, 1.0 equiv), carbohydrate donor 51 (40.0 mg, 10 μmol, 10 equiv) and 4 Å MS (250 mg) in CH₂Cl₂ (2.0 ml) at 0° C. was added Ph₃PAuOTf (0.05 M in CH₂CO₂, 2.0 μmol, 40 μL, 0.2 equiv) dropwise over 5 min. The reaction mixture was stirred at this temperature for 15 min, and then quenched with Et₃N (10 μL). The resulting mixture was filtered through Celite® and concentrated under reduced pressure. The residue was purified by preparative thin layer chromatography (silica gel, hexanes:EtOAc 1:2) to give the title compound (6.4 mg, 9.0 μmol, 90%) as an orange foam. Trx20: R_{f}=0.24 (silica gel, EtOAc:hexanes 2:1); [α]_{D}²⁵=+77.5 (c=0.2, CH₂CO₂); FT-IR (film): νₘₐₓ=3465, 2937, 2852, 1737, 1620, 1388, 1236, 1095, 1012, 912, 733 cm⁻¹; ¹H NMR (600 MHz, CDCl₃) δ=14.65 (s, 1H), 7.41 (s, 1H), 5.85 (t, J=3.1 Hz, 1H), 5.29 (d, J=4.1 Hz, 1H), 5.24 (d, J=4.1 Hz, 1H), 5.05 (s, 1H), 4.99 (q, J=6.4 Hz, 1H), 4.74 (t, J=3.5 Hz, 1H), 4.34 (dd, J=11.5, 4.8 Hz, 1H), 4.13 (dd, J=11.2, 5.0 Hz, 1H), 3.95 (td, J=12.2, 2.3 Hz, 1H), 3.88 (s, 1H), 3.79 (td, J=12.3, 2.3 Hz, 1H), 3.76 (s, 3H), 3.08-2.96 (m, 2H), 2.91 (d, J=6.0 Hz, 1H), 2.77 (d, J=6.0 Hz, 1H), 2.71 (t, J=6.4 Hz, 2H), 2.61 (s, 3H), 2.36 (s, 3H), 2.35-2.30 (m, 2H), 2.29-2.18 (m, 1H), 2.23 (s, 3H), 2.11-2.05 (m, 2H), 1.40 (d, J=13.5 Hz, 1H), 1.06 (d, J=6.4 Hz, 3H) ppm; ¹³C NMR (151 MHz, CDCl₃) δ=208.8, 204.3, 170.3, 163.0, 151.8, 142.3, 141.9, 135.2, 130.3, 115.6, 113.1, 113.0, 111.0, 103.1, 101.4, 96.2, 92.2, 78.2, 72.4, 70.7, 69.4, 68.4, 67.4, 67.4, 64.4, 60.9, 47.4, 38.8, 28.8, 27.1, 25.6, 23.6, 22.1, 21.3, 20.6, 14.6 ppm; HRMS (ESI-TOF) calcd for C₃₆H₄₀O₁₅Na⁺ [M+Na]⁺735.2259, found 735.2257.

### (4R,5S,6R,10S)-8-{[(1S,2R)-2-(1,3-Dioxan-2-yl)-12-hydroxy-7-methoxy-5-methyl-11-oxo-3a,4,8,9,10,11-hexahydro-13aH-spiro[2,4-epoxyfuro[3,2-b]naphtho[2,3-h]chromene-1,2′-oxiran]-13a-yl]oxy}-4,6-dimethyl-2-oxo-1,3,7-trioxaspiro[4.5]dec-10-ylacetate (Trx22)

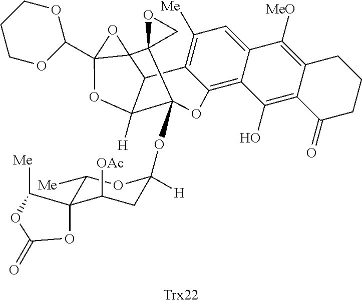

To a stirred solution of Trx12 (1.0 mg, 2.0 μmol, 1.0 equiv), carbohydrate donor 52 (10.0 mg, 10 μmol, 10 equiv) and 4 Å MS (50 mg) in CH₂Cl₂ (0.2 ml) at 0° C. was added Ph₃PAuOTf (0.05 M in CH₂CO₂, 0.4 μmol, 8 μL, 0.2 equiv) dropwise over 5 min. The reaction mixture was stirred at this temperature for 15 min, and then quenched with Et₃N (10 μL). The resulting mixture was filtered through Celite® and concentrated under reduced pressure. The residue was purified by preparative thin layer chromatography (silica gel, EtOAc) to give the title compound (1.3 mg, 1.8 μmol, 90%) as an orange foam. Trx22: R_{f}=0.24 (silica gel, EtOAc:hexanes 2:1); [α]_{D}²⁵=+97.5 (c=0.2, CH₂Cl₂); FT-IR (film): νₘₐₓ=3629, 2951, 2854, 1813, 1748, 1619, 1388, 1235, 1095, 1048, 1014, 996, 733 cm⁻¹; ¹H NMR (600 MHz, CDCl₃) δ=14.68 (s, 1H), 7.43 (s, 1H), 5.79 (dd, J=3.9, 2.2 Hz, 1H), 5.23 (d, J=4.1 Hz, 1H), 5.20 (d, J=4.1 Hz, 1H), 5.03-4.98 (m, 1H), 5.01 (s, 1H), 4.66-4.56 (m, 2H), 4.33 (dd, J=11.5, 4.7 Hz, 1H), 4.12 (dd, J=12.1, 4.1 Hz, 1H), 3.93 (td, J=12.2, 2.3 Hz, 1H), 3.82-3.72 (m, 1H), 3.76 (s, 3H), 3.10-2.96 (m, 2H), 2.90 (d, J=6.0 Hz, 1H), 2.76 (d, J=6.0 Hz, 1H), 2.72 (t, J=6.4 Hz, 2H), 2.60 (s, 3H), 2.35 (ddd, J=14.9, 3.9, 2.3 Hz, 1H), 2.26 (t, J=3.9 Hz, 1H), 2.25-2.18 (m, 1H), 2.23 (s, 3H), 2.10-2.06 (m, 2H), 1.60 (d, J=6.8 Hz, 3H), 1.40 (d, J=12.3 Hz, 1H), 1.34 (d, J=6.4 Hz, 3H) ppm; ¹³C NMR (151 MHz, CDCl₃) δ=204.4, 170.0, 162.9, 153.3, 151.6, 142.4, 141.8, 135.2, 130.4, 115.8, 113.1, 112.9, 111.1, 103.1, 101.4, 96.2, 91.0, 80.9, 80.6, 72.4, 70.6, 69.4, 68.3, 67.4, 67.4, 64.1, 60.9, 47.5, 38.8, 30.2, 25.5, 23.6, 22.1, 21.0, 20.6, 15.2, 14.9 ppm; HRMS (ESI-TOF) calcd for C₃₇H₄₀O₁₆Na⁺ [M+Na]⁺763.2209, found 763.2194.

### (1S,2R,13aS)-2-(1,3-dioxan-2-yl)-12-hydroxy-7-methoxy-5-methyl-11-oxo-3a,4,8,9,10,11-hexahydro-13aH-spiro[2,4-epoxyfuro[3,2-b]naphtho[2,3-h]chromene-1,2′-oxiran]-13a-yl 2,6-dideoxy-4-C-[(1R)-1-hydroxyethyl]-α-L-lyxo-hexopyranoside (Trx23)

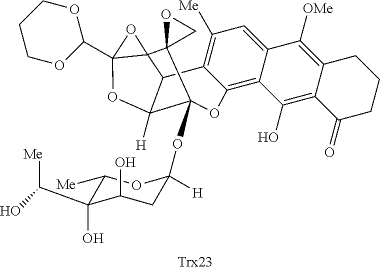 [SMILES: COc1c2c(c(O)c3c4c(c(C)cc13)C1C3OC5(C6OCCCO6)OC15[C@]1(CO1)[C@]3(OC1CC(O)C(O)([C@@H](C)O)C(C)O1)O4)C(=O)CCC2]

To a stirred solution of Trx22 (1.0 mg, 1.4 μmol, 1.0 equiv) in THF (0.15 ml) and ethylene glycol (15 μL) at 23° C. was added NaH (1.0 mg, 42 μmol, 30 equiv). After stirring at this temperature for 5 h, the reaction mixture was diluted with EtOAc (10 ml). The resulting mixture was washed sequentially with water (5 ml) and brine (5 ml), dried over anhydrous Na₂SO₄, and concentrated under reduced pressure. The residue was purified by preparative HPLC (Atlantis Prep T3 OBD column, 5 m, 19×150 mm, UV detection at 270 nm, gradient elution with 50→70% (1→2 min), then 70→100% (2→45 min) MeCN in H₂O, flow rate: 5 mL/min, 31.5→32.8 min) to give the title compound (0.8 mg, 1.19 μmol, 88%) as an orange foam. Trx23: R_{f}=0.50 (silica gel, EtOAc); [α]_{D}²⁵=+135.0 (c=0.1, CH₂Cl₂); FT-IR (film): νₘₐₓ=3488, 2938, 2857, 1621, 1389, 1096, 1049, 997 cm⁻¹; ¹H NMR (600 MHz, CDCl₃) δ=14.69 (s, 1H), 7.44 (s, 1H), 5.76 (d, J=3.2 Hz, 1H), 5.29 (d, J=4.1 Hz, 1H), 5.27 (d, J=4.1 Hz, 1H), 5.09 (s, 1H), 5.08 (d, J=6.6 Hz, 1H), 4.31 (dd, J=11.5, 4.9 Hz, 1H), 4.20-4.16 (m, 1H), 4.13-4.08 (m, 1H), 4.01-3.94 (m, 2H), 3.82-3.73 (m, 2H), 3.77 (s, 3H), 3.42 (d, J=11.4 Hz, 1H), 3.09-2.98 (m, 3H), 2.87 (d, J=5.8 Hz, 1H), 2.76-2.69 (m, 2H), 2.61 (s, 3H), 2.31-2.17 (m, 2H), 2.13-2.05 (m, 3H), 1.83 (s, 1H), 1.40 (d, J=14.8 Hz, 1H), 1.37 (d, J=6.5 Hz, 3H), 1.27 (d, J=6.5 Hz, 3H) ppm; ¹³C NMR (151 MHz, CDCl₃) δ=204.5, 162.9, 151.6, 142.4, 141.9, 135.2, 130.4, 115.9, 113.1, 113.0, 111.1, 103.1, 101.4, 96.0, 94.0, 71.6, 71.6, 71.0, 69.5, 68.6, 68.3, 67.5, 67.3, 65.1, 60.9, 48.1, 38.8, 32.4, 25.6, 23.6, 22.1, 20.6, 17.5, 13.4 ppm; HRMS (ESI-TOF) calcd for C₃₄H₄₀O₁₄Na⁺ [M+Na]⁺695.2310, found 695.2310.

### (1S,2S,3aS,4R,13aR,1′″S,2′″S,3a′″S,4′″R,13a′″R)-13a,13a′-[(2Z)-but-2-ene-1,4-diylbis(oxy)]bis[2-(1,3-dioxan-2-yl)-12-hydroxy-7-methoxy-5-methyl-3a,4,8,9,10,13a-hexahydro-11H-spiro[2,4-epoxyfuro[3,2-b]naphtho[2,3-h]chromene-1,2′-oxiran]-11-one] (Trx24) and (1S,2S,3aS,4R,13aR,1′″S,2′″S,3a′″S,4′R,13a′″R)-13a,13a′-[(2E)-but-2-ene-1,4-diylbis(oxy)]bis[2-(1,3-dioxan-2-yl)-12-hydroxy-7-methoxy-5-methyl-3a,4,8,9,10,13a-hexahydro-11H-spiro[2,4-epoxyfuro[3,2-b]naphtho[2,3-h]chromene-1,2′-oxiran]-11-one] (Trx25)

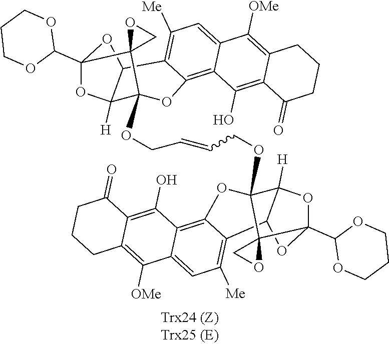

A suspension of Trx17 (2.0 mg, 3.7 μmol) and Grubbs I catalyst (0.0053 M in CH₂Cl₂, 70 μL, 0.37 μmol, 0.1 equiv) was stirred at 23° C. for 14 h before the resulting mixture was directly subjected to preparative 10 thin layer chromatography (silica gel, CH₂Cl₂:acetone 9:1) to afford (Z)-isomer Trx24 (0.6 mg, 0.57 μmol, 31%, a yellow foam) as the major product and (E)-isomer Trx25 (0.3 mg, 0.29 μmol, 16%, a yellow foam) as the minor product. Trx24 (Z): R_{f}=0.70 (silica gel, CH₂Cl₂:Et₂O 10:1); [α]_{D}²⁵=+209.9 (c=0.0667, CHCl₃); FT-IR (film): νₘₐₓ=2926, 1621, 1573, 1445, 1390, 1236, 1095 cm⁻¹; ¹H NMR (600 MHz, CDCl₃) δ=14.49 (s, 2H), 7.17 (d, J=1.2 Hz, 2H), 6.08-5.96 (m, 2H), 5.16-5.10 (m, 4H), 5.06 (s, 2H), 4.82 (d, J=5.1 Hz, 4H), 4.28 (dd, J=11.7, 4.8 Hz, 22H), 4.08 (dd, J=11.6, 4.8 Hz, 2H), 3.88 (td, J=12.0, 2.5 Hz, 2H), 3.74 (td, J=12.1, 2.5 Hz, 2H), 3.63 (s, 6H), 2.81 (d, J=5.9 Hz, 2H), 2.77 (td, J=5.8, 2.5 Hz, 4H), 2.67 (d, J=5.9 Hz, 2H), 2.51 (d, J=1.0 Hz, 6H), 2.50-2.45 (m, 2H), 2.30 (dt, J=17.2, 6.3 Hz, 2H), 2.24-2.14 (m, 2H), 1.91 (d, J=8.6 Hz, 2H), 1.35 (d, J=13.6 Hz, 2H) ppm; ¹³C NMR (151 MHz, CDCl₃) δ=204.3, 163.1, 151.5, 142.4, 142.1, 135.3, 130.2, 130.0, 115.7, 113.6, 113.4, 111.0, 103.1, 102.2, 96.4, 72.9, 69.4, 69.3, 67.6, 67.5, 66.0, 61.0, 48.4, 38.9, 25.8, 23.7, 22.2, 20.8 ppm; HRMS (ESI) calcd for C₅₆H₅₆O₂Na⁺ [M+Na]⁺1071.3257, found 1071.3234. Trx25 (E): R_{f}=0.72 (silica gel, CH₂Cl₂: Et₂O 10:1); [α]_{D}²⁵=+144.9 (c=0.055, CHCl₃); FT-IR (film): νₘₐₓ=2922, 2851, 1621, 1571, 1447, 1389, 1235, 1095 cm⁻¹; ¹H NMR (600 MHz, CDCl₃) δ=14.72 (s, 2H), 7.39 (s, 2H), 6.15-6.08 (m, 2H), 5.23 (d, J=4.1 Hz, 2H), 5.09 (s, 2H), 4.87 (d, J=4.1 Hz, 2H), 4.59 (dd, J=13.1, 3.1 Hz, 2H), 4.56-4.51 (m, 2H), 4.29 (dd, J=11.5, 4.8 Hz, 2H), 4.11 (dd, J=11.7, 4.9 Hz, 2H), 3.90 (td, J=12.0, 2.5 Hz, 2H), 3.78 (td, J=12.4, 2.3 Hz, 2H), 3.75 (s, 6H), 2.98 (t, J=6.2 Hz, 4H), 2.92 (d, J=5.8 Hz, 2H), 2.83 (d, J=5.8 Hz, 2H), 2.71-2.60 (m, 4H), 2.60 (d, J=1.0 Hz, 6H), 2.21 (ddd, J=17.2, 12.4, 7.5 Hz, 2H), 2.11-1.98 (m, 4H), 1.36 (d, J=13.7 Hz, 2H) ppm; ¹³C NMR (151 MHz, CDCl₃) δ=203.7, 162.8, 151.5, 141.9, 141.7, 134.8, 129.7, 129.6, 115.4, 113.7, 113.0, 110.4, 103.1, 102.1, 96.5, 72.1, 69.5, 68.9, 67.6, 67.5, 61.0, 60.9, 48.1, 38.6, 25.7, 23.37, 22.03, 20.87 ppm; HRMS (ESI) calcd for C₅₆H₅₆O₂₀K⁺ [M+K]⁺ 1087.2997, found 1087.2956.

### (1S,2S,3aS,4R,13aS)-2-(1,3-Dioxan-2-yl)-7-methoxy-5-methyl-11-oxo-3a,4,8,9,10,11-hexahydro-13aH-spiro[2,4-epoxyfuro[3,2-b]naphtho[2,3-h]chromene-1,2′-oxirane]-12,13a-diyl diacetate (Trx26)

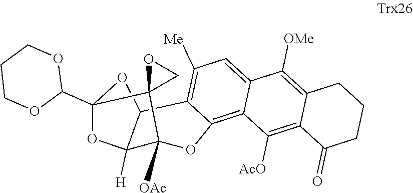

To a stirred solution of Trx12 (9.3 mg, 19 μmol, 1.0 equiv) in CH₂Cl₂ (0.2 ml) were sequentially added 4-dimethylaminopyridine (1.4 mg, 9.3 μmol, 0.5 equiv), Et₃N (145 mg, 200 μL, 2.87 mmol, 151 equiv) and Ac₂O (106 mg, 0.10 ml, 1.06 mmol, 57 equiv) at 23° C. The mixture was stirred at this temperature for 4 h before it was quenched with NaHCO₃ (sat. aq., 5 ml). The resulting mixture was stirred at this temperature for 0.5 h, and then extracted with CH₂Cl₂ (3×5 ml). The combined organic phases were dried over anhydrous Na₂SO₄, filtered and concentrated under reduced pressure. The residue was purified by flash column chromatography (silica gel, EtOAc:hexanes 4:1) to give the titled compound (4.9 mg, 8.8 μmol, 67%) as a pale yellow foam. Trx26: R_{f}=0.49 (silica gel, EtOAc); [α]_{D}²⁵=+264.6 (c=0.3, CH₂Cl₂); FT-IR (film): νₘₐₓ=2957, 1763, 1685, 1557, 1445, 1411, 1374, 1337, 1239, 1212, 1015 cm⁻¹; ¹H NMR (600 MHz, C₆D₆) δ=7.62-7.48 (m, 1H), 5.83-5.63 (m, 1H), 5.18-5.07 (m, 1H), 4.94-4.78 (m, 1H), 3.72 (dd, J=11.5, 4.7 Hz, 1H), 3.59-3.45 (m, 1H), 3.42-3.34 (m, 4H), 3.28-3.18 (m, 1H), 3.06 (td, J=12.0, 2.5 Hz, 1H), 2.97 (d, J=5.9 Hz, 1H), 2.70 (ddd, J=16.3, 6.9, 4.4 Hz, 1H), 2.44-2.35 (m, 4H), 2.33-2.23 (m, 5H), 1.76-1.72 (m, 3H), 1.72-1.63 (m, 1H), 1.50-1.36 (m, 2H) 0.36 (d, J=13.6, 1H) ppm; ¹³C NMR (151 MHz, C₆D₆) δ=195.5, 170.5, 168.5, 149.7, 149.3, 145.4, 140.2, 133.3, 133.0, 122.8, 117.7, 116.3, 115.2, 103.8, 101.8, 97.0, 71.3, 69.3, 69.1, 66.9, 66.7, 60.6, 47.7, 40.9, 25.6, 24.0, 21.9, 21.3, 21.2, 20.0 ppm; HRMS (ESI) calcd for C₃₀H₃₀O₁₂Na⁺ [M+Na]⁺605.1629, found 605.1637.

### (1S,2S,3aS,4R,13aR)-2-(1,3-Dioxan-2-yl)-7,13a-dimethoxy-5-methyl-11-oxo-3a,8,9,10,11,13a-hexahydro-4H-spiro[2,4-epoxyfuro[3,2-b]naphtho[2,3-h]chromene-1,2′-oxiran]-12-yl acetate (Trx27)

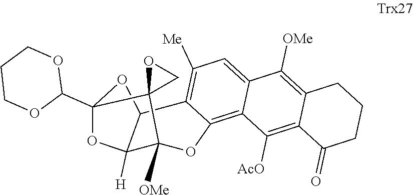

To a stirred solution of Trx13 (4.7 mg, 9.2 μmol, 1.0 equiv) in CH₂Cl₂ (0.1 ml) were sequentially added 4-dimethylaminopyridine (0.56 mg, 0.46 μmol, 0.5 equiv), Et₃N (290 mg, 0.4 ml, 2.87 mmol, 320 equiv) and Ac₂O (52.5 mg, 50 μl, 529 μmol, 58 equiv) at 23° C. The mixture was stirred at this temperature overnight before it was quenched with NaHCO₃ (sat. aq., 5 ml). The resulting mixture was extracted with CH₂Cl₂ (3×5 ml). The combined organic phases were dried over anhydrous Na₂SO₄, filtered and concentrated under reduced pressure. The residue was purified by flash column chromatography (silica gel, EtOAc:hexanes 4:1) to give the titled compound (4.9 mg, 8.8 μmol, 96%) as a pale yellow foam. Trx27: R_{f}=0.49 (silica gel, EtOAc); [α]_{D}²⁵=+264.6 (c=0.3, CH₂Cl₂); FT-IR (film): νₘₐₓ=2957, 1763, 1685, 1557, 1445, 1411, 1374, 1337, 1239, 1212, 1015 cm⁻¹; ¹H NMR (600 MHz, CDCl₃) δ=7.61-7.51 (m, 1H), 5.38-5.25 (m, 1H), 5.10-4.98 (m, 2H), 4.33-4.26 (m, 1H), 4.18-4.08 (m, 1H), 3.93-3.87 (m, 1H), 3.87-3.83 (m, 3H), 3.81-3.74 (m, 1H), 3.70-3.54 (m, 2H), 3.21 (dt, J=16.9, 6.3 Hz, 1H), 3.03-2.89 (m, 2H), 2.89-2.80 (m, 1H), 2.70-2.64 (m, 2H), 2.63-2.59 (m, 3H), 2.47-2.38 (m, 3H), 2.28-2.19 (m, 1H), 2.18-2.12 (m, 1H), 2.11-1.96 (m, 1H), 1.37 (d, J=13.6 Hz, 1H) ppm; ¹³C NMR (151 MHz, CDCl₃, rotamers) δ=196.8, 171.0, 170.8, 150.1, 150.0, 149.7, 149.6, 144.9, 144.7, 140.3, 140.2, 133.3, 133.1, 133.0, 122.2, 122.1, 117.3, 117.2, 116.1, 116.0, 114.6, 114.1, 103.6, 103.2, 103.1, 102.9, 96.2, 69.7, 69.4, 68.9, 68.6, 68.5, 68.2, 67.7, 67.6, 67.53, 67.51, 61.4, 52.6, 52.4, 48.2, 47.7, 41.0, 25.7, 24.13, 24.12, 22.2, 22.1, 21.4, 21.0, 20.8 ppm; HRMS (ESI-TOF) calcd for C₂₉H₃₀O₁₁Na⁺ [M+Na]⁺577.1680, found 577.1697.

### (1S,2S,3aS,4R,13aS)-2-(1,3-Dioxan-2-yl)-5-methyl-7,12-dioxo-3a,4,7,12-tetrahydro-13aH-spiro[2,4-epoxyfuro[3,2-b]naphtho[2,3-h]chromene-1,2′-oxirane]-11,13a-diyl diacetate (Trx28)

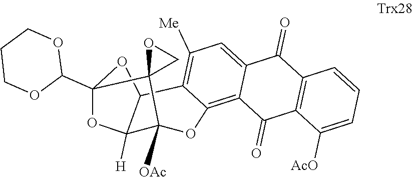

To a solution of Trx26 (5.4 mg, 9.3 μmol, 1.0 equiv) in EtOAc (0.2 ml) was added PhSeCl (2.1 mg, 11.1 μmol, 1.2 equiv) at 23° C. The mixture was stirred at this temperature for 24 h before it was diluted with EtOAc (5 ml) and quenched with H₂O (5 ml). The resulting mixture was extracted with EtOAc (3×5 ml). The combined organic phases were dried over anhydrous Na₂SO₄, filtered and concentrated under reduced pressure to give the crude phenylseleno ketone which was dissolved in CH₂Cl₂ (1 ml). To the stirred solution at 0° C. was added H₂O₂ (30 wt % in H₂O, 128 μL, 128 μmol, 13.8 equiv) and the resulting mixture was stirred at this temperature for 0.5 h. The mixture was quenched with Na₂SO₃ (sat. aq., 2 ml) and extracted with CH₂Cl₂ (3×5 ml) with no precautions to exclude air. At this stage, ¹H NMR spectroscopic analysis (CDCl₃, 600 MHz) revealed a mixture of methoxy acetate phenol Trx26c and quinone Trx28, with the former being converted to the latter upon exposure to air through acetate migration (Trx26d) and air oxidation (˜2 h). Removal of the solvent and purification of the residue by preparative thin layer chromatography (silica gel, EtOAc:hexanes 2:1) afforded Trx28 (2.54 mg, 4.49 μmol, 48% for the two steps) as a yellow foam. R_{f}=0.43 (silica gel, EtOAc:hexanes 2:1); [α]_{D}²⁵=+72.0 (c=0.1, CH₂Cl₂); FT-IR (film): νₘₐₓ=1771, 1677, 1594, 1458, 1333, 1270, 1194, 1096, 1040, 1001 cm⁻¹; ¹H NMR (600 MHz, CDCl₃) δ=8.15 (dt, J=7.9, 1.2 Hz, 1H), 7.77 (s, 1H), 7.74-7.66 (m, 1H), 7.38 (dt, J=8.0, 1.1 Hz, 1H), 5.57 (d, J=4.3 Hz, 1H), 5.32-5.19 (m, 1H), 5.07 (s, 1H), 4.37-4.24 (m, 1H), 4.20-4.14 (m, 1H), 3.95-3.86 (m, 1H), 3.80 (td, J=12.1, 2.6 Hz, 1H), 3.09 (d, J=5.9 Hz, 1H), 3.05 (d, J=5.9 Hz, 1H), 2.60 (s, 3H), 2.43 (d, J=1.1 Hz, 3H), 2.32-2.18 (m, 4H), 1.40 (d, J=13.7 Hz, 1H) ppm; ¹³C NMR (151 MHz, CDCl₃) δ=182.7, 180.0, 169.8, 168.6, 150.9, 149.7, 146.5, 135.0, 134.3, 134.0, 130.2, 126.8, 125.3, 123.7, 122.2, 120.7, 103.5, 101.3, 96.2, 71.9, 69.0, 68.9, 67.7, 67.5, 48.2, 25.7, 21.8, 21.4, 20.2 ppm; HRMS (ESI-TOF) calcd for C₂₉H₂₄NaO₁₂⁺ [M+Na]⁺587.1160, found 587.1166.

### (1S,2S,3aS,4R,13aR)-2-(1,3-Dioxan-2-yl)-13a-methoxy-5-methyl-7,12-dioxo-3a,7,12,13a-tetrahydro-4H-spiro[2,4-epoxyfuro[3,2-b]naphtho[2,3-h]chromene-1,2′-oxiran]-11-yl acetate (Trx29)

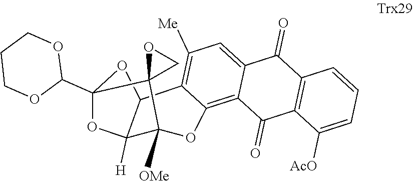

Trx28 was synthesized from Trx27 (2.0 mg, 3.6 μmol) following the same procedure as that used for the preparation of Trx28. Yield: 1.12 mg, 2.02 μmol, 56% for the two steps; yellow foam; R_{f}=0.60 (silica gel, EtOAc); [α]_{D}²⁵=+145.7 (c=0.1, CH₂Cl₂); FT-IR (film): νₘₐₓ=2921, 1772, 1676, 1593, 1445, 1335, 1271, 1192, 1097, 996 cm⁻¹; ¹H NMR (600 MHz, CDCl₃) δ=8.17 (dd, J=7.8, 1.3 Hz, 1H), 7.76 (s, 1H), 7.72 (t, J=7.9 Hz, 1H), 7.40 (dd, J=8.0, 1.3 Hz, 1H), 5.21 (d, J=4.0 Hz, 1H), 5.07 (s, 1H), 4.85 (d, J=4.0 Hz, 1H), 4.29 (dd, J=11.7, 4.8 Hz, 1H), 4.14 (dd, J=11.5, 4.9 Hz, 1H), 3.91 (td, J=12.3, 2.6 Hz, 1H), 3.82-3.77 (m, 1H), 3.76 (s, 3H), 2.99 (d, J=5.7 Hz, 1H), 2.92 (d, J=5.7 Hz, 1H), 2.60 (s, 3H), 2.42 (s, 3H), 2.27-2.16 (m, 1H), 1.38 (d, J=13.7 Hz, 1H) ppm; ¹³C NMR (151 MHz, CDCl₃) δ=182.7, 180.6, 169.9, 151.5, 149.7, 146.8, 134.9, 134.4, 134.1, 130.1, 126.8, 125.3, 124.3, 121.9, 120.8, 103.8, 102.4, 96.2, 72.0, 69.1, 68.7, 67.7, 67.5, 52.9, 48.3, 25.7, 21.4, 20.3 ppm; HRMS (ESI-TOF) calcd for C₂₈H₂₄NaO₁₁⁺ [M+Na]⁺ 559.1211, found 559.1222.

### (1S,2S,3aS,4R,13aR)-2-(1,3-Dioxan-2-yl)-11,13a-dihydroxy-5-methyl-3a,13a-dihydro-4H-spiro[2,4-epoxyfuro[3,2-b]naphtho[2,3-h]chromene-1,2′-oxirane]-7,12-dione (Trx30)

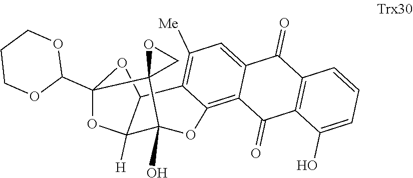

To a solution of Trx28 (2.1 mg, 3.7 μmol, 1.0 equiv) in THF (0.4 ml) at 0° C. was added LiOH (1.0 N in H₂O, 0.2 ml, 200 μmol, 54 equiv). The mixture was stirred at this temperature for 1 h before it was quenched with NaHCO₃ (sat. aq., 1 ml). The resulting mixture was extracted with CH₂Cl₂ (3×2 ml). The combined organic phases were dried over anhydrous Na₂SO₄, filtered and concentrated under reduced pressure. The residue was purified by preparative thin layer chromatography (silica gel, EtOAc:hexanes 2:1) to give the titled compound (1.4 mg, 2.9 μmol, 78%) as a pale yellow oil. Trx30: R_{f}=0.38 (silica gel, EtOAc:hexanes 2:1); [α]_{D}²⁵=+177.5 (c=0.1, CH₂Cl₂); FT-IR (film): νₘₐₓ=2851, 1719, 1672, 1634, 1591, 1559, 1454, 1356, 1318, 1268, 982 cm⁻¹; ¹H NMR (600 MHz, CDCl₃) δ=12.86 (s, 1H), 7.84 (s, 1H), 7.77 (dd, J=7.5, 1.3 Hz, 1H), 7.62 (dd, J=8.4, 7.5 Hz, 1H), 7.29 (dd, J=8.3, 1.2 Hz, 1H), 5.27 (d, J=3.9 Hz, 1H), 5.06 (s, 1H), 4.89 (d, J=3.9 Hz, 1H), 4.50 (s, 1H), 4.32-4.27 (m, 1H), 4.22-4.12 (m, 1H), 3.90 (td, J=12.1, 2.6 Hz, 1H), 3.80 (td, J=11.8, 2.5 Hz, 1H), 3.28 (d, J=5.3 Hz, 1H), 3.07 (d, J=5.3 Hz, 1H), 2.63 (s, 3H), 2.26-2.18 (m, 1H), 1.44-1.36 (m, 1H) ppm; ¹³C NMR (151 MHz, CDCl₃) δ=187.9, 182.4, 162.6, 152.7, 148.2, 136.1, 135.8, 132.7, 125.2, 124.3, 123.0, 119.2, 118.8, 117.1, 103.2, 99.0, 96.4, 73.8, 69.4, 69.3, 67.7, 67.5, 50.7, 25.7, 20.5 ppm; HRMS (ESI-TOF) calcd for C₂₅H₂₀NaO₁₀⁺ [M+Na]⁺503.0949, found 503.0964.

### (1S,2S,3aS,4R,13aR)-2-(1,3-Dioxan-2-yl)-11-hydroxy-13a-methoxy-5-methyl-3a,13a-dihydro-4H-spiro[2,4-epoxyfuro[3,2-b]naphtho[2,3-h]chromene-1,2′-oxirane]-7,12-dione (Trx31)

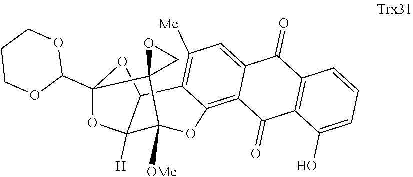

To a solution of Trx29 (1.0 mg, 1.8 μmol, 1.0 equiv) in THF (0.2 ml) at 0° C. was added LiOH (1.0 N in H₂O, 0.1 ml, 100 μmol, 56 equiv). The mixture was stirred at this temperature for 1 h before it was quenched with NaHCO₃ (sat. aq., 1 ml). The resulting mixture was extracted with CH₂Cl₂ (3×2 ml). The combined organic phases were dried over anhydrous Na₂SO₄, filtered and concentrated under reduced pressure. The residue was purified by flash column chromatography (silica gel, EtOAc:hexanes 2:1) to give the titled compound (0.74 mg, 1.5 μmol, 83%) as a pale yellow oil. Trx31: R_{f}=0.65 (silica gel, EtOAc); [α]_{D}²⁵=+156.0 (c=0.1, CH₂Cl₂); FT-IR (film): νₘₐₓ=2851, 1714, 1671, 1635, 1592, 1456, 1320, 1269, 1193, 1068, 954 cm⁻¹; ¹H NMR (600 MHz, CDCl₃) δ=7.84 (s, 1H), 7.77 (dd, J=7.5, 1.2 Hz, 1H), 7.63 (t, J=7.9 Hz, 1H), 7.30 (dd, J=8.3, 1.2 Hz, 1H), 5.24 (d, J=4.0 Hz, 1H), 5.09 (s, 1H), 4.89 (d, J=4.0 Hz, 1H), 4.29 (dd, J=11.7, 5.0 Hz, 1H), 4.14 (dd, J=11.6, 4.9 Hz, 1H), 3.90 (td, J=12.1, 2.6 Hz, 1H), 3.81-3.77 (m, 1H); 3.80 (s, 3H), 3.02 (d, J=5.7 Hz, 1H), 2.87 (d, J=5.7 Hz, 1H), 2.63 (s, 3H), 2.27-2.17 (m, 1H), 1.39 (d, J=13.7 Hz, 1H) ppm; ¹³C NMR (151 MHz, CDCl₃) δ=187.8, 182.5, 162.6, 152.5, 148.1, 136.1, 135.7, 132.7, 125.1, 124.3, 122.8, 119.1, 118.8, 117.1, 103.9, 102.6, 96.2, 71.8, 69.1, 68.6, 67.7, 67.5, 53.2, 48.2, 25.7, 20.5 ppm; HRMS (ESI-TOF) calcd for C₂₆H₂₂NaO₁₀⁺ [M+Na]⁺517.1105, found 517.1118.

### (1S,2S,3aS,4R,13aS)-10-Chloro-2-(1,3-dioxan-2-yl)-5-methyl-7,12-dioxo-3a,4,7,12-tetrahydro-13aH-spiro[2,4-epoxyfuro[3,2-b]naphtho[2,3-h]chromene-1,2′-oxirane]-11,13a-diyl diacetate (Trx32)

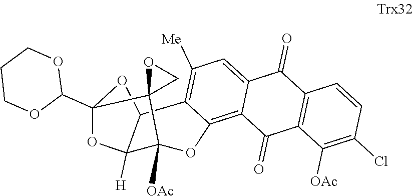

To a solution of Trx26 (3.8 mg, 6.5 μmol, 1.0 equiv) in EtOAc (0.2 ml) was added PhSeCl (12.4 mg, 65 μmol, 10.0 equiv) at 23° C. The mixture was stirred at this temperature for 72 h before it was diluted with EtOAc (5 ml) and quenched with H₂O (5 ml). The resulting mixture was extracted with EtOAc (3×5 ml). The combined organic phases were dried over anhydrous Na₂SO₄, filtered and concentrated under reduced pressure. The residue was purified by preparative thin layer chromatography (silica gel, EtOAc:hexanes 4:1) to afford Trx32 (1.8 mg, 3.0 μmol, 46%) as a yellow foam. R_{f}=0.44 (silica gel, EtOAc:hexanes 2:1); [α]_{D}²⁵=+67.7 (c=0.1, CH₂Cl₂); FT-IR (film): νₘₐₓ=1781, 1678, 1597, 1580, 1463, 1368, 1333, 1272, 1187, 1096 cm⁻¹; ¹H NMR (600 MHz, CDCl₃) δ=8.10 (d, J=8.4 Hz, 1H), 7.81-7.75 (m, 2H), 5.57 (d, J=4.0 Hz, 1H), 5.25 (d, J=4.0 Hz, 1H), 5.08 (s, 1H), 4.29 (dd, J=11.6, 4.8 Hz, 1H), 4.16 (dd, J=11.6, 4.9 Hz, 1H), 3.90 (td, J=12.1, 2.5 Hz, 1H), 3.80 (td, J=12.0, 2.5 Hz, 1H), 3.08 (d, J=6.1 Hz, 1H), 3.05 (d, J=5.8 Hz, 1H), 2.60 (s, 3H), 2.48 (s, 3H), 2.26 (s, 3H), 2.26-2.19 (m, 1H), 1.40 (d, J=13.7 Hz, 1H) ppm; ¹³C NMR (151 MHz, CDCl₃) δ=181.9, 179.1, 168.7, 168.6, 151.0, 146.8, 146.2, 136.2, 134.8, 134.4, 132.5, 128.1, 125.7, 123.9, 122.3, 120.5, 103.4, 101.3, 96.2, 71.8, 69.0, 68.8, 67.7, 67.5, 48.2, 25.6, 21.8, 21.0, 20.2 ppm; HRMS (ESI-TOF) calcd for C₂₉H₂₃ClNaO₁₂⁺ [M+Na]⁺ 621.0770, found 621.0781.

### (1S,2S,3aS,4R,13aR)-10-Chloro-2-(1,3-dioxan-2-yl)-13a-methoxy-5-methyl-7,12-dioxo-3a,7,12,13a-tetrahydro-4H-spiro[2,4-epoxyfuro[3,2-b]naphtho[2,3-h]chromene-1,2′-oxiran]-11-yl acetate (Trx33)

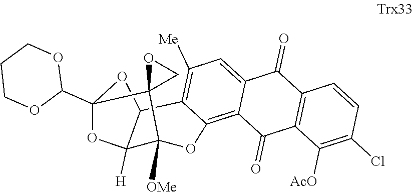

To a solution of Trx27 (4.4 mg, 7.9 μmol, 1.0 equiv) in EtOAc (0.3 ml) was added PhSeCl (15.2 mg, 79 μmol, 10.0 equiv) at 23° C. The mixture was stirred at this temperature for 72 h before it was diluted with EtOAc (5 ml) and quenched with H₂O (5 ml). The resulting mixture was extracted with EtOAc (3×5 ml). The combined organic phases were dried over anhydrous Na₂SO₄, filtered and concentrated under reduced pressure. The residue was purified by preparative thin layer chromatography (silica gel, EtOAc:hexanes 4:1) to afford Trx33 (1.9 mg, 3.3 μmol, 42%) as a yellow foam. R_{f}=0.67 (silica gel, EtOAc); [α]_{D}²⁵=+164.0 (c=0.1, CH₂Cl₂); FT-IR (film): νₘₐₓ=2852, 1781, 1677, 1595, 1580, 1456, 1336, 1273, 1187, 995 cm⁻¹; ¹H NMR (600 MHz, CDCl₃) δ=8.11 (d, J=8.4 Hz, 1H), 7.79 (d, J=8.4 Hz, 1H), 7.75 (s, 1H), 5.21 (d, J=4.0 Hz, 1H), 5.07 (s, 1H), 4.86 (br s, 1H), 4.29 (dd, J=11.5, 4.9 Hz, 1H), 4.14 (dd, J=11.5, 4.9 Hz, 1H), 3.89 (td, J=12.0, 2.5 Hz, 1H), 3.79 (td, J=12.1, 2.6 Hz, 1H), 3.76 (s, 3H), 2.99 (d, J=5.7 Hz, 1H), 2.91 (d, J=5.7 Hz, 1H), 2.61 (s, 3H), 2.47 (s, 3H), 2.22 (qt, J=12.7, 5.0 Hz, 1H), 1.41-1.36 (m, 1H) ppm; ¹³C NMR (151 MHz, CDCl₃) δ=182.0, 179.7, 168.6, 151.6, 147.1, 146.1, 136.2, 134.7, 134.5, 132.5, 128.1, 125.7, 124.4, 122.0, 120.6, 103.9, 102.5, 96.2, 71.7, 69.0, 68.6, 67.7, 67.5, 52.9, 48.2, 25.7, 21.0, 20.3 ppm; HRMS (ESI-TOF) calcd for C₂₈H₂₃ClNaO₁₁⁺ [M+Na]⁺593.0821, found 593.0827.

### 2-(p-Tolylselanyl)-3,4-dihydronaphthalen-1(2H)-one (III)

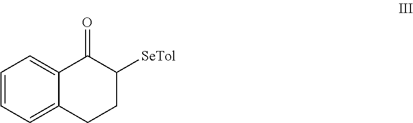

To a stirred solution of α-tetralone (77.0 mg, 0.529 mmol, 1.0 equiv) in EtOAc (2 ml) at 23° C. was added tolylselenyl chloride (Schmid and Garratt, 1983) (109 mg, 0.529 mmol, 1.0 equiv). The mixture was stirred at this temperature for 2 h before it was directly subjected to preparative thin layer chromatography (silica gel, Et₂O:CH₂Cl₂:hexanes 1:3:9) to afford the title compound as a colorless oil (70.2 mg, 0.222 mmol, 42%) and recovered starting material (23.5 mg, 31%). III: R_{f}=0.35 (silica gel, Et₂O:CH₂Cl₂:hexanes 1:3:9); FT-IR (film): νₘₐₓ=3065, 3021, 2923, 1672, 1598, 1488, 1454, 1431, 1350, 1298; ¹H NMR (600 MHz, CDCl₃) δ=8.06 (dd, J=7.8, 1.4 Hz, 1H), 7.52-7.46 (m, 3H), 7.32 (t, J=7.6 Hz, 1H), 7.23 (d, J=7.6 Hz, 1H), 7.11 (d, J=7.8 Hz, 2H), 4.26-4.13 (m, 1H), 3.24 (ddd, J=16.3, 10.9, 4.6 Hz, 1H), 2.87 (dt, J=17.0, 4.5 Hz, 1H), 2.50 (ddt, J=14.2, 11.0, 4.4 Hz, 1H), 2.40-2.29 (m, 4H) ppm; ¹³C NMR (151 MHz, CDCl₃) δ=193.7, 143.0, 138.7, 135.9, 133.6, 131.4, 130.2, 128.8, 128.3, 127.0, 123.9, 48.9, 29.4, 27.1, 21.4 ppm; HRMS (ESI-TOF) calcd for [M+H]⁺Cl₇H₁₇OSe⁺ 317.0439, found 317.0428.

### 2-Chloro-2-(p-tolylselanyl)-3,4-dihydronaphthalen-1(2H)-one (VIIIa)

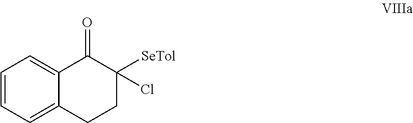

To a stirred solution of CSA (7.0 mg, 0.031 mmol, 1.0 equiv) and compound III (9.7, 0.031 mmol, 1.0 equiv) in EtOAc (0.3 ml) at 23° C. was added PhSeCl (11.8 mg, 0.062 mmol, 2.0 equiv). The reaction mixture was stirred at this temperature for 2 h before it was directly subjected to preparative thin layer chromatography (Et₂O:CH₂Cl₂:hexanes 1:3:9) to afford the title compound as a colorless oil (2.2 mg, 6.3 μmol, 20%) and recovered starting material (5.7 mg, 0.018 mmol, 59%). This product proved rather labile to prolonged reaction times and subsequent manipulation. The reaction was therefore stopped before completion and caution was taken during isolation to minimize decomposition. III: R_{f}=0.44 (silica gel, Et₂O:CH₂Cl₂:hexanes 1:3:9); FT-IR (film): νₘₐₓ=3398, 2920, 2851, 1686, 1599, 1488, 1455, 1388, 1290, 1234, 1218, 1015; ¹H NMR (600 MHz, CDCl₃) δ=8.12 (dd, J=7.8, 1.4 Hz, 1H), 7.51 (dd, J=8.1, 1.9 Hz, 3H), 7.37 (dd, J=8.5, 6.3 Hz, 1H), 7.24 (d, J=7.4 Hz, 1H), 7.17 (d, J=7.8 Hz, 2H), 3.21 (ddd, J=17.2, 10.0, 5.1 Hz, 1H), 3.02 (dt, J=17.2, 4.9 Hz, 1H), 2.75-2.64 (m, 2H), 2.39 (s, 3H) ppm; ¹³C NMR (151 MHz, CDCl₃) δ=186.9, 142.1, 140.5, 138.0, 134.0, 132.5, 130.1, 129.4, 128.6, 127.4, 122.1, 75.4, 40.4, 28.4, 21.6 ppm; HRMS (ESI-TOF) calcd for [M+Na]⁺ Cl₇H₁₅ClOSeNa⁺ 372.9869, found 372.9896.

Data from HPLC Traces for Determining the Enantiomeric Ratio of the α-Hydroxylation with Oxaziridine Reagents:

The HPLC analysis was carried out on Chiralcel OD-H, 25° C.; flow rate: 1 mL/min; hexanes/isopropanol: 99.5/0.5; detector 254 nm.

TABLE 4
Optimization of α-Hydroxylation of Bromide 18 with Oxaziridine Reagents^{a}

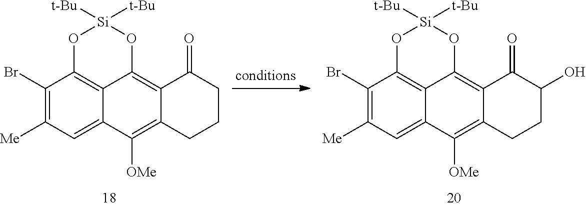 [SMILES: COc1c2c(c3c4c(c(Br)c(C)cc14)O[Si](C(C)(C)C)(C(C)(C)C)O3)C(=O)CCC2 ; COc1c2c(c3c4c(c(Br)c(C)cc14)O[Si](C(C)(C)C)(C(C)(C)C)O3)C(=O)C(O)CC2]

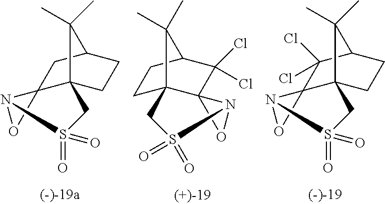

| | | | ϑ | | rsm | yield | er |
|---|---|---|---|---|---|---|---|
| entry | oxaziridine | base | (° C.) | t (h) | (%)^{b} | (%)^{c} | (R):(S)^{d} |
| 1 | (−)-19a | NHMDS | −78→0 | 2 | 41 | 33 | 1.9:1 |
| 2 | (+)-19 | NHMDS | −78 | 1 | — | 54 | 21:1 |
| 3 | (+)-19 | LHMDS | −78 | 1 | — | 55 | 21:1 |
| 4 | (+)-19 | LDA | −78 | 0.5 | 9 | 66 | 14:1 |
| 5^{e} | (+)-19 | LDA | −78 | 0.5 | 30 | 37 | 3.2:1 |
| 6 | (−)-19 | LTMP | −78 | 0.5 | 7 | 77 | 1:27 |

^{a}Reactions were carried out on 0.10 mmol scale, with 1.5 equiv base and 1.5 equiv oxaziridine in THF;
^{b}recovered starting material;
^{c}isolated yield;
^{d}absolute configuration of 20 was determined by Mosher ester analysis;
^{e}HMPA as additive.

### 6. Example 7—Synthesis of Trioxacarcin Analogues Trx34 and Trx35

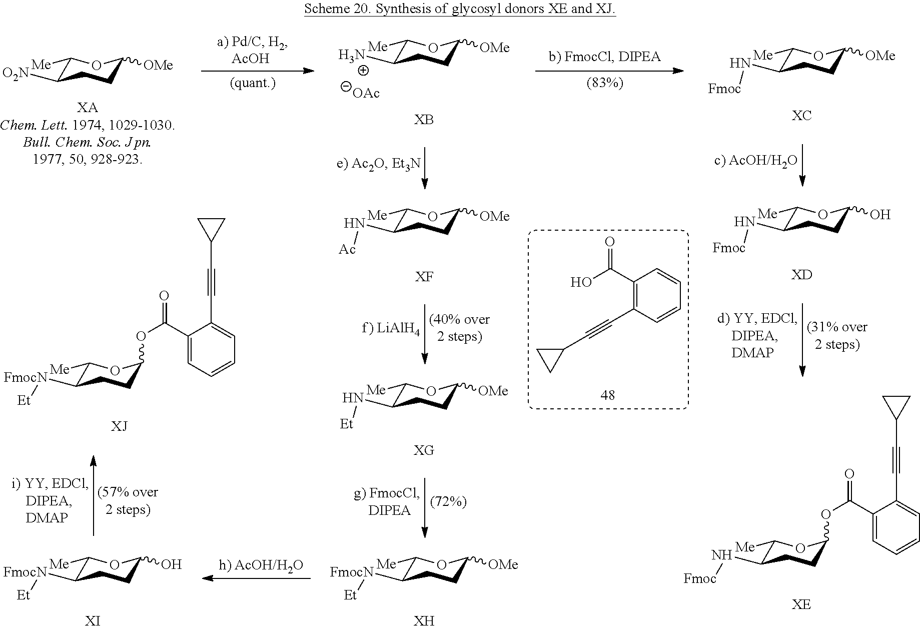

### (2S,3R)-6-Methoxy-2-methyltetrahydro-2H-pyran-3-aminiumacetate (XB)

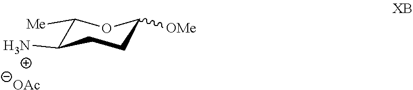

To a stirred solution of nitro compound XA (1.85 g, 10.6 mmol, 1.0 equiv) in MeOH (36 mL) and AcOH (4 mL) was added Pd(OH)₂ (370 mg, 20% w/w on carbon) at room temperature. The reaction was degassed and purged with hydrogen using a hydrogen balloon. The reaction mixture was stirred at the same temperature for another 24 h before it was filtered through Celite. The filtrate was concentrated under vacuum and the resulting residue (2.17 g, 10.6 mmol, quant. yield) was used directly for the next step without further purification.

XB (mixture of α:β 1:1): [α]_{D}²⁵=−70.9 (c=1.9, CH₃OH); FT-IR (film): νₘₐₓ 3363, 2935, 2849, 2587, 2160, 1633, 1539, 1395, 1124, 1053, 1010, 973 cm⁻¹; ¹H NMR (600 MHz, CDCl₃, α:β ca. 1:2, rotamers around Fmoc group) δ=4.67 (d, J=3.0 Hz, 1H), 4.42 (dd, J=9.1, 2.2 Hz, 1H), 3.76 (dq, J=9.6, 6.2 Hz, 1H), 3.50 (dd, J=9.2, 6.2 Hz, 1H), 3.46-3.43 (m, 4H), 3.35 (s, 3H), 2.82 (td, J=10.6, 10.1, 4.2 Hz, 1H), 2.72 (ddd, J=11.3, 9.1, 4.3 Hz, 1H), 2.09 (dt, J=12.0, 3.8 Hz, 1H), 1.92 (s, 6H), 1.88 (dd, J=6.4, 3.3 Hz, 2H), 1.82 (ddd, J=15.2, 8.1, 3.3 Hz, 2H), 1.64-1.55 (m, 1H), 1.51 (dtd, J=13.2, 9.0, 4.4 Hz, 1H), 1.32 (d, J=6.2 Hz, 3H), 1.26 (d, J=6.3 Hz, 3H) ppm; ¹³C NMR (151 MHz, CDCl₃, α:β ca. 1:2, rotamers around Fmoc group) δ=179.4, 103.8, 98.9, 98.6, 74.6, 67.4, 56.5, 54.9, 53.8, 53.4, 31.1, 30.0, 28.7, 24.9, 23.6, 18.5, 18.3 ppm.

### 9H-Fluoren-9-ylmethyl[(2S,3R)-6-methoxy-2-methyltetrahydro-2H-pyran-3-yl]carbamate (XC)

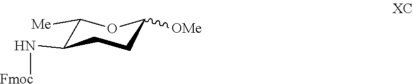

To a stirred solution of ammonium salt XB (84.7 mg, 0.413 mmol, 1.0 equiv) in CH₂Cl₂ (4 mL) were added DIPEA (170 μL, 126 mg, 0.909 mmol, 2.2 equiv) and FmocCl (259 mg, 0.454 mmol, 1.1 quiv) at 0° C. The reaction mixture was stirred at this temperature for 0.5 h before it was quenched by addition of a saturated aqueous solution of NH₄Cl (2 mL). Then, the resulting mixture was extracted with CH₂Cl₂ (2×4 mL). The combined organic phases were dried over anhydrous Na₂SO₄, filtered, and concentrated under reduced pressure. The crude residue was purified by flash column chromatography (silica gel, EtOAc:CH₂Cl₂ 1:10 to 1:6, v/v) to give the product (126 mg, 0.343 mmol, 83%) as a white solid.

XC (mixture of α:β 1:1): R_{f}=0.34, 0.39 (silica gel, EtOAc:CH₂Cl_{2=1:6}, v/v); [α]_{D}²⁵=−59.3 (c=1.08, CHCl₃); FT-IR (film): νₘₐₓ=3319, 3065, 2951, 2888, 2833, 1686, 1540, 1450, 1310, 1251, 1053, 757, 739 cm⁻¹; ¹H NMR (600 MHz, CDCl₃, α:β ca. 1:2, rotamers around Fmoc group) δ=7.77 (d, J=7.6 Hz, 3H), 7.58 (d, J=7.3 Hz, 3H), 7.40 (dd, J=8.4, 6.6 Hz, 3H), 7.32 (t, J=7.5 Hz, 3H), 4.66 (s, 1H), 4.53-4.29 (m, 4H), 4.22 (d, J=7.0 Hz, 2H), 3.57-3.52 (m, 1H), 3.48 (s, 1.5H), 3.38-3.29 (m, 4H), 1.89-1.73 (m, 5H), 1.64 (dd, J=11.4, 5.7 Hz, 1.5 H), 1.29-1.23 (m, 1.5H), 1.20 (d, J=6.2 Hz, 3H); ¹³C NMR (151 MHz, CDCl₃, α:β ca. 1:2, rotamers around Fmoc group) δ=156.0, 144.1, 144.0, 141.5, 127.8, 127.7, 127.2, 127.2, 125.1, 125.1, 124.8, 120.2, 120.1, 120.1, 102.6, 97.5, 68.6, 66.6, 56.4, 54.7, 52.6, 47.5, 30.7, 29.6, 25.8, 18.6, 18.4 ppm.

### 9H-Fluoren-9-ylmethyl[(2S,3R)-6-hydroxy-2-methyltetrahydro-2H-pyran-3-yl]carbamate (XD)

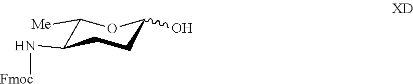

A stirred solution of XC (44.3 mg, 0.121 mmol) in H₂O/AcOH (1.5 mL/0.5 mL) was heated to 80° C. for 0.5 h before toluene (3 mL) was added. The solvents were removed under reduced pressure to give the crude hemiacetal XD (35.0 mg) as a white powder, which was used without further purification.

### (5R,6S)-5-{[(9H-Fluoren-9-ylmethoxy)carbonyl]amino}-6-methyltetrahydro-2H-pyran-2-yl 2-(cyclopropylethynyl)benzoate (XE)

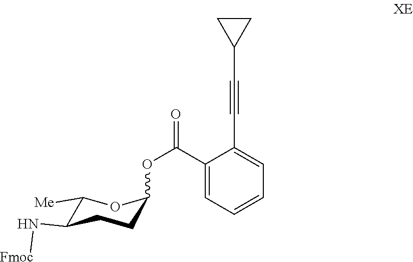

To a stirred suspension of lactol XD (13.9 mg, 0.043 mmol, 1.0 equiv) and benzoic acid 48 (13.9 mg, 0.086 mmol, 2.0 equiv) in CH₂Cl₂(2 mL) at 25° C. were subsequently added DIPEA (20 μL, 14.8 mg, 0.129 mmol, 3.0 equiv), DMAP (5.2 mg, 0.043 mmol, 1.0 equiv) and EDCI (16.4 mg, 0.0.086 mmol, 2.0 equiv). Then, the reaction mixture was stirred at this temperature for 20 min and then quenched by the addition of a saturated aqueous solution of NH₄Cl (1 mL). The resulting mixture was extracted with CH₂Cl₂ (2 mL) and the combined organic phases were dried over anhydrous Na₂SO₄, filtered, and concentrated under reduced pressure. The crude residue was purified by flash column chromatography (silica gel, EtOAc:hex 1:4, v/v) to give benzoate XE (7.9 mg, 0.015 mmol, 31% over 2 steps) as a colorless oil.

XE: R_{f}=0.57 (silica gel, EtOAc:hexanes=1:3, v/v); [α]⁵=−17.4 (c=0.5, CHCl₃); FT-IR (film): νₘₐₓ=3332, 2934, 2473, 2231, 1718, 1539, 1451, 1426, 1244, 1029, 758 cm⁻¹; ¹H NMR (600 MHz, CDCl₃, α:β ca. 1:1, rotamers around Fmoc group, signals of a and § anomers are reported in groups) δ=¹H NMR (600 MHz, CDCl₃) δ=7.97-7.87 (m, 1H), 7.82-7.73 (m, 2H), 7.63-7.53 (m, 2H), 7.51-7.46 (m, 1H), 7.45-7.37 (m, 3H), 7.36-7.28 (m, 3H), 6.39-5.81 (m, 1H), 4.63-4.47 (m, 2H), 4.45 (d, J=8.4 Hz, 1H), 4.22 (d, J=8.1 Hz, 1H), 3.87-3.46 (m, 1H), 2.19 (d, J=13.9 Hz, 1H), 2.04 (s, OH), 1.83 (d, J=33.6 Hz, 0H), 1.53-1.39 (m, 1H), 1.32-1.17 (m, 6H), 0.93-0.82 (m, 6H) ppm; ¹³C NMR (151 MHz, CDCl₃, a mixture of anomeric isomers, rotamers around Fmoc group) δ=¹³C NMR (151 MHz, CDCl₃) δ=165.1, 144.1, 144.0, 144.0, 141.6, 134.9, 134.5, 132.1, 131.9, 131.2, 130.9, 130.7, 128.0, 128.0, 127.9, 127.4, 127.3, 127.3, 127.2, 125.2, 125.1, 125.0, 124.6, 120.2, 120.2, 120.2, 99.8, 99.2, 94.6, 92.4, 76.3, 75.1, 74.7, 71.4, 66.8, 66.5, 60.6, 52.3, 51.9, 51.7, 47.6, 47.6, 31.8, 29.9, 29.4, 28.9, 28.6, 22.9, 22.9, 21.3, 19.2, 18.9, 18.6, 14.4, 14.3, 9.3, 9.2, 9.1, 9.0, 1.0, 0.9 ppm.

### N-[(2S,3R)-6-Methoxy-2-methyltetrahydro-2H-pyran-3-yl]acetamide (XF)

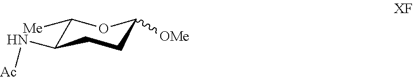

To a solution of ammonium salt XB (279 mg, 1.36 mmol, 1.0 equiv) in CH₂Cl₂(10 mL) were added Et₃N (470 μL, 341 mg, 3.38 mmol 2.5 equiv) and Ac₂O (150 μL, 162 mg, 1.63 mmol, 1.2 equiv) at 25° C. The reaction mixture was stirred at this temperature for 0.5 h before it was quenched by addition of a saturated aqueous solution of NaHCO₃ (4 mL). The resulting mixture was stirred at 25° C. for 1 h and then extracted with CH₂Cl₂ (2×10 mL). The combined organic phases were dried over anhydrous Na₂SO₄, filtered, and concentrated under reduced pressure to give a crude (218 mg, 1.17 mmol) as brown foam. The crude was used without further purification.

### (2S,3R)—N-Ethyl-6-methoxy-2-methyltetrahydro-2H-pyran-3-amine (XG)

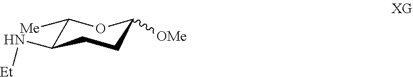

To a stirred solution of acetamide XF (138 mg, 0.74 mmol, 1.0 quiv) in THF (7 mL) at 25° C. was added LiAlH₄ (1.0 M in THF, 2.2 mL, 4.95 mmol, 3.0 equiv). The reaction mixture was stirred at 60° C. for 0.5 h and then cooled to 25° C. The reaction mixture was poured into a saturated aqueous solution of NaHCO₃ (10 mL) at 0° C. The resulting mixture was stirred at 25° C. for 0.5 h and then extracted with EtOAc (2×3 mL). The combined organic phases were dried over anhydrous Na₂SO₄, filtered, and concentrated under reduced pressure to give a crude product. The crude residue was purified by flash column chromatography (silica gel, EtOAc:MeOH:Et₃N 100:10:0.25, ν/ν/ν) to give amine XG (60 mg, 0.347 mmol, 40% over two steps) as a colorless oil.

XG (α:β ca. 3:1): R_{f}=0.10 (silica gel, EtOAc:MeOH:Et₃N 100:10:0.1, ν/ν/ν); [α]_{D}²⁵=−80.4 (c=0.44, CHCl₃); FT-IR (film): νₘₐₓ=3284, 3079, 2932, 1653, 1557, 1447, 1370, 1126, 1057, 982, 932 cm⁻¹; ¹H NMR (600 MHz, CDCl₃, α:β ca. 3:1, only the major α-anomer was reported) δ=4.67-4.60 (m, 1H), 3.64-3.52 (m, 1H), 3.33 (s, 3H), 2.75 (dt, J=11.4, 7.2 Hz, 1H), 2.59 (dq, J=11.3, 7.1 Hz, 1H), 2.25 (ddd, J=11.3, 9.2, 4.2 Hz, 1H), 1.88-1.80 (m, 1H), 1.79 (ddd, J=4.5, 3.0, 1.5 Hz, 1H), 1.72 (tt, J=13.6, 4.0 Hz, 1H), 1.58-1.52 (m, 1H), 1.24 (d, J=6.2 Hz, 3H), 1.09 (t, J=7.1 Hz, 3H); ¹³C NMR (151 MHz, CDCl₃, α:β ca. 4:1) δ=102.8 (minor), 97.6, 76.1 (minor), 69.3, 59.6, 59.4 (minor), 56.3 (minor), 54.4, 41.9 (minor), 41.5, 30.9 (minor), 29.9, 29.0 (minor), 25.1, 18.94 (minor), 18.86, 16.0, 15.9 (minor) ppm.

### 9H-Fluoren-9-ylmethyl ethyl[(2S,3R)-6-methoxy-2-methyltetrahydro-2H-pyran-3-yl]carbamate (XH)

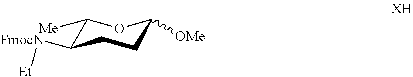

To a stirred solution of amine XG (31.7 mg, 0.183 mmol, 1.0 equiv) in CH₂Cl₂ (2 mL) were added DIPEA (40 μL, 30 mg, 0.24 mmol, 1.3 equiv) and FmocCl (52 mg, 0.202 mmol, 1.1 equiv) at 0° C. The reaction mixture was stirred at this temperature for 1 h before it was quenched by the addition of a saturated aqueous solution of NH₄Cl (2 mL). The resulting mixture was extracted with CH₂Cl₂ (2×3 mL) and the combined organic phases were dried over anhydrous Na₂SO₄, filtered, and concentrated under reduced pressure. The crude residue was purified by flash column chromatography (silica gel, EtOAc:hexanes 1:5, v/v) to give Fmoc-protected glycoside XH (51.7 mg, 0.138 mmol, 72% yield) as a colorless oil.

XH (mixture of α and β): R_{f}=0.36, 0.44 (silica gel, EtOAc:hexanes 1:3 v/v); [α]_{D}²⁵=−34.3 (c=1.97, CHCl₃); FT-IR (film): νₘₐₓ=3494, 3066, 2974, 2933, 2895, 2830, 1694, 1450, 1419, 1270, 1126, 1055, 983, 740 cm⁻¹; ¹H NMR (600 MHz, CDCl₃, a mixture of anomeric isomers, rotamers around Fmoc group) δ=4.67-4.60 (m, 1H), 3.64-3.52 (m, 1H), 3.33 (s, 3H), 2.75 (dt, J=11.4, 7.2 Hz, 1H), 2.59 (dq, J=11.3, 7.1 Hz, 1H), 2.25 (ddd, J=11.3, 9.2, 4.2 Hz, 1H), 1.88-1.80 (m, 1H), 1.79 (ddd, J=4.5, 3.0, 1.5 Hz, 1H), 1.72 (tt, J=13.6, 4.0 Hz, 1H), 1.58-1.52 (m, 1H), 1.24 (d, J=6.2 Hz, 3H), 1.09 (t, J=7.1 Hz, 3H);

¹³C NMR (151 MHz, CDCl₃ a mixture of anomeric isomers, rotamers around Fmoc group) 6=156.5, 155.9, 155.6, 144.5, 144.4, 144.3, 144.2, 144.2, 141.6, 141.6, 141.6, 141.6, 141.5, 127.8, 127.8, 127.8, 127.7, 127.7, 127.4, 127.2, 127.1, 127.1, 125.0, 124.9, 124.9, 124.8, 124.7, 124.4, 120.2, 120.1, 120.1, 120.0, 102.8, 97.6, 97.6, 66.9, 66.7, 65.9, 65.3, 56.3, 56.1, 54.7, 54.7, 50.5, 47.7, 47.7, 47.6, 34.8, 31.7, 31.6, 30.3, 29.8, 27.0, 25.9, 25.4, 23.3, 22.8, 22.8, 20.8, 18.5, 18.3, 18.2, 15.6, 15.0, 14.3, 14.3 ppm.

### 9H-Fluoren-9-ylmethyl ethyl[(2S,3R)-6-hydroxy-2-methyltetrahydro-2H-pyran-3-yl]carbamate (XI)

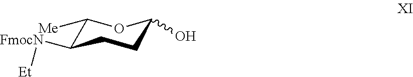

A stirred solution of glycoside XH (194 mg, 0.49 mmol) in H₂O/AcOH (1.5 mL/1.5 mL) was stirred at 80° C. for 16 h before toluene (5 mL) was added and the solvents were removed under reduced pressure. The crude residue was then purified by flash column chromatography (silica gel, EtOAc:hexanes 8:1, v/v) to give hemiacetal XI (129 mg, 0.368 mmol, 72% yield) as a colorless oil.

### (5R,6S)-5-{Ethyl[(9H-fluoren-9-ylmethoxy)carbonyl]amino}-6-methyltetrahydro-2H-pyran-2-yl 2-(cyclopropylethynyl)benzoate (XJ)

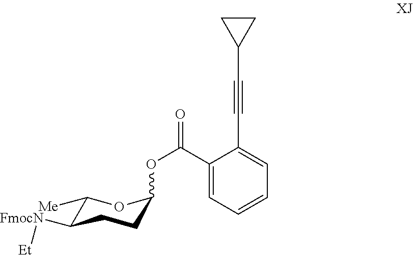

To a stirred solution of lactol XI (129 mg, 0.368 mmol, 1.0 equiv), o-alkynyl benzoic acid 48 (90.0 mg, 0.556 mmol, 1.5 equiv) in CH₂Cl₂ (4 mL) at 25° C. were added DIPEA (190 μL, 141 mg, 1.1 mmol, 3.0 quiv), DMAP (44.8 mg, 0.368 mmol, 1.0 equiv) and EDC (141 mg, 0.735 mmol, 2.0 equiv). The reaction mixture was stirred at this temperature for 2 h and then quenched by the addition of a saturated aqueous solution of NH₄Cl (2 mL). The resulting mixture was extracted with CH₂Cl₂ (2×3 mL) and the combined organic phases were dried over anhydrous Na₂SO₄, filtered, and concentrated under reduced pressure. The crude residue was purified by flash column chromatography (silica gel, EtOAc:hexanes 10:1, v/v) to give benzoate XJ (160 mg, 0.291 mmol, 79% yield) as a colorless oil.

XJ (mixture of α:β 1:1): R_{f}=0.16 (silica gel, EtOAc:hexanes 1:3, v/v); [α]_{D}²⁵=−2.7 (c=0.44, CH₂Cl₂); FT-IR (film): νₘₐₓ=3067, 2799, 2935, 2230, 1730, 1696, 1451, 1271, 1060, 758 cm⁻¹; ¹H NMR (600 MHz, methanol-d4, a mixture of anomeric isomers, rotamers around Fmoc group) δ=8.10-7.94 (m, 1H), 7.85 (d, J=7.9 Hz, 1H), 7.83-7.73 (m, 1H), 7.68-7.54 (m, 2H), 7.52-7.40 (m, 3H), 7.40-7.22 (m, 4H), 6.30-5.24 (m, 1H), 4.93-4.76 (m, 1H), 4.71-4.49 (m, 1H), 4.29-4.16 (m, 1H), 3.24-3.03 (m, 2H), 2.95-2.25 (m, 2H), 2.11-1.91 (m, 1H), 1.85-1.66 (m, 1H), 1.66-1.39 (m, 2H), 1.10-0.85 (m, 6H), 0.84-0.67 (m, 3H), 0.60 (br s, 2H) ppm; ¹³C NMR (151 MHz, methanol-d4, a mixture of anomeric isomers, rotamers around Fmoc group) δ=166.5, 166.1, 165.9, 157.2, 145.8, 145.6, 145.5, 145.4, 143.1, 142.9, 135.3, 135.0, 134.8, 133.12, 133.06, 133.0, 132.9, 132.3, 131.3, 131.2, 131.0, 128.9, 128.8, 128.7, 128.40, 128.39, 128.2, 128.14, 128.07, 126.0, 125.7, 125.2, 125.1, 121.9, 121.7, 121.0, 120.9, 100.5, 95.8, 95.5, 93.5, 75.3, 75.1, 74.5, 74.3, 67.6, 67.4, 48.8, 48.5, 31.6, 31.4, 31.3, 30.5, 26.5, 26.4, 23.5, 18.59, 18.56, 17.9, 15.3, 14.1, 9.29, 9.27, 9.25, 9.22, 9.14, 9.07, 1.3, 1.24, 1.20, 1.17. ppm.

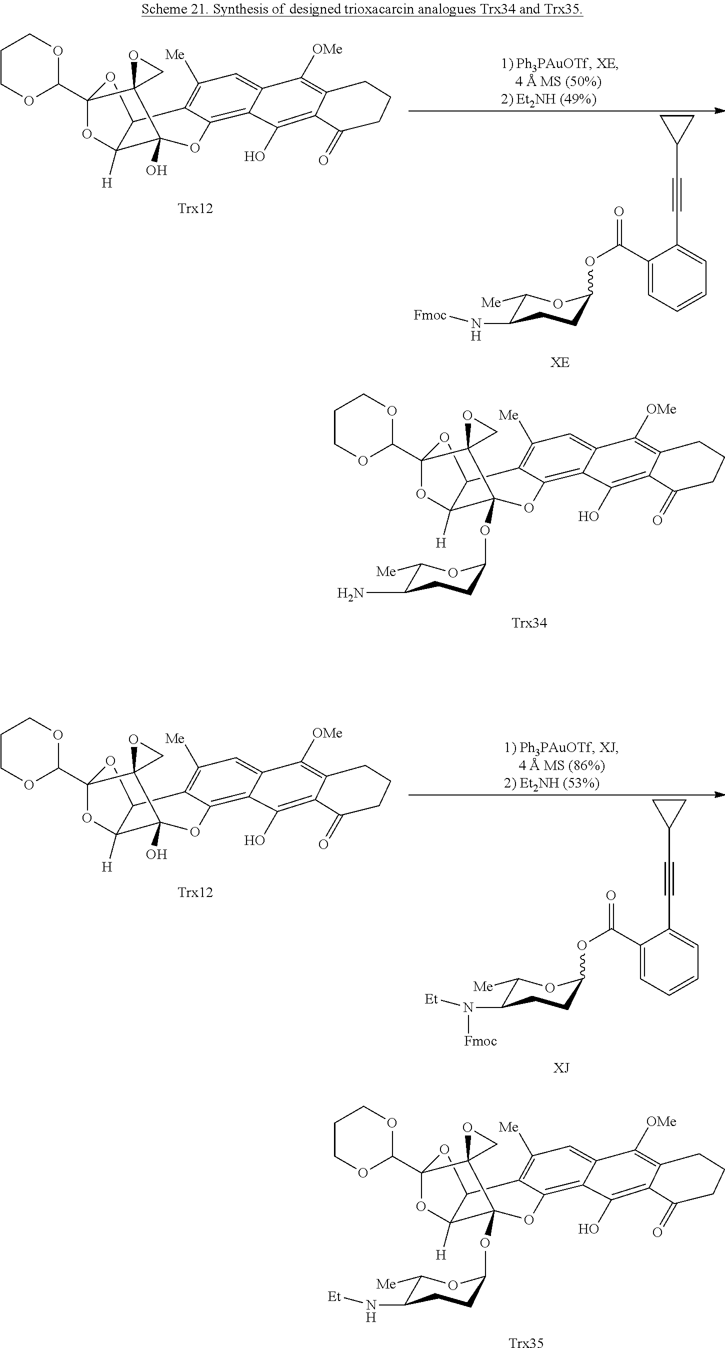

### (1S,2S,13aS)-13a-{[(2S,5R,6S)-5-Amino-6-methyltetrahydro-2H-pyran-2-yl]oxy}-2-(1,3-dioxan-2-yl)-12-hydroxy-7-methoxy-5-methyl-3a,4,8,9,10,13a-hexahydro-11H-spiro[2,4-epoxyfuro[3,2-b]naphtho[2,3-h]chromene-1,2′-oxiran]-11-one (Trx34)

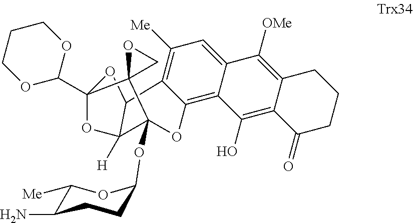

To a mixture of hemiacetal Trx12 (3.7 mg, 7.43 μmol, 1.0 equiv), glycosyl donor XE (12.4 mg, 23.8 μmol, 3.0 equiv) and flame-dried 4 Å molecular sieves (20 mg) in CH₂Cl₂ (1 mL) at −40° C. was added freshly prepared PPh₃AuOTf (0.05 M in CH₂Cl₂, 30 μL, 1.5 μmol, 0.2 equiv). The reaction mixture was stirred at this temperature for 0.5 h before the reaction was quenched by addition of NaHCO₃ (sat. aq., 1 mL). The crude mixture was filtered through a short pad of Celite and extracted with CH₂Cl₂ (2×1 mL). The combined organic phases were dried over anhydrous Na₂SO₄, filtered and concentrated under reduced pressure. The residue was purified by preparative thin layer chromatography (silica gel, EtOAc) to give the glycosylation product (3.1 mg, 3.7 μmol, 50% yield) as a yellow foam.

To a mixture of the obtained glycosylation adduct (3.1 mg, 3.7 mol) in THF (250 L) at 23° C., was added Et₂NH (20 μL). The mixture was stirred at this temperature for 6 h before it was diluted with EtOAc (2 mL) and quenched by addition of NH₄Cl (sat. aq., 1 mL). The mixture was extracted with EtOAc (2 mL) and the organic phase was dried over anhydrous Na₂SO₄, filtered and concentrated under reduced pressure. The residue was purified by preparative thin layer chromatography (silica gel, EtOAc:Et₃N=10:1, v/v) to give the titled compound as a yellow foam (1.1 mg, 1.8 mol, 49% yield).

Trx34: R_{f}=0.11 (silica gel, EtOAc:Et₃N=10:1, v/v); [α]_{D}²⁵=+42.7 (c=0.11, CHCl₃); FT-IR (film): νₘₐₓ=3311, 2952, 2922, 2852, 1622, 1619, 1570, 1445, 1388, 1260, 1093, 1014, 996, 982 cm⁻¹; ¹H NMR (600 MHz, CDCl₃): δ=14.66 (s, 1H), 7.40 (s, 1H), 5.72 (s, 1H), 5.36 (d, J=4.1 Hz, 1H), 5.22 (d, J=4.1 Hz, 1H), 5.11 (s, 1H), 4.32 (dd, J=11.6, 4.9 Hz, 1H), 4.12 (dd, J=11.5, 5.0 Hz, 1H), 3.94 (td, J=12.0, 2.5 Hz, 1H), 3.86 (s, 1H), 3.83-3.75 (m, 1H), 3.76 (s, 3H), 3.06-2.99 (m, 2H), 2.93 (d, J=6.0 Hz, 1H), 2.82 (d, J=6.0 Hz, 1H), 2.71 (dd, J=7.2, 5.6 Hz, 2H), 2.60 (s, 3H), 2.28-2.17 (m, 1H), 2.07 (dq, J=13.6, 7.1 Hz, 3H), 1.87-1.78 (m, 3H), 1.39 (d, J=13.6 Hz, 1H), 1.23 (d, J=6.1 Hz, 3H) ppm; ¹³C NMR (151 MHz, CDCl₃): δ=204.5, 163.3, 152.2, 142.5, 142.2, 135.4, 130.3, 115.6, 113.37, 113.36, 111.1, 103.1, 101.7, 96.4, 93.0, 72.4, 69.6, 68.7, 67.7, 67.6, 61.0, 53.8, 47.8, 39.0, 30.5, 27.8, 25.8, 23.7, 22.3, 20.8, 18.7, 14.3 ppm.

### (1S,2S,13aS)-2-(1,3-Dioxan-2-yl)-13a-{[(2S,5R,6S)-5-(ethylamino)-6-methyltetrahydro-2H-pyran-2-yl]oxy}-12-hydroxy-7-methoxy-5-methyl-3a,4,8,9,10,13a-hexahydro-11H-spiro[2,4-epoxyfuro[3,2-b]naphtho[2,3-h]chromene-1,2′-oxiran]-11-one (Trx35)

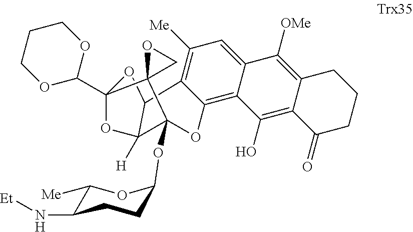

To a mixture of hemiacetal Trx12 (5.0 mg, 10 μmol, 1.0 equiv), glycosyl donor XJ (18.7 mg, 34 μmol, 3.4 equiv) and flame-dried 4 Å molecular sieves (20 mg) in CH₂Cl₂ (1 mL) at −40° C. was added freshly prepared PPh₃AuOTf (0.05 M in CH₂Cl₂, 40 μL, 2.0 μmol, 0.2 equiv). The reaction mixture was stirred at this temperature for 0.5 h before it was quenched by addition of saturated aqueous NaHCO₃ (1 mL). The crude mixture was filtered through a short pad of Celite, extracted with CH₂Cl₂ (2 mL). The combined organic phases were dried over anhydrous Na₂SO₄, filtered and concentrated under reduced pressure. The residue was purified by preparative thin layer chromatography (silica gel, EtOAc) to give the glycosylation product (7.4 mg, 8.6 μmol, 86% yield) as a yellow foam.

To a mixture of the obtained glycosylation adduct (6.3 mg, 7.3 mol) in THF (500 L) at 23° C., was added Et₂NH (50 μL). The mixture was stirred at this temperature for 6 h before it was diluted with EtOAc (2 mL) and quenched by addition of NH₄Cl (sat. aq., 1 mL). The mixture was extracted with EtOAc (2 mL) and the organic phase was dried over anhydrous Na₂SO₄, filtered and concentrated under reduced pressure. The residue was purified by preparative thin layer chromatography (silica gel, EtOAc:Et₃N 10:1, v/v) to give the titled compound as a yellow foam (2.2 mg, 3.9 mol, 53% yield).

Trx35: R_{f}=0.30 (silica gel, EtOAc:Et₃N=10:1, v/v); [α]_{D}²⁵=+68.6 (c=0.22, CHCl₃); FT-IR (film): νₘₐₓ=3374, 2957, 2926, 2853, 1702, 1619, 1570, 1445, 1388, 1235, 1179, 1094, 996, 984 cm⁻¹; ¹H NMR (600 MHz, CDCl₃): δ=14.65 (s, 1H), 7.40 (s, 1H), 5.69 (s, 1H), 5.38 (d, J=4.1 Hz, 1H), 5.21 (d, J=4.1 Hz, 1H), 5.08 (s, 1H), 4.31 (dd, J=11.9, 4.7 Hz, 1H), 4.11 (dd, J=11.5, 4.7 Hz, 1H), 4.01 (dd, J=9.4, 6.2 Hz, 1H), 3.94 (t, J=12.5 Hz, 1H), 3.82-3.76 (m, 1H), 3.76 (s, 3H), 2.91 (d, J=6.1 Hz, 1H), 2.86-2.78 (m, 2H), 2.74-2.68 (m, 2H), 2.65-2.61 (m, 1H), 2.60 (s, 3H), 2.32 (d, J=7.5 Hz, 1H), 2.22 (dt, J=10.2, 5.0 Hz, 1H), 2.12-2.04 (m, 3H), 1.94 (s, 1H), 1.79 (t, J=7.9 Hz, 2H), 1.38 (d, J=13.6 Hz, 1H), 1.29-1.20 (m, 3H), 1.12 (t, J=7.1 Hz, 3H) ppm; ¹³C NMR (151 MHz, CDCl₃): δ=204.5, 163.3, 152.2, 142.5, 142.3, 135.3, 130.2, 115.6, 113.4, 113.4, 111.1, 103.1, 101.7, 96.4, 92.9, 72.3, 69.6, 68.6, 67.7, 67.5, 61.0, 59.6, 54.0, 47.8, 41.5, 39.0, 29.4, 25.8, 24.6, 23.7, 22.3, 20.8, 19.1, 15.9 ppm.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference:
Abul-Hajj, J. Org. Chem., 51:3380-3382, 1986.
Bellamy et al., Cancer Res., 48:6365-6370, 1988.
Chari et al., Angew. Chem. Int. Ed., 53:3796-3827, 2014.
Davies, et al., Org. Lett., 7:5233-5236, 2005.
Davis, et al., J. Am. Chem. Soc., 112:6679-6690, 1990.
Davis and Haque, J. Org. Chem., 51:4083-4085, 1986.
de Sousa, et al., Tetrahedron, 58:4643-4654, 2002.
Dosio et al., Recent Pat. Anti Canc., 9:35-65, 2014.
Evans et al., J. Am. Chem. Soc., 113:7613-7630, 1991.
Fitzner et al., Anal. Bioanal. Chem., 390:1139-1147, 2008.
Fujimoto and Morimoto, J. Antibiot., 36:1216-1221, 1983.
Gaoni, J. Chem. Soc. C, 2925-2934, 1968.
Gerber et al., Nat. Prod. Rep., 30:625-639, 2013.
Grellepois et al., Org. Lett., 7:5219-5222, 2005.
Kende, et al., Tetrahedron, 58:61-74, 2002.
Kleinpeter, E.; Seidl, P. R. J. Phys. Org. Chem., 17: 680-685, 2004.
Kleinpeter et al., J. Phys. Org. Chem. 18:272, 2005.
Li et al., Tetrahedron Lett., 49:3604-3608, 2008.
Li et al., Chem.—Eur J., 16:1871-1882, 2010.
Ma et al., J. Org. Chem., 76:9748, 2011.
Marigo et al., J. Am. Chem. Soc., 127:6964-6965, 2005.
Muller et al., Biochem. Biophys. Res. Commun., 230:254-257, 1997.
Naruse et al., Tetrahedron Lett., 29:1417-1420, 1988.
Nicolaou, et al., J. Am. Chem. Soc., 131:14812-14826, 2009.
Nicolaou et al., Org. Lett., 13:3924-3927, 2011.
Nicolaou, Chem. Biol., 21:1031-1045, 2014.
Nicolaou et al., J. Am. Chem. Soc., 137:8716-8719, 2015a.
Nicolaou et al., Angew. Chem., Int. Ed., 54:3074-3078, 2015b.
Nicolaou et al., J. Am. Chem. Soc., 138:3118-3124, 2016a.
Nicolaou, et al., J. Am. Chem. Soc., 138:7532-7535, 2016b.
Nie et al., J. Am. Chem. Soc., 136:4157-4160, 2014.
Pérez-Arnaiz et al., J. Phys. Chem. B, 118:1288-1295, 2014.
Pfoh et al., Nucleic Acids Res., 36:3508-3514, 2008.
Sapra and Shor, Pharmacol. Ther, 138:452-469, 2013.
Schmid and Garratt, J. Org. Chem., 48:4169, 1983.
Sharpless, et al., J. Am. Chem. Soc., 95:6137-6139, 1973.
Sievers and Senter, Annu. Rev. Med., 64:15-29, 2013.
Tang et al., J. Am. Chem. Soc., 135:18396-18405, 2013.
Tsuda and Hosoi, Chem. Pharm. Bull., 33:1745-1748, 1985.
Tsuda, et al., Chem. Pharm. Bull., 39:1365-1373, 1991.
Wasserman et al., Tetrahedron Lett., 27:4909-4912, 1986.
Yang et al., J. Am. Chem. Soc., 131:12076-12077, 2009.
Zhang et al., Angew. Chem., Int. Ed., 50:4933-4936, 2011.

1. A compound of the formula: 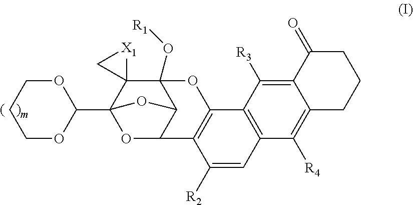
wherein:
m is 0 or 1;
R₁ is alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 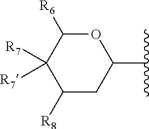
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
X₁ is —O— or —NRₐ—, wherein Rₐ is hydrogen, alkyl_{(C≤8)}, or substituted alkyl_{(C≤8)};
provided that when R₂ is methyl, R₃ is hydroxy, R₄ is methoxy, and X₁ is O, then R₁ is not acetyl, alkyl_{(≤12)}, or substituted alkyl_{(C≤12)};
a compound of the formula: 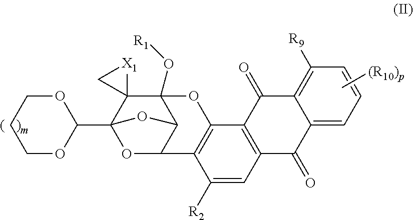
wherein:
m is 0 or 1;
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 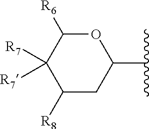
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₉ is hydroxy, alkoxy_{(C≤8)}, substituted alkoxy_{(C≤8)}, acyloxy_{(C≤8)}, or substituted acyloxy_{(C≤8)};
R₁₀ is hydrogen, halo, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)};
p is 1, 2, 3, or 4; and
X₁ is —O— or —NRₐ—, wherein Rₐ is hydrogen, alkyl_{(C≤8)}, or substituted alkyl_{(C≤8)}; or
a compound of the formula: 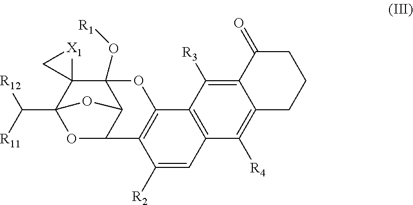
wherein:
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 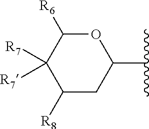
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
R₁₁ is hydrogen, alkyl_{(C≤8)}, substituted alkyl_{(C≤8)}, alkoxy_{(C≤8)}, or substituted alkoxy_{(C≤8)};
R₁₂ is —O(CH₂)_{q}R₁₃, wherein:
q is 1, 2, 3, 4, or 5;
R₁₃ is amino, alkylamino_{(C≤8)}, substituted alkylamino_{(C≤8)}, dialkylamino_{(C≤8)}, or substituted dialkylamino_{(C≤8)}; or
a group of the formula: 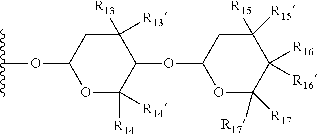
wherein:
R₁₃, R₁₃′, R₁₄, R₁₄′, R₁₅, R₁₅′, R₁₆, R₁₆′, R₁₇, and R₁₇′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)}; and
a group of the formula: 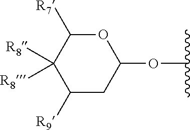
wherein:
R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₈″ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered hetero-cycloalkanediyl_{(C≤12)} or substituted heterocyclo-alkanediyl_{(C≤12)} which is optionally substituted with 1, 2, or 3 oxo groups; R₁ and R₂ are taken together and are alkanediyl_{(C≤8)} or substituted alkanediyl_{(C≤8)};
X₁ is —O— or —NRₐ—, wherein Rₐ is hydrogen, alkyl_{(C≤8)}, or substituted alkyl_{(C≤8)};
a compound of the formula: 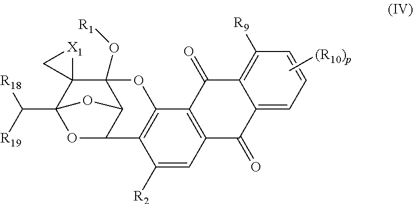
wherein:
R₁ is hydrogen, alkyl_{(C≤8)}, alkenyl_{(C≤8)}, acyl_{(C≤8)}, or a substituted version of the last three groups; or
a group of the formula: 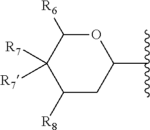
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₉ is hydroxy, alkoxy_{(C≤8)}, substituted alkoxy_{(C≤8)}, acyloxy_{(C≤8)}, or substituted acyloxy_{(C≤8)};
R₁₀ is hydrogen, halo, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)};
p is 1, 2, 3, or 4;
R₁₈ is hydrogen, alkyl_{(C≤8)}, substituted alkyl_{(C≤8)}, alkoxy_{(C≤8)}, or substituted alkoxy_{(C≤8)};
R₁₉ is —O(CH₂)_{q}R₁₃, wherein:
q is 1, 2, 3, 4, or 5;
R₁₃ is amino, alkylamino_{(C≤8)}, substituted alkylamino_{(C≤8)}, dialkylamino_{(C≤8)}, or substituted dialkylamino_{(C≤8)}; or
a group of the formula: 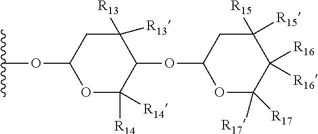
wherein:
R₁₃, R₁₃′, R₁₄, R₁₄′, R₁₅, R₁₅′, R₁₆, R₁₆′, R₁₇, and R₁₇′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)}; and
a group of the formula: 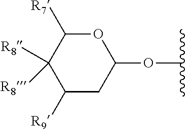
wherein:
R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₈″ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups; R₁ and R₂ are taken together and are alkanediyl_{(C≤8)} or substituted alkanediyl_{(C≤8)};
X₁ is —O— or —NRₐ—, wherein Rₐ is hydrogen, alkyl_{(C≤8)}, or substituted alkyl_{(C≤8)}; or
a compound of the formula: 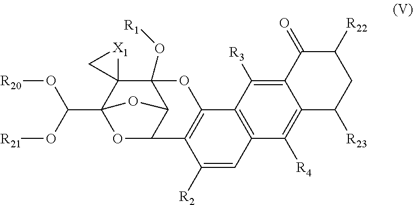
wherein:
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 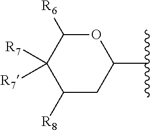
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
R₂₀ is alkyl_{(C≤8)}, substituted alkyl_{(C≤8)}, acyl_{(C≤8)}, or substituted acyl_{(C≤8)};
R₂₁ is alkyl_{(C≤8)} or substituted alkyl_{(C≤8)};
R₂₂ is hydrogen, hydroxy, alkoxy_{(C≤8)}, substituted alkoxy_{(C≤8)}, acyloxy_{(C≤8)}, or substituted acyloxy_{(C≤8)};
R₂₃ is a group of the formula: 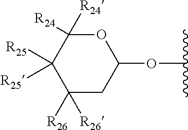
wherein:
R₂₄, R₂₄′, R₂₆, and R₂₆′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₂₅ and R₂₅′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups; and
X₁ is —O— or —NRₐ—, wherein Rₐ is hydrogen, alkyl_{(C≤8)}, or substituted alkyl_{(C≤8)};
or a pharmaceutically acceptable salt thereof.

2. The compound of claim 1 further defined as: 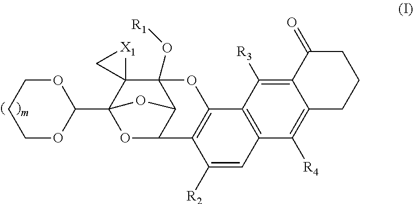
wherein:
m is 0 or 1;
R₁ is alkyl_{(C≤8)}, alkenyl_{(C≤8)}, acyl_{(C≤8)}, or a substituted version of the last three groups; a group of the formula —(CH₂)ₙ_{₁}O(CH₂)₂R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 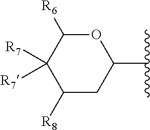
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
X₁ is —O— or —NRₐ—, wherein Rₐ is hydrogen, alkyl_{(C≤8)}, or substituted alkyl_{(C≤8)};
provided that when R₂ is methyl, R₃ is hydroxy, R₄ is methoxy, and X₁ is O, then R₁ is not acetyl, alkyl_{(≤12)}, or substituted alkyl_{(C≤12)};
or a pharmaceutically acceptable salt thereof.

3. The compound of claim 2 further defined as: 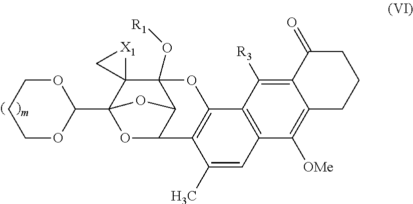
wherein:
m is 0 or 1;
R₁ is alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 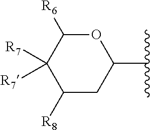
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₃ is hydrogen, hydroxy, alkoxy_{(C≤8)}, substituted alkoxy_{(C≤8)}, acyloxy_{(C≤8)}, or substituted acyloxy_{(C≤8)};
X₁ is —O— or —NRₐ—, wherein Rₐ is hydrogen, alkyl_{(C≤8)}, or substituted alkyl_{(C≤8)};
provided that when R₃ is hydroxy and X₁ is O, then R₁ is not acetyl, alkyl_{(≤12)}, or substituted alkyl_{(C≤12)};
or a pharmaceutically acceptable salt thereof.

4. The compound of claim 2 further defined as: 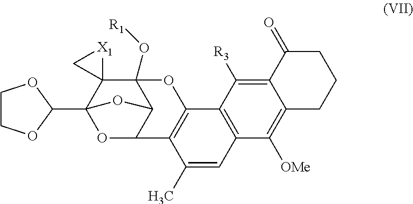
wherein:
R₁ is alkyl_{(C≤8)}, alkenyl_{(C≤8)}, acyl_{(C≤8)}, or a substituted version of the last three groups; a group of the formula —(CH₂)ₙ_{₁}O(CH₂)₂R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 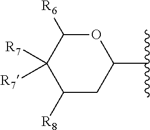
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₃ is hydrogen, hydroxy, alkoxy_{(C≤8)}, substituted alkoxy_{(C≤8)}, acyloxy_{(C≤8)}, or substituted acyloxy_{(C≤8)};
X₁ is —O— or —NRₐ—, wherein Rₐ is hydrogen, alkyl_{(C≤8)}, or substituted alkyl_{(C≤8)};
or a pharmaceutically acceptable salt thereof.

5. The compound of claim 2 further defined as: 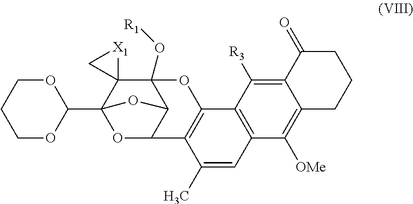
wherein:
R₁ is alkenyl_{(C≤8)} or substituted alkenyl_{(C≤8)}; a group of the formula —(CH₂)ₙ_{₁}O(CH₂)ₙ_{₂}R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 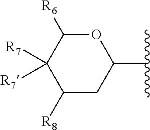
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₃ is hydrogen, hydroxy, alkoxy_{(C≤8)}, substituted alkoxy_{(C≤8)}, acyloxy_{(C≤8)}, or substituted acyloxy_{(C≤8)};
X₁ is —O— or —NRₐ—, wherein Rₐ is hydrogen, alkyl_{(C≤8)}, or substituted alkyl_{(C≤8)};
provided that when R₃ is hydroxy and X₁ is O, then R₁ is not acetyl, alkyl(12), or substituted alkyl_{(C≤12)};
or a pharmaceutically acceptable salt thereof.

6. The compound of claim 1 further defined as: 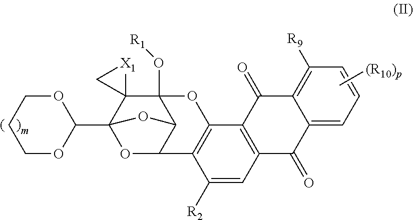
wherein:
m is 0 or 1;
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 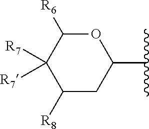
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₉ is hydroxy, alkoxy_{(C≤8)}, substituted alkoxy_{(C≤8)}, acyloxy_{(C≤8)}, or substituted acyloxy_{(C≤8)};
R₁₀ is hydrogen, halo, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)};
p is 1, 2, 3, or 4; and
X₁ is —O— or —NRₐ—, wherein Rₐ is hydrogen, alkyl_{(C≤8)}, or substituted alkyl_{(C≤8)};
or a pharmaceutically acceptable salt thereof.

7. The compound of claim 6 further defined as: 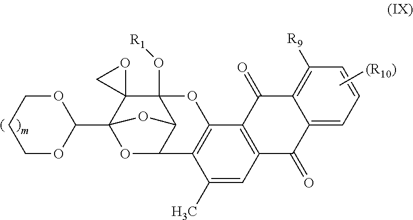
wherein:
m is 0 or 1;
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 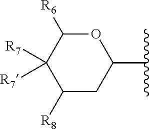
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₉ is hydroxy, alkoxy_{(C≤8)}, substituted alkoxy_{(C≤8)}, acyloxy_{(C≤8)}, or substituted acyloxy_{(C≤8)};
R₁₀ is hydrogen, halo, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)};
p is 1, 2, 3, or 4; and
or a pharmaceutically acceptable salt thereof.

8. The compound of claim 1 further defined as: 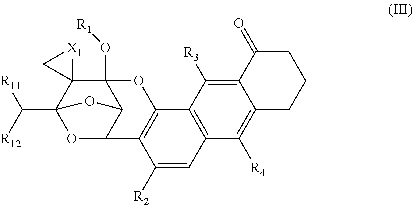
wherein:
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 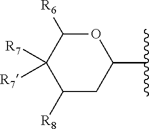
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
R₁₁ is hydrogen, alkyl_{(C≤8)}, substituted alkyl_{(C≤8)}, alkoxy_{(C≤8)}, or substituted alkoxy_{(C≤8)};
R₁₂ is —O(CH₂)_{q}R₁₃, wherein:
q is 1, 2, 3, 4, or 5;
R₁₃ is amino, alkylamino_{(C≤8)}, substituted alkylamino_{(C≤8)}, dialkylamino_{(C≤8)}, or substituted dialkylamino_{(C≤8)}; or
a group of the formula: 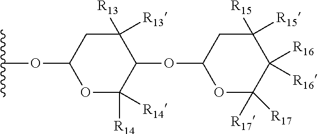
wherein:
R₁₃, R₁₃′, R₁₄, R₁₄′, R₁₅, R₁₅′, R₁₆, R₁₆′, R₁₇, and R₁₇′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)};
a group of the formula: 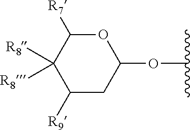
wherein:
R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₈″ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups; R₁ and R₂ are taken together and are alkanediyl_{(C≤8)} or substituted alkanediyl_{(C≤8)}; and
X₁ is —O— or —NRₐ—, wherein Rₐ is hydrogen, alkyl_{(C≤8)}, or substituted alkyl_{(C≤8)};
or a pharmaceutically acceptable salt thereof.

9. The compound of claim 8 further defined as: 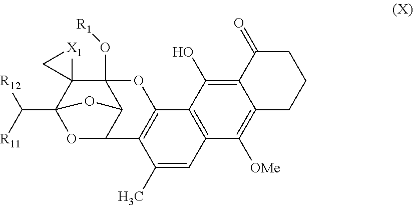
wherein:
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 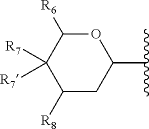
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₁₁ is hydrogen, alkyl_{(C≤8)}, substituted alkyl_{(C≤8)}, alkoxy_{(C≤8)}, or substituted alkoxy_{(C≤8)};
R₁₂ is —O(CH₂)_{q}R₁₃, wherein:
q is 1, 2, 3, 4, or 5;
R₁₃ is amino, alkylamino_{(C≤8)}, substituted alkylamino_{(C≤8)}, dialkylamino_{(C≤8)}, or substituted dialkylamino_{(C≤8)}; or
a group of the formula: 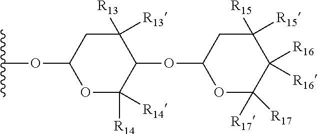
wherein:
R₁₃, R₁₃′, R₁₄, R₁₄′, R₁₅, R₁₅′, R₁₆, R₁₆′, R₁₇, and R₁₇′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)};
a group of the formula: 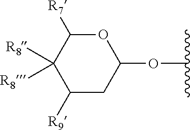
wherein:
R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₈′ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups; R₁ and R₂ are taken together and are alkanediyl_{(C≤8)} or substituted alkanediyl_{(C≤8)}; and
X₁ is —O— or —NRₐ—, wherein Rₐ is hydrogen, alkyl_{(C≤8)}, or substituted alkyl_{(C≤8)};
or a pharmaceutically acceptable salt thereof.

10. The compound of claim 1 further defined as: 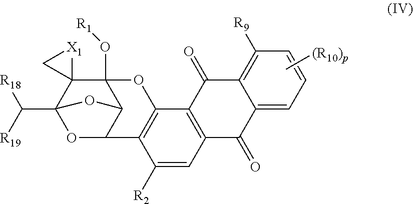
wherein:
R₁ is hydrogen, alkyl_{(C≤8)}, alkenyl_{(C≤8)}, acyl_{(C≤8)}, or a substituted version of the last three groups; or
a group of the formula: 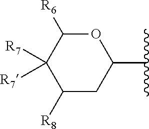
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₉ is hydroxy, alkoxy_{(C≤8)}, substituted alkoxy_{(C≤8)}, acyloxy_{(C≤8)}, or substituted acyloxy_{(C≤8)};
R₁₀ is hydrogen, halo, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)};
p is 1, 2, 3, or 4;
R₁₈ is hydrogen, alkyl_{(C≤8)}, substituted alkyl_{(C≤8)}, alkoxy_{(C≤8)}, or substituted alkoxy_{(C≤8)};
R₁₉ is —O(CH₂)_{q}R₁₃, wherein:
q is 1, 2, 3, 4, or 5;
R₁₃ is amino, alkylamino_{(C≤8)}, substituted alkylamino_{(C≤8)}, dialkylamino_{(C≤8)}, or substituted dialkylamino_{(C≤8)}; or
a group of the formula: 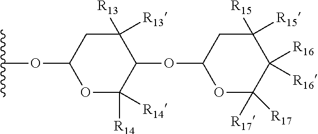
wherein:
R₁₃, R₁₃′, R₁₄, R₁₄′, R₁₅, R₁₅′, R₁₆, R₁₆′, R₁₇, and R₁₇′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)};
a group of the formula: 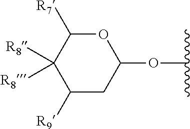
wherein:
R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₈′ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups; R₁ and R₂ are taken together and are alkanediyl_{(C≤8)} or substituted alkanediyl_{(C≤8)}; and
X₁ is —O— or —NRₐ—, wherein Rₐ is hydrogen, alkyl_{(C≤8)}, or substituted alkyl_{(C≤8)};
or a pharmaceutically acceptable salt thereof.

11. (canceled)

12. The compound of claim 1 further defined as: 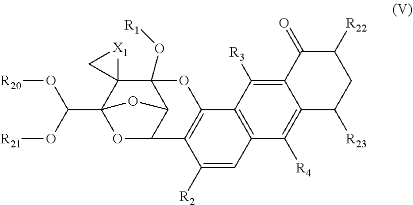
wherein:
R₁ is hydrogen, alkyl_{(C≤8)}, alkenyl_{(C≤8)}, acyl_{(C≤8)}, or a substituted version of the last three groups; a group of the formula —(CH₂)ₙ_{₁}O(CH₂)ₙ_{₂}R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}; or
a group of the formula: 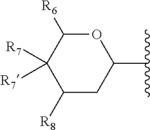
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups;
R₂ is alkyl_{(C≤8)} or substituted alkyl_{(C≤8)};
R₃ is hydrogen, hydroxy, alkoxy_{(C≤8)}, substituted alkoxy_{(C≤8)}, acyloxy_{(C≤8)}, or substituted acyloxy_{(C≤8)};
R₄ is hydroxy, alkoxy_{(C≤8)}, substituted alkoxy_{(C≤8)}, acyloxy_{(C≤8)}, or substituted acyloxy_{(C≤8)};
R₂₀ is alkyl_{(C≤8)}, substituted alkyl_{(C≤8)}, acyl_{(C≤8)}, or substituted acyl_{(C≤8)};
R₂₁ is alkyl_{(C≤8)} or substituted alkyl_{(C≤8)};
R₂₂ is hydrogen, hydroxy, alkoxy_{(C≤8)}, substituted alkoxy_{(C≤8)}, acyloxy_{(C≤8)}, or substituted acyloxy_{(C≤8)};
R₂₃ is a group of the formula: 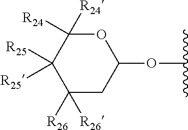
wherein:
R₂₄, R₂₄′, R₂₆, and R₂₆′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₂₅ and R₂₅′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups; and
X₁ is —O— or —NRₐ—, wherein Rₐ is hydrogen, alkyl_{(C≤8)}, or substituted alkyl_{(C≤8)};
or a pharmaceutically acceptable salt thereof.

13.-19. (canceled)

20. The compound of claim 1,
wherein R₁ is hydrogen, alkyl_{(C≤8)}, acyl_{(C≤8)}, alkenyl_{(C≤8)}, or R₁ is a group of the formula —(CH₂)ₙ_{₁}O(CH₂)ₙ_{₂}R₅, wherein:
n₁ and n₂ are each independently 1, 2, 3, 4, or 5; and
R₅ are amino, alkylamino_{(C≤8)}, substituted alkylamino_{(C≤8)}, dialkylamino_{(C≤12)}, or substituted dialkylamino_{(C≤12)}, or
R₁ is a group of the formula: 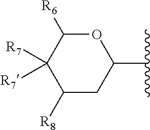
wherein:
R₆ and R₈ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₇ and R₇′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}, or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups.

21.-22. (canceled)

23. The compound of claim 1, wherein the compound is further defined as: 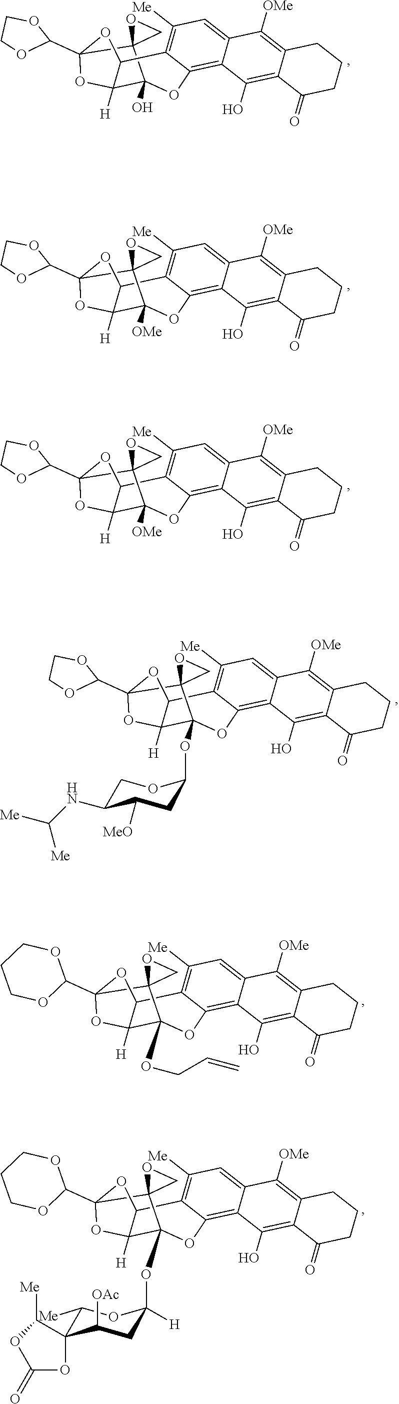 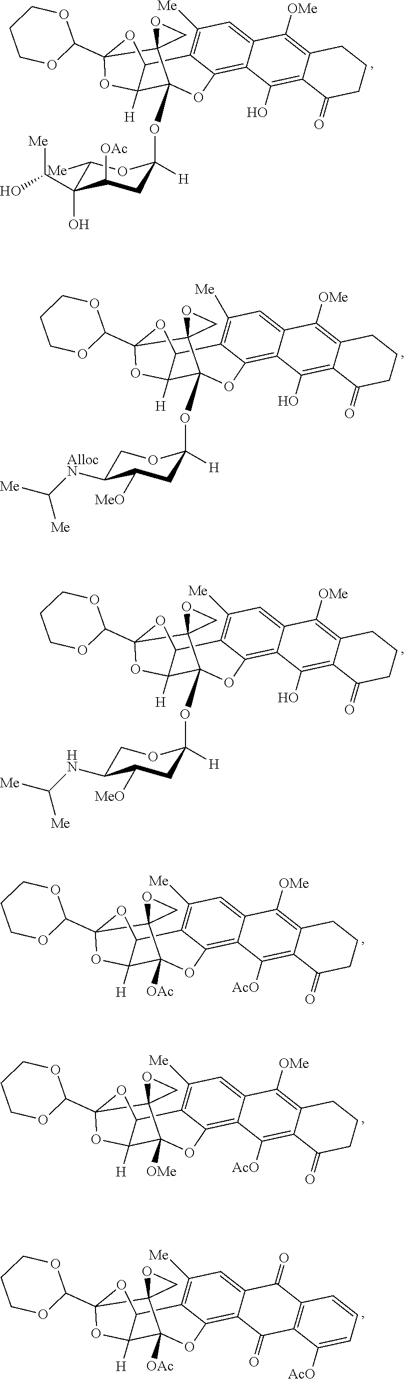 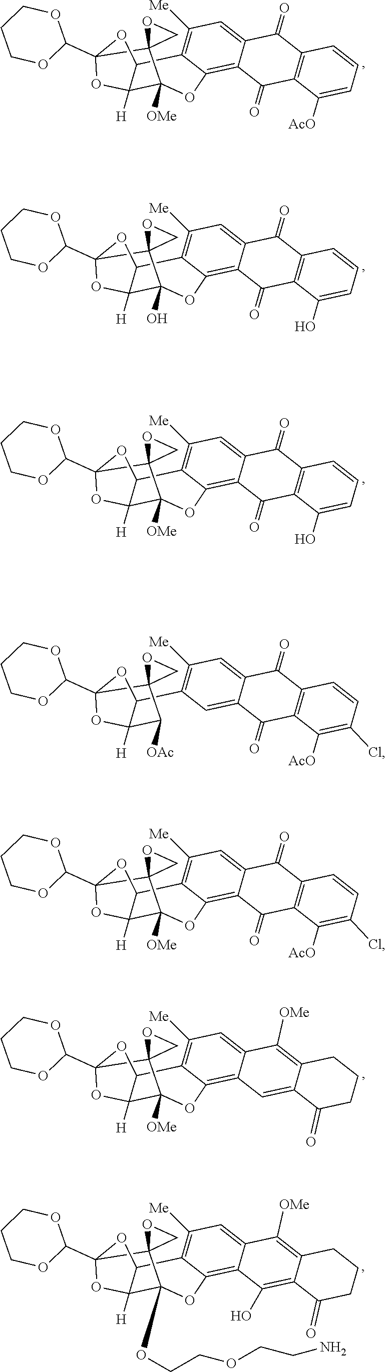 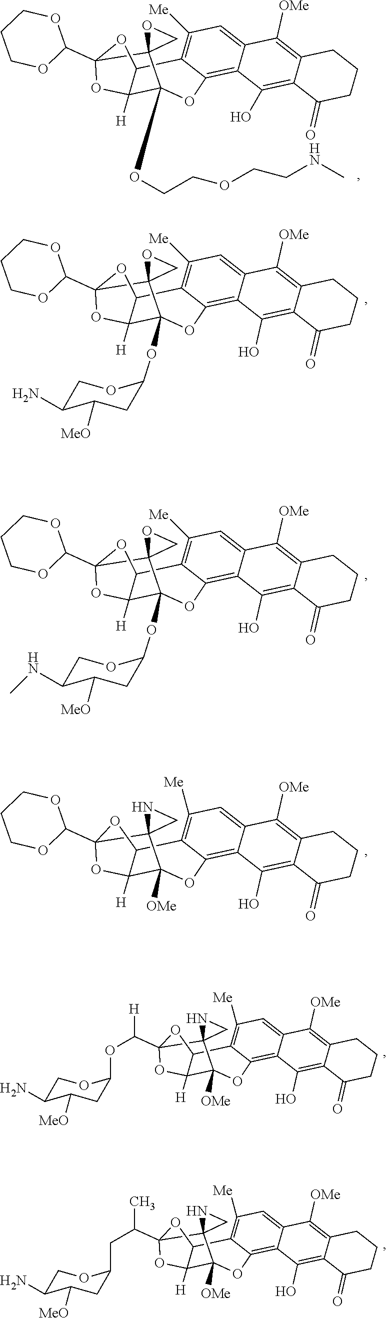 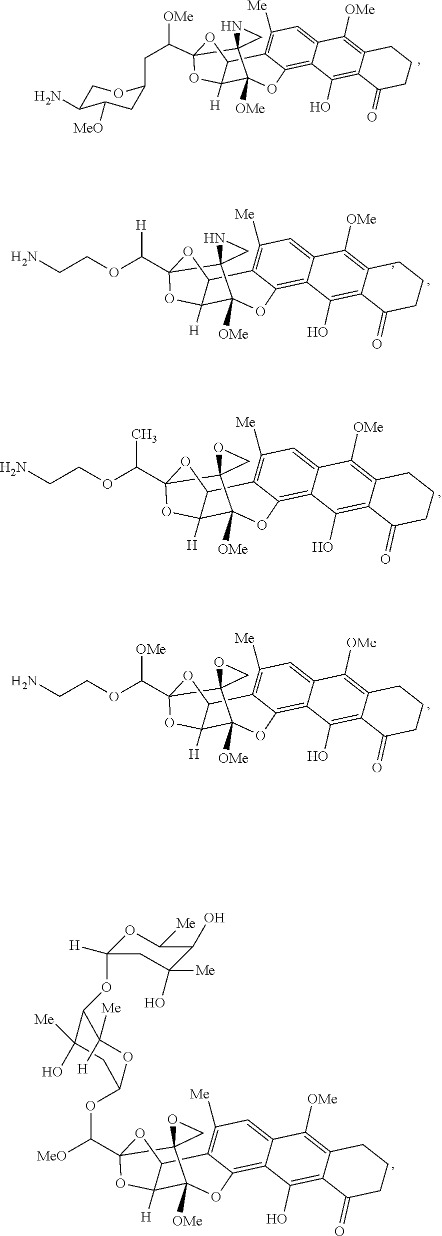 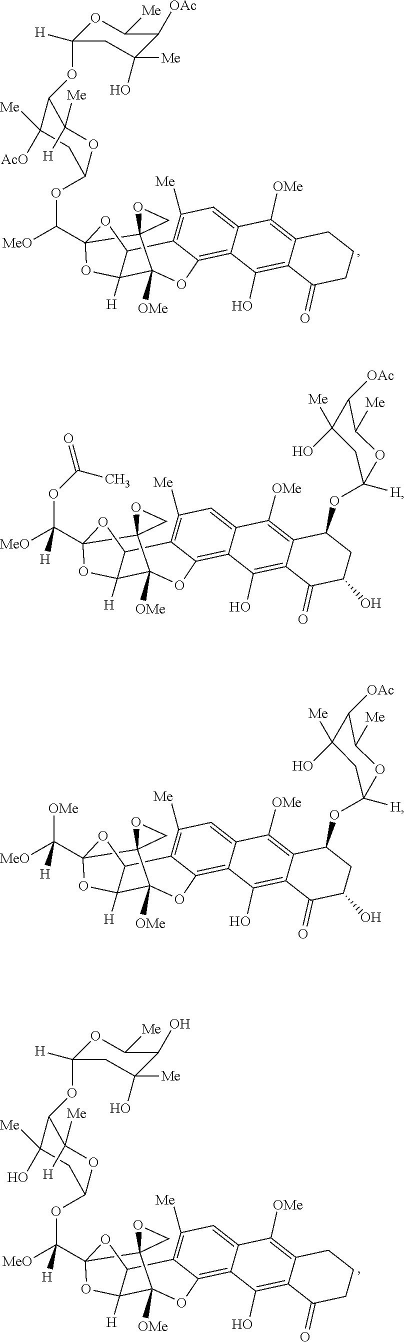 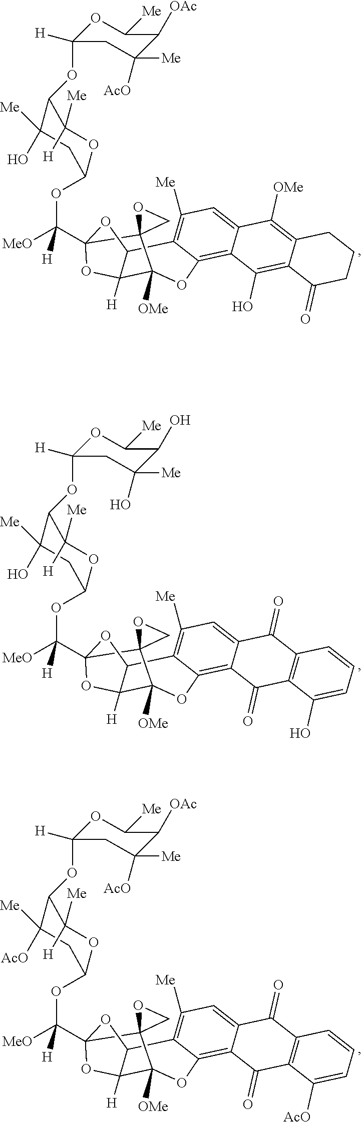 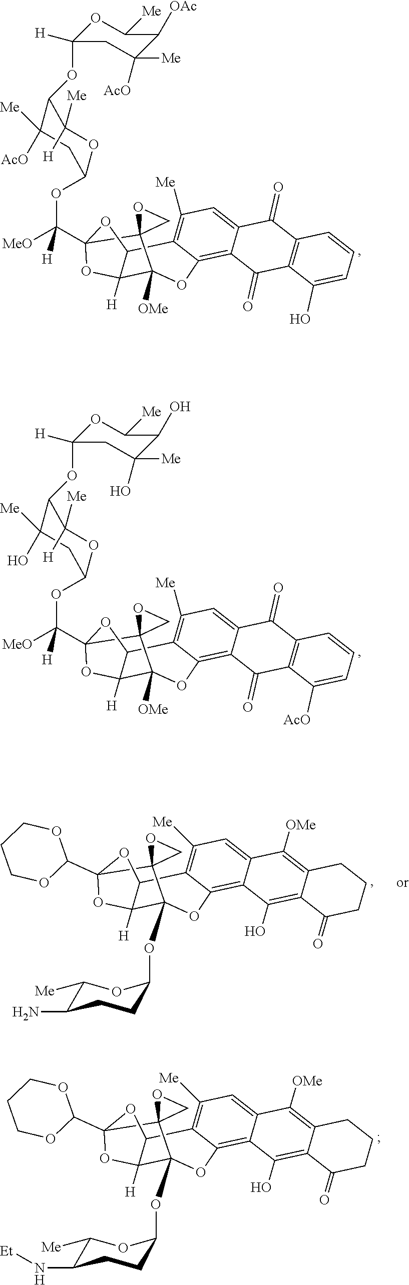
or a pharmaceutically acceptable salt thereof.

24. (canceled)

25. A dimer of the formula:
Y₁-L-Y₁′ (XIII)
wherein:
Y₁ and Y₁′ are each independently a compound of the formula: 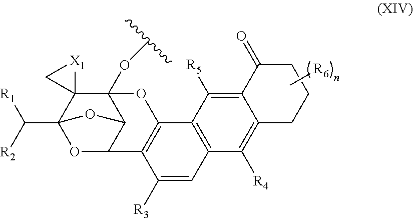
wherein:
R₁ and R₂ are each independently hydrogen, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; a group of the formula: —O(CH₂)_{q}R₁₃, wherein:
q is 1, 2, 3, 4, or 5;
R₁₃ is amino, alkylamino_{(C≤8)}, substituted alkylamino_{(C≤8)}, dialkylamino_{(C≤8)}, or substituted dialkylamino_{(C≤8)}; or
a group of the formula: 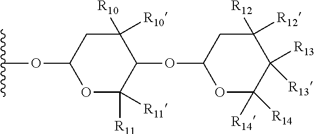
wherein:
R₁₀, R₁₀′, R₁₁, R₁₁′, R₁₂, R₁₂′, R₁₃, R₁₃′, R₁₄, and R₁₄′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)}; or
a group of the formula: 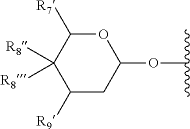
wherein:
R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₈′ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted heterocycloalkanediyl_{(C≤12)} which is optionally substituted with 1, 2, or 3 oxo groups; R₁ and R₂ are taken together and are alkanediyl_{(C≤8)} or substituted alkanediyl_{(C≤8)};
R₃ is alkyl_{(C≤8)} or substituted alkyl_{(C≤8)};
R₄ is hydrogen, hydroxy, alkoxy_{(C≤8)}, substituted alkoxy_{(C≤8)}, acyloxy_{(C≤8)}, or substituted acyloxy_{(C≤8)};
R₅ is hydroxy, alkoxy_{(C≤8)}, substituted alkoxy_{(C≤8)}, acyloxy_{(C≤8)}, or substituted acyloxy_{(C≤8)};
R₆ is hydrogen, hydroxy, alkoxy_{(C≤8)}, substituted alkoxy_{(C≤8)}, acyloxy_{(C≤8)}, or substituted acyloxy_{(C≤8)}, or a group of the formula: 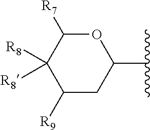
wherein:
R₇ and R₉ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₈ and R₈′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted hetero-cycloalkanediyl_{(C≤12)} which is optionally substituted with 1, 2, or 3 oxo groups;
n is 1, 2, 3, 4, 5 or 6; and
X₁ is —O— or —NRₐ—, wherein Rₐ is hydrogen, alkyl_{(C≤8)}, or substituted alkyl_{(C≤8)}; or
a compound of the formula: 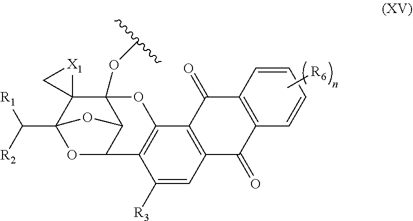
wherein:
R₁ and R₂ are each independently hydrogen, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; a group of the formula: —O(CH₂)_{q}R₁₃, wherein:
q is 1, 2, 3, 4, or 5;
R₁₃ is amino, alkylamino_{(C≤8)}, substituted alkylamino_{(C≤8)}, dialkylamino_{(C≤8)}, or substituted dialkylamino_{(C≤8)}; or
a group of the formula: 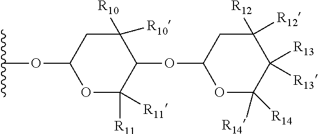
wherein:
R₁₀, R₁₀′, R₁₁, R₁₁′, R₁₂, R₁₂′, R₁₃, R₁₃′, R₁₄, and R₁₄′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, and substituted acyloxy_{(C≤8)};
a group of the formula: 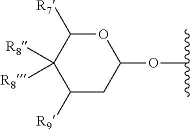
wherein:
R₇′ and R₉′ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₈′ and R₈′″ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted hetero-cycloalkanediyl_{(C≤12)} which is optionally substituted with 1, 2, or 3 oxo groups; R₁ and R₂ are taken together and are alkanediyl_{(C≤8)} or substituted alkanediyl_{(C≤8)};
R₁ and R₂ are taken together and are alkoxydiyl_{(C≤8)} or substituted alkoxydiyl_{(C≤8)};
R₃ is alkyl_{(C≤8)} or substituted alkyl_{(C≤8)};
R₄ is hydrogen, hydroxy, alkoxy_{(C≤8)}, substituted alkoxy_{(C≤8)}, acyloxy_{(C≤8)}, or substituted acyloxy_{(C≤8)};
R₅ is hydroxy, alkoxy_{(C≤8)}, substituted alkoxy_{(C≤8)}, acyloxy_{(C≤8)}, or substituted acyloxy_{(C≤8)};
R₆ is hydrogen, hydroxy, alkoxy_{(C≤8)}, substituted alkoxy_{(C≤8)}, acyloxy_{(C≤8)}, or substituted acyloxy_{(C≤8)}, or a group of the formula: 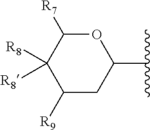
wherein:
R₇ and R₉ are each independently hydrogen, hydroxy, alkyl_{(C≤8)}, substituted alkyl_{(C≤8)}, alkoxy_{(C≤8)}, substituted alkoxy_{(C≤8)}, acyloxy_{(C≤8)}, or substituted acyloxy_{(C≤8)}; and
R₈ and R₈′ are hydrogen, hydroxy, alkyl_{(C≤8)}, substituted alkyl_{(C≤8)}, alkoxy_{(C≤8)}, substituted alkoxy_{(C≤8)}, acyl_{(C≤8)}, or substituted acyl_{(C≤8)}; or a group of the formula: —NR_{b}R_{c}, wherein R_{b} and R_{c} are each independently hydrogen, monovalent amine protecting group, alkyl_{(C≤8)}, or substituted alkyl_{(C≤8)}, or R_{b} and R_{c} are taken together and are a divalent amine protecting group; or R₇ and R₇′ are taken together and are a 4 to 10 membered heterocycloalkanediyl_{(C≤12)} or substituted hetero-cycloalkanediyl_{(C≤12)} which is optionally substituted with 1, 2, or 3 oxo groups; and
n is 1, 2, 3, or 4; and
X₁ is —O— or —NRₐ—, wherein Rₐ is hydrogen, alkyl_{(C≤8)}, or substituted alkyl_{(C≤8)}; and
L is alkanediyl_{(C≤12)}, alkenediyl_{(C≤12)}, alkynediyl_{(C≤12)}, arenediyl_{(C≤12)}, heteroarenediyl_{(C≤12)}, heterocycloalkanediyl_{(C≤12)}, alkoxydiyl_{(C≤12)}, alkylaminodiyl_{(C≤12)}, or a substituted version of any of these groups; or a linker comprising an amino acid chain containing from 1 to 20 amino acids;
or a pharmaceutically acceptable salt thereof.

26.-28. (canceled)

29. The compound of claim 25, wherein the compound is further defined as: 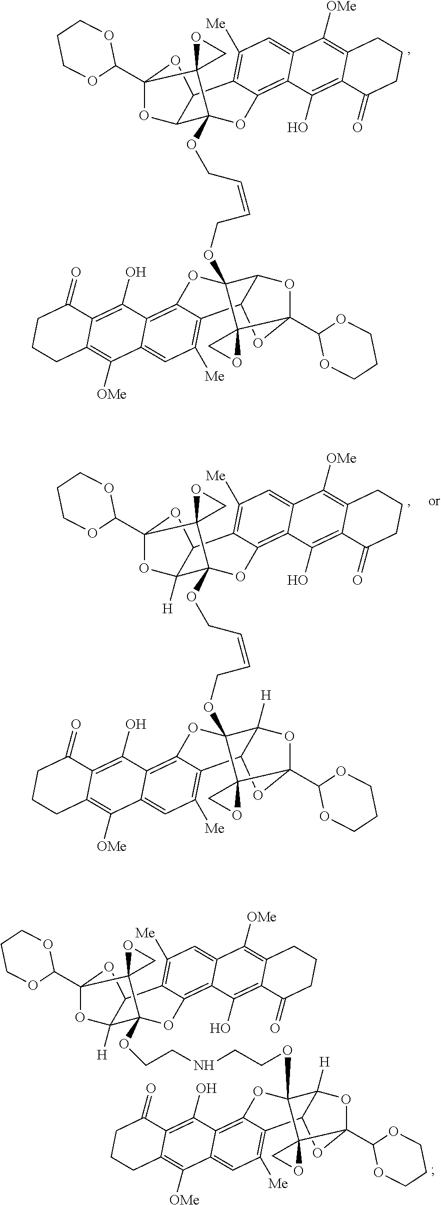
or a pharmaceutically acceptable salt thereof.

30. A pharmaceutical composition comprising a compound of claim 1 and a pharmaceutically acceptable carrier.

31. A method of treating a disease or disorder in a patient in need thereof comprising administering to the patient a pharmaceutically effective amount of a compound or composition of claim 1.

32. (canceled)

33. A conjugate of the formula:
(A-L)ₙ-X (XVI)
wherein:
A is a compound or dimer of claim 1;
L is a covalent bond or a linker;
n is 1, 2, 3, 4, 5, 6, 7, 8, 9, 10, 11, or 12; and
X is a cell targeting moiety.

34. A method of preparing a compound of the formula: 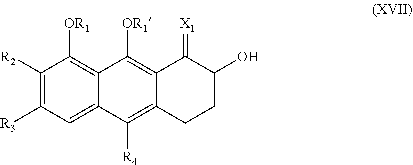
wherein:
R₁ and R₁′ are each independently hydroxy protecting groups or are taken together and are —SiR_{b}R_{c}—, wherein R_{b} and R_{c} are each independently alkyl_{(C≤8)} or substituted alkyl_{(C≤8)};
R₂ is hydrogen or halo;
R₃ is alkyl_{(C≤8)} or substituted alkyl_{(C≤8)};
R₄ is hydroxy, alkoxy_{(C≤8)}, or substituted alkoxy_{(C≤8)}; and
X₁ is O, S, or NR_{c}, wherein R_{c} is hydrogen, alkyl_{(C≤8)}, or substituted alkyl_{(C≤8)};
comprising reacting an oxaziridine reagent and a compound of the formula: 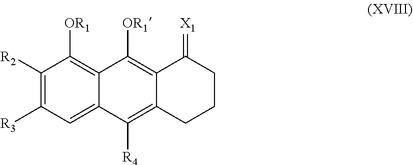
wherein:
R₁ and R₁′ are each independently hydroxy protecting groups or are taken together and are —SiR_{b}R_{c}—, wherein R_{b} and R_{c} are each independently alkyl_{(C≤8)} or substituted alkyl_{(C≤8)};
R₂ is hydrogen or halo;
R₃ is alkyl_{(C≤8)} or substituted alkyl_{(C≤8)};
R₄ is hydroxy, alkoxy_{(C≤8)}, or substituted alkoxy_{(C≤8)}; and
X₁ is O, S, or NR_{c}, wherein R_{c} is hydrogen, alkyl_{(C≤8)}, or substituted alkyl_{(C≤8)};
in the presence of a base under conditions sufficient to achieve a reaction.

35. The method of claim 34, wherein the oxaziridine reagent is further defined by the formula: 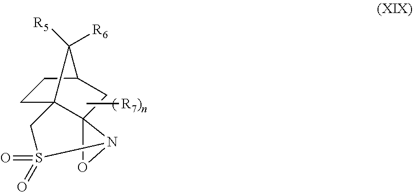
wherein:
R₅ and R₆ are each independently alkyl_{(C≤8)} or substituted alkyl_{(C≤8)};
R₇ is hydrogen, halo, alkyl_{(C≤8)}, or substituted alkyl_{(C≤8)}; and
n is 1, 2, 3, 4, or 5.
